SEQUENCE LISTING (28 sequences)

SEQ ID NO: 1 - DNA, 1521 bp, synthetic construct
  misc_feature 1..1521: note = Engineered hARSA coding sequence
  source 1..1521: mol_type = other DNA
  CDS 1..1521: note = Engineered hARSA coding sequence
atgggagcccctagatctctgctgctggctctggctgctggactggcagttgccagacctcctaacatcgtgctgatcttcgccgacgatctcggctacggcgatctgggctgttacggacaccccagcagcaccacacctaacctggatcaacttgccgctggcggcctgagattcaccgatttctacgtgcccgtgtctctgtgcaccccttctagagctgctctgctgacaggcagactccctgtgcggatgggaatgtatcctggcgtgctggtgcctagctctagaggcggactgcctctggaagaagtgacagttgccgaagtgctggccgccagaggatatctgactggcatggccggaaagtggcacctcggagttggaccagaaggcgcttttctgcctcctcaccagggcttccaccggtttctgggcatcccttactctcacgatcagggcccctgccagaacctgacctgttttcctcctgccacaccttgcgacggcggctgtgatcaaggactggtgccaattcctctgctggccaacctgagcgtggaagctcaacctccttggctgccaggactggaagcccggtatatggccttcgctcacgacctgatggccgacgctcagagacaggacagaccattcttcctgtactacgccagccaccacacacactaccctcagtttagcggccagagcttcgccgagagatctggcagaggacctttcggcgacagcctgatggaactggatgccgctgtgggcacactgatgacagccatcggagatctgggactgctggaagagacactggtcatcttcaccgccgacaacggccccgagacaatgagaatgagcagaggcggctgtagcggcctgctgagatgtggcaagggcaccacatatgaaggcggcgtgagagaacctgctctggccttttggcctggccatattgctccaggcgtgacacacgagctggcctcttctctggatctgctgcctacactggcagctcttgctggtgctcccctgcctaatgtgaccctggatggcttcgatctgagcccactgctgctcggcacaggcaagtctccaagacagagcctgttcttctaccctagctaccccgacgaagtgcggggagtgtttgccgtgcggaccggaaagtataaggcccacttcttcacccaaggcagcgcccactctgacaccacagctgatcctgcttgtcacgccagctctagcctgacagcccatgaacctccactgctgtacgacctgagcaaggaccccggcgagaactacaatctgcttggcggagttgccggcgctacacctgaagttctgcaggccctgaaacagctccagctgctgaaagcccagctggacgctgccgtgacatttggacctagtcaggtggccagaggcgaggatcctgctctgcagatctgttgtcaccctggctgcacacccagacctgcctgctgtcattgtcctgatccacacgcc

SEQ ID NO: 2 - protein, 507 aa, synthetic construct
  REGION 1..507: note = Synthetic Construct
MGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 3 - DNA, 1527 bp, synthetic construct
  misc_feature 1..1527: note = Engineered hARSA coding sequence
  source 1..1527: mol_type = other DNA
  CDS 1..1527: note = Engineered hARSA coding sequence
atgtctatgggagcccctagatctctgctgctggctctggctgctggactggcagttgccagacctcctaacatcgtgctgatcttcgccgacgatctcggctacggcgatctgggctgttacggacaccccagcagcaccacacctaacctggatcaacttgccgctggcggcctgagattcaccgatttctacgtgcccgtgtctctgtgcaccccttctagagctgctctgctgacaggcagactccctgtgcggatgggaatgtatcctggcgtgctggtgcctagctctagaggcggactgcctctggaagaagtgacagttgccgaagtgctggccgccagaggatatctgactggcatggccggaaagtggcacctcggagttggaccagaaggcgcttttctgcctcctcaccagggcttccaccggtttctgggcatcccttactctcacgatcagggcccctgccagaacctgacctgttttcctcctgccacaccttgcgacggcggctgtgatcaaggactggtgccaattcctctgctggccaacctgagcgtggaagctcaacctccttggctgccaggactggaagcccggtatatggccttcgctcacgacctgatggccgacgctcagagacaggacagaccattcttcctgtactacgccagccaccacacacactaccctcagtttagcggccagagcttcgccgagagatctggcagaggacctttcggcgacagcctgatggaactggatgccgctgtgggcacactgatgacagccatcggagatctgggactgctggaagagacactggtcatcttcaccgccgacaacggccccgagacaatgagaatgagcagaggcggctgtagcggcctgctgagatgtggcaagggcaccacatatgaaggcggcgtgagagaacctgctctggccttttggcctggccatattgctccaggcgtgacacacgagctggcctcttctctggatctgctgcctacactggcagctcttgctggtgctcccctgcctaatgtgaccctggatggcttcgatctgagcccactgctgctcggcacaggcaagtctccaagacagagcctgttcttctaccctagctaccccgacgaagtgcggggagtgtttgccgtgcggaccggaaagtataaggcccacttcttcacccaaggcagcgcccactctgacaccacagctgatcctgcttgtcacgccagctctagcctgacagcccatgaacctccactgctgtacgacctgagcaaggaccccggcgagaactacaatctgcttggcggagttgccggcgctacacctgaagttctgcaggccctgaaacagctccagctgctgaaagcccagctggacgctgccgtgacatttggacctagtcaggtggccagaggcgaggatcctgctctgcagatctgttgtcaccctggctgcacacccagacctgcctgctgtcattgtcctgatccacacgcc

SEQ ID NO: 4 - protein, 509 aa, synthetic construct
  REGION 1..509: note = Synthetic Construct
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 5 - DNA, 7141 bp, synthetic construct
  misc_feature 1..7141: note = Production plasmid for AAV. CB7.CI.hARSAco.RBG
  repeat_region 1..130: note = 5' ITR
  regulatory 198..579: note = promoter - CMV IE promoter; regulatory_class = promoter
  regulatory 582..862: note = promoter - CB promoter; regulatory_class = promoter
  regulatory 836..839: note = TATA_signal - TATA; regulatory_class = TATA_box
  misc_feature 956..1928: note = Intron - chicken beta-actin intron
  misc_feature 1962..3488: note = Engineered ARSA coding sequence (hARSAco)
  regulatory 3539..3665: note = polyA_signal - Rabbit globin poly A; regulatory_class = polyA_signal_sequence
  repeat_region 3754..3883: note = 3' ITR
  rep_origin 4060..4498: note = f1 ori
  rep_origin 4527..5169: note = pUC origin of replication
  misc_feature 5844..6649: note = Kan-r
  source 1..7141: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgaattcggtaccacaggccaccatgtctatgggagcccctagatctctgctgctggctctggctgctggactggcagttgccagacctcctaacatcgtgctgatcttcgccgacgatctcggctacggcgatctgggctgttacggacaccccagcagcaccacacctaacctggatcaacttgccgctggcggcctgagattcaccgatttctacgtgcccgtgtctctgtgcaccccttctagagctgctctgctgacaggcagactccctgtgcggatgggaatgtatcctggcgtgctggtgcctagctctagaggcggactgcctctggaagaagtgacagttgccgaagtgctggccgccagaggatatctgactggcatggccggaaagtggcacctcggagttggaccagaaggcgcttttctgcctcctcaccagggcttccaccggtttctgggcatcccttactctcacgatcagggcccctgccagaacctgacctgttttcctcctgccacaccttgcgacggcggctgtgatcaaggactggtgccaattcctctgctggccaacctgagcgtggaagctcaacctccttggctgccaggactggaagcccggtatatggccttcgctcacgacctgatggccgacgctcagagacaggacagaccattcttcctgtactacgccagccaccacacacactaccctcagtttagcggccagagcttcgccgagagatctggcagaggacctttcggcgacagcctgatggaactggatgccgctgtgggcacactgatgacagccatcggagatctgggactgctggaagagacactggtcatcttcaccgccgacaacggccccgagacaatgagaatgagcagaggcggctgtagcggcctgctgagatgtggcaagggcaccacatatgaaggcggcgtgagagaacctgctctggccttttggcctggccatattgctccaggcgtgacacacgagctggcctcttctctggatctgctgcctacactggcagctcttgctggtgctcccctgcctaatgtgaccctggatggcttcgatctgagcccactgctgctcggcacaggcaagtctccaagacagagcctgttcttctaccctagctaccccgacgaagtgcggggagtgtttgccgtgcggaccggaaagtataaggcccacttcttcacccaaggcagcgcccactctgacaccacagctgatcctgcttgtcacgccagctctagcctgacagcccatgaacctccactgctgtacgacctgagcaaggaccccggcgagaactacaatctgcttggcggagttgccggcgctacacctgaagttctgcaggccctgaaacagctccagctgctgaaagcccagctggacgctgccgtgacatttggacctagtcaggtggccagaggcgaggatcctgctctgcagatctgttgtcaccctggctgcacacccagacctgcctgctgtcattgtcctgatccacacgcctgatgaacagcctgaggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacaccatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 6 - DNA, 2211 bp, synthetic construct
  misc_feature 1..2211: note = AAVhu68 vp1
  source 1..2211: mol_type = other DNA
  CDS 1..2211
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 7 - protein, 736 aa, synthetic construct
  REGION 1..736: note = Synthetic Construct
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, synthetic construct
  REGION 1..736: note = modified hu68vp1
  VARIANT 23: note = Xaa may be W (Trp, tryptophan), or oxidated W.
  VARIANT 35: note = Xaa may be Asn, or deamidated to Asp, isoAsp, or Asp/isoAsp
  VARIANT 57: note = Xaa may be Asn, or deamidated to Asp, isoAsp, or Asp/isoAsp
  VARIANT 66: note = Xaa may be Asn, or deamidated to Asp, isoAsp, or Asp/isoAsp
  VARIANT 94: note = Xaa may be Asn, or deamidated to Asp, isoAsp, or Asp/isoAsp
  VARIANT 97: note = Xaa may be D (asp, aspartic acid), or isomerized D
  VARIANT 107: note = Xaa may be D (asp, aspartic acid), or isomerized D
  VARIANT 113: note = Xaa can be any naturally occurring amino acid
  VARIANT 149: note = Xaa may be S (Ser, serine), or Phosphorilated S
  VARIANT 149: note = Xaa may be S (Ser, serine), or Phosphorylated S
  VARIANT 247: note = Xaa may be W (Trp, tryptophan), or oxidated W (e.g., kynurenine).
  VARIANT 253: note = Xaa may be Asn, or deamidated to Asp, isoAsp, or Asp/isoAsp
  VARIANT 259: note = Xaa represents Q, or Q deamidated to glutamic acid (alpha-glutamic acid), gamma-glutamic acid (Glu), or a blend of alpha- and gamma-glutamic acid
  VARIANT 270: note = Xaa may be Asn, or deamidated to Asp, isoAsp, or Asp/isoAsp
  VARIANT 297: note = Xaa represents D (Asp, aspartic acid) or amindated D to N (Asn, asparagine)
  VARIANT 304: note = Xaa may be Asn, or deamidated to Asp, isoAsp, or Asp/isoAsp
  VARIANT 306: note = Qualifier ValueMISC_FEATURE - Xaa may be W (Trp, tryptophan), or oxidated W (e.g., kynurenine)
  VARIANT 314: note = Xaa may be Asn, or deamidated to Asp, isoAsp, or Asp/isoAsp
  VARIANT 319: note = Xaa may be Asn, or deamidated to Asp, isoAsp, or Asp/isoAsp
  VARIANT 329: note = Xaa may be Asn, or deamidated to Asp, isoAsp, or Asp/isoAsp
  VARIANT 332: note = Xaa may be K (lys, lysine), or acetylated K
  VARIANT 336: note = Xaa may be Asn, or deamidated to Asp, isoAsp, or Asp/isoAsp
  VARIANT 384: note = Xaa may be D (asp, aspartic acid), or isomerized D.
  VARIANT 404: note = Xaa may be M (Met, Methionine), or oxidated M.
  VARIANT 409: note = Xaa may be Asn, or deamidated to Asp, isoAsp, or Asp/isoAsp
  VARIANT 436: note = Xaa may be M (Met, Methionine), or oxidated M.
  VARIANT 452: note = Xaa may be Asn, or deamidated to Asp, isoAsp, or Asp/isoAsp
  VARIANT 477: note = Xaa may be Asn, or deamidated to Asp, isoAsp, or Asp/isoAsp
  VARIANT 499: note = Xaa may be S (Ser, serine), or Phosphorylated S
  VARIANT 512: note = Xaa may be Asn, or deamidated to Asp, isoAsp, or Asp/isoAsp
  VARIANT 515: note = Xaa may be Asn, or deamidated to Asp, isoAsp, or Asp/isoAsp
  VARIANT 518: note = Xaa may be M (Met, Methionine), or oxidated M
  VARIANT 524: note = Xaa may be M (Met, Methionine), or oxidated M.
  VARIANT 559: note = Xaa may be M (Met, Methionine), or oxidated M.
  VARIANT 569: note = Xaa may be T (Thr, threonine), or Phosphorylated T
  VARIANT 586: note = Xaa may be S (Ser, serine), or Phosphorylated S
  VARIANT 599: note = Xaa represents Q, or Q deamidated to glutamic acid (alpha-glutamic acid), gamma-glutamic acid (Glu), or a blend of alpha- and gamma-glutamic acid
  VARIANT 605: note = Xaa may be M (Met, Methionine), or oxidated M.
  VARIANT 619: note = Xaa may be W (Trp, tryptophan), or oxidated W (e.g., kynurenine)
  VARIANT 628: note = Xaa may be Asn, or deamidated to Asp, isoAsp, or Asp/isoAsp
  VARIANT 640: note = Xaa may be M (Met, Methionine), or oxidated M.
  VARIANT 651: note = Xaa may be Asn, or deamidated to Asp, isoAsp, or Asp/isoAsp
  VARIANT 663: note = Xaa may be Asn, or deamidated to Asp, isoAsp, or Asp/isoAsp
  VARIANT 666: note = Xaa may be K (lys, lysine), or acetylated K
  VARIANT 689: note = Xaa may be K (lys, lysine), or acetylated K
  VARIANT 693: note = Xaa may be K (lys, lysine), or acetylated K
  VARIANT 695: note = Xaa may be W (Trp, tryptophan), or oxidated W.
  VARIANT 709: note = Xaa may be Asn, or deamidated to Asp, isoAsp, or Asp/isoAsp
  VARIANT 735: note = Xaa may be Asn, or deamidated to Asp, isoAsp, or Asp/isoAsp
MAADGYLPDWLEDNLSEGIREWXALKPGAPQPKAXQQHQDNARGLVLPGYKYLGPGXGLDKGEPVXEADAAALEHDKAYDQQLKAGDNPYLKYXHAXAEFQERLKEXTSFGGXLGRAVFQAKKRLLEPLGLVEEAAKTAPGKKRPVEQXPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTXALPTYXNHLYKXISNSTSGGSSXDNAYFGYSTPWGYFDFNRFHCHFSPRXWQRLINXNXGFRPKRLXFKLFXIQVKEVTDNXGVXTIAXNLTSTVQVFTDSDYQLPYVLGSAHEGCLPPFPADVFMIPQYGYLTLNXGSQAVGRSSFYCLEYFPSQXLRTGXNFQFSYEFENVPFHSSYAHSQSLDRLXNPLIDQYLYYLSKTIXGSGQNQQTLKFSVAGPSNMAVQGRXYIPGPSYRQQRVSTTVTQNNNXEFAWPGASSWALXGRXSLXNPGPAXASHKEGEDRFFPLSGSLIFGKQGTGRDNVDADKVXITNEEEIKTXNPVATESYGQVATNHQXAQAQAQTGWVQNQGILPGXVWQDRDVYLQGPIXAKIPHTDGXFHPSPLMGGFGXKHPPPQILIKXTPVPADPPTAFXKDXLNSFITQYSTGQVSVEIEWELQXENSXRXNPEIQYTSNYYKSXNVEFAVNTEGVYSEPRPIGTRYLTRXL

SEQ ID NO: 9 - DNA, 2211 bp, synthetic construct
  misc_feature 1..2211: note = AAV9 vp1 coding sequence
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 10 - protein, 736 aa, synthetic construct
  REGION 1..736: note = Encoded AAV9 vp1 amino acid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - DNA, 2211 bp, synthetic construct; identical to SEQ ID NO: 9
  misc_feature 1..2211: note = AAVhu31 vp1 coding sequence
  source 1..2211: mol_type = other DNA

SEQ ID NO: 12 - protein, 736 aa, synthetic construct
  REGION 1..736: note = Encoded AAVhu31 vp1 amino acid sequence
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 13 - DNA, 2211 bp, synthetic construct
  misc_feature 1..2211: note = AAVhu32 vp1 coding sequence
  source 1..2211: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 14 - protein, 736 aa, synthetic construct
  REGION 1..736: note = Encoded AAVhu32 vp1 amino acid sequence
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 423 aa, Homo sapiens
MGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 16 - DNA, 666 bp, synthetic construct
  misc_feature 1..666: note = chicken beta actin promoter with a cytomegalovirus enhancer (CB7)
  source 1..666: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 17 - DNA, 973 bp, synthetic construct
  misc_feature 1..973: note = chicken beta-actin intron
  source 1..973: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 18 - DNA, 282 bp, synthetic construct
  misc_feature 1..282: note = CB promoter
  source 1..282: mol_type = other DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 19 - DNA, 382 bp, synthetic construct
  misc_feature 1..382: note = CMV Immediate early Promoter
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 20 - DNA, 22 bp, synthetic construct
  misc_feature 1..22: note = miR183
  misc_feature 1..22: note = miRNA183
  source 1..22: mol_type = other DNA
agtgaattctaccagtgccata

SEQ ID NO: 21 - DNA, 23 bp, synthetic construct
  misc_feature 1..23: note = miRNA target sequence
  source 1..23: mol_type = other DNA
agcaaaaatgtgctagtgccaaa

SEQ ID NO: 22 - DNA, 24 bp, synthetic construct
  misc_feature 1..24: note = miRNA target sequence
  source 1..24: mol_type = other DNA
agtgtgagttctaccattgccaaa

SEQ ID NO: 23 - DNA, 23 bp, synthetic construct
  misc_feature 1..23: note = miRNA target sequence
  source 1..23: mol_type = other DNA
agggattcctgggaaaactggac

SEQ ID NO: 24 - DNA, 16 bp, synthetic construct
  misc_feature 1..16: note = Spacer
  source 1..16: mol_type = other DNA
atgacttaaaccaggt

SEQ ID NO: 25 - DNA, 145 bp, synthetic construct
  source 1..145: mol_type = other DNA; note = AAV2 5' ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 26 - DNA, 145 bp, synthetic construct
  source 1..145: mol_type = other DNA; note = AAV2 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 27 - DNA, 3883 bp, synthetic construct
  source 1..3883: mol_type = other DNA; note = vector genome CB7.CI.hARSAco.RBG
  repeat_region 1..130: note = 5' ITR
  regulatory 198..579: note = cytomegalovirus immediate early enhancer; regulatory_class = enhancer
  regulatory 582..862: note = chicken beta actin promoter; regulatory_class = promoter
  regulatory 836..839: note = TATA signal; regulatory_class = TATA_box
  misc_feature 956..1928: note = chicken beta-actin intron
  misc_feature 1962..3488: note = Engineered ARSA coding sequence
  regulatory 3539..3665: note = Rabbit globin poly A; regulatory_class = polyA_signal_sequence
  repeat_region 3754..3883: note = 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgaattcggtaccacaggccaccatgtctatgggagcccctagatctctgctgctggctctggctgctggactggcagttgccagacctcctaacatcgtgctgatcttcgccgacgatctcggctacggcgatctgggctgttacggacaccccagcagcaccacacctaacctggatcaacttgccgctggcggcctgagattcaccgatttctacgtgcccgtgtctctgtgcaccccttctagagctgctctgctgacaggcagactccctgtgcggatgggaatgtatcctggcgtgctggtgcctagctctagaggcggactgcctctggaagaagtgacagttgccgaagtgctggccgccagaggatatctgactggcatggccggaaagtggcacctcggagttggaccagaaggcgcttttctgcctcctcaccagggcttccaccggtttctgggcatcccttactctcacgatcagggcccctgccagaacctgacctgttttcctcctgccacaccttgcgacggcggctgtgatcaaggactggtgccaattcctctgctggccaacctgagcgtggaagctcaacctccttggctgccaggactggaagcccggtatatggccttcgctcacgacctgatggccgacgctcagagacaggacagaccattcttcctgtactacgccagccaccacacacactaccctcagtttagcggccagagcttcgccgagagatctggcagaggacctttcggcgacagcctgatggaactggatgccgctgtgggcacactgatgacagccatcggagatctgggactgctggaagagacactggtcatcttcaccgccgacaacggccccgagacaatgagaatgagcagaggcggctgtagcggcctgctgagatgtggcaagggcaccacatatgaaggcggcgtgagagaacctgctctggccttttggcctggccatattgctccaggcgtgacacacgagctggcctcttctctggatctgctgcctacactggcagctcttgctggtgctcccctgcctaatgtgaccctggatggcttcgatctgagcccactgctgctcggcacaggcaagtctccaagacagagcctgttcttctaccctagctaccccgacgaagtgcggggagtgtttgccgtgcggaccggaaagtataaggcccacttcttcacccaaggcagcgcccactctgacaccacagctgatcctgcttgtcacgccagctctagcctgacagcccatgaacctccactgctgtacgacctgagcaaggaccccggcgagaactacaatctgcttggcggagttgccggcgctacacctgaagttctgcaggccctgaaacagctccagctgctgaaagcccagctggacgctgccgtgacatttggacctagtcaggtggccagaggcgaggatcctgctctgcagatctgttgtcaccctggctgcacacccagacctgcctgctgtcattgtcctgatccacacgcctgatgaacagcctgaggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 3468 bp, synthetic construct
  source 1..3468: mol_type = other DNA; note = expression cassette CB7.CI.hARSAco.RBG
  regulatory 1..382: note = cytomegalovirus immediate early enhancer; regulatory_class = enhancer
  regulatory 385..665: note = chicken beta actin promoter; regulatory_class = promoter
  regulatory 639..642: note = TATA signal; regulatory_class = TATA_box
  misc_feature 759..1731: note = chicken beta actin intron
  misc_feature 1765..3291: note = Engineered ARSA coding sequence
  regulatory 3342..3468: note = Rabbit globin poly A; regulatory_class = polyA_signal_sequence
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgaattcggtaccacaggccaccatgtctatgggagcccctagatctctgctgctggctctggctgctggactggcagttgccagacctcctaacatcgtgctgatcttcgccgacgatctcggctacggcgatctgggctgttacggacaccccagcagcaccacacctaacctggatcaacttgccgctggcggcctgagattcaccgatttctacgtgcccgtgtctctgtgcaccccttctagagctgctctgctgacaggcagactccctgtgcggatgggaatgtatcctggcgtgctggtgcctagctctagaggcggactgcctctggaagaagtgacagttgccgaagtgctggccgccagaggatatctgactggcatggccggaaagtggcacctcggagttggaccagaaggcgcttttctgcctcctcaccagggcttccaccggtttctgggcatcccttactctcacgatcagggcccctgccagaacctgacctgttttcctcctgccacaccttgcgacggcggctgtgatcaaggactggtgccaattcctctgctggccaacctgagcgtggaagctcaacctccttggctgccaggactggaagcccggtatatggccttcgctcacgacctgatggccgacgctcagagacaggacagaccattcttcctgtactacgccagccaccacacacactaccctcagtttagcggccagagcttcgccgagagatctggcagaggacctttcggcgacagcctgatggaactggatgccgctgtgggcacactgatgacagccatcggagatctgggactgctggaagagacactggtcatcttcaccgccgacaacggccccgagacaatgagaatgagcagaggcggctgtagcggcctgctgagatgtggcaagggcaccacatatgaaggcggcgtgagagaacctgctctggccttttggcctggccatattgctccaggcgtgacacacgagctggcctcttctctggatctgctgcctacactggcagctcttgctggtgctcccctgcctaatgtgaccctggatggcttcgatctgagcccactgctgctcggcacaggcaagtctccaagacagagcctgttcttctaccctagctaccccgacgaagtgcggggagtgtttgccgtgcggaccggaaagtataaggcccacttcttcacccaaggcagcgcccactctgacaccacagctgatcctgcttgtcacgccagctctagcctgacagcccatgaacctccactgctgtacgacctgagcaaggaccccggcgagaactacaatctgcttggcggagttgccggcgctacacctgaagttctgcaggccctgaaacagctccagctgctgaaagcccagctggacgctgccgtgacatttggacctagtcaggtggccagaggcgaggatcctgctctgcagatctgttgtcaccctggctgcacacccagacctgcctgctgtcattgtcctgatccacacgcctgatgaacagcctgaggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

(19) United States
(12) Patent Application Publication
(10) Pub. No.: US 2025/0177495 A1
(43) Pub. Date: Jun. 5, 2025

(54) COMPOSITIONS USEFUL IN TREATMENT OF METACHROMATIC LEUKODYSTROPHY

(71) Applicant: The Trustees of the University of Pennsylvania, Philadelphia, PA (US)

(72) Inventors: Juliette Hordeaux, Philadelphia, PA (US); James M. Wilson, Philadelphia, PA (US)

(73) Assignee: The Trustees of the University of Pennsylvania, Philadelphia, PA (US)

(21) Appl. No.: 18/727,953

(22) PCT Filed: Jan. 10, 2023
(86) PCT No.: PCT/US2023/060376

§ 371 Date: Jul. 10, 2024

Related U.S. Application Data
(60) Provisional application No. 63/341,636, filed on May 13, 2022.
(60) Provisional application No. 63/331,367, filed on Apr. 15, 2022.
(60) Provisional application No. 63/297,958, filed on Jan. 10, 2022.

(51) Int. Cl. A61K 38/46; A61K 48/00; A61P 3/00

(52) U.S. Cl. CPC A61K 38/465; A61K 48/0033; A61K 48/0066; A61K 48/0075; A61P 3/00

(57) ABSTRACT

Provided is a recombinant adeno-associated virus (rAAV) having an AAVhu68 capsid and a vector genome which comprises a nucleic acid sequence encoding a functional human arylsulfatase A (ARSA). Also provided are a production system useful for producing the rAAV, a pharmaceutical composition comprising the rAAV, and a method of treating a subject having metachromatic leukodystrophy, or ameliorating symptoms of metachromatic leukodystrophy, or delaying progression of metachromatic leukodystrophy via administrating an effective amount of the rAAV to a subject in need thereof.

### REFERENCE TO AN ELECTRONIC SEQUENCE LISTING

The electronic sequence listing filed herewith named “22-9955PCT.xml” with size of 102,745 bytes, created on date of Jan. 28, 2025, and the contents of the electronic sequence listing (e.g., the sequences and text therein) are incorporated herein by reference in entirety.

### BACKGROUND OF THE INVENTION

Metachromatic Leukodystrophy (MLD) is a monogenic autosomal recessive sphingolipid storage disease caused by mutations in the gene encoding the lysosomal enzyme ARSA (Von Figura et al., 2001; Gieselmann and Krageloh-Mann, 2010). ARSA deficiency leads to accumulation of its natural substrates, which are sulfated galactosphingolipids (galactosylceramide-3-O-sulfate and galactosylsphingosine-3-O-sulfate), commonly referred to as sulfatides. Sulfatides accumulate within the lysosomes of oligodendrocytes, microglia, and certain types of neurons in the Central Nervous System (CNS), in addition to Schwann cells and macrophages in the Peripheral Nervous System (PNS) (Peng and Suzuki, 1987). While the PNS and CNS are mainly affected, sulfatide storage also occurs in visceral organs; most notably, the kidney, liver (Toda et al., 1990), and gallbladder (Rodriguez-Waitkus et al., 2011; McFadden and Ranganathan, 2015).

MLD patients (i.e., those who carry a mutation on both alleles) typically have ARSA enzyme activity that is 0-10% of control values in synthetic substrate-based assays. ARSA mutation carriers, who have a single mutated ARSA allele and one normal allele, are clinically unaffected and usually have ARSA enzyme activity that is approximately 10% of control values, while asymptomatic individuals with pseudodeficiency (PD, another genetically distinct form of ARSA deficiency) alleles have ARSA enzyme activity that is approximately 10-20% of healthy controls (Gomez-Ospina, 2017). Clinically, three forms of MLD can be distinguished based on age of symptom onset that span a broad continuous spectrum of disease severity: a rapidly progressive severe late infantile form, a juvenile form, and a late onset slowly progressive adult form comprising 50-60%, 20-30%, and 15-20% of MLD diagnoses, respectively (Gomez-Ospina, 2017, Wang et al., 2011). Infantile MLD is considered an orphan disease. Late infantile MLD has an onset before 30 months of age and is the most severe form of the disease. The late infantile form has a uniform clinical presentation and a rapidly progressive, predictable disease course. Juvenile MLD is characterized by an age of onset between the age of 30 months and 16 years with a median age of onset of 6 years 2 months (Kehrer et al., 2011a) to 10 years (Mahmood et al., 2010), depending on the study. In order to better characterize the clinical phenotype, a subset of juvenile MLD patients has been described, referred to as early juvenile MLD, who have a clinical onset ≤6 years of age and who have a similar, although less rapid, initial disease evolution compared to children with late infantile MLD (Biffi et al., 2008; Chen et al., 2016; Sessa et al., 2016). The early juvenile and late infantile phenotypes are collectively referred to as early onset MLD (Sessa et al., 2016). In late juvenile MLD patients (i.e., those with symptom onset between 7-16 years of age), behavioral issues, attention deficit, or cognitive decline usually develops first, sometimes in combination with gait disturbances.

There is no approved curative or disease-modifying therapy for MLD. Since MLD is caused by defective ARSA, various investigational approaches aim to correct the biochemical defect by replacing functional ARSA in affected neural tissue of the CNS. Enzyme replacement therapy (ERT) and hematopoietic stem cell transplantation (HSCT) rely on providing normal enzyme to ARSA-deficient cells, while gene therapy approaches are based on the overexpression of wild-type ARSA in different cell types (Patil and Maegawa, 2013). The efficacy of Hematopoietic Stem Cell Transplantation (HSCT) using umbilical cord blood (UCB), allogeneic peripheral blood stem cells, or allogeneic bone marrow depends on the MLD phenotype and the timing of intervention relative to the disease state of the patient (Patil and Maegawa, 2013; van Rappard et al., 2015). Bone marrow transplant (BMT) requires availability of a human leukocyte antigen-matched sibling donor for the best outcome (Boucher et al., 2015) and carries risks of transplant- and conditioning-related complications, such as graft versus host disease (GvHD), infections, and death. Umbilical Cord Blood (UCB) transplantation provides an alternative to BMT with the advantage of quicker availability, lower risk of GvHD, lower mortality, higher rates of full-donor chimerism, and better correction of enzymatic defect (Batzios and Zafeiriou, 2012; Martin et al., 2013). However, BMT is not widely available in Europe. Brain engraftment is slow, often taking many months for cells to engraft, migrate to the CNS, differentiate, and restore enzyme levels. Moreover, physiological enzyme levels achieved with HSCT may not be sufficient to correct the deficit throughout the CNS. This may explain why transplant is not efficacious in rapidly progressive early onset MLD, and may not correct or stabilize all aspects of the disease even when performed pre-symptomatically (de Hosson et al., 2011; Martin et al., 2013; Boucher et al., 2015).

Thus, there remains a substantial unmet need for fast-onset therapies that can halt or prevent disease progression in these patients.

In addition to HSCT, various other cell-based approaches exist that (over)express ARSA and deliver enzyme to affected cells and treat the neurological manifestations of MLD, including microencapsulated recombinant cells, oligodendrocyte and neural progenitor cells, and embryonic stem cells. These cell therapies have shown considerable clearance of sulfatide storage in animal models (Patil and Maegawa, 2013), but are still untested in humans.

Ex vivo lentiviral gene therapy has been attempted which combines hematopoietic stem cell transplant with gene therapy (HSC-GT) (Biffi et al., 2013) by transducing autologous CD34+ cells with a human ARSA-encoding lentiviral vector and re-administering the gene-corrected cells to the patient. While this therapy is promising for patients identified at a pre-symptomatic stage (after diagnosis in an older affected sibling), it has not been shown to be efficacious in patients who are already symptomatic. Unfortunately, most new MLD diagnoses are made after symptom onset because newborn screening is not yet available, making it an unlikely therapeutic option for many MLD patients. Additionally, there are risks inherent to the myeloablative conditioning regimen and risk of insertional mutagenesis associated with these integrating vectors.

A pharmacological-toxicological study in NHPs demonstrated significant dose-limiting toxicity (Zerah et al., 2015) due to brain inflammation (encephalitis) localized around injection sites. A Phase 1/2 clinical study to assess safety and efficacy of AAVrh10-mediated ARSA gene transfer in the brain of children affected with early onset MLD is ongoing (NCT01801709) (Aubourg, 2016) likewise involved intra-cerebral vector administration at 12 sites in the white matter of the brain (Zerah et al., 2015). Results of the trial have not been published, except in abstract form, with preliminary reports suggesting lack of efficacy at preventing onset or stopping disease progression (Sevin et al., 2018). The reasons for the lack of efficacy have not been discussed by the sponsor of the trial. In addition to AAVrh10-mediated gene therapy, an intra-cerebrally delivered lentiviral gene therapy is also recruiting patients with any form of MLD (NCT03725670).

Enzyme replacement therapy (ERT) is now the Standard of Care (SOC) for several Lysosomal Storage Diseases (LSDs) (Sands, 2014) and relies on the ability of cells to take up infused enzyme via mannose-6-phosphate receptors (Ghosh et al., 2003). In MLD, ERT reduces sulfatide storage in the kidneys, peripheral nerves, and CNS in Arsa^{-/-} mice (Matzner et al., 2005). In an aggravated MLD mouse model with immune tolerance to human ARSA and supra-normal sulfatide synthesis, improvements in MLD symptoms and reduction in sulfatide storage was seen only in mice treated at early time points, suggesting that IV-administered ERT may not work in patients with advanced symptoms (Matthes et al., 2012). In the same model, continuous IT infusion of recombinant ARSA to bypass the BBB (Stroobants et al., 2011) resulted in complete reversal of sulfatide storage and correction of CNS dysfunction, while other non-clinical studies in mice result in reduced sulfatide storage and improved functional outcomes (Matzner et al., 2009; Piguet et al., 2012). However, in humans, the extent of metabolic correction with ERT will unlikely be sufficient and timely to arrest the rapid cerebral demyelination that occurs in early onset MLD (Rosenberg et al., 2016). Since the BBB restricts access to the CNS of most large proteins, it is believed that ERT will likely only work when delivered directly to the CNS (Abbott, 2013), and the short half-life will require frequent administration. This hypothesis is bearing out in ERT clinical trials that attempted to overcome these limitation through frequent high dose IV administration (NCT00681811) or IT injection (Giugliani et al., 2018). However, results in late infantile MLD patients with IV-administered ERT have been disappointing (NCT00418561), along with IT-administered ERT in early onset and late juvenile MLD (NCT01510028).

Small molecule-based treatments can potentially overcome limitations of current therapies for MLD (e.g., by crossing the BBB) and may also address different pathogenic mechanisms of the disease. Warfarin (Coumadin) is an anti-coagulant that has been tested as a substrate-reducing agent in a small cohort of late infantile MLD patients. There was no beneficial effect on urinary sulfatide levels or levels of the brain biomarkers N-acetylaspartate and myo-inositol (Patil and Maegawa, 2013).

The limited benefit, restricted population, short therapeutic window, and associated risks of HSCT and HSC-GT combined with the overall disappointing non-clinical results obtained with other investigational approaches represent a significant unmet clinical need for other viable treatment options, especially for early onset MLD patients.

What is desirable are alternative therapeutics for treatment of conditions associated with abnormal ARSA gene and/or Metachromatic Leukodystrophy.

### SUMMARY OF THE INVENTION

Provided herein is a therapeutic, recombinant, and replication-defective adeno-associated virus (rAAV) which is useful for treating a disease associated with an Arylsulfatase A gene (ARSA) mutation (for example, Metachromatic Leukodystrophy, i.e., MLD, or ARSA pseudodeficiency) in a subject in need thereof. The rAAV is desirably replication-defective and carries a vector genome comprising inverted terminal repeats (ITR) and a nucleic acid sequence encoding a functional human Arylsulfatase A (hARSA) under the control of regulatory sequences which direct the hARSA expression in a target cell. In certain embodiment, the rAAV further comprises an AAVhu68 capsid in which the vector genome is packaged. In certain embodiments, the vector genome is entirely exogenous to the AAVhu68 capsid, as it contains no AAVhu68 genomic sequences.

In certain embodiments, pharmaceutical composition for use in treating metachromatic leukodystrophy or a disease associated with a arylsulfatase A (ARSA) gene mutation are provided. The composition may comprise a recombinant adeno-associated virus (rAAV) comprising an AAVhu68 capsid; and a vector genome comprising: a 5′ AAV inverted terminal repeats (ITR), a CB7 promoter comprising a CMV IE enhancer and a CB promoter, and a nucleic acid sequence encoding a functional human Arylsulfatase A (hARSA) operably linked to regulatory sequences comprising the CB7 promoter which direct the hARSA expression, a polyA signal, and a 3′ AAV ITR wherein the hARSA coding sequence comprises a sequence of nucleotide (nt) 1 to nt 1521 of SEQ ID NO: 1, or a sequence at least 95% to 99.9% identical thereto which encodes a functional hARSA; and at least one aqueous buffer, at least one carrier, at least one excipient and/or a least one preservative, said composition being deliverable in a single therapeutic dose via intrathecal administration. In certain embodiments, the regulatory elements further comprise one or more of a Kozak sequence, an intron, a further enhancer, and/or a TATA signal. In certain embodiments, the hARSA coding sequence is SEQ ID NO: 1 or SEQ ID NO: 3. In certain embodiment, the vector genome comprises a sequence of nt 1 to nt 3883 of SEQ ID NO: 5. In certain embodiments, the AAVhu68 capsid is produced from a sequence encoding the amino acid sequence of SEQ ID NO: 7. In certain embodiments, the composition comprises an artificial cerebrospinal fluid comprising buffered saline and one or more of sodium, calcium, magnesium, potassium, or mixtures thereof; and a surfactant. In certain embodiments, the composition further comprises at least one surfactant, optionally present at 0.0005% to about 0.001% of the pharmaceutical composition. In certain embodiments, the composition is at a pH in the range of 6.5 to 8.5. In certain embodiments, the composition is suitable for an intra-cisterna magna injection (ICM) or intracerebroventricular administration. In certain embodiments, the single dose comprises 3×10¹⁰ genome copies (GC)/gram of brain mass to 3.5×10¹¹ GC/gram of brain mass. In certain embodiments, the dose is: (a) about 3.3×10¹⁰ genome copies (GC)/gram of brain mass; (b) about 1.1×10¹¹ genome copies (GC)/gram of brain mass; or (c) about 3.3×10¹¹ genome copies (GC)/gram of brain mass.

In certain embodiments, use of an rAAV.hARSA in the manufacture of a medicament for the therapeutic treatment of Metachromatic Leukodystrophy or a disease associated with a Arylsulfatase A (ARSA) gene mutation is provided. The medicament may be delivered via intrathecal administration of a single dose comprising 3×10¹⁰ genome copies (GC)/gram of brain mass to 3.5×10¹¹ GC/gram of brain mass to a patient. In certain embodiments, the dose is: (a) about 3.3×10¹⁰ genome copies (GC)/gram of brain mass; (b) about 1.1×10¹¹ genome copies (GC)/gram of brain mass; or (c) about 3.3×10¹¹ genome copies (GC)/gram of brain mass.

In certain embodiments, a method of treating a subject having metachromatic leukodystrophy or a disease associated with a Arylsulfatase A (ARSA) gene mutation is provided. The method comprises administering a single dose of a recombinant AAV to the subject by ICM injection, wherein the recombinant AAV comprises an AAVhu68 capsid and a vector genome packaged therein, said vector genome comprising AAV ITRs, an hARSA coding sequence comprising SEQ ID NO: 1, or a sequence at least 95% identical thereto that encodes a functional hARSA, and regulatory sequences which direct expression of the functional hARSA in a target cell, wherein the single dose is 3×10¹⁰ genome copies (GC)/gram of brain mass to 3.5×10¹¹ GC/gram of brain mass, or optionally, (i) about 3.3×10¹⁰ genome copies (GC)/gram of brain mass; (ii) about 1.1×10¹¹ GC/gram of brain mass; or (iii) about 3.3×10¹¹ GC/gram of brain mass.

These and other aspects of the invention are apparent from the following detailed description of the invention.

### BRIEF DESCRIPTION OF THE DRAWINGS

FIG. 1 provides the engineered hARSA coding sequence (SEQ ID NO: 1, i.e., nt 7 to nt 1527 of SEQ ID NO: 3 and nt 1968 to nt 3488 of SEQ ID NO: 5).

FIG. 2 provides a linear map of the AAV.CB7.CI.hARSAco.rBG vector genome. The vector genome is to express an engineered version of human ARSA (hARSAco) under the control of the ubiquitous CB7 promoter. CB7 is a hybrid promoter element comprising, at a minimum, a CMV IE enhancer and a chicken BA promoter. ARSA, arylsulfatase A; BA, β-actin; CMV IE, cytomegalovirus immediate-early; ITR, inverted terminal repeats; PolyA, polyadenylation; and rBG, rabbit β-globin.

FIG. 3 provides a linear map of the cis plasmid, termed pENN.AAV.CB7.CI.hARSAco.rBG.KanR. BA, β-actin; bp, base pairs; CMV IE, cytomegalovirus immediate-early; hARSAco, human arylsulfatase A (engineered); ITR, inverted terminal repeat; KanR, kanamycin resistance; Ori, origin of replication; PolyA, polyadenylation; rBG, rabbit β-globin. A vector genome with a 130-bp flop-oriented AAV-ITR sequence at each end of the linear molecule, shortened by 15 bp from the terminal of the intact 145-bp ITR, is shown. The AAV.CB7.CI.hARSAco.rBG vector genome encapsulated in the AAV capsid can comprise the intact 145-bp ITR, instead of the 130-bp ITR.

FIG. 4 provides a linear map of the trans plasmid pAAV2/hu68.KanR. AAV2, adeno-associated virus serotype 2; AAVhu68, adeno-associated virus serotype hu68; bp, base pairs; Cap, capsid; KanR, kanamycin resistance; Ori, origin of replication; Rep, replicase.

FIG. 5A and FIG. 5B provide an adenovirus helper plasmid pAdDeltaF6(KanR). FIG. 5A shows derivation of the helper plasmid pAdΔF6 from parental plasmid pBHG10 through intermediates pAdΔF1 and pAdΔF5. FIG. 5B shows that the ampicillin resistance gene in pAdΔF6 was replaced by the kanamycin resistance gene to generate pAdΔF6(Kan).

FIG. 6 provides a manufacturing process flow diagram for producing AAVhu68.hARSAco vector. AAV, adeno-associated virus; AEX, anion exchange; CRL, Charles River Laboratories; ddPCR, droplet digital polymerase chain reaction; DMEM, Dulbecco's modified Eagle medium; DNA, deoxyribonucleic acid; FFB, final formulation buffer; GC, genome copies; HEK293, human embryonic kidney 293 cells; ITFFB, intrathecal final formulation buffer; PEI, polyethylenimine; SDS-PAGE, sodium dodecyl sulfate polyacrylamide gel electrophoresis; TFF, tangential flow filtration; USP, United States Pharmacopeia; WCB, working cell bank.

FIG. 7 provides a manufacturing process flow diagram for AAVhu68.hARSAco vector. Ad5, adenovirus serotype 5; AUC, analytical ultracentrifugation; BDS, bulk drug substance; BSA, bovine serum albumin; CZ, Crystal Zenith; ddPCR, droplet digital polymerase chain reaction; E1A, early region 1A (gene); ELISA, enzyme-linked immunosorbent assay; FDP, filled drug product; GC, genome copies; HEK293, human embryonic kidney 293 cells; ITFFB, intrathecal final formulation buffer; KanR, kanamycin resistance (gene); MS, mass spectrometry; NGS, next-generation sequencing; qPCR, quantitative polymerase chain reaction; SDS-PAGE, sodium dodecyl sulfate polyacrylamide gel electrophoresis; TCID₅₀, 50% tissue culture infective dose; UPLC, ultra-performance liquid chromatography; USP, United States Pharmacopeia.

FIG. 8 shows transgene product expression (ARSA enzyme activity) in the brain of mice administered AAVhu68.CB7.CI.hARSAco.rBG (GTP-207) or vehicle. On Day 0, C57BL/6J (WT) mice were ICV-administered either AAVhu68.CB7.CI.hARSAco.rBG (GTP-207) (1.0×10¹⁰ GC or 1.0×10¹¹ GC) or control article (PBS [vehicle]). At necropsy on Day 21, brains were collected for an ARSA enzyme activity assay to evaluate transgene product expression. Error bars represent the standard deviation.

FIG. 9 shows transgene product expression (ARSA enzyme activity) in serum of mice administered AAVhu68.CB7.CI.hARSAco.rBG (GTP-207) or vehicle. On Day 0, C57BL/6J (WT) mice were ICV-administered either AAVhu68.CB7.CI.hARSAco.rBG (GTP-207) (1.0×10¹⁰ GC or 1.0×10¹¹ GC) or control article (PBS [vehicle]). On Day 7 and at necropsy on Day 21, serum was collected for an ARSA enzyme activity assay to evaluate transgene product expression. Error bars represent the standard deviation.

FIG. 10 shows transgene product expression (ARSA enzyme activity) in the liver of mice administered AAVhu68.CB7.CI.hARSAco.rBG (GTP-207) or vehicle. On Day 0, C57BL/6J (WT) mice were ICV-administered either AAVhu68.CB7.CI.hARSAco.rBG (GTP-207) (1.0×10¹⁰ GC or 1.0×10¹¹ GC) or control article (PBS [vehicle]). At necropsy on Day 21, livers were collected for an ARSA enzyme activity assay to evaluate transgene product expression. Error bars represent the standard deviation.

FIG. 11 shows antibodies against the transgene product (anti-Human ARSA Antibodies) in serum of mice following ICV administration of AAVhu68.CB7.CI.hARSAco.rBG (GTP-207) or vehicle. On Day 0, C57BL/6J (WT) mice were ICV-administered either AAVhu68.CB7.CI.hARSAco.rBG (GTP-207) (1.0×10¹⁰ GC or 1.0×10¹¹ GC) or control article (PBS [vehicle]). On Day 7 and at necropsy on Day 21, serum was collected, and antibodies against the transgene product (anti-human ARSA antibodies) were measured by ELISA. Error bars represent the standard deviation.

FIG. 12 shows transgene product expression (HA IF) in neurons and oligodendrocytes in the brain of mice administered AAVhu68.CB7.CI.hARSAco-HA.rBG or vehicle. On Day 0, C57BL/6J (WT) mice were ICV-administered either AAVhu68.CB7.CI.hARSAco-HA.rBG (1.0×10¹⁰ GC or 1.0×10¹¹ GC) or control article (PBS [vehicle]). On Day 21 post vector administration, mice were necropsied, and brain tissue was collected. Tissues were sectioned and immunostained to visualize human ARSA (green; anti-HA antibody) and oligodendrocytes (red: anti-OLIG2 antibody). Representative images of the brain cortex are shown at 20× magnification with 500 ms exposure. Cropped and zoomed-in views (bottom row) show oligodendrocytes from the subcortical white matter expressing ARSA.

FIG. 13 shows transgene product expression (ARSA enzyme activity) in serum of mice administered AAVhu68.CB7.CI.hARSAco-HA.rBG or vehicle. On Day 0, C57BL/6J (WT) mice were ICV-administered either AAVhu68.CB7.CI.hARSAco-HA.rBG (1.0×10¹⁰ GC or 1.0×10¹¹ GC) or control article (PBS [vehicle]). On Day 7 and at necropsy on Day 21, serum was collected for an ARSA enzyme activity assay to evaluate transgene product expression. Error bars represent the standard deviation.

FIG. 14 shows transgene product expression (ARSA enzyme activity) in the liver of mice administered AAVhu68.CB7.CI.hARSAco-HA.rBG or vehicle. On Day 0, C57BL/6J (WT) mice were ICV-administered either AAVhu68.CB7.CI.hARSAco-HA.rBG (1.0×10¹⁰ GC or 1.0×10¹¹ GC) or control article (PBS [vehicle]). At necropsy on Day 21, livers were collected for an ARSA enzyme activity assay to evaluate transgene product expression. Error bars represent the standard deviation.

FIG. 15 shows body weights of NHPs following ICM AAV administration. Adult NHPs (N=2) received a single ICM administration of AAVhu68.CB7.CI.hARSAco-HA.rBG at dose of 3.0×10¹³ GC. Body weights were measured at the indicated time points.

FIG. 16 shows CSF leukocyte counts in NHPs following ICM AAV administration. Adult NHPs (N=1 female RA2397 and N=1 male RA2477) received a single ICM administration of AAVhu68.CB7.CI.hARSAco-HA.rBG at dose of 3.0×10¹³ GC. CSF leukocyte counts were evaluated at the indicated time points. The dotted line indicates the cutoff threshold for lymphocytic pleocytosis in rhesus macaques (≥6 WBC/L CSF).

FIGS. 17A and 17B show transgene product expression (ARSA enzyme activity) in cerebrospinal fluid (CSF) and serum of NHPs following ICM AAV administration. Adult NHPs (N=2) received a single ICM administration of AAVhu68.CB7.CI.hARSAco-HA.rBG at dose of 3.0×10¹³ GC. Transgene product expression in CSF and serum was measured by an ARSA enzyme activity assay on the indicated days.

FIG. 18 shows transgene product expression (ARSA enzyme activity) in tissues of NHPs following ICM AAV administration. Adult NHPs (N=1 female RA2397, N=1 male RA2477) received a single ICM administration of AAVhu68.CB7.CI.hARSAco-HA.rBG at the dose of 3.0×10¹³ GC. Two animals from an unrelated study that received AAV9 (RA2172, female) or AAV9-PHPB (RA2145, male) encoding Green Fluorescent Protein (GFP) intravenously (2.0×10¹³ GC/Kg) were included as controls for endogenous levels of ARSA activity in rhesus macaques. Human ARSA protein was measured by ELISA in the indicated tissues collected at necropsy on Day 21.

FIG. 19 shows transgene product expression (HA Tag IHC) in the spinal cord and peripheral nerves of NHPs following ICM AAV administration. Adult rhesus macaques received a single ICM administration of AAVhu68.CB7.CI.hARSAco-HA.rBG at a dose of 3.0×10¹³ GC (N=2). Nervous system tissues were collected at necropsy on Day 21 for IHC staining using an antibody recognizing the hemagglutinin (HA) tag (brown precipitate). Representative images from animal RA2397 of the dorsal root ganglia (DRG), spinal cord motor neurons, and peripheral nerves of the AAV-treated rhesus macaques are shown.

FIG. 20A and FIG. 20B show transgene product expression (HA Tag IF) in the trigeminal ganglia (TRG) and peripheral nerves of NHPs following ICM AAV administration. Adult rhesus macaques received a single ICM administration of AAVhu68.CB7.CI.hARSAco-HA.rBG at a dose of 3.0×10¹³ GC (N=2). Nervous system tissues were collected at necropsy on Day 21 for IF staining using an antibody recognizing the HA tag (red staining). Representative images are shown for (FIG. 20A) median nerve sections from an untreated age-matched rhesus macaques from another study versus an AAV-treated animal RA2397 in this study and (FIG. 20B) the TRG and peripheral nerves of RA2397 rhesus macaques.

FIG. 21 shows body weights of NHPs following ICM AAV administration. Adult NHPs (N=2/group) received a single ICM administration of AAVhu68.CB7.CI.hARSAco.rBG (GTP-207) at dose of 3.0×10¹² GC (low dose), 1.0×10¹³ GC (mid-dose), or 3.0×10¹³ GC (high dose). Body weights were measured at the indicated time points.

FIG. 22 shows CSF leukocyte counts in NHPs following ICM AAV administration. Adult NHPs (N=2/group) received a single ICM administration of AAVhu68.CB7.CI.hARSAco.rBG (GTP-207) at dose of 3.0×10¹² GC (low dose), 1.0×10¹³ GC (mid-dose), or 3.0×10¹³ GC (high dose). CSF leukocyte counts were evaluated at the indicated time points.

FIGS. 23A and 23B shows DRG and spinal cord pathology findings in NHPs following ICM AAV administration. Adult NHPs (N=2/group) received a single ICM administration of AAVhu68.CB7.CI.hARSAco.rBG (GTP-207) at dose of 3.0×10¹² GC (low dose), 1.0×10¹³ GC (mid-dose), or 3.0×10¹³ GC (high dose). DRG and spinal cord tissues (cervical, thoracic, and lumbar) were collected at necropsy and histopathologic evaluation was performed. Findings of neuronal cell body degeneration with mononuclear cell infiltrates for each DRG segment and findings of axonopathy in the dorsal white matter tracts of the spinal cord were assigned the following severity scores: Grade 1=minimal, Grade 2=mild, Grade 3=moderate, Grade 4=marked; Grade 5=severe.

FIGS. 24A and 24B show transgene product expression (ARSA enzyme activity) in CSF and serum of NHPs following ICM AAV administration. Adult NHPs (N=2/group) received a single ICM administration of AAVhu68.CB7.CI.hARSAco.rBG (GTP-207) at dose of 3.0×10¹² GC (low dose), 1.0×10¹³ GC (mid-dose), or 3.0×10¹³ GC (high dose). Human ARSA protein was measured by ELISA in the CSF and plasma on the indicated study days.

FIGS. 25A and 25B shows antibodies against the transgene product (anti-human ARSA antibodies) in CSF and serum of NHPs following ICM AAV administration. Adult NHPs (N=2/group) received a single ICM administration of AAVhu68.CB7.CI.hARSAco.rBG (GTP-207) at dose of 3.0×10¹² GC (low dose), 1.0×10¹³ GC (mid-dose), or 3.0×10¹³ GC (high dose). Anti-human ARSA antibodies were measured by ELISA in the CSF and serum on the indicated study days.

FIG. 26 shows transgene product expression (human ARSA immunohistochemistry) in the brain of NHPs following ICM AAV administration. Adult cynomolgus macaques received a single ICM administration of AAVhu68.CB7.CI.hARSAco.rBG (GTP-207) at a dose of 3.0×10¹³ GC (high dose) (N=2). Untreated age-matched cynomolgus macaques served as a control (N=2). Animals were necropsied 42±2 days post treatment, and brains were obtained for IHC using an antibody recognizing human ARSA (brown precipitate). Representative images of sections through the brain's cortex, hippocampus, thalamus, and cerebellum for one AAV-treated animal (right panels) is shown, along with sections from an untreated control for signal comparison (left panels).

FIG. 27 shows transgene product expression (human ARSA immunohistochemistry) in the spinal cord and dorsal root ganglia of NHPs following ICM AAV administration. Adult cynomolgus macaques received a single ICM administration of AAVhu68.CB7.CI.hARSAco.rBG (GTP-207) at a dose of 3.0×10¹³ GC (high dose) (N=2). Untreated age-matched cynomolgus macaques served as a control (N=2). Animals were necropsied 42±2 days post treatment, and sections of the cervical, thoracic, and lumbar spinal cord and DRG were obtained for IHC using an antibody recognizing human ARSA (brown precipitate). Representative images of sections for one AAV-treated animal (right panels) are shown, along with sections from an untreated control for signal comparison (left panels).

FIGS. 28A and 28B show body weights of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Body weights were measured monthly until necropsy at ˜9 months of age (Groups 3-4) or ˜15 months of age (Groups 1-2). Data are presented as mean±the standard deviation. *p<0.05, **p<0.01, ***p<0.001, ****p<0.0001 based on a 2-way ANOVA using Sidak's multiple comparisons test.

FIG. 29 shows body weights of AAV-GAL3ST1-treated Arsa−/− mice. On Study Day 0, AAV-GAL3ST1-treated adult (˜3-month-old) male Arsa−/− mice were enrolled in the natural history study (N=5, Group 5). Age-matched male C57BL/6J (wild type) mice were also included as controls (N=6, Group 6). Body weights were measured monthly until necropsy at ˜9 months of age. Data are presented as mean±the standard deviation. **p<0.01 based on a 2-way ANOVA using Sidak's multiple comparisons test.

FIG. 30 shows clinical scoring assessments of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). A standardized clinical assessment was performed on each animal every other week until necropsy at Study Week 27 (Study Day 180; Groups 3 and 4) or Study Week 52 (Study Day 360; Groups 1 and 2). (A) Mean clinical scores for all animals throughout the study and (B) a comparison of clinical scores for individual animals at Study Week 28 versus Study Week 52 are presented. Error bars represent the standard deviation. *p<0.05, ***p<0.001, ****p<0.0001 based on a 2-way ANOVA using Sidak's multiple comparisons test.

FIG. 31 shows clinical scoring assessments of AAV-GAL3ST1-treated Arsa−/− mice. On Study Day 0, AAV-GAL3ST1-treated adult (˜3-month-old) male Arsa−/− mice were enrolled in the natural history study (N=5, Group 5). Age-matched male C57BL/6J (wild type) mice were also included as controls (N=6, Group 6). A standardized clinical assessment was performed on each animal every other week until necropsy on Study Week 27 (Study Day 180). Data are presented as the mean score±the standard deviation.

FIG. 32 shows ledge test of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). The ledge test was performed on each animal every other week until necropsy at Study Week 27 (Study Day 180; Groups 3 and 4) or Study Week 52 (Study Day 360; Groups 1 and 2). Data are presented as the mean score±the standard deviation. *p<0.05, **p<0.01, ***p<0.001, ****p<0.0001 based on a 2-way ANOVA using Sidak's multiple comparisons test.

FIG. 33 shows ledge test of AAV-GAL3ST1-treated Arsa−/− mice. On Study Day 0, AAV-GAL3ST1-treated adult (˜3-month-old) male Arsa−/− mice were enrolled in the natural history study (N=5, Group 5). Age-matched male C57BL/6J (wild type) mice were also included as controls (N=6, Group 6). The ledge test was performed on each animal every other week until necropsy at Study Week 27 (Study Day 180). Data are presented as the mean score±the standard deviation. **p<0.01 based on a 2-way ANOVA using Sidak's multiple comparisons test.

FIG. 34A-FIG. 34B show RotaRod analysis of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). The RotaRod assessment was performed on each animal every month until necropsy on Study Day 180 (Groups 3 and 4) or Study Day 360 (Groups 1 and 2). (A) Mean latencies to fall for all animals throughout the study and (B) mean latencies to fall on Study Day 360 (Groups 1 and 2 only) are presented. Error bars represent the standard deviation.

FIG. 35 shows RotaRod analysis of AAV-GAL3ST1-treated Arsa−/− Mice. On Study Day 0, AAV-GAL3ST1-treated adult (˜3-month-old) male Arsa−/− mice were enrolled in the natural history study (N=5, Group 5). Age-matched male C57BL/6J (wild type) mice were also included as controls (N=6, Group 6). The RotaRod assessment was performed on each animal every month until necropsy on Study Day 180. Data are presented as the mean latency to fall for all animals in each group±the standard deviation.

FIGS. 36A and 36B show catwalk gait analysis of untreated Arsa−/− mice measuring base of support. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Gait analysis was performed on mice every 60 days, measuring base of support using the CatWalk XT system. (FIG. 36A) Mean base of support for the fore limbs and (FIG. 36B) mean base of support for the hind limbs are presented. Data are presented as the means±the standard error of the mean. *p<0.05 based on a 2-way ANOVA using Sidak's multiple comparisons test. *p<0.05, ****p<0.0001 based on a 2-way ANOVA using Sidak's multiple comparisons test.

FIG. 37 shows catwalk gait analysis of untreated Arsa−/− mice measuring cadence. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Gait analysis was performed on mice every 60 days, measuring cadence using the CatWalk XT system. Data are presented as the means±the standard error of the mean. *p<0.05 based on a 2-way ANOVA using Sidak's multiple comparisons test.

FIG. 38 shows catwalk gait analysis of untreated Arsa−/− mice measuring step sequence. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Gait analysis was performed on mice every 60 days, measuring step sequence using the CatWalk XT system. Data are presented as the means±the standard error of the mean. *p<0.05 based on a 2-way ANOVA using Sidak's multiple comparisons test.

FIG. 39 shows catwalk gait analysis of untreated Arsa−/− mice measuring stride length. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Gait analysis was performed on mice every 60 days, measuring stride length for each limb (right front, right hind, left front, and left hind) using the CatWalk XT system. Data are presented as the means±the standard error of the mean. *p<0.05, **p<0.01, ****p<0.0001 based on a 2-way ANOVA using Sidak's multiple comparisons test. **p<0.01 based on a 2-way ANOVA using Sidak's multiple comparisons test. ****p<0.0001 based on a 2-way ANOVA using Sidak's multiple comparisons test.

FIG. 40 shows catwalk gait analysis of untreated Arsa−/− mice measuring maximum contact area. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Gait analysis was performed on mice every 60 days, measuring maximum contact area for each limb (right front, right hind, left front, and left hind) using the CatWalk XT system. Data are presented as the means±the standard error of the mean. *p<0.05 based on a 2-way ANOVA using Sidak's multiple comparisons test.

FIG. 41 shows lysosomal-associated membrane protein 1 (LAMP-1) IHC in the brain of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Mice were necropsied at ˜9 months of age or ˜15 months of age. Brains were collected, sectioned, and stained to evaluate lysosomal storage lesions (LAMP-1 IHC; brown precipitate). Representative images of the cortex, cerebellum, and brainstem are presented.

FIGS. 42A and 42B show quantification of LAMP-1-positive area in brain and spinal cord of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Mice were necropsied at ˜9 months of age or ˜15 months of age. Brain and spinal cord were collected, sectioned, and stained to evaluate lysosomal storage lesions (LAMP-1 IHC). The percent LAMP-1-positive area was quantified using image analysis software. *p<0.05, ***p<0.001, ****p<0.0001 based on a 2-way ANOVA using Sidak's multiple comparisons test.

FIG. 43 shows GFAP IHC in the brain of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Mice were necropsied at ˜9 months of age or ˜15 months of age. Brains were collected, sectioned, and stained to evaluate astrogliosis/neuroinflammation (GFAP IHC; brown precipitate). Representative images of the cortex, hippocampus, cerebellum, brainstem, and spinal cord are presented.

FIGS. 44A and 44B shows quantification of glial fibrillary acidic protein (GFAP)-positive area in brain and spinal cord of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Mice were necropsied at ˜9 months of age or ˜15 months of age. Brain and spinal cord were collected, sectioned, and stained to evaluate astrogliosis/neuroinflammation (GFAP IHC). The percent GFAP-positive area was quantified using image analysis software. *p<0.05 based on a 2-way ANOVA using Sidak's multiple comparisons test.

FIG. 45 shows histological evaluation of sulfatide storage by Alcian blue staining in brain and kidney of untreated Arsa−/− mice. On Study Day 0, untreated adult (˜3-month-old) male and female Arsa−/− mice were enrolled in the natural history study (N=10, Group 1; N=8 Group 3). Age-matched male and female C57BL/6J (wild type) mice were included as a control (N=13, Group 2; N=10 Group 4). Mice were necropsied at ˜9 months of age or ˜15 months of age. Brain and kidney were collected, sectioned, and stained to evaluate sulfatide storage (Alcian Blue staining; blue precipitate). Representative images of the cortex and kidney from mice in Groups 1 and 2 are presented. Arrows denote sulfatide deposits in the brain.

FIG. 46 shows histological evaluation of sulfatide storage by Alcian blue staining in kidney, brain, sciatic nerve, and spinal cord of AAV-GAL3ST1-treated Arsa−/− mice. On Study Day 0, AAV-GAL3ST1-treated adult male Arsa−/− mice (˜3-month-old) were enrolled in the natural history study (N=5, Group 5). Age-matched male C57BL/6J (wild type) mice were also included as controls (N=6, Group 6). Necropsies were performed at ˜9 months of age. Kidney, sciatic nerve, brain, and spinal cord were collected, sectioned, and stained to evaluate sulfatide storage (Alcian blue staining; blue precipitate). Representative images from mice in Groups 5 and 6 are presented.

FIGS. 47A-47C shows sulfatide analysis on brain tissue from untreated Arsa−/− mice and AAV-GAL3ST1-treated Arsa−/− mice. On Study Day 0, untreated adult (˜3-month-old) male and female Arsa−/− mice were enrolled in the natural history study (N=10, Group 1; N=8 Group 3), and age-matched male and female C57BL/6J wild type mice (WT) were included as a control (N=13, Group 2; N=10 Group 4). Additionally, on Study Day 0, AAV-GAL3ST1-treated adult (2-3-month-old) male Arsa−/− mice were enrolled in the natural history study (N=5, Group 5), and age-matched male C57BL/6J wild type mice (WT AAV-GAL3ST1-treated) were included as controls (N=6, Group 6). Mice were necropsied at ˜9 months of age (Groups 3-6) or ˜15 months of age (Groups 1-2), and a subset of animals (N=9 from Group 1; N=3 from Group 2; N=6 from Group 3; N=2 from Group 4; N=5 from Group 5; N=2 from Group 6) were assessed for sulfatide storage in the brain by LC/MS. **p<0.01, ***p<0.001 based on a 2-way ANOVA using Sidak's multiple comparisons test.

FIGS. 48A and 48B shows sulfatide analysis in kidney of untreated Arsa−/− mice and AAV-GAL3ST1-yreated Arsa−/− mice. On Study Day 0, untreated adult (˜3-month-old) male and female Arsa−/− mice were enrolled in the natural history study (N=10, Group 1; N=8 Group 3), and age-matched male and female C57BL/6J wild type mice (WT) were included as a control (N=13, Group 2; N=10 Group 4). Additionally, on Study Day 0, AAV-GAL3ST1-treated adult (2-3-month-old) male Arsa−/− mice were enrolled in the natural history study (N=5, Group 5), and age-matched male C57BL/6J wild type mice (WT AAV-GAL3ST1-treated) were included as controls (N=6, Group 6). Mice were necropsied at ˜9 months of age (Groups 3-6) or ˜15 months of age (Groups 1-2), and a subset of animals (N=9 from Group 1; N=3 from Group 2; N=6 from Group 3; N=2 from Group 4; N=5 from Group 5; N=2 from Group 6) were assessed for sulfatide storage in the kidney by LC/MS. *p<0.05, **p<0.01, ****p<0.0001 based on a 2-way ANOVA using Sidak's multiple comparisons test.

FIGS. 49A to 49C show sulfatide analysis in liver of untreated Arsa−/− mice and AAV-GAL3ST1-treated Arsa−/− mice. On Study Day 0, untreated adult (˜3-month-old) male and female Arsa−/− mice were enrolled in the natural history study (N=10, Group 1; N=8 Group 3), and age-matched male and female C57BL/6J wild type mice (WT) were included as a control (N=13, Group 2; N=10 Group 4). Additionally, on Study Day 0, AAV-GAL3ST1-treated adult (2-3-month-old) male Arsa−/− mice were enrolled in the natural history study (N=5, Group 5), and age-matched male C57BL/6J wild type mice (WT AAV-GAL3ST1-treated) were included as controls (N=6, Group 6). Mice were necropsied at ˜9 months of age (Groups 3-6) or ˜15 months of age (Groups 1-2), and a subset of animals (N=9 from Group 1; N=3 from Group 2; N=6 from Group 3; N=2 from Group 4; N=5 from Group 5; N=2 from Group 6) were assessed for sulfatide storage in the liver by LC/MS. *p<0.05, **p<0.01 based on a 2-way ANOVA using Sidak's multiple comparisons test.

FIG. 50 shows evaluation of endogenous ARSA protein in tissue of untreated Arsa−/− mice by western blot and enzyme activity. Brain tissue lysate from one homozygous (ARSA KO) and one WT litter mate from each ARSA line was used to evaluate ARSA protein using western blot (Anti-ARSA/ASA antibody [EPR11039](ab174844), Abcam, 1:1000, 54 kDa). Results demonstrate absence of 54 kDa ARSA protein in the knockout animals including the line 407047 (highlighted in red). HSP 90α/β (SC-13119, Santa Cruz Biotechnology, 1:5000, 90 kDa) was used as loading control. Untreated adult (˜3-month-old) male and female Arsa−/− mice were enrolled in the natural history study (N=10, Group 1; N=8 Group 3). Age-matched male and female C57BL/6J wild type mice (WT) were included as a control (N=13, Group 2; N=10 Group 4). At 4 months of age (Study Day 128), N=2 animals in Groups 3 and 4 each were necropsied to evaluated ARSA enzyme activity in the serum and tissues (brain, spinal cord, liver, kidney, spleen). In this p-nitrocatechol based assay, activities were measured in tissue samples in the presence (non-specific activity), and absence (total activity) of silver nitrate, an ARSA inhibitor. Specific ARSA activity, excluding enzyme activity due to other sulfatases present in tissue, was determined by subtracting non-specific from total activity values. Data are presented as the mean standard deviation.

FIG. 51 shows LAMP-1 IHC in the cortex and hippocampus. On Study Day 0, adult male Arsa^{-/-} mice received a single ICV injection of AAVhu68.CB7.CI.hARSAco.rBG (GTP-207; N=2) at a dose of 4.5×10¹⁰ GC or PBS (vehicle N=1). Age-matched male C57BL/6J (wild type) mice were treated with PBS (vehicle) and included as a control (N=1). Mice were necropsied on Day 30. Brains were collected, sectioned, and stained to evaluate lysosomal storage lesions in N=1 per group (LAMP-1 IHC; brown precipitate). Representative images of LAMP-1 IHC in cortex and hippocampus are presented.

FIG. 52 shows LAMP-1 IHC in the cerebellum and brainstem. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Age-matched male C57BL/6J (wild type) mice were treated with PBS (vehicle) and included as a control (N=1). Mice were necropsied on Day 30. Brains were collected, sectioned, and stained to evaluate lysosomal storage lesions in N=1 per group (LAMP-1 IHC; brown precipitate). Representative images of LAMP-1 IHC in cerebellum and brain stem are presented.

FIG. 53 shows GFAP IHC in the cortex and hippocampus. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Age-matched male C57BL/6J (wild type) mice were treated with PBS (vehicle) and included as a control (N=1). Mice were necropsied on Day 30. Brains were collected, sectioned, and stained to evaluate astrogliosis/neuroinflammation in N=1 per group (GFAP IHC; brown precipitate). Representative images of GFAP IHC in cortex and hippocampus are presented.

FIG. 54 shows GFAP IHC in the cerebellum and brain stem. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Age-matched male C57BL/6J (wild type) mice were treated with PBS (vehicle) and included as a control (N=1). Mice were necropsied on Day 30. Brains were collected, sectioned, and stained to evaluate astrogliosis/neuroinflammation in N=1 per group (GFAP IHC; brown precipitate). Representative images of GFAP IHC the cerebellum and brain stem are presented.

FIG. 55 shows transgene product expression (human ARSA immunohistochemistry) in the cortex and hippocampus. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Age-matched male C57BL/6J (wild type) mice were treated with PBS (vehicle) and included as a control (N=1). Mice were necropsied on Day 30. Brains were collected, sectioned, and stained to evaluate ARSA protein expression in N=1 per group (ARSA IHC; brown precipitate). Representative images of human ARSA IHC in the cortex and hippocampus are presented.

FIG. 56 shows transgene product expression (human ARSA immunohistochemistry) in the cerebellum and brain stem. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Age-matched male C57BL/6J (wild type) mice were treated with PBS (vehicle) and included as a control (N=1). Mice were necropsied on Day 30. Brains were collected, sectioned, and stained to evaluate ARSA protein expression in N=1 per group (ARSA IHC; brown precipitate). Representative images of human ARSA in the cerebellum and brain stem are presented.

FIG. 57 shows transgene product expression (human ARSA immunohistochemistry) in the liver and heart. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle (N=1). Age-matched male C57BL/6J (wild type) mice were treated with PBS (vehicle) and included as a control (N=1). Mice were necropsied on Day 30. Liver and heart were collected, sectioned, and stained to evaluate ARSA protein expression in N=1 per group (ARSA IHC; brown precipitate). Representative images of human ARSA IHC in liver and heart are presented.

FIG. 58 shows sulfatide analysis on brain tissue from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched C57BL/6J (wild type) mice were included as a control (N=1). Mice were necropsied on Day 30 and brains were collected and assessed for sulfatide storage by LC/MS in N=1 per group.

FIG. 59 shows sulfatide analysis on sciatic nerve tissue from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched male C57BL/6J (wild type) mice were included as a control (N=1). Mice were necropsied on Day 30 and sciatic verves were collected and assessed for sulfatide storage by LC/MS in N=1 per group.

FIG. 60 shows sulfatide analysis on liver tissue from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched male C57BL/6J (wild type) mice were included as a control (N=1). Mice were necropsied on Day 30 and livers were collected and assessed for sulfatide storage by LC/MS in N=1 per group.

FIGS. 61A to 61C shows sulfatide analysis on spleen tissue from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched male C57BL/6J (wild type) mice were included as a control (N=1). Mice were necropsied on Day 30 and spleens were collected and assessed for sulfatide storage by LC/MS in N=1 per group.

FIG. 62 shows sulfatide analysis on kidney tissue from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched male C57BL/6J (wild type) mice were included as a control (N=1). Mice were necropsied on Day 30 and kidneys were collected and assessed for sulfatide storage by LC/MS in N=1 per group.

FIG. 63 shows sulfatide analysis on heart Tissue from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched male C57BL/6J (wild type) mice were included as a control (N=1). Mice were necropsied on Day 30 and hearts were collected and assessed for sulfatide storage by LC/MS in N=1 per group.

FIG. 64 shows sulfatide analysis on quadriceps muscle tissue from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched male C57BL/6J (wild type) mice were included as a control (N=1). Mice were necropsied on Day 30 and quadriceps were collected and assessed for sulfatide storage by LC/MS in N=1 per group.

FIGS. 65A and 65B show sulfatide analysis on plasma from Arsa^{-/-} mice and wild-type control mice. On Study Day 0, adult male Arsa^{-/-} mice received a single ICV injection of AAVhu68.CB7.CI.hARSAco.rBG (GTP-207 N=2) at a dose of 4.5×10¹⁰ GC or PBS (vehicle N=1). Untreated age-matched male C57BL/6J (wild type) mice were included as a control (N=1). Mice were necropsied on Day 30 and plasma was collected and assessed for sulfatide storage by LC/MS in N=1 per group.

FIG. 66 shows ARSA enzyme activity in tissues of Arsa^{-/-} and wild-type control mice. On Study Day 0, adult male Arsa^{-/-} mice received a single ICV injection of AAVhu68.CB7.CI.hARSAco.rBG (GTP-207) at a dose of 4.5×10¹⁰ GC (N=2) or PBS (vehicle; N=1). Age-matched male C57BL/6J (wild type) mice were also administered PBS (vehicle) and included as a control (N=2). Mice were necropsied on Day 30 and ARSA enzyme activity was measured in the tissues (brain, heart, spinal cord, liver, kidney, spleen).

FIG. 67 shows ARSA enzyme activity in serum of Arsa^{-/-} and wild-type control mice. On Study Day 0, adult male Arsa^{-/-} mice received a single ICV injection of AAVhu68.CB7.CI.hARSAco.rBG (GTP-207) at a dose of 4.5×10¹⁰ GC (N=2) or PBS (vehicle; N=1). Age-matched male C57BL/6J (wild type) mice were also administered PBS (vehicle) and included as a control (N=2). Mice were necropsied on Day 30 and ARSA enzyme activity was measured in the serum.

FIG. 68 shows survival. On Day −7 (baseline), 4-5-month-old Arsa^{-/-} mice or wild type mice were enrolled. On Day 0, animals received AAVhu68.CB7.CI.hARSAco.rBG (GTP-207) at 1 of 3 doses or PBS as a vehicle control (N=5 males and 5 females per group). Data points show death events (unscheduled only). *p<0.05 Log-rank (Mantel-Cox) test comparing each group to Arsa^{-/-} PBS control. Abbreviations: LD, low dose (1.3×10¹⁰ GC); MD, mid-dose (4.5×10¹⁰ GC); HD, high dose (1.3×10¹¹ GC) FIG. 69 shows body weights. On Day −7 (baseline), 4-5-month-old Arsa^{-/-} mice or wild type mice were enrolled. On Day 0, mice received AAVhu68.CB7.CI.hARSAco.rBG (GTP-207) at 1 of 3 doses or PBS as a vehicle control (N=5 males and 5 females per group). Data points show the mean with standard error of mean. Abbreviations: BL, baseline; LD, low dose (1.3×10¹⁰ GC); MD, mid-dose (4.5×10¹⁰ GC); HD, high dose (1.3×10¹¹ GC).

FIG. 70 shows clinical scoring assessments. On Day −7 (baseline), 4-5-month-old Arsa mice or wild type mice were enrolled. On Day 0, mice received AAVhu68.CB7.CI.hARSAco.rBG (GTP-207) at 1 of 3 doses or PBS as a vehicle control (N=5 males and 5 females per group). Data points show mean clinical scores with standard error of the mean. ***p<0.001, ****p<0.0001 based on a mixed effect model comparing each group to Arsa^{-/-} PBS control. Abbreviations: BL, baseline; LD, low dose (1.3×10¹⁰ GC); MD, mid-dose (4.5×10¹⁰ GC); HD, high dose (1.3×10¹¹ GC).

FIG. 71 shows ledge test. On Day −7 (baseline), 4-5-month-old Arsa^{-/-} mice or wild type mice were enrolled. On Day 0, they received AAVhu68.CB7.CI.hARSAco.rBG (GTP-207) at 1 of 3 doses or PBS as a vehicle control (N=5 males and 5 females per group). Data points show mean ledge test scores with a standard error of mean. *p<0.05, **p<0.01, ****p<0.0001 based on a mixed effect model comparing each group to Arsa^{-/-} PBS control. Abbreviations: BL, baseline; LD, low dose (1.3×10¹⁰ GC); MD, mid-dose (4.5×10¹⁰ GC); HD, high dose (1.3×10¹¹ GC); ns, not significant.

FIG. 72 shows RotaRod analysis. On Day −7 (baseline), 4-5-month-old Arsa^{-/-} mice or wild type mice were enrolled. On Day 0, mice received AAVhu68.CB7.CI.hARSAco.rBG (GTP-207) at 1 of 3 doses or PBS as a vehicle control (N=5 males and 5 females per group). Data points show the mean accelerated RotaRod latency to fall in seconds with the standard error of the mean. **p<0.01, ****p<0.0001 based on a mixed effect model comparing each group to the Arsa^{-/-} PBS control followed by multiple comparison test at each timepoint. Abbreviations: BL, baseline; LD, low dose (1.3×10¹⁰ GC); MD, mid-dose (4.5×10¹⁰ GC); HD, high dose (1.3×10¹¹ GC); ns, not significant.

FIG. 73 shows catwalk gait analysis, base of support. On Day −7 (baseline), 4-5-month-old Arsa^{-/-} mice or wild type mice were enrolled. On Day 0, mice received AAVhu68.CB7.CI.hARSAco.rBG (GTP-207) at 1 of 3 doses or PBS as a vehicle control (N=5 males and 5 females per group). Data points show mean base of support of hind limbs in cm with the standard deviation. *p<0.05, **p<0.01, ***p<0.001, ****p<0.0001 based on a two-way ANOVA with a post hoc multiple comparison Dunnett's test comparing each group to the Arsa^{-/-} vehicle-treated control. Abbreviations: BL, baseline; LD, low dose (1.3×10¹⁰ GC); MD, mid-dose (4.5×10¹⁰ GC); HD, high dose (1.3×10¹¹ GC).

FIGS. 74A and 74B show catwalk gait analysis, duration (FIG. 74A), and average speed (FIG. 74B). On Day −7 (baseline), 4-5-month-old Arsa^{-/-} mice or wild type mice were enrolled. On Day 0, mice received AAVhu68.CB7.CI.hARSAco.rBG (GTP-207) at 1 of 3 doses or PBS as vehicle control (N=5 males and 5 females per group). Data points show mean duration (s) or speed (cm/s) with the standard deviation. *p<0.05, **p<0.01, ***p<0.001, ****p<0.0001 based on a two-way ANOVA with post hoc multiple comparison Dunnett's test comparing each group to Arsa^{-/-} PBS control.

FIG. 75 shows catwalk gait analysis, stride length. On Day −7 (baseline), 4-5-month-old Arsa^{-/-} mice or wild type mice were enrolled. On Day 0, mice received AAVhu68.CB7.CI.hARSAco.rBG (GTP-207) at 1 of 3 doses or PBS as a vehicle control (N=5 males and 5 females per group). Data points show mean stride length (cm) with the standard deviation. *p<0.05, **p<0.01, ***p<0.001, ****p<0.0001 based on a two-way ANOVA with post hoc multiple comparison Dunnett's test comparing each group to Arsa^{-/-} PBS control. Abbreviations: BL, baseline; LD, low dose (1.3×10¹⁰ GC); MD, mid-dose (4.5×10¹⁰ GC); HD, high dose (1.3×10¹¹ GC).

FIG. 76A shows transgene product expression—ARSA enzyme activity in brain (left panel), liver (middle) and heart (right panel). On Day −7 (baseline), 4-5-month-old Arsa^{-/-} mice or wild type mice were enrolled. On Day 0, mice received AAVhu68.CB7.CI.hARSAco.rBG (GTP-207) at 1 of 3 doses or PBS as vehicle control (N=5 males and 5 females per group). Data points show mean ARSA enzyme activity with the standard error of mean. Abbreviations: LD, low dose (1.3×10¹⁰ GC); MD, mid-dose (4.5×10¹⁰ GC); HD, high dose (1.3×10¹¹ GC); 4NC 4-nitrocatechol released from 4-nitrocatechol sulfate artificial substrate.

FIG. 76B shows quantification of sulfatides in the brain of Arsa^{-/-} mice administered AAVhu68.CB7.CI.hARSAco.rBG (GTP-207) or vehicle. On Day −7 (baseline), 4-5-month-old Arsa^{-/-} mice or wild type mice were enrolled. On Day 0, mice received AAVhu68.CB7.CI.hARSAco.rBG (GTP-207) at 1 of 3 doses or PBS as vehicle control (N=5 males and 5 females per group). At necropsy brain tissue from was processed for LC-MS analysis to determine the effect of AAVhu68.CB7.CI.hARSAco.rBG (GTP-207) treatment on storage of sulfatide species (left, Brain C16:0; right, brain C18:0). Bars represent group means. *p<0.05, ***p<0.001 1-way ANOVA and post hoc Dunn's multiple comparisons test (each group compared to Arsa^{-/-} vehicle).

FIGS. 77A and 77B show body weights of Arsa^{-/-} mice administered AAVhu68.CB7.CI.hARSAco.rBG (GTP-207) or vehicle. FIG. 77A shows body weight males. FIG. 77B shows body weight females. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (intrathecal final formulation buffer (ITFFB)) as controls. Animals were weighed once per week. Error bars represent the standard error of mean. Two-way ANOVA followed by Dunnett's multiple comparison test, alpha of 0.05 (each group compared to Arsa^{-/-} vehicle controls): WT vehicle statistically different from Arsa^{-/-} vehicle between Study Day 21 (*p=0.02) and Study Day 180 (****p<0.0001) in males, and between Study Day 84 (*p=0.03) and Study Day 180 (****p<0.0001) in females.

FIG. 78 shows clinical scoring assessments of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At baseline, Study Day 90, and Study Day 180 a standardized clinical assessment was performed on each animal by a blinded operator. Data are mean±/−standard error for cumulative deficit score from five measured parameters. ****p<0.0001 2-way ANOVA and post hoc Dunnett's multiple comparisons test compared to vehicle treated Arsa^{-/-} group.

FIGS. 79A and 79B shows transgene expression and anti-transgene antibodies in serum of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. On baseline, Study Day 14, and Study Day 60 serum was collected for analysis of ARSA enzyme activity to evaluate transgene expression using a 4-nitrocatechol sulfate substrate (FIG. 79A), and for analysis of anti-hARSA antibodies by ELISA (FIG. 79B). Data are mean±/−standard error. ****p<0.0001 2-way ANOVA and post hoc multiple comparisons test (each group compared to Arsa^{-/-} vehicle). Abbreviations: ITFFB, intrathecal final formulation buffer; 4-NC, 4-nitrocatechol.

FIG. 80 shows transgene expression in the brain of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, or 1.3×10¹⁰ GC, 4.5×10⁹ GC (Study Day 0). Age-matched Arsa^{-/-} mice and WT mice were ICV-administered vehicle (ITFFB) as controls. Mice in groups 1 and 2 were necropsied on Study Day 0 (Baseline) and groups 3 to 8 were necropsied on Study Day 180+/−5. Brains were collected from the mice and tissue from rostral brain was assayed for ARSA enzyme activity to evaluate transgene expression (generation of 4-NC/mg tissue/5 hrs). Bars represent group means. *p<0.05, **p<0.01, ***p<0.001 1-way ANOVA and post hoc Dunn's multiple comparisons test (each group compared to Arsa^{-/-} vehicle).

FIG. 81 shows transgene expression in the liver of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (groups 1 and 2) or Study Day 180+/−5), a portion of the liver was collected, and ARSA enzyme activity assayed to evaluate transgene expression (generation of 4-NC/mg tissue/5 hrs). Bars represent group means. *p<0.05, **p<0.01, 1-way ANOVA and post hoc Dunn's multiple comparisons test (each group compared to Arsa^{-/-} vehicle).

FIG. 82 shows transgene expression in the heart of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (groups 1 and 2) or Study Day 180+/−5), a portion of the heart was collected, and ARSA enzyme activity assayed to evaluate transgene expression (generation of 4-NC/mg tissue/5 hrs). The bars represent groups' means. *p<0.05, **p<0.01, ****p<0.0001 1-way ANOVA and post hoc Dunn's multiple comparisons test (each group compared to Arsa^{-/-} vehicle).

FIG. 83 shows hARSA IHC in brain of WT and Arsa^{-/-} mice administered vehicle. At 4 months of age, Arsa^{-/-} mice and WT mice were ICV-administered vehicle (ITFFB) as controls. At necropsy (Study Day 180+/−5), the caudal portion of the brain was collected and processed for hARSA IHC in a subset of animals in each group (qualitative analysis only). Representative images of hARSA IHC from vehicle treated WT and Arsa^{-/-} mice. Top panel: Group 3-WT vehicle. Bottom panel: Group 4—Arsa^{-/-} Vehicle. Rostral portion of the brain is missing as it was collected for biochemical assays.

FIG. 84 shows hARSA IHC in brain of Arsa^{-/-} mice administered AAVhu68.CB7.CI.hARSAco.rBG (GTP-207) (Group 5 & 6). At 4 months of age, Arsa^{-/-} mice were ICV-administered AAVhu68.CB7.CI.hARSAco.rBG (GTP-207) at a dose of 1.3×10¹¹ GC or 4.5×10¹⁰ GC (Study Day 0). At necropsy (Study Day 180+/−5), the caudal portion of the brain was collected and processed for hARSA IHC. Representative images of hARSA IHC from AAVhu68.CB7.CI.hARSAco.rBG (GTP-207) administered Arsa^{-/-} mice. Top panel: Group 5—Arsa^{-/-} 1.3×10¹¹ GC. Bottom panel: Group 6—Arsa^{-/-} 4.5×10¹⁰ GC. Rostral portion of the brain is missing as it was collected for biochemical assays.

FIG. 85 shows hARSA IHC in brain of Arsa^{-/-} mice administered AAVhu68.CB7.CI.hARSAco.rBG (GTP-207) (Group 7 & 8). At 4 months of age, Arsa^{-/-} mice were ICV-administered AAVhu68.CB7.CI.hARSAco.rBG (GTP-207) at a dose of 1.3×10¹⁰ GC or 4.5×10⁹ GC (Study Day 0). At necropsy (Study Day 180+/−5), the caudal portion of the brain was collected and processed for hARSA IHC. Representative images of hARSA IHC from AAVhu68.CB7.CI.hARSAco.rBG (GTP-207) administered Arsa^{-/-} mice. Top panel: Group 7—Arsa^{-/-} 1.3×10¹⁰ GC. Bottom panel: Group 8—Arsa^{-/-} 4.5×10⁹ GC. Rostral portion of the brain is missing as it was collected for biochemical assays.

FIGS. 86A and 86B show blood urea nitrogen (BUN; FIG. 86A) and magenesium (Mg; FIG. 86B) levels in Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (at Baseline Study Day 0 (groups 1 and 2) or at Study Day 180) serum was collected to evaluate BUN and magnesium (Mg) levels as part of a serum chemistry panel. The bars represent groups' means. *p<0.05, **p<0.01, ***p<0.001 1-way ANOVA and post hoc Dunn's multiple comparisons test (each group compared to Arsa^{-/-} vehicle).

FIG. 87 shows quantitative scoring of LAMP-1 IHC in brain, spinal cord, and sciatic nerve of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (Groups 1 and 2), or Study Day 180+/−5), the caudal portion of the brain was collected and processed for LAMP-1 IHC. *p<0.05, ***p<0.001, ****p<0.0001 1-way ANOVA and post hoc Dunn's multiple comparisons test (each group compared to Arsa¹-vehicle).

FIG. 88 shows quantitative scoring of GFAP IHC in brain and spinal cord of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (Groups 1 and 2), or Study Day 180+/−5), the caudal portion of the brain was collected and processed for GFAP IHC. *p<0.05, **p<0.01, ***p<0.001, ****p<0.0001 1-way ANOVA and post hoc Dunn's multiple comparisons test (each group compared to Arsa^{-/-} vehicle).

FIG. 89 shows quantification of sulfatide C16:0 in the plasma of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. On Study Day 170, plasma was collected and analyzed for sulfatide C16:0 using LC-MS. Bars represent group means. *p<0.05, ***p<0.001 ****p<0.001 1-way ANOVA and post-hoc Dunn's multiple comparisons test (each group compared to Arsa^{-/-} vehicle).

FIG. 90 shows quantification of sulfatides in the brain of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (Groups 1 and 2), or Study Day 180+/−5) rostral brain tissue from was processed for LC-MS analysis to determine the effect of AAVhu68.CB7.CI.hARSAco.rBG (GTP-207) treatment on storage of multiple sulfatide species. Bars represent group means. *p<0.05, **p<0.01, ***p<0.001 ****p<0.001 1-way ANOVA and post hoc Dunn's multiple comparisons test (each group compared to Arsa^{-/-} vehicle).

FIGS. 91A and 91B show quantification of sulfatides in the spinal cord of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (Groups 1 and 2), or Study Day 180+/−5) spinal cord was collected and processed to assess the effect of AAVhu68.CB7.CI.hARSAco.rBG (GTP-207) treatment on storage of multiple sulfatide species. Bars represent group means. *p<0.05, ****p<0.001 1-way ANOVA and post hoc Dunn's multiple comparisons test (each group compared to Arsa^{-/-} vehicle).

FIGS. 92A to 92C show quantification of sulfatides in the liver of Arsa^{-/-} mice administered AAVhu68.CB7.CI.hARSAco.rBG (GTP-207) or vehicle. At 4 months of age, Arsa−/− mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (Groups 1 and 2), or Study Day 180+/−5) a piece of liver was collected and used for LC-MS analysis to determine the effect of AAVhu68.CB7.CI.hARSAco.rBG (GTP-207) treatment on storage of multiple sulfatide species. Bars represent group means. *p<0.05, **p<0.01, ***p<0.001 ****p<0.001 1-way ANOVA and post hoc Dunn's multiple comparisons test (each group compared to Arsa^{-/-} vehicle).

FIG. 93 shows quantification of sulfatides in the kidney of Arsa^{-/-} mice administered AAVhu68.CB7.CI.hARSAco.rBG (GTP-207) or vehicle. At 4 months of age, Arsa^{-/-} mice were ICV-administered AAVhu68.CB7.CI.hARSAco.rBG (GTP-207) at a dose of 1.3×10¹¹ GC, 4.5×10¹⁰ GC, 1.3×10¹⁰ GC, or 4.5×10⁹ GC (Study Day 0). Age-matched Arsa^{-/-} mice and WT mice were ICV-administered vehicle (ITFFB) as controls. At necropsy (Baseline Study Day 0 (Groups 1 and 2), or Study Day 180+/−5), one kidney was collected and used for LC-MS analysis to determine the effect of AAVhu68.CB7.CI.hARSAco.rBG (GTP-207) treatment on storage of multiple sulfatide species. Bars represent group means. *p<0.05, **p<0.01, ***p<0.001 ****p<0.001 1-way ANOVA and post hoc Dunn's multiple comparisons test (each group compared to Arsa^{-/-} vehicle).

FIG. 94 shows a typical sensory nerve action potential waveform. A typical median nerve SNAP recorded from digit II of a healthy NHP. Sensory nerve conduction velocity was calculated by dividing the physical distance between the stimulation cathode and the recording site at digit II by the onset latency (i.e., the time between the stimulus and the onset of the SNAP). The SNAP amplitude was calculated as the difference in electrical voltage at the SNAP onset versus the SNAP peak. Abbreviations: NHP, non-human primate; SNAP, sensory nerve action potential.

FIGS. 95A and 95B show sensory nerve action potential (SNAP) amplitudes and nerve conduction velocities, respectively, in NHPs following ICM administration of AAVhu68.CB7.CI.hARSAco.rBG (GTP-207) (Day 90 Cohort). Juvenile NHPs received a single ICM administration of either vehicle (ITFFB; N=1) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Sensory nerve conduction testing was performed at BL and on Days 28±3, 60±3, and 90±4. SNAP amplitudes and conduction velocities of the right and left median nerves are presented. The shaded areas (8.5-58.4 pV for SNAP amplitude and 40.3-53.5 m/s for velocity) indicate values within two standard deviations of the baseline average of all animals in the study.

FIGS. 96A and 96B show SNAP amplitudes and nerve conduction velocities, respectively, in NHPs following ICM Administration of AAVhu68.CB7.CI.hARSAco.rBG (GTP-207) (Day 180 Cohort). Juvenile NHPs received a single ICM administration of either vehicle (ITFFB; N=1) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Sensory nerve conduction testing was performed at BL and on Days 28±3, 60±3, 90±4, 120±4, 150±4, and 180±5. SNAP amplitudes and conduction velocities of the right and left median nerves are presented. The shaded areas (8.5-58.4 pV for SNAP amplitude and 40.3-53.5 m/s for velocity) indicate values within two standard deviations of the baseline average of all animals in the study.

FIGS. 97A and 97B show body weights of NHPs following ICM administration of AAVhu68.CB7.CI.hARSAco.rBG (GTP-207) in a 90 day cohort (FIG. 97A) or an 180 day cohort (FIG. 97B). Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Body weights were monitored at BL and on Days 0, 7±1, 14±2, 28±3, 60±3, 90±4, 120±4, 150±4, and 180±5.

FIGS. 98A and 98B shows alanine aminotransferase levels in NHPs following ICM administration of AAVhu68.CB7.CI.hARSAco.rBG (GTP-207) in a 90 day cohort (FIG. 98A) or an 180 day cohort (FIG. 98B). Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Serum was collected at BL and on Days 0, 7±1, 14±2, 28±3, 60±3, 90±4, 120±4, 150±4, and 180±5. Alanine aminotransferase (ALT) levels were measured.

FIGS. 99A and 99B show leukocyte counts in cerebrospinal fluid of NHPs following ICM administration of AAVhu68.CB7.CI.hARSAco.rBG (GTP-207) in a 90 day cohort (FIG. 99A) or an 180 day cohort (FIG. 99B). Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). CSF was collected on Days 0, 7±1, 14±2, 28±3, 60±3, 90±4, 120±4, 150±4, and 180±5. Leukocytes were quantified as the number of WBCs per μl of CSF.

FIGS. 100A to 100C show DRG/TRG neuronal degeneration severity scores after ICM administration of AAVhu68.CB7.CI.hARSAco.rBG (GTP-207) to NHPs in a 90 day cohort (FIG. 100A), a day 180 cohort (FIG. 100B); FIG. 100C shows day 90 and Day 180 cohorts. Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Severity grade scores for all ITFFB- and AAVhu68.CB7.CI.hARSAco.rBG (GTP-207)-treated animals necropsied on Day 90 or Day 180 are presented in each DRG segment (cervical, thoracic, and lumbar) and in TRG for findings of neuronal degeneration/necrosis in the ganglion. For each DRG segment and TRG, the following scores were assigned: Severity Grade 1=minimal, Severity Grade 2=mild, Severity Grade 3=moderate, Severity Grade 4=marked; Severity Grade 5=severe. *p<0.05 based on a Kruskal-Wallis test followed by Dunn's multiple comparison test.

FIGS. 101A to 101C show spinal cord axonopathy severity scores after ICM administration of AAVhu68.CB7.CI.hARSAco.rBG (GTP-207) to NHPs in a 90 day cohort (FIG. 101A), a day 180 cohort (FIG. 101B); FIG. 101C shows day 90 and Day 180 cohorts. Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Severity grade scores for all ITFFB- and AAVhu68.CB7.CI.hARSAco.rBG (GTP-207)-treated animals necropsied on Day 90 or Day 180 are presented for axonopathy in the dorsal white matter tracts of the spinal cord (cervical, thoracic, and lumbar segments). For each finding, the following scores were assigned: Severity Grade 1=minimal, Severity Grade 2=mild, Severity Grade 3=moderate, Severity Grade 4=marked; Severity Grade 5=severe. *p<0.05, **p<0.01, and ****p<0.0001 based on a Kruskal-Wallis test followed by a multiple comparisons Dunn's test comparing each GTP-207-treated Group to the vehicle-treated control group.

FIGS. 102A to 102C show peripheral nerve axonopathy severity scores after ICM administration of AAVhu68.CB7.CI.hARSAco.rBG (GTP-207) to NHPs in a 90 day cohort (FIG. 102A), a day 180 cohort (FIG. 102B); FIG. 102C shows day 90 and Day 180 cohorts. Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Severity grade scores for all ITFFB- and AAVhu68.CB7.CI.hARSAco.rBG (GTP-207)-treated animals necropsied on Day 90 or Day 180 are presented for axonopathy in the peripheral nerves (left and right proximal median nerves, distal median nerves, peroneal nerves, sciatic nerves, and tibial nerves—8 nerves and 10 scores per animal). For each finding, the following scores were assigned: Severity Grade 1=minimal, Severity Grade 2=mild, Severity Grade 3=moderate, Severity Grade 4=marked; Severity Grade 5=severe. *p<0.05, **p<0.01, and ****p<0.0001 based on a Kruskal-Wallis test followed by a multiple comparisons Dunn's test comparing each AAVhu68.CB7.CI.hARSAco.rBG (GTP-207)-treated Group to the vehicle-treated control group.

FIGS. 103A and 103B show vector pharmacokinetics as determined by measuring vector genome DNA concentration in cerebrospinal fluid (CSF) and serum (Blood) of NHPs following ICM administration of AAVhu68.CB7.CI.hARSAco.rBG (GTP-207). Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). CSF and blood were collected on Days 0, 7±1, 14±2, 28±3, 60±3, 90±4, and 180±5. AAVhu68.CB7.CI.hARSAco.rBG (GTP-207) vector genomes were quantified by TaqMan qPCR. The dashed lines indicate the LOD of the assay (CSF: 25 copies/12 L; blood: 50 copies/g DNA).

FIGS. 104A and 104B show vector excretion in urine (FIG. 104A) and feces (FIG. 104B) of NHPs following ICM administration of AAVhu68.CB7.CI.hARSAco.rBG (GTP-207), as measured using vector genome DNA concentration. Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Urine and feces were collected at BL and on Days 5±2, 28±3, 60±3, 90±4, 120±4, 150±4, and 180±5. AAVhu68.CB7.CI.hARSAco.rBG (GTP-207) vector genomes were quantified by TaqMan qPCR. The dashed lines indicate the LOD of the assay (urine: 25 copies/12 μL; feces: 50 copies/g DNA).

FIGS. 105A and 105B shows transgene product expression (ARSA enzyme activity) in serum (FIG. 105A) and cerebrospinal fluid (CSF, FIG. 105B) of NHPs following ICM administration of AAVhu68.CB7.CI.hARSAco.rBG (GTP-207). Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Serum and CSF were collected at the indicated days and analyzed for transgene product expression (ARSA enzyme activity). Error bars represent the standard deviation.

FIGS. 106A and 106B show transgene product expression (ARSA Enzyme Activity) in serum (Day 14; FIG. 106A) and cerebrospinal fluid (FIG. 106B, Day 7) of NHPs following ICM administration of AAVhu68.CB7.CI.hARSAco.rBG (GTP-207). Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). Serum collected on Day 14 and CSF collected on Day 7 were analyzed for transgene product expression (ARSA enzyme activity). Empty shapes indicate animals that were negative for serum-circulating NAbs against the vector capsid at the time of treatment, while shaded cells indicate animals that were positive for serum-circulating NAbs against the vector capsid at the time of treatment. Error bars represent the standard deviation.

FIGS. 107A and 107B show antibodies against the transgene product (anti-human ARSA antibodies) in serum and cerebrospinal fluid of NHPs following ICM administration of AAVhu68.CB7.CI.hARSAco.rBG (GTP-207). Juvenile NHPs received a single ICM administration of either vehicle (ITFFB; N=1/group) or AAVhu68.CB7.CI.hARSAco.rBG (GTP-207) at a dose of 3.0×10¹² GC (low dose), 1.0×10¹³ GC (mid-dose), or 3.0×10¹³ GC (high dose) (N=3/group). CSF and serum were collected on the indicated days, and antibodies against the transgene product (anti-human ARSA antibodies) were measured by ELISA. Error bars represent the standard deviation.

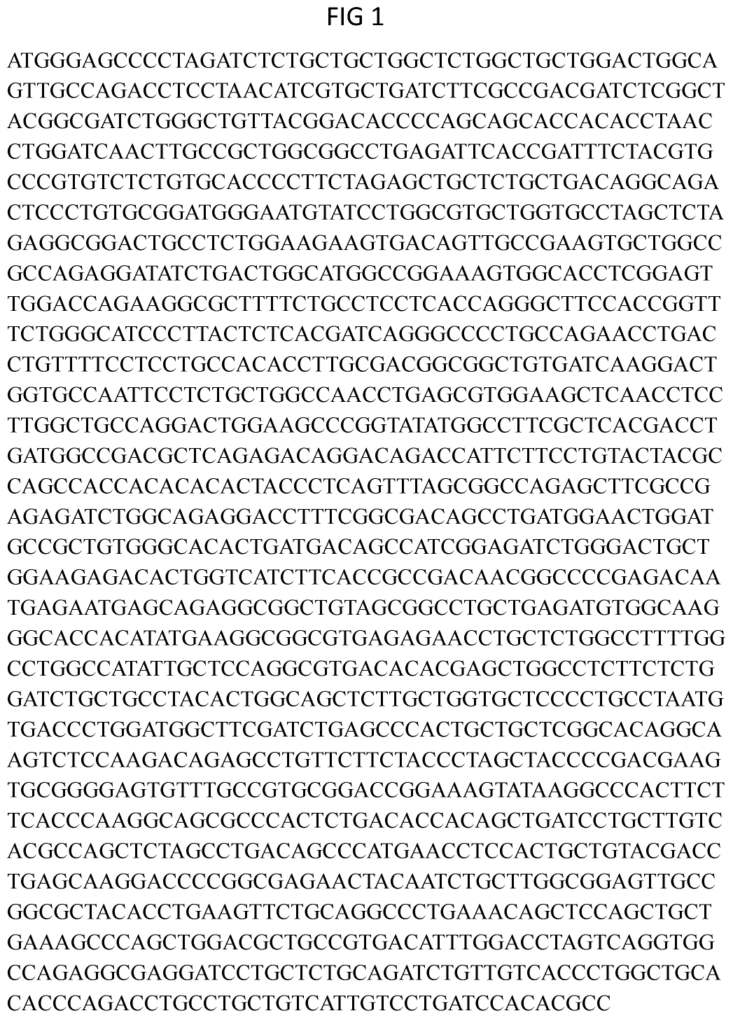

### DETAILED DESCRIPTION OF THE INVENTION

Compositions and methods for treating a disease caused by mutation(s) in the Arylsulfatase A (ARSA) gene and/or deficiencies in normal levels of functional Arylsulfatase A (e.g., Metachromatic Leukodystrophy (MLD)) are provided herein. In certain embodiments, also provided are compositions and methods for treating disease(s) or symptom(s) caused by mutation(s) in the ARSA gene and/or deficiencies in normal levels of functional Arylsulfatase A. An effective amount of a recombinant adeno-associated virus (rAAV) having an AAVhu68 capsid and packaged therein a vector genome encoding a functional human Arylsulfatase A (hARSA) protein is delivered to a subject in need. Desirably, this rAAV is formulated with an aqueous buffer. In certain embodiments, the suspension is suitable for intrathecal injection. In certain embodiments, the rAAV vector is termed as AAVhu68.hARSAco, in which the hARSA coding sequence is an engineered hARSA coding sequence (termed as “hARSAco” or “hARSA” unless specified, for example, nucleotide (nt) 55 to nt 1521 of SEQ ID NO: 1, SEQ ID NO: 3, or a sequence at least about 95% to about 99.9% identical thereto). In certain embodiment, the hARSAco is SEQ ID NO: 1. In certain embodiment, the hARSAco is SEQ ID NO: 3. In certain embodiments, the rAAV vector is termed AAVhu68.CB7.hARSAco, in which the engineered hARSA coding sequence is under the control of regulatory sequences which include a CB7 promoter. As used herein, a CB7 promoter or promoter element refers to a human cytomegalovirus (CMV) immediate early (IE) enhancer, including a C4 enhancer, a chicken beta actin (CB) promoter, optionally an intron, and optional spacer sequences linking the elements. See, e.g., a promoter comprising the CB7 having the sequence of SEQ ID NO: 16. In certain embodiments, a CB7 promoter or promoter element refers to a human cytomegalovirus (CMV) immediate early (IE) enhancer, a chicken beta actin (CB) promoter, an intron which comprises chicken beta actin intron with rabbit beta globin splicing donor (i.e., chimeric intron), and optional spacer sequences linking the elements of the hybrid promoter. In certain embodiments, a CB7 promoter or promoter element refers to a human cytomegalovirus (CMV) immediate early (IE) enhancer (SEQ ID NO: 19), a chicken beta actin (CB) promoter (SEQ ID NO: 18), optionally an intron (SEQ ID NO: 17), and optional spacer sequences linking the elements of the hybrid promoter. In certain embodiments, a CB7 promoter or promoter element refers to a human cytomegalovirus (CMV) immediate early (IE) enhancer (SEQ ID NO: 31), a chicken beta actin (CB) promoter (SEQ ID NO: 32), optionally a chimeric intron (SEQ ID NO: 33), and optional spacer sequences linking the elements of the hybrid promoter. In certain embodiments, a CB7 promoter or promoter element comprises the nucleic acid sequence of SEQ ID NO: 29. In certain embodiments, a CB7 promoter or promoter element comprises the nucleic acid sequence of SEQ ID NO: 30. Preferably, the spacer sequences are non-coding and in certain embodiments, may be of different lengths. In certain embodiments, the compositions are delivered intrathecally. In certain embodiments, the intrathecal administration is an intra-cisterna magna injection (ICM).

Nucleic acid sequences encoding capsid of a clade F adeno-associated virus (AAV), which is termed herein AAVhu68, are utilized in the production of the AAVhu68 capsid and recombinant AAV (rAAV) carrying the vector genome. Additional details relating to AAVhu68 are provided in WO 2018/160582 and in this detailed description. The AAVhu68 vectors described herein are well suited for delivery of the vector genome comprising the engineered hARSA coding sequence to cells within the central nervous system (CNS), including brain, hippocampus, motor cortex, cerebellum, and motor neurons, and the peripheral nervous system (PNS), including nerves and ganglia outside the brain and the spinal cord. These vectors may be used for targeting other cells within the CNS and/or PNS and certain other tissues and cells, for example, kidney or liver or gallbladder.

I. Arylsulfatase A (hARSA)

Arylsulfatase A (ARSA) has an enzymatic activity of hydrolyzing cerebroside sulfate (i.e., the following reaction: a cerebroside 3-sulfate+H₂O=a cerebroside+sulfate). Two isoforms of human ARSA (hARSA) protein (UniProtKB-P15289, ARSA_HUMAN) have been identified: P51608-1, SEQ ID NO: 2; and P51608-2, SEQ ID NO: 15. Throughout this specification, reference to ARSA is hARSA unless otherwise specified.

As used herein, a functional hARSA protein refers to an isoform, a natural variant, a variant, a polymorph, or a truncation of a hARSA protein which has at least about 10% of the enzymatic activity (i.e., enzyme activity) of the wildtype hARSA protein (for example, P51608-1, SEQ ID NO: 2; or P51608-2, SEQ ID NO: 15). See, OMIM #607574 (omim.org/entry/607574), genecards.org/cgi-bin/carddisp.pl?gene=ARSA and uniprot.org/uniprot/P15289, each of the webpages is incorporated herein by reference in its entirety. In certain embodiments, the functional hARSA protein has at least about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 1.5-fold, 2-fold, 3-fold, 4-fold, 5-fold, 10-fold or more of the enzymatic activity of the wildtype hARSA protein (for example, P51608-1, SEQ ID NO: 2; or P51608-2, SEQ ID NO: 15). In certain embodiments, the functional hARSA protein has about 10% to about 15%, about 10% to about 20%, about 10% to about 25%, about 10% to about 30%, about 10% to about 50%, about 10% to about 75%, about 10% to about 90%, about 10% to about 100%, about 10% to about 3-fold, about 15% to about 20%, about 15% to about 25%, about 15% to about 30%, about 15% to about 50%, about 15% to about 75%, about 15% to about 90%, about 15% to about 100%, about 15% to about 3-fold, about 20% to about 25%, about 20% to about 30%, about 20% to about 50%, about 20% to about 75%, about 20% to about 90%, about 20% to about 100%, about 20% to about 3-fold, about 25% to about 30%, about 25% to about 50%, about 25% to about 75%, about 25% to about 90%, about 25% to about 100%, about 25% to about 3-fold, about 50% to about 75%, about 50% to about 90%, about 50% to about 100%, about 50% to about 3-fold, about 75% to about 90%, about 75% to about 100%, or about 75% to about 3-fold of the enzymatic activity of the wildtype hARSA protein (for example, P51608-1, SEQ ID NO: 2; or P51608-2, SEQ ID NO: 15). Method(s) of measuring the hARSA enzymatic activity (for example, via synthetic substrate-based assays and/or via sulfatide loading assay) can be found in the Examples as well as in various publications, such as Kreysing et al., High residual arylsulfatase A (ARSA) activity in a patient with late-infantile metachromatic leukodystrophy. Am J Hum Genet. 1993 August; 53(2):339-46; Lee-Vaupel M and Conzelmann E. A simple chromogenic assay for arylsulfatase A. Clin Chim Acta. 1987 Apr. 30; 164(2):171-80; Bohringer et al., Enzymatic characterization of novel arylsulfatase A variants using human arylsulfatase A-deficient immortalized mesenchymal stromal cells. Hum Mutat. 2017 November; 38(11):1511-1520. doi: 10.1002/humu.23306. Epub 2017 Sep. 6; and Francesco Morena, et al., A new analytical bench assay for the determination of arylsulfatase a activity toward galactosyl-3-sulfate ceramide: implication for metachromatic leukodystrophy diagnosis. Anal Chem. 2014 Jan. 7; 86(1):473-81. doi: 10.1021/ac4023555. Epub 2013 Dec. 11.

In certain embodiments, the functional hARSA protein comprises (i) a signal peptide, and (ii) an amino acid sequence of amino acid (aa) 19 to aa 507 of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In certain embodiments, the functional hARSA protein comprises (i) a signal peptide, and (ii) an amino acid sequence of SEQ ID NO: 15 (i.e., aa 85 to aa 507 of SEQ ID NO: 2) or an amino acid sequence at least about 90% (e.g., at least about 90%, 91%, 92%, 93%, 94%, 95%, 96%, 97%, 98%, 99%, or 99.9%) identical thereto. In certain embodiments, the functional hARSA protein comprises (i) a signal peptide, (ii) an amino acid sequence of amino acid (aa) 19 to aa 444 of SEQ ID NO: 2 or an amino acid sequence at least about 90% (e.g., at least about 90%, 91%, 92%, 93%, 94%, 95%, 96%, 97%, 98%, 99%, or 99.9%) identical thereto, and (iii) an amino acid sequence of aa 448 to aa 507 of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In a further embodiment, the amino acid sequence of (ii) may be linked to the amino acid sequence of (iii) by disulfide bond(s). Other chemical bond(s) may be utilized, for example, covalent bond, and noncovalent bond (including hydrogen, ionic, hydrophobic, and Van Der Waals bonding). In yet a further embodiment, the link between the amino acid sequences of (ii) and (iii) is formed by a combination of the bonds described. In another embodiment, the link between the amino acid sequences of (ii) and (iii) is a peptide linker (see, e.g., parts.igem.org/Protein_domains/Linker). In certain embodiments, the functional hARSA protein comprises (i) a signal peptide, (ii) an amino acid sequence of amino acid (aa) 85 to aa 444 of SEQ ID NO: 2 or an amino acid sequence at least about 90% (e.g., at least about 90%, 91%, 92%, 93%, 94%, 95%, 96%, 97%, 98%, 99%, or 99.9%) identical thereto, and (iii) an amino acid sequence of aa 448 to aa 507 of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In a further embodiment, the amino acid sequence of (ii) may be linked to the amino acid sequence of (iii) by disulfide bond(s). Other chemical bond(s) may be utilized, for example, covalent bond, and noncovalent bond (including hydrogen, ionic, hydrophobic, and Van Der Waals bonding). In yet a further embodiment, the link between the amino acid sequences of (ii) and (iii) is formed by a combination of the bonds described. In another embodiment, the link between the amino acid sequences of (ii) and (iii) is a peptide linker (see, e.g., parts.igem.org/Protein_domains/-Linker). In certain embodiments, the functional hARSA protein comprises (i) a signal peptide, and (ii) an amino acid sequence of amino acid (aa) 23 to aa 348 of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In certain embodiments, the functional hARSA protein comprises (i) a signal peptide, and (ii) an amino acid sequence of amino acid (aa) 19 to aa 448 of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In certain embodiments, the functional hARSA protein comprises (i) a signal peptide, and (ii) an amino acid sequence of amino acid (aa) 448 to aa 507 of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In certain embodiments, the functional hARSA protein with the identity specified has its modifications outside of the aa 85 to aa 507 based on the numbering in SEQ ID NO: 2, and/or outside of any one or more of the aa 29, 69, 123, 125, 150, 229, 281, 282 based on the numbering in SEQ ID NO: 2, and/or outside of any of hARSA conserved domain(s) (for example, the sulfatase domain with Pfam:PF00884), and/or outside of aa 19 to aa 444 based on the numbering in SEQ ID NO: 2, and/or outside of aa 448 to aa 507 based on the numbering in SEQ ID NO: 2, and/or outside of aa 23 to aa 348 based on the numbering in SEQ ID NO: 2 or any combination thereof. See. e.g., von Bülow R et al, Crystal structure of an enzyme-substrate complex provides insight into the interaction between human arylsulfatase A and its substrates during catalysis, J Mol Biol. 2001 Jan. 12; 305(2):269-77.

In certain embodiments, the functional hARSA protein has an amino acid sequence of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In certain embodiment, the functional hARSA protein has an amino acid sequence of SEQ ID NO: 4 or an amino acid sequence at least about 90% (e.g., at least about 90%, 91%, 92%, 93%, 94%, 95%, 96%, 97%, 98%, 99%, or 99.9%) identical thereto.

As used herein, a signal peptide (sometimes referred to as signal sequence, targeting signal, localization signal, localization sequence, transit peptide, leader sequence or leader peptide) is a short peptide (usually 15-30 amino acids long) present at the N-terminus of the majority of newly synthesized proteins that are destined towards the secretory pathway (Blobel G, Dobberstein B (December 1975). “Transfer of proteins across membranes. I. Presence of proteolytically processed and unprocessed nascent immunoglobulin light chains on membrane-bound ribosomes of murine myeloma”. J Cell Biol. 67 (3): 835-51). These proteins include those that reside either inside certain organelles (the endoplasmic reticulum, golgi or endosomes), secreted from the cell, or inserted into most cellular membranes. In certain embodiments, the signal peptide has an amino acid sequence of aa 1 to aa 18 of SEQ ID NO: 2 or an amino acid sequence of aa 1 to aa 20 of SEQ ID NO: 4. In certain embodiments, the signal peptide is from another protein which is secreted by a CNS cell (for example, a neuron), a PNS cell, or another cell (such as a kidney cell, or a liver cell). The signal peptide is preferably of human origin or a derivative of a human signal peptide, and is about 15 to about 30 amino acids, preferably about 17 to 25 amino acids, or about 18 amino acids in length. In certain embodiments, the signal peptide is the native signal peptide (amino acids 1 to 18 of SEQ ID NO: 2). In certain embodiments, the functional hARSA protein comprises an exogenous leader sequence in the place of the native signal peptide. In another embodiment, the signal peptide may be from a human IL2 or a mutated signal peptide. In another embodiment, a human serpinF1 secretion signal may be used as a signal peptide. Such chimeric hARSA proteins comprising an exogenous signal peptide and the mature portion of the hARSA (e.g., aa 19 to 507 of SEQ ID NO:2, aa 19 to aa 444 of SEQ ID NO: 2, aa 85 to aa 507 of SEQ ID NO: 2, aa 23 to aa 348 of SEQ ID NO: 2, or aa 448 to 507 of SEQ ID NO: 2) is included in the various embodiments described herein when reference is made to a functional hARSA protein.

Provided herein is a nucleic acid sequence encoding a functional hARSA protein, termed as hARSA coding sequence or ARSA coding sequence or hARSA or ARSA. In certain embodiments, the hARSA coding sequence is a modified or engineered (hARSA or hARSAco). In certain embodiments, the hARSA coding sequence has a sequence of nucleotide (nt) 55 to nt 1521 of SEQ ID NO: 1, or a sequence at least 95% to 99.9% identical thereto. In certain embodiments, the hARSA coding sequence is nt 55 to nt 1521 of SEQ ID NO: 1 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto. In certain embodiments, the hARSA coding sequence is SEQ ID NO: 1 or a sequence at least 95% to 99.9% identical thereto. In certain embodiments, the hARSA coding sequence is SEQ ID NO: 1 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto. In certain embodiments, the hARSA coding sequence is SEQ ID NO: 3 or a sequence at least 95% to 99.9% identical thereto. In certain embodiments, the hARSA coding sequence is SEQ ID NO: 3 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto.

Transcript variants of hARSA (which is also hARSA coding sequence) can be found as NCBI Reference Sequences NM_000487.5, NM_001085425.2, NM_001085426.2, NM_001085427.2, NM 001085428.2, NM 001362782.1, AB448736.1, AK092752.1, AK098659.1, AK301098.1, AK310564.1, AK315011.1, BC014210.2, B1770997.1, BM818814.1, BP306351.1, BQ184813.1, BU632196.1, BX648618.1, CA423492.1, CN409235.1, CR456383.1, DA844740.1, DB028013.1, GQ891416.1, KU177918.1, KU177919.1, and X52151.1. Each of the NCBI Reference Sequences is incorporated herein by reference in its entirety. In certain embodiments, the modified or engineered hARSA coding sequence shares less than about 70%, 75%, 80%, 85%, 90%, 91%, 92%, 93%, 94%, 95%, 96%, 97%, 98%, 99%, or 99.9% identity to one of the NCBI Reference Sequences. In certain embodiments, the modified or engineered hARSA coding sequence shares about 50%, 60%, 70%, 75%, 80%, 85%, 90%, 91%, 92%, 93%, 94%, 95%, 96%, 97%, 98%, 99%, or 99.9% identity to one of the NCBI Reference Sequences.

A “nucleic acid” or a “nucleotide”, as described herein, can be RNA, DNA, or a modification thereof, and can be single or double stranded, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “Clustal Omega” “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “Clustal Omega”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

### II. Metachromatic Leukodystrophy (MLD)

Provided herein are rAAV, vector, methods and compositions useful in treating a disease or an abnormal condition caused by mutation(s) of Arylsulfatase A (ARSA) gene and/or deficiencies in normal levels of functional Arylsulfatase A, termed as “disease” herein, for example, Metachromatic leukodystrophy (MLD). See, e.g., omim.org/entry/250100.

Metachromatic Leukodystrophy (MLD) can be classified into the following types: early onset MLD which includes infantile MLD (typically begins equal to or earlier than 30 months of age) and early juvenile MLD (usually begins between 30 months of age to 6 years of age (including 6 years); juvenile MLD which includes early juvenile MLD and late juvenile MLD (usually begins between 7 years of age and 16 years of age, including 16 year old); and adult MLD (with an onset later than 16 years of age). Late infantile MLD patients have a devastating disease course with rapid and predictable decline that is homogeneous in the presentation of both motor and cognitive impairment (Kehrer et al., 2011a; Sessa et al., 2016). The majority of these children die before 5 years of age with a mean survival in 98 patients of 4.2 years and a 5 year survival of 25%. The phenotype of children with early juvenile MLD (symptom onset between months and 6 years of age) is very similar to that of children with late infantile MLD, although early juvenile MLD patients may have a less rapid initial disease evolution (Biffi et al., 2008; Chen et al., 2016; Sessa et al., 2016). However, once overt symptoms appear, in particular when early juvenile MLD patients lose the ability to walk independently, their disease course can deteriorate as rapidly as late infantile MLD patients. These children also have similar signs and symptoms as late infantile MLD patients with neuromuscular difficulties developing first, either in isolation or concurrent with behavioral and cognitive symptoms (Groeschel et al., 2011; Kehrer et al., 2014). The early juvenile and late infantile phenotypes are collectively referred to as early onset MLD (Sessa et al., 2016).

In certain embodiments, the rAAV, vector, composition and method described herein are useful in treating MLD, early onset MLD, infantile MLD, late infantile MLD, juvenile MLD, early juvenile MLD, late juvenile MLD, or adult MLD. In certain embodiments, the rAAV, vector, compositions and methods described herein may ameliorate disease symptom and/or delay disease progression in a subject. In certain embodiments, the rAAV, vector, compositions and methods described herein are useful in treating late infantile and early juvenile MLD.

In certain embodiments, the subject or patient of the rAAV, vector, method or composition described herein has MLD, or is diagnosed with MLD. In certain embodiments, the subject or patient of rAAV, vector, the method or composition described herein is diagnosed with late infantile MLD or early juvenile MLD. The diagnosis of MLD may be made through both genetic and biochemical testing. Genetic testing can identify mutations in the ARSA, while biochemical testing includes sulfatase enzyme activity and urinary sulfatide excretion. An magnetic resonance imaging (MRI) can confirm a diagnosis of MLD. An MRI shows imaging of a person's brain and can show the presence and absence of myelin. There is a classic pattern of myelin loss in the brains of individuals affected by MLD. As the disease progresses, imaging shows accumulating injury to the brain. In young children, the initial brain imaging can be normal.

In certain embodiments, the subject of the rAAV, vector, method or composition described herein is a human less than 18 years old (e.g., less than about 1, 2, 3, 4, 5, 6, 7, 8, 9, 10, 11 month(s) old, or less than about 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18 year(s) old). Additionally or alternatively, the subject is a newborn or a human more than 1 month old (e.g., more than about 2, 3, 4, 5, 6, 7, 8, 9, 10, 11 month(s) old, or more than about 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18 year(s) old). In certain embodiments, the patient is about 1, 2, 3, 4, 5, 6, 7, 8, 9, 10, 11 month(s) old, or about 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18 year(s) old. In certain embodiments, the patient is about 30 months to about 7 years of age. In certain embodiments, the patient is from about 30 months to 16 years of age, from 7 years to 16 years of age, or from 16 years to 40 years of age.

“Patient” or “subject”, as used herein interchangeably, means a male or female mammalian animal, including a human, a veterinary or farm animal, a domestic animal or pet, and animals normally used for clinical research. In one embodiment, the subject of these rAAV, vector, methods and compositions is a human patient. In one embodiment, the subject of these rAAV, vector, methods and compositions is a male or female human. In certain embodiments, the subject of these rAAV, vector, methods and compositions is diagnosed with Metachromatic Leukodystrophy and/or with symptoms of Metachromatic Leukodystrophy.

Disease symptoms (e.g., MLD symptoms, compared to a healthy control without MLD) may include, but are not limited to the following: decreased concentration and/or level and/or biological activity of ARSA (for example, in serum or in CSF), increased urine sulfatides, CNS myelination (demyelination load and pattern), white matter atrophy as measured by MRI, an abnormal (decreased or increased) neuronal metabolite N-acetylaspartate (NAA), myo-inositol (ml), choline (Cho) and/or lactate (Lac) levels (for example, as measured by proton magnetic resonance spectroscopy (MRS)), increased CSF sulfatide and lyso-sulfatide levels, abnormal Visual evoked potentials (VEPs), abnormal Brainstem auditory evoked responses (BAERs), gall-bladder wall thickening (for example, via ultrasound evaluation); impaired motor function (for example, measured by the Gross Motor Function Classification for Metachromatic Leukodystrophy (GMFC-MLD) or Gross Motor Function Measure (GMFM)), delayed Motor milestones achievement (as defined by World Health Organization [WHO] criteria) assessed by age at achievement, age at loss, and percentage of children maintaining or acquiring motor milestones, impaired cognitive function (for example, Total Intelligence Quotient [IQ] and sub-domain IQ measured by the Bayley Scale of Infant Development [BSID-III], Wechsler Intelligence Scale for Children, Fifth Edition [WISC-V]), increased lifespan (compared to a patient), an abnormal result of neurological clinical exam (NCE), a reduced nerve conduction velocity (NCV) of the ulnar, deep peroneal, median, sural nerves, an earlier age-at-onset and higher frequency of seizures captured by a seizure diary, impaired behavior function (for example, measured by Vineland Adaptive Behavior Scales, Third Edition (Vineland-III)), a lower Lansky Performance Index, a decreased Pediatric Quality of Life Inventory (for example, PedsQL and PedsQL-IS), and/or a decreased caregiver/parent quality of life.

In certain embodiments, disease symptoms (e.g., MLD symptoms, compared to a healthy control without MLD) may include abnormal properties (for example biomarker activity, electrophysiological activity, and/or imaging parameters) and clinical observations (for example, impaired gross and fine motor function, impaired cognitive and language development, abnormal neurological exam findings, impaired behavioral and milestone development, and caregiver/parent-reported outcomes and decreased quality of life assessments).

The abnormal properties include but are not limited to functional impairment of myelin-producing oligodendrocytes and Schwann cells, peripheral nerve conduction abnormalities, peripheral neuropathy with slow nerve conduction velocities (NCVs), brain magnetic resonance imaging (MRI) showing a typical white matter (for example, the splenium of the corpus callosum and parieto-occipital white matter, projection fibers, cerebellar white matter, basal ganglia, and the thalamus) pattern (for example, a “tigroid pattern” of radiating stripes with bands of normal signal intensity within the abnormal white matter, see, e.g., Gieselmann and Krageloh-Mann, 2010; Martin et al., 2012; van Rappard et al., 2015); U-fiber involvement and cerebellar changes, white matter demyelination, bilateral areas of white matter hypodensity, especially in the frontal lobes, and cerebral atrophy reflecting loss of myelin), abnormal levels of the brain biomarkers N-acetylaspartate and myo-inositol.

The clinical observations include but are not limited to gross motor disturbances that manifest as clumsiness, toe walking, and frequent falls; fine motor skills; gait abnormalities; spastic paraparesis or ataxic movement; neuromuscular difficulties; neurologic symptoms (signs of weakness, loss of coordination progressing to spasticity and incontinence); hypotonia, and depressed deep tendon reflexes; seizures; dementia; epilepsy; difficulty urinating spasticity; feeding difficulties; pain in the extremities; impaired language function; impaired cognitive skills; impaired vision and hearing; losing previously acquired motor and cognitive milestones; decline in school or job performance, inattention, abnormal behaviors, psychiatric symptoms, intellectual impairment, uncontrolled laughter, cortical disturbances (e.g., apraxia, aphasia, agnosia), alcohol or drug use, poor money management, emotional lability, inappropriate affect, and neuropsychiatric symptoms (including psychosis, schizophrenia, delusions, and hallucinations).

Disease progression refers to subject's age of onset, frequency of appearance, severity, or recurrence, of a disease symptom. A delay in disease progression normally means an elevated age of onset, a lower frequency of appearance, a decreased severity, or less recurrence, of a disease symptom.

As described above, the terms “increase” “decrease” “reduce” “ameliorate” “elevate” “lower” “higher” “less” “more” “improve” “delay” “impair” “abnormal” “thick” or any grammatical variation thereof, or any similar terms indication a change, means a variation of about 5 fold, about 2 fold, about 1 fold, about 90%, about 80%, about 70%, about 60%, about 50%, about 40%, about 30%, about 20%, about 10%, about 5% compared to the corresponding reference (e.g., untreated control or a subject in normal condition without MLD), unless otherwise specified.

The compositions and methods herein provide a fast-acting, disease-modifying treatment to symptomatic early onset patients for whom no standard of care exists (HSCT and HSC-GT are not efficacious); and/or provide a therapy that can preserve or correct both CNS pathologies and peripheral nerve function, the latter of which is not corrected by HSCT and causes progressive fine and gross motor function loss and respiratory failure; and/or provide an alternative treatment option to HSC-GT, which requires harsh myeloablative conditioning, is only efficacious when performed prior to onset of symptoms, and may not substantially address peripheral neuropathy in all patients.

In certain embodiments, the patient receives a co-therapy for which they would not have been eligible without the rAAV, vector, composition or method described herein. Such co-therapies may include enzyme replacement therapy (ERT) and hematopoietic stem cell transplantation (HSCT) via umbilical cord blood (UCB), allogeneic peripheral blood stem cells, or allogeneic bone marrow.

Optionally, an immunosuppressive co-therapy may be used in a subject in need. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 3, 4, 5, 6, 7, or more days prior to or after the gene therapy administration. Such immunosuppressive therapy may involve administration of one, two or more drugs (e.g., glucocorticoids, prednelisone, micophenolate mofetil (MMF) and/or sirolimus (i.e., rapamycin)). Such immunosuppressive drugs may be administrated to a subject in need once, twice or for more times at the same dose or an adjusted dose. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

### III. Expression Cassette

Provided herein is a nucleic acid sequence comprising a hARSA coding sequence encoding a functional hARSA protein and regulatory sequences which directs the hARSA expression in a target cell, also termed as an expression cassette. As used herein, an “expression cassette” refers to a nucleic acid molecule which comprises a coding sequence (e.g., a hARSA coding sequence), promoter, and may include other regulatory sequences therefor. The regulatory sequences necessary are operably linked to the hARSA coding sequence in a manner which permits its transcription, translation and/or expression in target cell. As used herein, “operably linked” sequences include both expression control sequences that are contiguous with the hARSA coding sequence and expression control sequences that act in trans or at a distance to control the hARSA coding sequence. Such regulatory sequences typically include, e.g., one or more of a promoter, an enhancer, an intron, a Kozak sequence, a polyadenylation sequence, and a TATA signal. In certain embodiment, the promoter is a chicken beta actin promoter with a cytomegalovirus enhancer (CB7) promoter (e.g., nt 198 to nt 862 of SEQ ID NO: 5, also termed as hSyn or Syn herein). However, in certain embodiments, other promoters, or an additional promoter, may be selected.

In certain embodiments, the regulatory sequences direct hARSA expression in a target cell. In certain embodiment, a target cell is a nervous system cell, an oligodendrocyte, a microglia, a Central Nervous System (CNS) cell, a neuron in the CNS, a Peripheral Nervous System (PNS) cell, a Schwann cell, a macrophage in the PNS, or a cell in visceral organs (for example, a kidney cell, a liver cell and a gallbladder cell). In certain embodiment, the target cell may be a central nervous system cell. In certain embodiments, the target cell is one or more of an excitatory neuron, an inhibitory neuron, a glial cell, a cortex cell, a frontal cortex cell, a cerebral cortex cell, a spinal cord cell. In certain embodiments, the target cell is a peripheral nervous system (PNS) cell, for example a retina cell. Other cells other than those from nervous system may also be chosen as a target cell, such as a monocyte, a B lymphocyte, a T lymphocyte, a NK cell, a lymph node cell, a tonsil cell, a bone marrow mesenchymal cell, a stem cell, a bone marrow stem cell, a heart cell, an epithelium cell, a esophagus cell, a stomach cell, a fetal cut cell, a colon cell, a rectum cell, a liver cell, a kindly cell, a lung cell, a salivary gland cell, a thyroid cell, an adrenal cell, a breast cell, a pancreas cell, an islet of Langerhans cell, a gallbladder cell, a prostate cell, a urinary bladder cell, a skin cell, a uterus cell, a cervix cell, a testis cell, or any other cell which expresses a functional hARSA protein in a subject without MLD. See, genecards.org/cgi-bin/carddisp.pl?gene=ARSA&keywords=arsa #expression.

In certain embodiments, the regulatory sequences comprise a ubiquitous promoter. In certain embodiments, the regulatory sequences in the vector genome (within the expression cassette which is flanked by the ITR sequences) comprise at the 5′ end a CB7 promoter (a CMV IE enhancer (C4)+linker sequences+a CB promoter) operably linked to the hARSA sequences and at the 3′ end, a polyadenylation site. In certain embodiments, the regulatory elements further comprise one or more of at least one of a Kozak sequence, intron, a second or further enhancer, and a TATA signal.

In certain embodiments, an additional or alternative promoter sequence may be included as part of the expression control sequences (regulatory sequences), e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

In addition to a promoter, an expression cassette may contain one or more other appropriate transcription initiation sequences, transcription termination sequences, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the regulatory sequences comprise one or more expression enhancers. In one embodiment, the regulatory sequences contain two or more expression enhancers. These enhancers may be the same or may differ from one another. For example, an enhancer may include a CMV immediate early enhancer (SEQ ID NO: 19). This enhancer may be present in two copies which are located adjacent to one another. Alternatively, the dual copies of the enhancer may be separated by one or more sequences. In still another embodiment, the expression cassette further contains an intron, e.g., the chicken beta-actin intron (SEQ ID NO: 17). In certain embodiments, the intron is a chimeric intron (CI)—a hybrid intron consisting of a human beta-globin splice donor and immunoglobulin G (IgG) splice acceptor elements. Other suitable introns include those known in the art, e.g., such as are described in WO 2011/126808. Examples of suitable polyA sequences include, e.g., Rabbit globin poly A, SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619). In certain embodiments, no WPRE sequence is present.

Optionally, in certain embodiments, in addition to the hARSA coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently process^{e}d into a miRNA duplex, and further into a “mature” single stranded miRNA molecule. This mature miRNA guides a multiprotein complex, niRISC, which identifies target site, e.g., in the 3′ UTR regions, of target mRNAs based upon their complementarity to the mature miRNA.

In certain embodiments, the expression cassette may further comprises a dorsal root ganglion (drg)-specific miRNA detargetting sequences to modulate expression levels in the CNS or peripheral dorsal root ganglia. In certain embodiments, the expression cassette or vector genome comprises one or more miRNA target sequences in the untranslated region (UTR) 3′ to a gene product coding sequence. In certain embodiments, there are at least one target sequence specific for miR-183 and/or miR-182. In certain embodiments, at least two drg-specific miRNA target sequences are located in both 5′ and 3′ to the hARSA coding sequence. In certain embodiments, the miRNA target sequence for the at least first and/or at least second miRNA target sequence for the expression cassette mRNA or DNA positive strand is selected from (i) AGTGAATTCTACCAGTGCCATA (miR183, SEQ ID NO: 20); (ii) AGCAAAAATGTGCTAGTGCCAAA (SEQ ID NO: 21), (iii) AGTGTGAGTTCTACCATTGCCAAA (SEQ ID NO: 22); and (iv) AGGGATTCCTGGGAAAACTGGAC (SEQ ID NO: 23). In certain embodiments, the construct further comprises at least two tandem repeats comprise at least a first miRNA target sequence and at least a second miRNA target sequence which may be the same or different. In certain embodiments, the tandem miRNA target sequences are continuous or are separated by a spacer of 1 to 10 nucleic acids, wherein said spacer is not an miRNA target sequence. In certain embodiments, there are at least two drg-specific miRNA target sequences located at 3′ to the hARSA coding sequence. In certain embodiments, the start of the first of the at least two drg-specific miRNA tandem repeats is within 20 nucleotides from the 3′ end of the hARSA-coding sequence. In certain embodiments, the start of the first of the at least two drg-specific miRNA tandem repeats is at least 100 nucleotides from the 3′ end of the hARSA-coding sequence. In certain embodiments, the miRNA tandem repeats comprise 200 to 1200 nucleotides in length. In certain embodiments, there are at least two drg-specific miRNA target sequences located at 5′ to the hARSA coding sequence. In certain embodiments, two or more consecutive miRNA target sequences are continuous and not separated by a spacer. In certain embodiments, two or more of the miRNA target sequences are separated by a spacer and each spacer is independently selected from one or more of (A) GGAT; (B) CACGTG; or (C) GCATGC. In certain embodiments, the spacer located between the miRNA target sequences may be located 3′ to the first miRNA target sequence and/or 5′ to the last miRNA target sequence. In certain embodiments, the spacers between the miRNA target sequences are the same.

See, Provisional U.S. Patent Application No. 62/783,956, filed Dec. 21, 2018, and International Application No. PCT/US2019/067872, filed Dec. 20, 2019, which are hereby incorporated by reference. In certain embodiments, no miR sequences are included in an expression cassette or vector genome.

### IV. AAVhu68

The AAVhu68 serotype, which was selected as the capsid for AAVhu68.CB7.CI.hARSAco.RBG, has two encoded amino acid differences as compared to another Clade F capsid, AAV9, with differences at positions 67 and 157, based on the numbering of the VP1 protein, shown in SEQ ID NO: 7. In contrast, the other Clade F AAV (AAV9, hu31, hu31) have an Ala at position 67 and an Ala at position 157. In certain embodiments, the AAV capsid stereotype may be selected from AAVhu31 vp1 (SEQ ID NOs: 11 and 12) or AAVhu32 vp1 (SEQ ID NOs: 13 and 14). See, e.g., WO 2022/082109, providing engineered AAVhu68 coding sequences, WO 2018/160582; WO 2019/169004; and WO 2019/168961, all of which are incorporated herein by reference in their entireties.

AAVhu68 displays transduction characteristics in the nervous systems of NHPs and mice. This includes widespread transduction of cortical neurons (data not shown) and a small subset of myelin-producing oligodendrocytes. In addition, AAVhu68 transduces motor neurons with axons projecting into the PNS and DRG sensory neurons with axons projecting into the spinal cord and peripheral nerves (data not shown). Transduction was observed in lower motor neurons of the ventral horn and sensory neurons of the DRG. The transduced motor neurons have axons that contribute to the peripheral nerves. Thus, the AAVhu68 capsid targets cells in the CNS and PNS, which are both affected in MLD patients. Additionally, while newly synthesized ARSA can be transported directly from the trans-Golgi network to the lysosome, it can also be secreted and taken up by other cells via mannose-6-phosphate receptors where it is subsequently trafficked to the lysosomes. Thus, the underlying defect can be cross-corrected by rAAVhu68.hARSA expressing ARSA enzyme supplied to neighboring cells of the CNS that lack functional enzyme.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321.

In certain embodiments, an AAVhu68 capsid is further characterized by one or more of the following. AAVhu68 capsid proteins comprise: AAVhu68 vp1 proteins produced by expression from a nucleic acid sequence which encodes the amino acid sequence of 1 to 736 of SEQ ID NO: 7, vp1 proteins produced from SEQ ID NO: 6, or vp1 proteins produced from a nucleic acid sequence at least 70% identical to SEQ ID NO: 6 which encodes the predicted amino acid sequence of 1 to 736 of SEQ ID NO: 7; AAVhu68 vp2 proteins produced by expression from a nucleic acid sequence which encodes the predicted amino acid sequence of at least about amino acids 138 to 736 of SEQ ID NO: 7, vp2 proteins produced from a sequence comprising at least nucleotides 412 to 2211 of SEQ ID NO: 6, or vp2 proteins produced from a nucleic acid sequence at least 70% identical to at least nucleotides 412 to 2211 of SEQ ID NO: 6 which encodes the predicted amino acid sequence of at least about amino acids 138 to 736 of SEQ ID NO: 7; and/or AAVhu68 vp3 proteins produced by expression from a nucleic acid sequence which encodes the predicted amino acid sequence of at least about amino acids 203 to 736 of SEQ ID NO: 7, vp3 proteins produced from a sequence comprising at least nucleotides 607 to 2211 of SEQ ID NO: 6, or vp3 proteins produced from a nucleic acid sequence at least 70% identical to at least nucleotides 607 to 2211 of SEQ ID NO: 6 which encodes the predicted amino acid sequence of at least about amino acids 203 to 736 of SEQ ID NO: 7.In certain embodiments, an AAVhu68 capsid comprises: (i) heterogenous populations of AAVhu68 vp1 proteins, AAVhu68 vp2 proteins, and AAVhu68 vp3 proteins produced from a nucleic acid sequence encoding SEQ ID NO: 7, wherein the AAVhu68vp1 proteins comprise a glutamic acid at position 67 and a valine at position 157 and the AAVhu68vp2 proteins comprise a valine at position 157 based on the numbering of SEQ ID NO: 7; or (ii) heterogenous populations of AAVhu68 vp1, AAVhu68 vp2 and AAVhu68 vp3 proteins, wherein the AAVhu68 vp1 proteins are amino acids 1 to 736 of SEQ ID NO: 7 (vp1) which comprise a glutamic acid at position 67 and a valine at position 157 and further comprise subpopulations of vp1 proteins comprising modified amino acids based on the amino acids positions in SEQ ID NO: 7, wherein the AAVhu68 vp2 proteins are amino acids 138 to 736 of SEQ ID NO: 7 (vp2) which comprise a valine at position 157 and further comprise subpopulations of vp2 proteins comprising modified amino acids based on the amino acid positions in SEQ ID NO: 7, and wherein the AAVhu68 vp3 proteins are amino acids 203 to 736 of SEQ ID NO: 7 (vp3), which comprise subpopulations of vp3 proteins comprising modified amino acids based on the amino acid positions in SEQ ID NO: 7, wherein the AAVhu68 vp1, AAVhu68 vp2 and AAV hu68 vp3 proteins in (i) and (ii) comprise at least 50% to 100% deamidated asparagines (N) in asparagine-glycine pairs at each of positions 57, 329, 452, 512, relative to the amino acids in SEQ ID NO: 7, wherein the deamidated asparagines are deamidated to aspartic acid, isoaspartic acid, an interconverting aspartic acid/isoaspartic acid pair, or combinations thereof, as determined using mass spectrometry. In certain embodiments, the AAVhu68 capsid comprises: (a) a subpopulation of vp1 proteins in which 75% to 100% of the N at position 57 of the vp1 proteins are deamidated, as determined using mass spectrometry; and/or (b) subpopulations of vp1 proteins, vp2 proteins, and/or vp3 proteins in which 75% to 100% of the N at position 329, based on the numbering of SEQ ID NO:2, are deamidated as determined using mass spectrometry; and/or (c) subpopulations of vp1 proteins, vp2 proteins, and/or vp3 proteins in which 75% to 100% of the N at position 452, based on the numbering of SEQ ID NO: 7, are deamidated as determined using mass spectrometry; and/or (d) subpopulations of vp1 proteins, vp2 proteins, and/or vp3 proteins in which 75% to 100% of the N at position 512, based on the numbering of SEQ ID NO: 7, are deamidated as determined using mass spectrometry.

The AAVhu68 vp1, vp2 and vp3 proteins are typically expressed as alternative splice variants encoded by the same nucleic acid sequence which encodes the full-length vp1 amino acid sequence (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence (about aa 203 to 736) without the vp1-unique region (about aa 1 to about aa 137) and/or vp2-unique regions (about aa 1 to about aa 202), or a strand complementary thereto, the corresponding mRNA or tRNA (for example, the mRNA transcribed from about nucleotide (nt) 607 to about nt 2211 of SEQ ID NO: 6), or a sequence at least 70% to at least 99% (e.g., at least 85%, at least 90%, at least 95%, at least 97%, at least 98% or at least 99%) identical to SEQ ID NO: 6 which encodes aa 203 to 736 of SEQ ID NO: 7. Additionally, or alternatively, the vp1-encoding and/or the vp2-encoding sequence may be co-expressed with the nucleic acid sequence which encodes the AAVhu68 vp2 amino acid sequence of SEQ ID NO: 7 (about aa 138 to 736) without the vp1-unique region (about aa 1 to about 137), or a strand complementary thereto, the corresponding mRNA or tRNA (for example, the mRNA transcribed from nt 412 to 2211 of SEQ ID NO: 6), or a sequence at least 70% to at least 99% (e.g., at least 85%, at least 90%, at least 95%, at least 97%, at least 98% or at least 99%) identical to SEQ ID NO: 6 which encodes about aa 138 to 736 of SEQ ID NO: 7.

As described herein, a rAAVhu68 has a rAAVhu68 capsid produced in a production system expressing capsids from an AAVhu68 nucleic acid sequence which encodes the vp1 amino acid sequence of SEQ ID NO: 7, and optionally additional nucleic acid sequences, e.g., encoding a vp3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 7. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 6, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 7 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 6). In certain embodiments, also provided is a nucleic acid sequence which encodes the AAVhu68 vp2 amino acid sequence of SEQ ID NO: 7 (about aa 138 to 736) without the vp1-unique region (about aa 1 to about 137), or a strand complementary thereto, the corresponding mRNA or tRNA (nt 412 to 2211 of SEQ ID NO: 6).

However, other nucleic acid sequences which encode the amino acid sequence of SEQ ID NO: 7 may be selected for use in producing rAAVhu68 capsids. In certain embodiments, the nucleic acid sequence has the nucleic acid sequence of SEQ ID NO: 6 or a sequence at least 70% to 99% identical, at least 75%, at least 80%, at least 85%, at least 90%, at least 95%, at least 97%, at least 99%, identical to SEQ ID NO: 6 which encodes SEQ ID NO: 7. In certain embodiments, the nucleic acid sequence has the nucleic acid sequence of SEQ ID NO: 6 or a sequence at least 70% to 99%, at least 75%, at least 80%, at least 85%, at least 90%, at least 95%, at least 97%, at least 99%, identical to about nt 412 to about nt 2211 of SEQ ID NO: 6 which encodes the vp2 capsid protein (about aa 138 to 736) of SEQ ID NO: 7. In certain embodiments, the nucleic acid sequence has the nucleic acid sequence of about nt 607 to about nt 2211 of SEQ ID NO: 6 or a sequence at least 70% to 99%, at least 75%, at least 80%, at least 85%, at least 90%, at least 95%, at least 97%, at least 99%, identical to nt 607 to about nt 2211 of SEQ ID NO: 6 which encodes the vp3 capsid protein (about aa 203 to 736) of SEQ ID NO: 7.

It is within the skill in the art to design nucleic acid sequences encoding this AAVhu68 capsid, including DNA (genomic or cDNA), or RNA (e.g., mRNA). In certain embodiments, the nucleic acid sequence encoding the AAVhu68 vp1 capsid protein is provided in SEQ ID NO: 6. See, WO 2018/160582 which is incorporated herein by reference in its entirety. In certain embodiments, the AAVhu68 capsid is produced using a nucleic acid sequence of SEQ ID NO: 6 or a sequence at least 70%, at least 75%, at least 80%, at least 85%, at least 90%, at least 95%, at least 97%, at least 99%, which encodes the vp1 amino acid sequence of SEQ ID NO: 7 with a modification (e.g., deamidated amino acid) as described herein. In certain embodiments, the vp1 amino acid sequence is reproduced in SEQ ID NO: 7.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 7 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified.

For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 based on the numbering of SEQ ID NO: 7 (AAVhu68) may be deamidated based on the total vp1 proteins may be deamidated based on the total vp1, vp2 and vp3 proteins). Such percentages may be determined using 2D-gel, mass spectrometry techniques, or other suitable techniques.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly. The deamidation of N may occur through its C-terminus residue's backbone nitrogen atom conducts a nucleophilic attack to the Asn's side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate.

As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio.

In certain embodiments, a rAAV has an AAV capsid having vp1, vp2 and vp3 proteins having subpopulations comprising combinations of two, three, four or more deamidated residues at the positions set forth in the table provided in Example 11 and incorporated herein by reference. Deamidation in the rAAV may be determined using 2D gel electrophoresis, and/or mass spectrometry (MS), and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule+0.984 Da (the mass difference between —OH and —NH₂ groups). The percent deamidation of a particular peptide is determined by the mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It is understood by one of skill in the art that a number of variations on these illustrative methods can be used. For example, suitable mass spectrometers may include, e.g, a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur that do not result in conversion of one amino acid to a different amino acid residue. Such modifications may include acetylated residues, isomerizations, phosphorylations, or oxidations.

Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternative one or more other amide amino acids may be changed to a non-amide amino acid to reduce deamidation of the AAV. In certain embodiments, a mutant AAV capsid as described herein contains a mutation in an arginine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

As used herein, an “AAV9 capsid” is a self-assembled AAV capsid composed of multiple AAV9 vp proteins. The AAV9 vp proteins are typically expressed as alternative splice variants encoded by a nucleic acid sequence of SEQ ID NO: 9 or a sequence at least 70%, at least 75%, at least 80%, at least 85%, at least 90%, at least 95%, at least 97%, at least 99% thereto, which encodes the vp1 amino acid sequence of GenBank accession: AAS99264. In certain embodiments, “AAV9 capsid” includes an AAV having an amino acid sequence which is 99% identical to AAS99264 or 99% identical to SEQ ID NO: 10. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809. See, also, WO 2019/169004; and WO 2019/168961, all of which are incorporated herein by reference in their entireties.

Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point.

Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

V. rAAV

Provided herein is a therapeutic, recombinant, and replication-defective adeno-associated virus (rAAV) which is useful for treating a disease associated with an Arylsulfatase A gene (ARSA) mutation or caused by deficiencies in normal levels of functional Arylsulfatase A (for example, Metachromatic Leukodystrophy (MLD)) in a subject in need thereof. The rAAV is desirably replication-defective and carries a vector genome comprising inverted terminal repeats (ITR) and a nucleic acid sequence encoding a functional human Arylsulfatase A (hARSA) under the control of regulatory sequences which direct the hARSA expression in a target cell. In certain embodiments, the hARSA coding sequence comprises a sequence of nucleotide (nt) 55 to nt 1521 of SEQ ID NO: 1, or a sequence at least 95% to 99.9% identical thereto which encodes a functional hARSA. In certain embodiments, the vector genome comprises inverted terminal repeats (ITR) and an expression cassette as described in Part III. In a further embodiment, the rAAV comprises an AAV capsid.

The AAV capsid may be selected based on the target cell. In certain embodiment, the AAV capsid is suitable for delivery of the vector genome in nervous system (for example, CNS or PNS). In certain embodiments, the AAV capsid is suitable for delivery of the vector genome in a neuron, a nervous system cell, an oligodendrocyte, a microglia, a Central Nervous System (CNS) cell, a neuron in the CNS, a Peripheral Nervous System (PNS) cell, a Schwann cell, a macrophage in the PNS, or a cell in visceral organs (for example, a kidney cell, a liver cell and a gallbladder cell). In certain embodiments, the AAV capsid is suitable for delivery of the vector genome in another target cell as described herein.

In certain embodiments, the AAV capsid is selected from a cy02 capsid, a rh43 capsid, an AAV8 capsid, a rh01 capsid, an AAV9 capsid, an rh8 capsid, a rh10 capsid, a bb01 capsid, a hu37 capsid, a rh02 capsid, a rh20 capsid, a rh39 capsid, a rh64 capsid, an AAV6 capsid, an AAV1 capsid, a hu44 capsid, a hu48 capsid, a cy05 capsid a hu11 capsid, a hu32 capsid, a pi2 capsid, or a variation thereof. In certain embodiments, the AAV capsid is a Clade F capsid, such as AAV9 capsid, AAVhu68 capsid, AAV-PHP.B capsid, hu31 capsid, hu32 capsid, or a variation thereof. See, e.g., WO 2005/033321 published Apr. 14, 2015, WO 2018/160582, and US 2015/0079038, each of which is incorporated herein by reference in its entirety. In certain embodiments, the AAV capsid is a non-cade F capsid, for example a Clade A, B, C, D, or E capsid. In certain embodiment, the non-Clade F capsid is an AAV1 or a variation thereof. In certain embodiment, the AAV capsid transduces a target cell other than the nervous system cells. In certain embodiments, the AAV capsid is a Clade A capsid (e.g., AAV1, AAV6), a Clade B capsid (e.g., AAV 2), a Clade C capsid (e.g., hu53), a Clade D capsid (e.g., AAV7), or a Clade E capsid (e.g., rh10). Still, other AAV capsid may be chosen.

In certain embodiment, the rAAV comprises an AAVhu68 capsid in which the vector genome is packaged. In certain embodiments, the AAVhu68 capsid is produced from a sequence encoding the predicted amino acid sequence of SEQ ID NO: 7.

See, Part V for more details. In certain embodiments, the vector genome is entirely exogenous to the AAVhu68 capsid, as it contains no AAVhu68 genomic sequences.

The functional hARSA is described in Part I. In certain embodiments, the functional hARSA has a signal peptide and a sequence of amino acid (aa) 19 to aa 507 of SEQ ID NO: 2. In certain embodiments, the native hARSA signal peptide is used, e.g., aa 1 to aa 18 of SEQ ID NO: 2. In certain embodiments, the signal peptide has an amino acid sequence of aa 1 to aa 20 of SEQ ID NO: 4. In certain embodiment the functional hARSA has an amino acid sequence of SEQ ID NO: 2 or SEQ ID NO: 4.

In certain embodiments, the hARSA coding sequence is about 95% to 100% identical to nucleotide (nt) 55 to nt 1521 of SEQ ID NO: 1. In certain embodiments, the hARSA-coding sequence is SEQ ID NO: 1 or SEQ ID NO: 3. In a further embodiment, the hARSA coding sequence encodes a sequence of amino acid (aa) 19 to aa 507 of SEQ ID NO: 2. In yet a further embodiment, the hARSA coding sequence encodes a sequence of SEQ ID NO: 2 or SEQ ID NO: 4. See, Part I for more details about hARSA coding sequence.

In certain embodiments, the regulatory sequences direct hARSA expression in nervous system cells. In certain embodiments, the regulatory sequences comprise a ubiquitous promoter, for example, a CB7 promoter. In a further embodiment, the regulatory elements comprise one or more of a Kozak sequence, a polyadenylation sequence, an intron, an enhancer, and a TATA signal. In certain embodiments, the regulatory sequences comprise one or more of the following: a regulatory element derived from the chicken β-actin (BA) promoter and human cytomegalovirus immediate-early enhancer (CMV IE) (for example, CB7 promoter, nt 198 to nt 862 of SEQ ID NO: 5), a chimeric intron consisting of a chicken BA splice donor and a rabbit β-globin (rBG) splice acceptor element (for example, CI, nt 956 to nt 1928 of SEQ ID NO: 5), and polyadenylation (PolyA) signal derived from the rBG gene (for example, rBG, nt 3539 to nt 3665 of SEQ ID NO: 5). In certain embodiments, the vector genome has a sequence of nucleotide (nt) 1 to nt 3883 of SEQ ID NO: 5. See, Part III for more details.

In certain embodiments, the rAAV or a composition comprising the rAAV is administrable to a subject in need thereof to ameliorate symptoms of a disease associated with an ARSA mutation or caused by deficiencies in normal levels of functional Arylsulfatase A (for example, MLD), and/or to delay progression of a disease associated with an ARSA mutation or caused by deficiencies in normal levels of functional Arylsulfatase A (for example, MLD). See, part II for more details.

In certain embodiments, the rAAV as described herein is suitable for administration to a patient via an intra-cisterna magna injection (ICM), including via a CT-guided sub-occipital injection into the cisterna magna. In certain embodiments, the rAAV as described herein is suitable for administration to a subject who is 7 years of age or younger. In certain embodiments, the rAAV as described herein is suitable for administration to a subject in need thereof to ameliorate symptoms of Metachromatic Leukodystrophy or a disease associated with Arylsulfatase A (ARSA) gene mutation, and/or to delay progression of Metachromatic Leukodystrophy or a disease associated with Arylsulfatase A (ARSA) gene mutation. See, Part II and Part VIII for more details. In certain embodiments, the rAAV as described herein is administered in a single dose.

In certain embodiment, the vector genome is a single-stranded AAV vector genome. In certain embodiments, a rAAV vector may be utilized in the invention which contains self-complementary (sc) AAV vector genome.

The regulatory control elements necessary are operably linked to the gene (e.g., hARSA coding sequence) in a manner which permits its transcription, translation and/or expression in a cell which takes up the rAAV. As used herein, “operably linked” sequences include both expression control sequences that are contiguous with the gene of interest and expression control sequences that act in trans or at a distance to control the gene of interest. Such regulatory sequences typically include, e.g., one or more of a promoter, an enhancer, an intron, a polyA, a self-cleaving linker (e.g., furin, furin-F2A, an IRES). The examples below utilize CB7 promoter for expression of hARSA. However, in certain embodiments, other promoters, or an additional promoter, may be selected. In certain embodiments, an additional or alternative promoter sequence may be included as part of the expression control sequences (regulatory sequences), e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. In addition to a promoter, a vector may contain one or more other appropriate transcription initiation sequences, transcription termination sequences, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the regulatory sequences comprise one or more expression enhancers. In one embodiment, the regulatory sequences contain two or more expression enhancers. These enhancers may be the same or may differ from one another. For example, an enhancer may include a CMV immediate early enhancer (SEQ ID NO: 19). This enhancer may be present in two copies which are located adjacent to one another. Alternatively, the dual copies of the enhancer may be separated by one or more sequences. In still another embodiment, the expression cassette further contains an intron, e.g., the chicken beta-actin intron (SEQ ID NO: 17). In certain embodiments, the intron is a chimeric intron (CI)—a hybrid intron consisting of a human beta-globin splice donor and immunoglobulin G (IgG) splice acceptor elements. Other suitable introns include those known in the art, e.g., such as are described in WO 2011/126808. Examples of suitable polyA sequences include, e.g., SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619). In certain embodiments, no WPRE sequence is present.

In certain embodiments, in addition to the hARSA coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external “A” elements is deleted. The shortened ITR is reverted back to the wild type length of 145 base pairs during vector DNA amplification using the internal A element as a template. In other embodiments, the full-length AAV 5′ and 3′ ITRs are used. In still other embodiments, longer or shorter AAV ITRs may be selected. However, ITRs from other AAV sources may be selected. Where the source of the ITRs is from AAV2 and the AAV capsid is from another AAV source, the resulting vector may be termed pseudotyped. However, other configurations of these elements may be suitable. In certain embodiments the 5′ ITR sequence
includes: ttggccactc cctctctgcg cgctcgctcg ctcactgagg ccgggcgacc aaaggtcgcc cgacgcccgg gctttgcccg ggcggcctca gtgagcgagc gagcgcgcag agagggagtg gccaactcca tcactagggg ttcct [SEQ ID NO: 25]

In certain embodiments the 3′ ITR sequence includes: aggaa cccctagtga tggagttggc cactccctct ctgcgcgctc gctcgctcac tgaggccggg cgaccaaagg tcgcccgacg cccgggcttt gcccgggcgg cctcagtgag cgagcgagcg cgcagagagg gagtggccaa [SEQ ID NO: 26]

In certain embodiments, vector genomes are constructed which comprise a 5′ AAV ITR-promoter—optional enhancer—optional intron—hARSA coding sequence—polyA-3′ ITR, termed as AAV.promoter.optional enhancer.optional intron.hARSA or hARSAco.polyA. In certain embodiments, the ITRs are from AAV2. In certain embodiments, more than one promoter is present. In certain embodiments, the enhancer is present in the vector genome. In certain embodiments, more than one enhancer is present. In certain embodiments, an intron is present in the vector genome. In certain embodiments, the enhancer and intron are present. In certain embodiments, the intron is a chimeric intron (CI)— a hybrid intron consisting of a human beta-globin splice donor and immunoglobulin G (IgG) splice acceptor elements. In certain embodiments, the polyA is an SV40 poly A (i.e., a polyadenylation (PolyA) signal derived from Simian Virus 40 (SV40) late genes). In certain embodiments, the polyA is a rabbit beta-globin (RBG) poly A. In certain embodiments, the vector genome comprises a 5′ AAV ITR-CB7 promoter—hARSA coding sequence—poly A-3′ ITR. See, e.g., the expression cassette of SEQ ID NO: 28 (hybrid promoter through poly).

As used herein, a vector genome or a rAAV comprising the vector genome is illustrated herein as AAV.promoter (optional).Kozak (optional).intron (optional).hARSA coding sequence (e.g., hARSA, hARSAco). miRNA (optional).polyA (optional).Stuffer (optional). In certain embodiments, a rAAV is illustrated herein as AAVcapsid.promoter (optional).Kozak (optional).intron (optional).hARSA coding sequence. miRNA (optional).polyA (optional).Stuffer (optional).

In another aspect, a production system useful for producing the rAAV is provided. In this system, cells were cultured which comprises a nucleic acid sequence encoding an AAVhu68 capsid protein, a vector genome as described herein and sufficient AAV rep functions and helper functions to permit packaging of the vector genome into the AAV capsid. In certain embodiments, the vector genome has a sequence comprising nt 1 to nt 3883 of SEQ ID NO: 5 (SEQ ID NO: 27). In certain embodiments, the expression cassette has a sequence comprising nt 198 to nt 3665 of SEQ ID NO: 5 (SEQ ID NO: 28). In certain embodiments, the cell culture is a human embryonic kidney 293 cell culture. In certain embodiments, the AAV rep is from an AAV different from AAVhu68, for example, from AAV2. In certain embodiments, the AAV rep coding sequence and cap genes are on the same nucleic acid molecule, wherein there is optionally a spacer between the rep sequence and cap gene. In a further embodiment, the spacer is atgacttaaaccaggt (SEQ ID NO: 24).

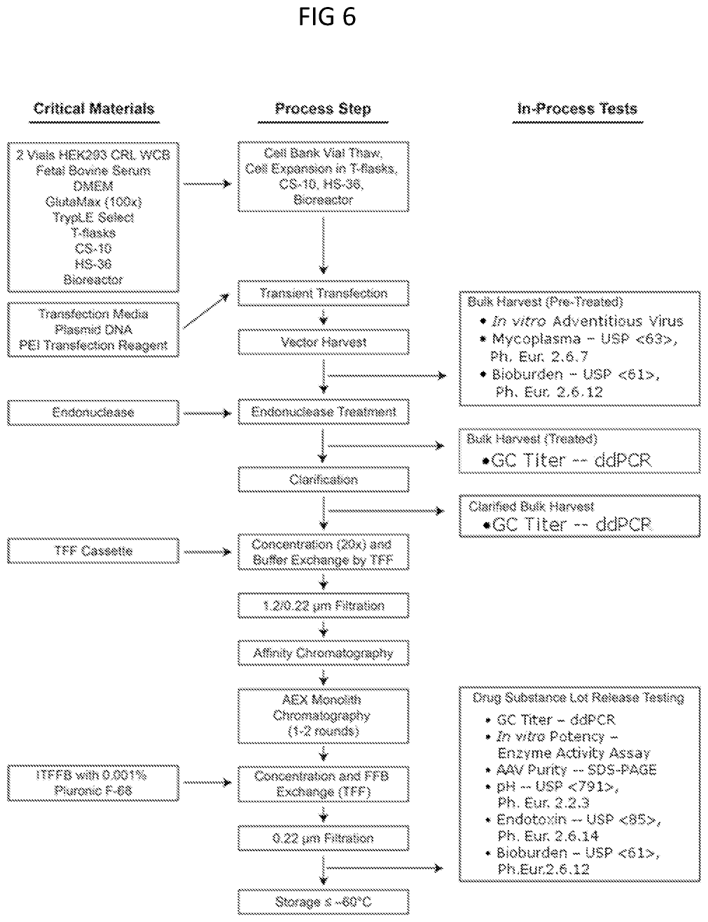

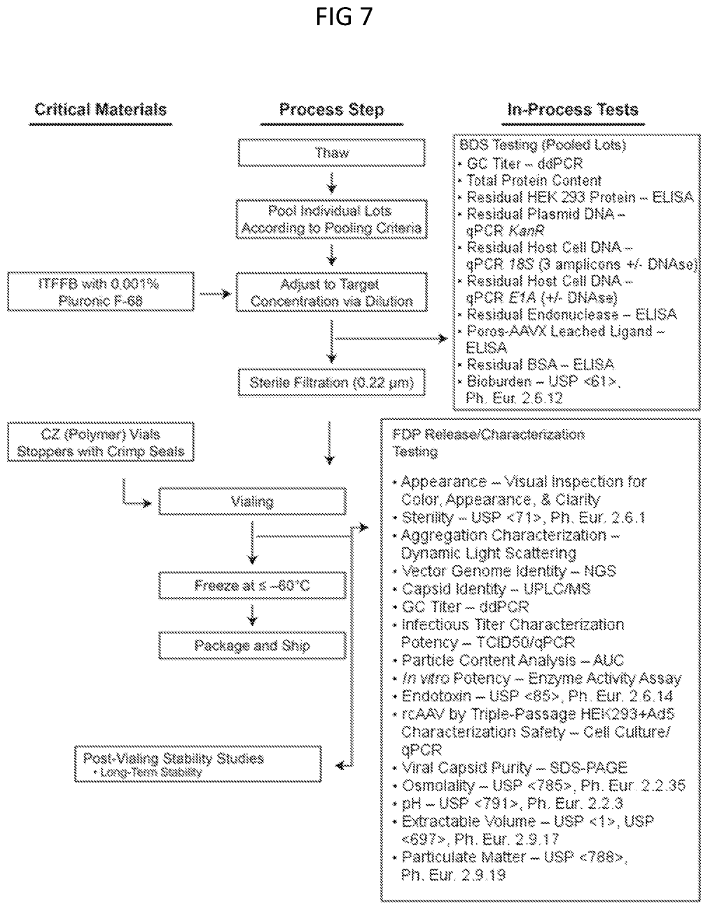

For use in producing an AAV viral vector (e.g., a recombinant (r) AAV), the vector genomes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art. An illustrative production process is provided in FIGS. 6-7. In certain embodiments, the plasmid has a sequence of SEQ ID NO: 5.

Methods for generating and isolating AAVs suitable for use as vectors are known in the art. See generally, e.g., Grieger & Samulski, 2005, Adeno-associated virus as a gene therapy vector: Vector development, production and clinical applications, Adv. Biochem. Engin/Biotechnol. 99: 119-145; Buning et al., 2008, Recent developments in adeno-associated virus vector technology, J. Gene Med. 10:717-733; and the references cited below, each of which is incorporated herein by reference in its entirety. For packaging a gene into virions, the ITRs are the only AAV components required in cis in the same construct as the nucleic acid molecule containing the gene. The cap and rep genes can be supplied in trans.

In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAVhu68 is provided. Such a cell culture contains a nucleic acid which expresses the AAVhu68 capsid protein in the host cell; a nucleic acid molecule suitable for packaging into the AAVhu68 capsid, e.g., a vector genome which contains AAV ITRs and a non-AAV nucleic acid sequence encoding a gene operably linked to regulatory sequences which direct expression of the gene in a host cell; and sufficient AAV rep functions and adenovirus helper functions to permit packaging of the vector genome into the recombinant AAVhu68 capsid. In one embodiment, the cell culture is composed of mammalian cells (e.g., human embryonic kidney 293 cells, among others) or insect cells (e.g., Spodoptera frugiperda (Sf9) cells). In certain embodiments, baculovirus provides the helper functions necessary for packaging the vector genome into the recombinant AAVhu68 capsid.

Optionally the rep functions are provided by an AAV other than AAVhu68. In certain embodiments, at least parts of the rep functions are from AAVhu68. In another embodiment, the rep protein is a heterologous rep protein other than AAVhu68rep, for example but not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Any of these AAVhu68 or mutant AAV capsid sequences may be under the control of exogenous regulatory control sequences which direct expression thereof in a host cell.

In one embodiment, cells are manufactured in a suitable cell culture (e.g., HEK 293 or Sf9) or suspension. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV vector genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, Adenovirus-adeno-associated virus hybrid for large-scale recombinant adeno-associated virus production, Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See, also, U.S. Provisional Patent Application No. 63/371,597, filed Aug. 16, 2022, entitled “Scalable Methods for Producing rAAV with Packaged Vector Genomes, and U.S. Provisional Patent Application No. 63/371,592, filed Aug. 16, 2022, entitled “Scalable Methods for Downstream Purification of Recombinant Adeno-associated Virus”, both incorporated by reference in their entirety.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. A two-step affinity chromatography purification at high salt concentration followed anion exchange resin chromatography are used to purify the vector drug product and to remove empty capsids. These methods are described in more detail in WO 2017/160360, International Patent Application No. PCT/US2016/065970, filed Dec. 9, 2016 and its priority documents, US Patent Application Nos. 62/322,071, filed Apr. 13, 2016 and 62/226,357, filed Dec. 11, 2015 and entitled “Scalable Purification Method for AAV9”, which is incorporated by reference herein.

To calculate empty and full particle content, VP3 band volumes for a selected sample (e.g., in examples herein an iodixanol gradient-purified preparation where #of genome copies (GC)=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAVhu68 particles having packaged genomic sequences from genome-deficient AAVhu68 intermediates involves subjecting a suspension comprising recombinant AAVhu68 viral particles and AAVhu68 capsid intermediates to fast performance liquid chromatography, wherein the AAVhu68 viral particles and AAVhu68 intermediates are bound to a strong anion exchange resin equilibrated at a pH of about 10.2, and subjected to a salt gradient while monitoring eluate for ultraviolet absorbance at about 260 nanometers (nm) and about 280 nm. Although less optimal for rAAVhu68, the pH may be in the range of about 10.0 to 10.4. In this method, the AAVhu68 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/hu68 serotype. Under these ionic conditions, a significant percentage of residual cellular DNA and proteins flow through the column, while AAV particles are efficiently captured.

The rAAV.hARSA is suspended in a suitable physiologically compatible composition (e.g., a buffered saline). This composition may be frozen for storage, later thawed and optionally diluted with a suitable diluent. Alternatively, the vector may be prepared as a composition which is suitable for delivery to a patient without proceeding through the freezing and thawing steps.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purincation steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

### VI. Other Vector

In one aspect, provided herein is a vector which is useful for treating a disease associated with an ARSA mutation or caused by deficiencies in normal levels of functional Arylsulfatase A (for example, MLD) in a subject in need thereof. The vector carries a nucleic acid sequence encoding a functional human Arylsulfatase A (hARSA) under the control of regulatory sequences which direct the hARSA expression in a target cell. In certain embodiments, the hARSA coding sequence is about 95% to 100% identical to SEQ ID NO: 1. Additionally or alternatively, the function hARSA protein has an amino acid sequence of SEQ ID NO: 2. In certain embodiments, the hARSA-coding sequence is SEQ ID NO: 1. In certain embodiments, the vector or a composition comprising the vector is administrable to a subject in need thereof to ameliorate symptoms of a disease associated with an ARSA mutation or caused by deficiencies in normal levels of functional Arylsulfatase A (for example, MLD), and/or to delay progression of a disease associated with an ARSA mutation or caused by deficiencies in normal levels of functional Arylsulfatase A (for example, MLD).

In certain embodiments, the vector comprises an expression cassette. In certain embodiments, the expression cassette comprises a nucleic acid sequence encoding a functional human Arylsulfatase A (hARSA) under control of regulatory sequences which direct the hARSA expression. In certain embodiments, the functional hARSA protein comprises a signal peptide and an amino acid sequence of amino acid (aa) 19 to aa 507 of SEQ ID NO: 2. In certain embodiments, the signal peptide has an amino acid sequence of aa 1 to aa 18 of SEQ ID NO: 2 or an amino acid sequence of aa 1 to aa 20 of SEQ ID NO: 4. In certain embodiments, the hARSA coding sequence has a sequence of nucleotide (nt) 55 to nt 1521 of SEQ ID NO: 1, or a sequence at least 95% to 99.9% identical thereto which encodes a functional hARSA. In certain embodiments, the hARSA coding sequence is SEQ ID NO: 1 or SEQ ID NO: 3. See, Parts I, and III for more details.

In certain embodiments, the vector is a viral vector selected from a recombinant parvovirus, a recombinant lentivirus, a recombinant retrovirus, or a recombinant adenovirus; or a non-viral vector selected from naked DNA, naked RNA, an inorganic particle, a lipid particle, a polymer-based vector, or a chitosan-based formulation. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

In certain embodiments, the vector is suitable for administration to a patient via an intra-cisterna magna injection (ICM), including via a CT-guided sub-occipital injection into the cisterna magna. In certain embodiments, the vector is suitable for administration to a subject who is 7 years of age or younger. In certain embodiments, the vector is suitable for administration to a subject in need thereof to ameliorate symptoms of Metachromatic Leukodystrophy or a disease associated with Arylsulfatase A (ARSA) gene mutation, and/or to delay progression of Metachromatic Leukodystrophy or a disease associated with Arylsulfatase A (ARSA) gene mutation. In certain embodiments, the vector is administered in a single dose. See, Part II and Part VIII for more details.

A “replication-defective virus” or “viral vector” refers to a synthetic or artificial viral particle in which an expression cassette containing a gene of interest (e.g., hARSA coding sequence) is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

### VII. Compositions

In a further aspect, provided herein is a composition comprising a rAAV or a vector as described herein and an aqueous suspension media. In certain embodiments, the aqueous composition is provided which comprises a formulation buffer and the rAAV or vector as described. In certain embodiments, the formulation buffer comprises: an artificial cerebrospinal fluid comprising buffered saline and one or more of sodium, calcium, magnesium, potassium, or mixtures thereof; and a surfactant. In certain embodiments, the formulation buffer comprises about 0.0005% to about 0.001% surfactant. In certain embodiments, the composition is at a pH of 7.2 to 7.8. In certain embodiments, AAV.CB7.CJ.hARSAco.rBG drug product consists of a non-replicating recombinant adeno-associated viral (rAAV) vector as described herein and a formulation buffer.

In certain embodiments, an aqueous pharmaceutical composition comprising a rAAV as described herein and a formulation buffer is provided. In certain embodiments, the formulation buffer comprises: an artificial cerebrospinal fluid comprising buffered saline and one or more of sodium, calcium, magnesium, potassium, or mixtures thereof; and a surfactant. In certain embodiments, the surfactant is present at 0.0005% to about 0.001% of the pharmaceutical composition. In certain embodiments, the composition is at a pH in the range of 7.5 to 7.8. In certain embodiments, the formulation buffer is suitable for intravenous delivery, intrathecal administration, or intracerebroventricular administration.

In certain embodiments, provided is a pharmaceutical composition comprising a vector as described and a formulation buffer. In certain embodiments, the formulation buffer is suitable for intravenous delivery, intrathecal administration, or intracerebroventricular administration.

In certain embodiments, the composition is suitable for administration to a patient via an intra-cisterna magna injection (ICM), including via a CT-guided sub-occipital injection into the cisterna magna. In certain embodiments, the composition is suitable for administration to a subject who is 7 years of age or younger. In certain embodiments, the composition is suitable for administration to a subject in need thereof to ameliorate symptoms of Metachromatic Leukodystrophy or a disease associated with Arylsulfatase A (ARSA) gene mutation, and/or to delay progression of Metachromatic Leukodystrophy or a disease associated with Arylsulfatase A (ARSA) gene mutation. In certain embodiments, the composition is administered in a single dose. In certain embodiments, the composition has an at least 2.50×10¹³GC rAAV per mL.

Provided herein are compositions containing at least one rAAV stock (e.g, an rAAVhu68 stock or a mutant rAAVhu68 stock) and an optional carrier, excipient and/or preservative. An rAAV stock refers to a plurality of rAAV vectors which are the same, e.g., such as in the amounts described below in the discussion of concentrations and dosage units.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. In one embodiment, the surfactant may be present in an amount up to about 0.0005% to about 0.001% (based on weight ratio, w/w %) of the suspension. In another embodiment, the surfactant may be present in an amount up to about 0.0005% to about 0.001% (based on volume ratio, v/v %) of the suspension. In yet another embodiment, the surfactant may be present in an amount up to about 0.0005% to about 0.001% of the suspension, wherein n % indicates n gram per 100 mL of the suspension. In yet another embodiment, the surfactant may be present in an amount up to about 0.0005% to about 0.001% (based on weight over volume ratio, v/w %) of the suspension.

As used herein, in certain embodiments, “%” upon referring to a concentration, is a weight ratio, for example, percentage of the substance (to be dissolved via a solvent into a solution) weight over the solvent's weight, or percentage of the substance (to be dissolved via a solvent into a solution) weight over the solution's weight. In certain embodiments, “%” upon referring to a concentration, is a volume ratio, for example, percentage of the substance (to be dissolved via a solvent into a solution) volume over the solvent's volume, or percentage of the substance (to be dissolved via a solvent into a solution) volume over the solution's volume. In certain embodiments, “%” upon referring to a concentration, indicates gram of the substance (to be dissolved via a solvent into a solution) per 100 mL of the solvent or solution. In certain embodiments, “%” upon referring to a concentration, is a weight over volume ratio, for example, percentage of the substance (to be dissolved via a solvent into a solution) weight over the solvent's volume, or percentage of the substance (to be dissolved via a solvent into a solution) weight over the solution's volume.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., brain, CSF, the liver (optionally via the hepatic artery), lung, heart, eye, kidney), oral, inhalation, intranasal, intrathecal, intratracheal, intraarterial, intraocular, intravenous, intramuscular, subcutaneous, intradermal, intraparenchymal, intracerebroventricular, intrathecal, ICM, lumbar puncture and other parenteral routes of administration. Routes of administration may be combined, if desired. Dosages of the viral vector depend primarily on factors such as the condition being treated, the age, weight and health of the patient, and can thus vary among patients. For example, a therapeutically effective human dosage of the viral vector is generally in the range of from about 25 to about 1000 microliters to about 100 mL of solution containing concentrations of from about 1×10⁹ to 1×10¹⁶ vector genome copies. In certain embodiments, a volume of about 1 mL to about 15 mL, or about 2.5 mL to about 10 mL, or about 5 mL suspension is delivered. In certain embodiments, a volume of about 1, about 2, about 3, about 4, about 5, about 6, about 7, about 8, about 9, about 10, about 11, about 12, about 13, about 14, or about 15 mL suspension is delivered. In certain embodiments, a dose of about 8.9×10¹² to 2.7×10¹⁴ GC total is administered in this volume. In certain embodiments, a dose of about 1.1×10¹⁰ GC/g brain mass to about 3.3×10¹¹ GC/g brain mass is administered in this volume. In certain embodiments, a dose of about 3.0×10⁹, about 4.0×10⁹, about 5.0×10⁹, about 6.0×10⁹, about 7.0×10⁹, about 8.0×10⁹, about 9.0×10⁹, about 1.0×10¹⁰, about 1.1×10¹⁰, about 1.5×10¹⁰, about 2.0×10¹⁰, about 2.5×10¹⁰, about 3.0×10¹⁰, about 3.3×10¹⁰, about 3.5×10¹⁰, about 4.0×10¹⁰, about 4.5×10¹⁰, about 5.0×10¹⁰, about 5.5×10¹⁰, about 6.0×10¹⁰, about 6.5×10¹⁰, about 7.0×10¹⁰, about 7.5×10¹⁰, about 8.0×10¹⁰, about 8.5×10¹⁰, about 9.0×10¹⁰, about 9.5×10¹⁰, about 1.0×10¹¹, about 1.1×10¹¹, about 1.5×10¹¹, about 2.0×10¹¹, about 2.5×10¹¹, about 3.0×10¹¹, about 3.3×10¹¹, about 3.5×10¹¹, about 4.0×10¹¹, about 4.5×10¹¹, about 5.0×10¹¹, about 5.5×10¹¹, about 6.0×10¹¹ about 6.5×10¹¹, about 7.0×10¹¹, about 7.5×10¹¹, about 8.0×10¹¹, about 8.5×10¹¹, about 9.0×10¹¹ GC per gram brain mass is administered in this volume.

| Subject Age | Assumed brain mass (g) |
|---|---|
| ≥4 to <9 months | 600 |
| ≥9 to <18 months | 1000 |
| ≥18 months to <3 years | 1100 |
| ≥3 years | 1300 |

The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an subject) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 1×10¹⁰ GC/g brain mass to about 1×10¹² GC/g brain mass. In certain embodiments, the dose may be in the range of about 3×10¹⁰ GC/g brain mass to about 5×10¹¹ GC/g brain mass.

In one embodiment, the viral constructs may be delivered in doses of from at least about least 1×10⁹ GC to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage may be adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other route of delivery.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrol® F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate·7H₂O), potassium chloride, calcium chloride (e.g., calcium chloride·2H₂O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/-article/2093316-overview. Optionally, for intrathecal delivery, a commercially available diluent may be used as a suspending agent, or in combination with another suspending agent and other optional excipients.

See, e.g., Elliotts B® solution [Lukare Medical]. Each 10 mL of Elliotts B Solution contains: Sodium Chloride, USP—73 mg; Sodium Bicarbonate, USP—19 mg; Dextrose, USP8 mg; Magnesium Sulfate·7H₂O, USP 3 mg; Potassium Chloride, USP— 3 mg; Calcium Chloride·2H₂O, USP—2 mg; Sodium Phosphate, dibasic·7H₂O, USP— 2 mg; Water for Injection, USP qs 10 mL.

Concentration of Electrolytes: Sodium 149 mEq/liter; Bicarbonate 22.6 mEq/liter; Potassium 4.0 mEq/liter; Chloride 132 mEq/liter; Calcium 2.7 mEq/liter; Sulfate 2.4 mEq/liter; Magnesium 2.4 mEq/liter; Phosphate 1.5 mEq/liter.

The formulae and molecular weights of the ingredients are:

| | MOLECULAR | MOLECULAR |
|---|---|---|
| INGREDIENT | FORMULA | WEIGHT |
| Sodium Chloride | NaCl | 58.44 |
| Sodium Bicarbonate | NaHCO₃ | 84.01 |
| Dextrose | C₆H₁₂O₆ | 180.16 |
| Magnesium Sulfate•7H2O | Mg₂SO₄•7H2O | 246.48 |
| Potassium Chloride | KCl | 74.55 |
| Calcium Chloride•2H2O | CaCl₂•2H₂O | 147.01 |
| Sodium Phosphate, dibasic•7H2O | Na₂HPO₄•7H₂O | 268.07 |

The pH of Elliotts B Solution is 6 to 7.5, and the osmolarity is 288 mOsmol per liter (calculated). In certain embodiments, the composition containing the rAAVhu68.hARSA is delivered at a pH in the range of 6.8 to 8, or 7.2 to 7.8, or 7.5 to 8. For intrathecal delivery, a pH above 7.5 may be desired, e.g., 7.5 to 8, or 7.8.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2.

In another embodiment, the formulation may contain a buffered saline aqueous solution comprising 1 mM Sodium Phosphate (Na₃PO₄), 150 mM sodium chloride (NaCl), 3 mM potassium chloride (KCl), 1.4 mM calcium chloride (CaCl₂)), 0.8 mM magnesium chloride (MgCl₂), and 0.001% poloxamer (e.g., Kolliphor®) 188, pH 7.2. See, e.g., harvardapparatus.com/harvard-apparatus-perfusion-fluid.html. In certain embodiments, Harvard's buffer is preferred due to better pH stability observed with Harvard's buffer. The table below provides a comparison of Harvard's buffer and Elliot's B buffer.

### Cerebrospinal Fluid (CSF) Compositions

| Component | Units | CSF | Elliot's B | Harvard's |
|---|---|---|---|---|
| Na⁺ | mEq/L | 117-137 | 149 | 150 |
| K⁺ | mEq/L | 2.3-4.6 | 4.0 | 3.0 |
| Mg⁺ | mEq/L | 2.2 | 2.4 | 0.8 |
| Ca²⁺ | mEq/L | 2.2 | 2.7 | 1.4 |
| Cl⁻ | mEq/L | 113-127 | 132 | 155 |
| HCO₃⁻ | mEq/L | 22.9 | 22.6 | 0 |
| Phos | mg/dL | 1.2-2.1 | 1.5 | 1.0 |
| Glucose | mg/dL | 45-80 | 80 | — |
| Pluronic | % | — | 0.001% (added) | 0.001% (added) |
| Osmolarity | mOsm/L | 295 | 288 | 290 |
| pH | | 7.31 | 6.0-7.5* | 7.2 (titrated to) |
| | | | Drift to 9+ | |
| | | | (8.2+ w/o | |
| | | | titratn) | |

In certain embodiments, the formulation buffer is artificial CSF with Pluronic F68. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna (i.e., intra cisterna magna, or ICM). In certain embodiments, the intrathecal administration is performed as described in US Patent Publication No. 2018-0339065 A1, published Nov. 29, 2019, which is incorporated herein by reference in its entirety. In certain embodiments, the CNS administration is performed using Ommaya Reservoir (also referred to as Ommaya device or Ommaya system).

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In certain embodiments, the final formulation buffer comprises an artificial cerebrospinal fluid comprising buffered saline and one or more of sodium, calcium, magnesium, potassium, or mixtures thereof; and a surfactant. In certain embodiments, the surfactant is about 0.0005% w/w to about 0.001% w/w of the suspension. In certain embodiments, the surfactant is Pluronic F68. In certain embodiments, the Pluronic F68 is present in an amount of about 0.0001% of the suspension. In certain embodiments, the composition is at a pH in the of 7.5 to 7.8 for intrathecal delivery.

In certain embodiments, treatment of the composition described herein has minimal to mild asymptomatic degeneration of DRG sensory neurons in animals and/or in human patients, well-tolerated with respect to sensory nerve toxicity and subclinical sensory neuron lesions.

In certain embodiment, the composition described herein is useful in improving functional and clinical outcomes in the subject treated. Such outcomes may be measured at about 30 days, about 60 days, about 90 days, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 2.5 years, about 3 years, about 3.5 years, about 4 years, about 4.5 years and then yearly up to the about 5 years after administration of the composition. Measurement frequency may be about every 1 month, about every 2 months, about every 3 months, about every 4 months, about every 5 months, about every 6 months, about every 7 months, about every 8 months, about every 9 months, about every 10 months, about every 11 months, or about every 12 months.

In certain embodiments, the composition described herein shows pharmacodynamics and clinical efficacy measured in treated subjects compared to untreated controls.

In certain embodiments, the pharmacodynamics efficacy, clinical efficacy, functional outcomes, clinical outcomes, disease amelioration, or disease progression may be assessed via one or more of the following: concentration and/or level and/or biological activity of ARSA (for example, in serum or in CSF), urine sulfatides, CNS myelination (demyelination load and pattern), white matter atrophy as measured by MRI, neuronal metabolite N-acetylaspartate (NAA), myo-inositol (ml), choline (Cho) and/or lactate (Lac) levels (for example, as measured by proton magnetic resonance spectroscopy (MRS)), CSF sulfatide and lyso-sulfatide levels, Visual evoked potentials (VEPs), Brainstem auditory evoked responses (BAERs), gall-bladder wall thickening (for example, via ultrasound evaluation); motor function (for example, measured by the Gross Motor Function Classification for Metachromatic Leukodystrophy (GMFC-MLD) or Gross Motor Function Measure (GMFM)), Motor milestones achievement (as defined by World Health Organization [WHO] criteria) assessed by age at achievement, age at loss, and percentage of children maintaining or acquiring motor milestones, cognitive function (for example, Total Intelligence Quotient [IQ] and sub-domain IQ measured by the Bayley Scale of Infant Development [BSID-III], Wechsler Intelligence Scale for Children, Fifth Edition [WISC-V]), lifespan (compared to a patient), neurological clinical exam (NCE), nerve conduction velocity (NCV) of the ulnar, deep peroneal, median, sural nerves, age-at-onset and frequency of seizures captured by a seizure diary, behavior function (for example, measured by Vineland Adaptive Behavior Scales, Third Edition (Vineland-III)), Lansky Performance Index, Pediatric Quality of Life Inventory (for example, PedsQL and PedsQL-IS), and caregiver/parent quality of life.

In certain embodiments, the pharmacodynamics efficacy, clinical efficacy, functional outcomes, clinical outcomes, disease amelioration, or disease progression may be assessed abnormal properties (for example biomarker activity, electrophysiological activity, and/or imaging parameters) and clinical observations (for example, gross and fine motor function, cognitive and language development, neurological exam findings, behavioral and milestone development, and caregiver/parent-reported outcomes and decreased quality of life assessments). Other disease amelioration or disease progression may be assessed, see, Parts II and VIII, relative section thereof is incorporated herein by reference in their entireties.

Alternatively or additionally, the pharmacodynamics efficacy, clinical efficacy, functional outcomes, or clinical outcomes may include biomarkers, for example, pharmacodynamics and biological activity of rAAVhu68.hARSAco.

### IIX. Methods

In another aspect, a method of treating a subject having a disease associated with an ARSA mutation or caused by deficiencies in normal levels of functional Arylsulfatase A (for example, MLD), or ameliorating symptoms of a disease associated with an ARSA mutation or caused by deficiencies in normal levels of functional Arylsulfatase A (for example, MLD), or delaying progression of a disease associated with an ARSA mutation or caused by deficiencies in normal levels of functional Arylsulfatase A (for example, MLD) is provided. The method comprises administrating an effective amount of a rAAV or a vector as described herein to a subject in need thereof. In certain embodiments, the vector or rAAV is administrable to a patient via an intra-cisterna magna injection (ICM), for example, CT-guided sub-occipital injection into the cisterna magna. In certain embodiments, a vector or a composition is provided which is administrable to a patient having Metachromatic Leukodystrophy who is 7 years of age or younger. In certain embodiments, the method involves delivering the rAAV or the vector to a human patient in a single dose. In certain embodiments, the rAAV is administered at a dose between 3.00×10¹⁰ genome copies (GC) per gram (GC/g) of brain mass and 1.00×10¹² GC/g of brain mass. In certain embodiments, following the administration, disease symptom of the subject is ameliorated and/or the disease progression is delayed.

Although nervous system-directed AAV gene therapy targets primarily neurons in vivo, the cross-correction potential opens the possibility to correct ARSA-deficient myelinating cells, which cannot be transduced in vivo by most gene therapy vectors (Cearley et al., 2008; Lawlor et al., 2009).

In certain embodiments, an “effective amount” herein is the amount which achieves amelioration of MLD symptoms and/or delayed MLD progression.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., brain, CSF, the liver (optionally via the hepatic artery), lung, heart, eye, kidney), oral, inhalation, intranasal, intrathecal, intratracheal, intraarterial, intraocular, intravenous, intramuscular, subcutaneous, intradermal, intraparenchymal, intracerebroventricular, intrathecal, ICM, lumbar puncture and other parenteral routes of administration. Routes of administration may be combined, if desired.

Dosages of the viral vector (for example, rAAV) depend primarily on factors such as the condition being treated, the age, weight and health of the patient, and can thus vary among patients. For example, a therapeutically effective human dosage of the viral vector is generally in the range of from about 25 to about 1000 microliters to about 100 mL of solution containing concentrations of from about 1×10⁹ to 1×10¹⁶ vector genome copies. In certain embodiments, a volume of about 1 mL to about 15 mL, or about 2.5 mL to about 10 mL, or about 5 mL suspension is delivered. In certain embodiments, a volume of about 1, about 2, about 3, about 4, about 5, about 6, about 7, about 8, about 9, about 10, about 11, about 12, about 13, about 14, or about 15 mL suspension is delivered. In certain embodiments, a dose of about 8.9×10¹² to 2.7×10¹⁴ GC total is administered in this volume. In certain embodiments, a dose of about 1.1×10¹⁰ GC/g brain mass to about 3.3×10¹¹ GC/g brain mass is administered in this volume. In certain embodiments, a dose of about 3.0×10⁹, about 4.0×10⁹, about 5.0×10⁹, about 6.0×10⁹, about 7.0×10⁹, about 8.0×10⁹, about 9.0×10⁹, about 1.0×10¹⁰, about 1.1×10¹⁰, about 1.5×10¹⁰, about 2.0×10¹⁰, about 2.5×10¹⁰, about 3.0×10¹⁰, about 3.3×10¹⁰, about 3.5×10¹⁰, about 4.0×10¹⁰, about 4.5×10¹⁰, about 5.0×10¹⁰, about 5.5×10¹⁰, about 6.0×10¹⁰, about 6.5×10¹⁰, about 7.0×10¹⁰, about 7.5×10¹⁰, about 8.0×10¹⁰, about 8.5×10¹⁰, about 9.0×10¹⁰, about 9.5×10¹⁰, about 1.0×10¹¹, about 1.1×10¹¹, about 1.5×10¹¹, about 2.0×10¹¹, about 2.5×10¹¹, about 3.0×10¹¹, about 3.3×10¹¹, about 3.5×10¹¹, about 4.0×10¹¹, about 4.5×10¹¹, about 5.0×10¹¹, about 5.5×10¹¹ about 6.0×10¹¹, about 6.5×10¹¹, about 7.0×10¹¹, about 7.5×10¹¹, about 8.0×10¹¹, about 8.5×10¹¹, about 9.0×10¹¹ GC per gram brain mass is administered in this volume.

The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an subject) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range.

In one embodiment, for human application the dose can range from 1×10¹⁰ to about 1×10¹⁵ GC per kg body weight including all integers or fractional amounts within the range.

In one embodiment, the effective amount of the vector is about 1×10⁹, 2×10⁹, 3×10⁹, 4×10⁹, 5×10⁹, 6×10⁹, 7×10⁹, 8×10⁹, or 9×10⁹ GC per kg body weight including all integers or fractional amounts within the range. In another embodiment, the effective amount of the vector is about 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per kg body weight including all integers or fractional amounts within the range. In another embodiment, the effective amount of the vector is about 1×10¹¹, 2×10¹¹, 3×10¹¹, 4×10¹¹, 5×10¹¹, 6×10¹¹, 7×10¹¹, 8×10¹¹, or 9×10¹¹ GC per kg body weight including all integers or fractional amounts within the range. In another embodiment, the effective amount of the vector is about 1×10¹², 2×10¹², 3×10¹², 4×10¹², 5×10¹², 6×10¹², 7×10¹², 8×10¹², or 9×10¹² GC per kg body weight including all integers or fractional amounts within the range. In another embodiment, the effective amount of the vector is about 1×10¹³, 2×10¹³, 3×10¹³, 4×10¹³, 5×10¹³, 6×10¹³, 7×10¹³, 8×10¹³, or 9×10¹³ GC per kg body weight including all integers or fractional amounts within the range. In another embodiment, the effective amount of the vector is about 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per kg body weight including all integers or fractional amounts within the range. In another embodiment, the effective amount of the vector is about 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per kg body weight including all integers or fractional amounts within the range.

In one embodiment, for human application the dose can range from 1×10¹⁰ to about 1×10¹⁵ GC per gram (g) brain mass including all integers or fractional amounts within the range. In one embodiment, the effective amount of the vector is about 1×10⁹, 2×10⁹, 3×10⁹, 4×10⁹, 5×10⁹, 6×10⁹, 7×10⁹, 8×10⁹, or 9×10⁹ GC per gram (g) brain mass including all integers or fractional amounts within the range. In another embodiment, the effective amount of the vector is about 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per gram (g) brain mass including all integers or fractional amounts within the range. In another embodiment, the effective amount of the vector is about 1×10¹¹, 2×10¹¹, 3×10¹¹, 4×10¹¹, 5×10¹¹, 6×10¹¹, 7×10¹¹, 8×10¹¹, or 9×10¹¹ GC per gram (g) brain mass including all integers or fractional amounts within the range. In another embodiment, the effective amount of the vector is about 1×10¹², 2×10¹², 3×10¹², 4×10¹², 5×10¹², 6×10¹², 7×10¹², 8×10¹², or 9×10¹² GC per gram (g) brain mass including all integers or fractional amounts within the range. In another embodiment, the effective amount of the vector is about 1×10¹³, 2×10¹³, 3×10¹³, 4×10¹³, 5×10¹³, 6×10¹³, 7×10¹³, 8×10¹³, or 9×10¹³ GC per gram (g) brain mass including all integers or fractional amounts within the range. In another embodiment, the effective amount of the vector is about 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per gram (g) brain mass including all integers or fractional amounts within the range. In another embodiment, the effective amount of the vector is about 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per gram (g) brain mass including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 1×10¹⁰ GC/g brain mass to about 3×10¹¹ GC/g brain mass. In certain embodiments, the dose may be in the range of about 1×10¹⁰ GC/g brain mass to about 2.5×10¹¹ GC/g brain mass. In certain embodiments, the dose may be in the range of about 5×10¹⁰ GC/g brain mass.

In one embodiment, the viral constructs may be delivered in doses of from at least about least 1×10⁹ GC to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage may be adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 8.5, pH 7 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other route of delivery.

In certain embodiments, treatment of the composition described herein has minimal to mild asymptomatic degeneration of DRG sensory neurons in animals and/or in human patients, well-tolerated with respect to sensory nerve toxicity and subclinical sensory neuron lesions.

In certain embodiments, the proposed population for the rAAV, vector, composition, and method consist of subjects with early onset late infantile and early juvenile MLD who have symptom onset <7 years of age and whose predictable and rapid decline supports a robust study design and evaluation of functional outcomes within a reasonable follow-up period.

Treatment via the rAAV, vector, composition or method is for disease symptom amelioration and delayed disease progression, including stabilizing the underlying pathology, thereby preventing disease onset and enabling normal or near-normal motor and cognitive development, or substantially preventing or delaying loss of skills (such as acquired developmental and motor milestones) and disease progression. Pre-symptomatic patients are eligible for this treatment.

The AAVhu68 capsid of AAV.hARSAco and the ICM ROA effectively transduces cortical neurons, a small subset of myelin-producing oligodendrocytes, motor neurons with axons projecting into the PNS, and DRG sensory neurons with axons projecting into both the spinal cord and peripheral nerves. Given the broad transduction profile in both the CNS and PNS, ARSA enzyme cross-correction may treat both the CNS manifestations and the peripheral neuropathy observed in many MLD patients, which is not addressed by HSC-GT or HSCT.

Given the nature of MLD, with CNS injury thought to be largely irreversible and the rapid disease progression in the early onset population, the rAAV, vector, composition or method as described herein confers the greatest potential for benefit in patients with no or mild to moderate disease. ICM-delivered AAV gene therapies, such as AAV. hARSAco, show rapid kinetic onset compared to that of HSC-based therapies, with peak ARSA expression in the CSF by 3 weeks after administration (See, Examples). As a result, AAVhARSAco may halt disease progression even in patients who already have some clinical signs of disease. Therefore, patients with early onset MLD who have mild to moderate signs and symptoms would be eligible for the treatment by the rAAV, vector, composition or method as described herein (termed as “treatment”), including those with mild gait abnormalities in patients who are ambulatory and are able to walk at least 10 steps independently, apparent delays in motor milestones acquisition (defined as >95th percentile for age in achieving a given milestone based on WHO criteria (Wijnhoven et al., 2004)), and mild signs on neurological exam.

Indicators of disease progression that are not commonly found in patients with mild to moderate symptoms, include, such as feeding difficulties requiring gastrostomy, development of seizures, low cognitive function, severe abnormalities found on neurological exam (such as very brisk reflexes, severe hypotonus or spasticity of the limbs, severe dysphagia, dyspraxia, or ataxia), and vision or hearing loss would result in exclusion from the trial. A delay in this disease progression, in certain embodiments, is shown as stabilization of disease at a low level of clinical function.

In certain embodiments, pharmacodynamic and efficacy outcomes of the methods is measured at 1, 3, and 6 months, and then every 6 months during the 2 year short-term follow-up period, except for those that require sedation and/or LP. During the long term follow up phase, evaluation frequency decreases to once every 12 months. The early time points and 6 month intervals for the first 2 years were also selected in consideration of the rapid rate of disease progression in untreated early onset MLD patients.

In certain embodiments, amelioration of a disease symptom or delay in disease progression is shown via assessing gross motor function. The GMFC-MLD is a validated, reliable, and simple tool for standardized assessment of gross motor function and decline over time in MLD patients (Kehrer et al., 2011b). It was modeled on a similar tool that assesses motor function in children with cerebral palsy and classifies children's motor function into one of five levels based on differences in self-initiated movements (Palisano et al., 2006). Kehrer et al. adapted the classification system to be relevant to patients with MLD and to provide a classification system in which distinctions between the levels would be considered meaningful in the daily life of children with MLD (the table below) (Kehrer et al., 2011a; Kehrer et al., 2011b).

The GMFC-MLD has been used to both describe the natural history of MLD (Kehrer et al., 2011a) and evaluate motor function after therapeutic intervention (Sessa et al., 2016). One potential limitation of the GMFC-MLD is that the tool was validated for children from 18 months of age onwards, as this represents the upper age limit when children normally learn to walk (Largo et al., 1985; WHO, 2006). However, the tool would still apply for children who achieve the walking milestone before this age.

TABLE
Gross Motor Function Classification System in Metachromatic Leukodystrophy

| | |
|---|---|
| Level 0 | Walking without support with quality of performance normal for age |
| Level 1 | Walking without support but with reduced quality of performance, i.e., instability |
| | when standing or walking |
| Level 2 | Walking with support. Walking without support not possible (fewer than five steps |
| Level 3 | Sitting without support and locomotion such as crawling or rolling. Walking with |
| | or without support not possible |
| Level 4 | (a) Sitting without support but no locomotion or |
| | (b) Sitting without support not possible, but locomotion such as crawling or rolling |
| Level 5 | No locomotion nor sitting without support, but head control is possible |
| Level 6 | Loss of any locomotion as well as loss of any head and trunk control |

The GMFM is included as a measurement for evaluating amelioration of a disease symptom or delay in disease progression. It is a standardized observational instrument designed and validated to measure change in gross motor function over time and after intervention in children with cerebral palsy (Russell et al., 1989; Lundkvist Josenby et al., 2009; Alotaibi et al., 2014). The GMFM is an 88-item tool that assesses motor function grouped across five functional domains: lying and rolling, sitting, crawling and kneeling, standing, and walking, running and jumping. Reference curves have also been developed for healthy children, who typically attain the most difficult skills on the scale (walking, running, jumping) by 5 years of age (Palisano et al., 2006). Although the tool is not validated for children with MLD, it has been proven useful for early onset MLD patients who received HSC-GT in demonstrating (near) normal gross motor development in subjects treated in the pre-symptomatic stage (Sessa et al., 2016; Fumagalli et al., 2017). One of the advantages of the 88-item instrument is that it contains a large amount of information about various aspects of motor function and the sub-domains can be summarized and reported separately. Due to a plateau effect, the tool may not be as informative in older early juvenile patients who may already have reached the maximum GMFM score prior to study enrolment (i.e., cannot measure acquisition of new skills), although it would still be able to show maintenance or loss of gross motor function over time.

Peripheral neuropathy is a common, painful, and progressively debilitating manifestation of MLD that can aggravate the fine and gross motor dysfunction in these patients (Gieselmann and Krageloh-Mann, 2010; van Rappard et al., 2015). HSC-based treatments do not appear to substantially ameliorate peripheral neuropathy (Boucher et al., 2015; van Rappard et al., 2016). The ability of AAV.hARSAco to transduce neurons, DRG, and peripheral nerve axons cells allow for expression of the ARSA enzyme within the brain and peripheral nerve dysfunction. Neurological examinations may be performed to assess clinical manifestations of peripheral neuropathy, and nerve conduction studies may be performed on representative motor and sensory nerves (deep peroneal nerve, median nerve, ulnar nerve, and sural nerve). As MLD is primarily a demyelinating disease, nerve conduction velocity is considered a relevant neurophysiologic parameter of the disease (Biffi et al., 2008) and may be measured.

Motor milestone development depends on the age and stage of disease at the time of subject enrollment. Depending on the age of the subject at enrollment, subjects may have achieved certain motor skills or not yet shown signs of motor milestone development. Assessments will track age-at-achievement and age-at-loss for all milestones. Motor milestone achievement will be defined for six gross milestones based on the WHO criteria outlined in the table below.

TABLE
World Health Organization Performance Criteria for Gross Motor Milestones

| Gross Motor Milestone | Multicenter Growth Reference Study Performance Criteria |
|---|---|
| Sitting without support | Child sits up straight with the head erect for at least 10 seconds. Child |
| | does not use arms or hands to balance body or support position. |
| Hands-and-knees crawling | Child alternately moves forward or backward on hands and knees. The |
| | stomach does not touch the supporting surface. There are continuous and |
| | consecutive movements, at least three in a row. |
| Standing with assistance | Child stands in upright position on both feet, holding onto a stable |
| | object (e.g., furniture) with both hands without leaning on it. The body |
| | does not ouch the stable object, and the legs support most of the body |
| | weight. Child thus stands with assistance for at least 10 seconds. |
| Walking with assistance | Child is in upright position with the back straight. Child makes sideways |
| | or forward steps by holding on a stable objects (e.g., furniture) with one |
| | of both hands. One leg moves forward while the other supports part of |
| | the body weight. Child takes at least five steps in this manner. |
| Standing alone | Childs stands in upright position on both feed (not on the toes) with the |
| | back straight. The legs support 100% of the child's weigh. There is no |
| | contact with a person or objects. Child stands alone for at least |
| | 10 seconds. |
| Walking alone | Child takes at least five steps independently in upright position with the |
| | back straight. One leg moves forward while the other supports most of |
| | the body weight. There is no contact with a person or object. |

Adapted from (Wijnhoven et al., 2004).

Neurocognitive and behavioral manifestations may be assessed to show amelioration of a disease symptom or delay in disease progression. Assessing these manifestations is especially important in children with early juvenile MLD, in whom behavioral and cognitive symptoms are an important manifestation of the disease that may develop simultaneously with motor dysfunction. Clinical scales may be used to quantify the effects of AAV.hARSAco on development of and changes in cognition, language, and motor function, which may be assessed using the BSID-III and the WISC-V with transition to age-appropriate assessment tools done according to the patient's estimated developmental age. Outcomes may be compared to the norms
of typically developing children and untreated children. Each proposed measure has been previously used in the MLD population (Clarke et al., 1989; Boucher et al., 2015; Sessa et al., 2016).
BSID-JJJ: This scale used primarily to assess the development of infants and toddlers, ages 1-42 months (Albers and Grieve, 2007). It consists of a standardized series of developmental play tasks. It derives a developmental quotient by converting raw scores of successfully completed items to scale scores and composite scores followed by a comparison of the scores with norms taken from typically developing children of the same age. The BSID-III has three main subtests. A Cognitive Scale includes such items as attention to familiar and unfamiliar objects, looking for a fallen object, and pretend play. A Language Scale assesses understanding and expression of language (e.g., the ability to follow directions and naming objects). A Motor Scale measures gross and fine motor skills (e.g., grasping, sitting, stacking blocks, and climbing stairs). Thus, the BSID-III can provide additional motor function information to complement the GMFC-MLD and GMFM.
WISC-V: This scale is an individually administered intelligence test or children between the ages of 6 and 16 years of age. It generates a Full Scale IQ that represents a child's general intellectual ability and provides five primary index scores: Verbal Comprehension Index, Visual Spatial Index, Fluid Reasoning Index, Working Memory Index, and Processing Speed Index. These indices represent a child's abilities in discrete cognitive domains.

Survival is included as a measurement for amelioration of a disease symptom or delay in disease progression. Death is expected in the first 5 years of life for the majority of patients diagnosed with late infantile MLD, with 5 year survival of 25% (Mahmood et al., 2010), although survival can extend into the second decade of life with current levels of supportive care (Gomez-Ospina, 2017). Thus, the 5 year follow-up may be sufficient to demonstrate a survival benefit in the late infantile population, although it may not be sufficiently long to assess survival in the early juvenile cohort. Importantly, with improved levels of supportive care, children with early onset MLD can now remain alive beyond 10 years of age, albeit it at a very low level of function.

While seizures are not usually a presenting symptom for the early onset population, it is a feature of later stages of the disease (Gieselmann and Krageloh-Mann, 2010; Mahmood et al., 2010). Parents may be asked to maintain a diary to record seizure activity (onset, frequency, length, and type of seizure), which enables assessing whether AAV.hARSAco can either prevent or delay onset of seizures or decrease the frequency of seizure events.

Measures of adaptive behavior along with parent and patient quality of life may be evaluated to show amelioration of a disease symptom or delay in disease progression using the tools that have been previously utilized in MLD patients (Martin et al., 2013; Boucher et al., 2015; Sessa et al., 2016):
Vineland-III: Assesses adaptive behavior from birth through adulthood (0-90 years) across five domains: communication, daily living skills, socialization, motor skills, and maladaptive behavior. Improvements from the Vineland-II to the Vineland-III incorporate questions to enable better understanding of developmental disabilities.
PedsQOL and PedsQL-IS: As is the case with severe pediatric diseases, the burden of the disease on the family is significant. The Pediatric Quality of Life Inventory™ is a validated a tool that assesses quality of life in children and their parents (by parent proxy reports). It has been validated in healthy children and adolescents and has been used in various pediatric diseases (Iannaccone et al., 2009; Absoud et al., 2011; Consolaro and Ravelli, 2016). Therefore, the PedsQL is included to evaluate the impact of AAV.hARSAco on the quality of life of the patient and their family. It can be applied to parents of children 2 years old and above and may therefore be informative as the children age over the 5 year follow-up period. The Pediatric Quality of Life Inventory™ Infant Scale (Vari et al., 2011) is a validated modular instrument completed by parents designed to measure health-related quality of life specifically for healthy and ill infants aged 1-24 months. It also provides the possibility for self-reporting by children aged 5 years and up.
Lansky Performance Index: A scale that measures the functional status of an individual and provides a score that represents the person's ability to carry out normal daily activities.

Effect of rAAV (e.g., AAV.hARSAco), vector, composition or method as described herein on disease pathology may be measured to show amelioration of a disease symptom or delay in disease progression, including changes in myelination, functional outcomes related to myelination, and potential disease biomarkers.

The primary hallmark of MLD, central and peripheral demyelination, may be examined to show amelioration of a disease symptom or delay in disease progression following rAAV administration. Central demyelination may be tracked by MRI measurements of white matter regions, changes in which are indicators of disease state and progression (Gieselmann and Krageloh-Mann, 2010; Martin et al., 2012; van Rappard et al., 2015). Central demyelination detected by MRI positively correlates with the degree of gross motor dysfunction (Groeschel et al., 2011). Peripheral demyelination may be measured indirectly via NCV studies on the motor nerves (deep peroneal, tibial, and ulnar nerves) and sensory nerves (sural and median nerves), which also provides a readout of peripheral neuropathy. NCV studies monitor for fluctuations indicative of a change in biologically active myelin (i.e., F-wave and distal latencies, amplitude, or presence or absence of a response).

In addition to measuring total demyelination scores and brain white matter atrophy, various brain neuronal metabolites, including NAA, ml, Cho, and Lac, may be measured over time using proton MRS. There is evidence that NAA levels strongly correlate with gross motor function, with the NAA signal intensity decreasing as the disease process advances (Kruse et al., 1993; Dali et al., 2010). Additionally, proton MRS studies have shown a decrease in the NAA/creatinine ratio and an increase in the Cho/creatinine ratio and ml and Lac levels during MLD disease evolution (Martin et al., 2012). Thus, neuronal metabolites may be evaluated as biomarkers showing amelioration of a disease symptom or delay in disease progression.

There is evidence that peripheral nerve and CSF sulfatide and lysosulfatide accumulation correlates with abnormalities in electrophysiological parameters and large myelinated fiber loss in the sural nerve (Dali et al., 2015). CSF (lyso)-sulfatide levels may therefore reflect disease severity in the PNS and could provide a marker to assess the impact of a therapy on the peripheral nervous system. CSF sulfatide and lyso-sulfatide levels may be included to show amelioration of a disease symptom or delay in disease progression.

Similar to seizures, vision loss is not a common presenting symptom in early onset MLD, but it does appear in the later stages of disease (Gieselmann and Krageloh-Mann, 2010; van Rappard et al., 2015). Tracking vision loss through the use of VEPs offers the opportunity to assess the ability of the rAAV as described herein to delay or prevent vision loss. VEPs may be used to objectively measure responses to visual stimuli as an indicator of central visual impairment or loss. Hearing loss is also common during disease progression, and early indications of auditory abnormalities may be measured via BAER testing.

One of the sequelae of MLD in visceral tissues involves sulfatide deposition in the gallbladder wall, resulting in gallbladder wall thickening and polyps that may require surgical intervention and can be visualized on ultrasound (Rodriguez-Waitkus et al., 2011; Kim et al., 2017). Gallbladder abnormalities are a common finding in MLD and predispose the patient to gallbladder carcinoma (van Rappard et al., 2016) and occur in all subtypes of MLD.

In certain embodiment, the assays listed below may be performed to show amelioration of a disease symptom and/or a delay in disease progression:

Hematology, Serum Chemistry, Coagulation, LFTs; Urinalysis; HepB/HepC/HIV Serology; Serum Biomarkers (ARSA); Vector DNA in serum and urine; Serum anti-AAVhu68 nAbs; ELISpot (capsid and ARSA); CSF Collection and Assessments; LP (to collect CSF); CSF Cytology and Chemistry; CSF Disease Biomarkers (ARSA, sulfatide, lyso-sulfatide); CSF anti-AAVhu68 nAbs; Vector DNA in CSF; Physical Exam (including length and weight); Neurological Exam; Vital Signsd; ECGd; Sensory Nerve Conduction Studies; GMFC-MLD; GMFM; BSID-IIIe; WISC-V; Vineland-IIIe; Lansky Performance Index; PedsQL; PedsQL-IS; Caregiver/Parent QoL Assessment; Motor Milestone Assessment; Training on Seizure Diary Completion; Review of Seizure Diary; Imaging Assessments; MRI; MRS; NCV Measurements; and VEP.

Related abbreviations are listed below: AAVhu68, adeno-associated virus serotype hu68; AE, adverse event; ARSA, Arylsulfatase A; BAER, brainstem auditory evoked response; BSID-III, Bayley Scales of Infant and Toddler Development, Third Edition; CSF, cerebrospinal fluid; DNA, deoxyribonucleic acid; ECG, electrocardiogram; ELISpot, enzyme-linked immunospot; GMFC-MLD, Gross Motor Function Classification in Metachromatic Leukodystrophy; GMFM, Gross Motor Function Measure; HepB, hepatitis B; HepC, hepatitis C; HIV, human immunodeficiency virus; ICM, intra-cisterna magna; LFTs, liver function tests; LP, lumbar puncture; MRI, magnetic resonance imaging; MRS, magnetic resonance spectroscopy; nAbs, neutralizing antibodies; NCV, nerve conduction velocity; PedsQL/PedQL-IS, Pediatric Quality of Life Inventory; QoL, Quality of Life; VEP, visual evoked potentials; Vineland-III, Vineland Adaptive Behavior Scales, Third Edition; WISC-V, Wechsler Intelligence Scale for Children, Fifth Edition.

The rAAV, vector, composition and methods provides supra-physiologic levels of the ARSA enzyme within days of administration to both the CNS and PNS, both of which are affected in MLD patients. The AAVhu68 capsid and ICM route were selected based upon the observation of superior transduction of neurons, DRG, and peripheral nerve axons cells. Although vector transduction of myelinating cells is limited, the cross-correction potential would allow for enzyme uptake by oligodendrocytes. Furthermore, AAV vector and ARSA enzyme can be transported along axons, expanding the expression of the therapeutic enzyme within the brain and to the periphery.

X. Apparatus and Method for Delivery of a Pharmaceutical Composition into Cerebrospinal Fluid

In certain embodiments, the AAV.CB7.CI.hARSAco.rBG is administered as a single dose via a computed tomography—(CT-) guided sub-occipital injection into the cisterna magna (intra-cisterna magna [ICM]).

Many animal models of monogenic CNS diseases have been successfully treated using AAV-mediated gene transfer, and several early human studies using a first-generation AAV vector demonstrated the safety of vector delivery to the brain (Janson et al., 2002; Mandel and Burger, 2004; Kaplitt et al., 2007; Mittermeyer et al., 2012; Bartus et al., 2014). However, the low efficiency of these vectors prevented the translation of efficacy in animal models into clinical benefits. With the advent of second-generation AAV vectors, the potential for gene transfer to the brain has been greatly enhanced. In particular, some clade F isolates, such as AAV9, have demonstrated extremely efficient brain transduction (Gray et al., 2013; Haurigot et al., 2013; Hinderer et al., 2014; Bell et al., 2015). Using these more efficient vectors, gene therapy has shown greatly enhanced potential to treat a variety of neurological disorders, and several programs utilizing second-generation vectors have progressed into the clinic (Haurigot et al., 2013; Hinderer et al., 2014; Bell et al., 2015; Gurda et al., 2016; Hinderer et al., 2016).

Early studies of CNS gene transfer were challenged not only by the low gene transfer efficiency of first-generation AAV vectors, but also limitations in the available delivery methods. Most early non-clinical and clinical studies utilized direct vector injection into the parenchyma of the brain or spinal cord (Vite et al., 2005; Worgall et al., 2008; Colle et al., 2010; Ellinwood et al., 2011; Tardieu et al., 2014). While this method yields robust transduction near the injection site, translating this approach to diseases affecting cells throughout the CNS was difficult because large numbers of vector injections were required to achieve widespread transgene delivery. An additional obstacle to CNS gene transfer was the finding that intraparenchymal vector injection could trigger inflammation at the injection site, which could promote adaptive immune responses against the transgene product (Worgall et al., 2008; Colle et al., 2010; Ellinwood et al., 2011; Ciesielska et al., 2013). Two alternative vector delivery methods have been developed to more safely and effectively target large regions of the CNS.

The first was based on the discovery that some AAV vectors, including AAV9, can transduce cells within the CNS after IV delivery (Foust et al., 2009). However, IV vector delivery has two critical limitations. First, the low efficiency of vector penetration into the CNS necessitates extremely large vector doses to achieve therapeutic levels of transgene expression, increasing the risk of systemic toxicity and potentially requiring quantities of vector that may not be feasible to manufacture for many patient populations (Gray et al., 2011; Hinderer et al., 2014; Gurda et al., 2016). Second, gene transfer to the CNS after IV vector delivery is profoundly limited by pre-existing NAbs to the vector capsid (Gray et al., 2011). Given the high prevalence of AAV NAbs in humans, this leaves a significant population of patients who would not be candidates for IV AAV treatment. In order to circumvent the limitations of IV AAV for targeting the CNS, IT vector delivery has been developed as an alternative approach. Using the CSF as a vehicle for vector dispersal, the IT ROA has the potential to achieve transgene delivery throughout the CNS and PNS with a single minimally invasive injection. Animal studies have demonstrated that by obviating the need to cross the blood-brain barrier, IT delivery results in substantially more efficient CNS gene transfer with much lower vector doses than those required for the IV approach (Gray et al., 2011; Hinderer et al., 2014). Since antibodies are present at very low levels in CSF, IT vector delivery is not affected by pre-existing NAbs to the AAV capsid, making this approach applicable to a broader patient population (Haurigot et al., 2013). IT AAV delivery can be performed using a variety of routes for CSF access. Lumbar puncture (LP) is the most common method for accessing CSF, and was therefore evaluated as a route for AAV administration in NHPs. Delivery of an AAV9 vector into the CSF via an LP was found to be at least 10-fold less efficient at transducing cells of the brain and spinal cord compared to injection of the vector more superiorly at the level of the cisterna magna (Hinderer et al., 2014).

The superior brain transduction achieved with a single ICM injection in NHPs resulted in the selection of this ROA for the clinical studies of AAV.CB7.CI.hARSAco.rBG. Once a common procedure, ICM injection (also known as suboccipital puncture) was ultimately supplanted by LPs in the pre-imaging era due to rare cases of injury to the brainstem or nearby blood vessels (Saunders and Riordan, 1929). Today, the procedure can be performed under real-time CT guidance, allowing for visualization of critical structures, such as the medulla, vertebral arteries, and posterior inferior cerebellar arteries during needle insertion (Pomerantz et al., 2005; Hinderer et al., 2014).

In one aspect, the vectors provided herein may be administered intrathecally via the method and/or the device provided in this section and described in WO 2018/160582, which is incorporated by reference herein. Alternatively, other devices and methods may be selected.

In certain embodiments, the method comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis.

On the day of treatment, the appropriate concentration of rAAVhu68.hARSAco is be prepared. A syringe containing 5.6 mL of rAAVhu68.hARSAco at the appropriate concentration is delivered to the procedure room. The following personnel are present for study drug administration: interventionalist performing the procedure; anesthesiologist and respiratory technician(s); nurses and physician assistants; CT (or operating room) technicians; site research coordinator. Prior to drug administration, a lumbar puncture is performed to remove a predetermined volume of CSF and then to inject iodinated contrast intrathecally (IT) to aid in visualization of relevant anatomy of the cisterna magna. Intravenous (IV) contrast may be administered prior to or during needle insertion as an alternative to the intrathecal contrast. The decision to used IV or IT contrast is at the discretion of the interventionalist. The subject is anesthetized, intubated, and positioned on the procedure table. The injection site is prepped and draped using sterile technique. A spinal needle (22-25 G) are advanced into the cisterna magna under fluoroscopic guidance. A larger introducer needle may be used to assist with needle placement. After confirmation of needle placement, the extension set are attached to the spinal needle and allowed to fill with CSF. At the discretion of the interventionalist, a syringe containing contrast material may be connected to the extension set and a small amount injected to confirm needle placement in the cisterna magna. After the needle placement is confirmed by CT guidance+/−contrast injection, a syringe containing 5.6 mL of rAAVhu68.hARSAco is connected to the extension set. The syringe contents are slowly injected over 1-2 minutes, delivering a volume of 5.0 mL. The needle are slowly removed from the subject.

In one embodiment, doses may be scaled by brain mass, which provides an approximation of the size of the CSF compartment. In a further embodiment, dose conversions are based on a brain mass of 0.4 g for an adult mouse, 90 g for a juvenile rhesus macaque, and 800 g for children 4-18 months of age. The following table provides illustrative doses for a murine MED study, NHP toxicology study, and equivalent human doses.

| Dose | | | |
|---|---|---|---|
| (GC/g brain mass) | Mouse (GC) | NHP (GC) | Human (GC) |
| 3.33 × 10¹¹ | 1.30 × 10¹¹ | 3.00 × 10¹³ | 2.70 × 10¹⁴ |
| 1.11 × 10¹¹ | 4.40 × 10¹⁰ | 1.00 × 10¹³ | 8.90 × 10¹³ |
| 3.33 × 10¹⁰ | 1.30 × 10¹⁰ | 3.00 × 10¹² | 2.70 × 10¹³ |
| 1.11 × 10¹⁰ | 4.40 × 10⁹ | — | 8.90 × 10¹² |

In certain embodiments, a rAAVhu68.hARSAco vector is administered to a subject in a single dose. In certain embodiments, multiple doses (for example 2 doses) may be desired. For example, for infants under 6 months, multiple doses delivered days, weeks, or months, apart may be desired.

In certain embodiments, a single dose of rAAVhu68.hARSAco vector is about 1×10⁹ GC to about 3×10¹¹ GC. In certain embodiments, the dose of rAAVhu68.HARSA is 1×10¹⁰ GC/brain mass to 3.33×10¹¹ GC/brain mass. In other embodiments, different doses may be selected.

The compositions can be formulated in dosage units to contain an amount of AAV that is in the range of about 1×10⁹ genome copies (GC) to about 5×10¹³ GC (to treat an average subject of 70 kg in body weight). In one embodiment, a spinal tap is performed in which from about 15 mL (or less) to about 40 mL CSF is removed and in which vector is admixed with the CSF and/or suspended in a compatible carrier and delivered to the subject. In one example, the vector concentration is about 3×10¹³ GC, but other amounts such as about 1×10⁹ GC, about 5×10⁹ GC, about 1×10¹⁰ GC, about 5×10¹⁰ GC, about 1×10¹¹ GC, about 5×10¹¹ GC, about 1×10¹² GC, about 5×10¹² GC, or about 1.0×10¹³ GC.

A co-therapy may be delivered with the rAAVhu68.hARSAco compositions provided herein. Co-therapies such as described earlier in this application are incorporated herein by reference.

In certain embodiments, a recombinant adeno-associated virus (rAAV) is provided which is useful for treating Metachromatic Leukodystrophy or a disorder associated with a hARSA gene defect. The rAAV may comprise: (a) an AAVhu68 capsid; and (b) a vector genome packaged in the AAV capsid of (a), wherein the vector genome comprises inverted terminal repeats (ITR) and a nucleic acid sequence encoding a functional human Arylsulfatase A (hARSA) under control of regulatory sequences which direct the hARSA expression, wherein the hARSA coding sequence comprises a sequence of nucleotide (nt) 55 to nt 1521 of SEQ ID NO: 1, or a sequence at least 95% to 99.9% identical thereto which encodes a functional hARSA. In certain embodiments, the functional protein comprises a signal peptide and an amino acid sequence of amino acid (aa) 19 to aa 507 of SEQ ID NO: 2. In certain embodiments, the signal peptide has an amino acid sequence of aa 1 to aa 18 of SEQ ID NO: 2 or an amino acid sequence of aa 1 to aa 20 of SEQ ID NO: 4. In certain embodiments, the regulatory sequences direct hARSA expression in nervous system cells. In certain embodiments, the regulatory sequences comprise a ubiquitous promoter, including a CB7 promoter. In certain embodiments, the regulatory elements comprise one or more of a Kozak sequence, a polyadenylation sequence, an intron, an enhancer, and a TATA signal. In certain embodiments, the hARSA coding sequence is at least 95% to 99.9% identical to SEQ ID NO: 1 and encodes a functional hARSA. In certain embodiments, the hARSA coding sequence is SEQ ID NO: 1 or SEQ ID NO: 3. In certain embodiments, the vector genome has a sequence of nt 1 to nt 3883 of SEQ ID NO: 5. In certain embodiments, the AAVhu68 capsid is produced from a sequence encoding the predicted amino acid sequence of SEQ ID NO: 7.

In certain embodiments, an aqueous pharmaceutical composition is provided which comprises one or more rAAV and/or vectors as described herein and a formulation buffer. In certain embodiments, a formulation buffer comprises: an artificial cerebrospinal fluid comprising buffered saline and one or more of sodium, calcium, magnesium, potassium, or mixtures thereof; and a surfactant. In certain embodiments, the surfactant is present at 0.0005% to about 0.001% of the pharmaceutical composition. In certain embodiments, the composition is at a pH in the range of 7.5 to 7.8. In certain embodiments, the formulation buffer is suitable for an intra-cisterna magna injection (ICM), intravenous delivery, intrathecal administration, or intracerebroventricular administration. In certain embodiments, a vector comprising an expression cassette, wherein the expression cassette comprises a nucleic acid sequence encoding a functional human Arylsulfatase A (hARSA) under control of regulatory sequences which direct the hARSA expression. The functional hARSA protein may comprises a signal peptide and an amino acid sequence of amino acid (aa) 19 to aa 507 of SEQ ID NO: 2. In certain embodiments, the signal peptide has an amino acid sequence of aa 1 to aa 18 of SEQ ID NO: 2 or an amino acid sequence of aa 1 to aa 20 of SEQ ID NO: 4. In certain embodiments, the hARSA coding sequence has a sequence of nucleotide (nt) 55 to nt 1521 of SEQ ID NO: 1, or a sequence at least 95% to 99.9% identical thereto which encodes a functional hARSA. In certain embodiments, the hARSA coding sequence is SEQ ID NO: 1 or SEQ ID NO: 3. In certain embodiments, the vector is a viral vector selected from a recombinant adeno-associated virus, a recombinant parvovirus, a recombinant lentivirus, a recombinant retrovirus, or a recombinant adenovirus; or a non-viral vector selected from naked DNA, naked RNA, an inorganic particle, a lipid particle, a polymer-based vector, or a chitosan-based formulation. In certain embodiments, a pharmaceutical composition is provided which comprises a vector as provided herein and a formulation buffer. In certain embodiments, the formulation buffer is suitable for intravenous delivery, an intra-cisterna magna injection (ICM) intrathecal administration, or intracerebroventricular administration.

In certain embodiments, a method of treating Metachromatic Leukodystrophy or a disease associated with Arylsulfatase A (ARSA) gene mutation is provided which comprises administering an effective amount of the rAAV, the pharmaceutical composition, and/or the vector to a subject in need thereof. In certain embodiments, the rAAV or the vector is administered via a CT-guided sub-occipital injection into the cisterna magna. In certain embodiments, the method involves delivering the rAAV, the pharmaceutical composition, or the vector in a single dose. In certain embodiments, the rAAV is administered at a dose between 3.00×10¹⁰ genome copies (GC) per gram (GC/g) of brain mass and 1.00×10¹² GC/g of brain mass.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, an “expression cassette” refers to a nucleic acid molecule which comprises a coding sequence, promoter, and may include other regulatory sequences therefor. In certain embodiments, a vector genome may contain two or more expression cassettes. In other embodiments, the term “transgene” may be used interchangeably with “expression cassette”. Typically, such an expression cassette for generating a viral vector contains the coding sequence for the gene product described herein flanked by packaging signals of the viral genome and other expression control sequences such as those described herein.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

As used herein, an “effective amount” refers to the amount of the rAAV composition which delivers and expresses in the target cells an amount of the gene product from the vector genome. An effective amount may be determined based on an animal model, rather than a human patient. Examples of a suitable murine or NHP model are described herein.

The term “translation” in the context of the present invention relates to a process at the ribosome, wherein an mRNA strand controls the assembly of an amino acid sequence to generate a protein or a peptide.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

As described above, the term “about” when used to modify a numerical value means a variation of ±10%, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

The following examples are illustrative only and are not intended to limit the present invention.

The vector AAVhu68.CB7.CI.hARSAco.rBG (also termed as AAV.CB7.CI.hARSAco.rBG or AAVhu68.hARSAco or AAV.hARSAco) was delivered into the CSF to achieve therapeutic ARSA expression levels and rescue several biomarkers of MLD.

### Example 1—AAV.hARSAco Vector

Components of an AAV.hARSAco are illustrated in the following table.

| | AAV.CB7.CI.hARSAco.rBG |
|---|---|
| Name: | (AAVhu68.CB7.CI.hARSAco.rBG) |
| Gene | Engineered human arylsulfatase A (ARSA) gene |
| Inserts: | |
| Control | Regulatory element derived from the chicken β-actin |
| Element: | (BA) promoter |
| | Human cytomegalovirus immediate-early |
| | enhancer (CMV IE) |
| Other | Chimeric intron consisting of a chicken BA splice donor |
| Elements: | and a rabbit β-globin (rBG) splice acceptor element |
| | Polyadenylation (PolyA) signal derived from the rBG gene |
| | Two inverted terminal repeat sequences (ITRs) |
| AAV | AAVhu68 |
| Serotype: | |

Vectors are constructed from cis-plasmids containing a coding sequence for human ARSA (SEQ ID NO: 1 and SEQ ID NO: 3) expressed from the chicken beta actin promoter with a cytomegalovirus enhancer (CB7; SEQ ID NO: 16) flanked by AAV2 inverted terminal repeats.

The vectors are packaged in an AAV serotype hu68 capsid (WO 2018/160582) by triple transfection of adherent HEK 293 cells and purified by iodixanol gradient centrifugation as previously described in Lock, M., et al. Rapid, Simple, and Versatile Manufacturing of Recombinant Adeno-Associated Viral Vectors at Scale. Human Gene Therapy 21, 1259-1271 (2010).

More particularly, AAV.CB7.CI.hARSAco.rBG is produced by triple plasmid transfection of HEK293 working cell bank (WCB) cells with the AAV cis plasmid (pENN.AAV.CB7.CI.hARSAco.rBG.KanR), the AAV trans plasmid encoding the AAV2 rep and AAVhu68 cap genes (pAAV2/hu68.KanR), and the helper adenovirus plasmid (pAdΔF6.KanR). In some embodiments, the size of the AAV.CB7.CI.hARSAco.rBG packaged vector genome is 3883 bases (nt 1 to nt 3883 of SEQ ID NO: 5) with 130-bp ITR shorted by 15 bp from the terminal of the intact 145-bp ITR. In some embodiments, the size of the AAV.CB7.CI.hARSAco.rBG packaged vector genome is 3913 bases (nt 1 to nt 3883 of SEQ ID NO: 5) with an intact 145-bp ITR.

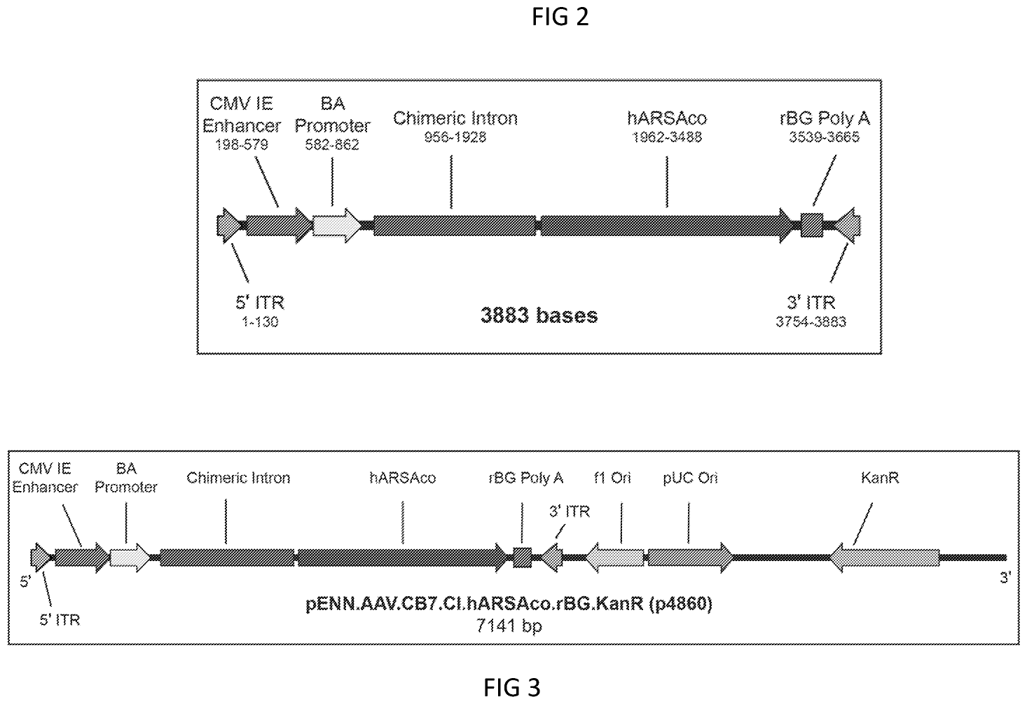

The cis plasmid (FIG. 2) contains the following vector genome sequence elements:

Inverted Terminal Repeat (ITR): The ITRs are identical, reverse complementary sequences derived from AAV2 (130 base pairs [bp], GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.

Human Cytomegalovirus Immediate-Early Enhancer (CMV IE): This enhancer sequence obtained from human-derived cytomegalovirus (382 bp, GenBank: K03104.1) increases expression of downstream transgenes.

Chicken β-Actin (BA) Promoter (SEQ ID NO: 18): This ubiquitous promoter (281 bp, GenBank: X00182.1) was selected to drive transgene expression in any cell type.

Chimeric Intron (CI): The hybrid intron consists of a chicken BA splice donor (973 bp, GenBank: X00182.1) and rabbit β-globin splice acceptor element. The intron is transcribed, but removed from the mature messenger ribonucleic acid (mRNA) by splicing, bringing together the sequences on either side of it. The presence of an intron in an expression cassette has been shown to facilitate the transport of mRNA from the nucleus to the cytoplasm, thus enhancing the accumulation of the steady level of mRNA for translation. This is a common feature in gene vectors intended for increased levels of gene expression.

Coding Sequence: The engineered complementary deoxyribonucleic acid (cDNA) of the human ARSA gene (SEQ ID NO: 1 or SEQ ID NO: 3) encodes arylsulfatase A, which is a lysosomal enzyme responsible for the desulfation of the sulfated galactosphingolipids, galactosylceramide-3-O-sulfate and galactosylsphingosine-3-O-sulfate (1527 bp; 509 amino acids [aa], GenBank: NP_000478.3).

Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail.

All component parts of the plasmid have been verified by direct sequencing.

The AAV2/hu68 trans plasmid (FIG. 3) is pAAV2/hu68.KanR. It is 8030 bp in length and encodes four wild type AAV2 replicase (Rep) proteins required for the replication and packaging of the AAV vector genome. The pAAV2/hu68.KanR plasmid also encodes three wild type AAVhu68 virion protein capsid (Cap) proteins, which assemble into a virion shell of the AAV serotype hu68 to house the AAV vector genome. The novel AAVhu68 sequence was obtained from human heart tissue DNA.

To create the pAAV2/hu68.KanR trans plasmid, the AAV9 cap gene from plasmid pAAV2/9n (which encodes the wild type AAV2 rep and AAV9 cap genes on a plasmid backbone derived from the pBluescript KS vector) was removed and replaced with the AAVhu68 cap gene.

The ampicillin resistance (AmpR) gene was also replaced with the kanamycin resistance (KanR) gene, yielding pAAV2/hu68.KanR. This cloning strategy relocated the AAV p5 promoter sequence (which normally drives rep expression) from the 5′ end of rep to the 3′ end of cap, leaving behind a truncated p5 promoter upstream of rep. This truncated promoter serves to down-regulate expression of rep and, consequently, maximize vector production. All component parts of the plasmid have been verified by direct sequencing.

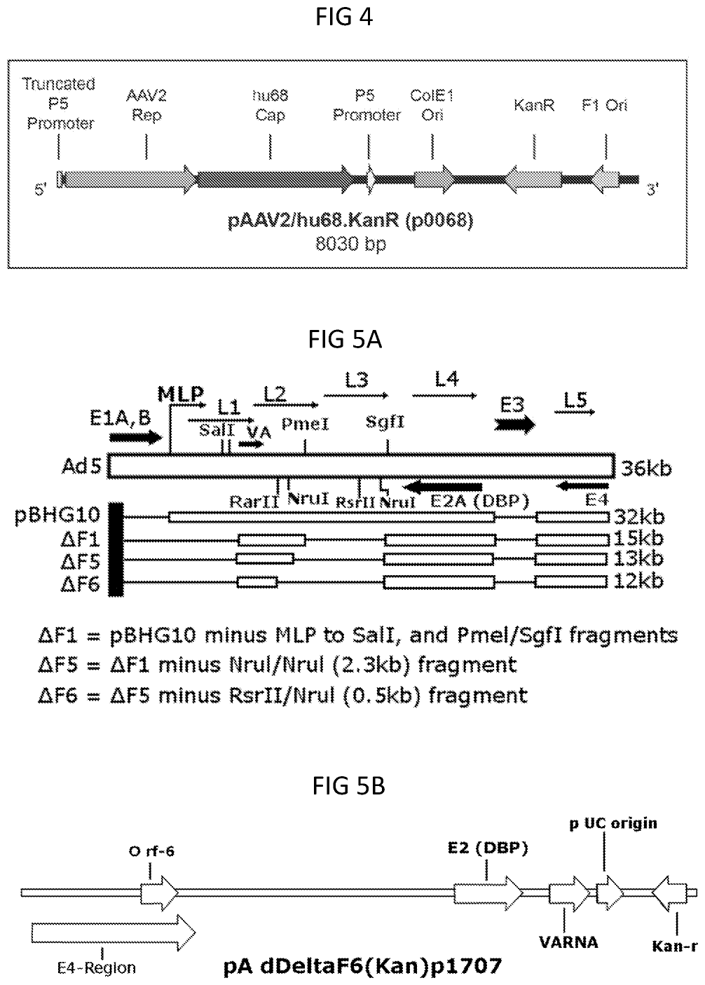

Plasmid pAdDeltaF6(KanR) (FIG. 4) was constructed and is 15,770 bp in size. The plasmid contains the regions of adenovirus genome that are important for AAV replication; namely, E2A, E4, and VA RNA (the adenovirus E1 functions are provided by the HEK293 cells). However, the plasmid does not contain other adenovirus replication or structural genes. The plasmid does not contain the cis elements critical for replication, such as the adenoviral ITRs; therefore, no infectious adenovirus is expected to be generated. The plasmid was derived from an E1, E3-deleted molecular clone of Ad5 (pBHG10, a pBR322-based plasmid). Deletions were introduced into Ad5 to eliminate expression of unnecessary adenovirus genes and reduce the amount of adenovirus DNA from 32 kb to 12 kb (FIG. 5A). Finally, the ampicillin resistance gene was replaced by the kanamycin resistance gene to create pAdeltaF6(KanR) (FIG. 5B). The E2, E4, and VA adenoviral genes that remain in this plasmid, along with E1, which is present in HEK293 cells, are necessary for AAV vector production.

AAV.CB7.CI.hARSAco.rBG is manufactured by transient transfection of HEK293 cells followed by downstream purification. A manufacturing process flow diagram is shown FIGS. 6 and 7. The major reagents entering into the preparation of the product are indicated on the left side of the diagram and in-process quality assessments are depicted on the right side of the diagram. A description of each production and purification step is also provided. Product manufacturing follows a linear flow of unit operations and utilizes disposable, closed bioprocessing systems unless otherwise specified. All steps of the production process involving cell culture, from cell seeding to harvest collection, are performed aseptically using sterile, single-use disposable tubing and bag assemblies. Cells are expanded using Corning flatware (T-Flasks, CellSTACKs [CS-10] and/or HYPERStacks [HS-36]). Cells are transfected in a bioreactor(s), and all open manipulations are performed in class II biological safety cabinets (BSCs) in an ISO Class 5 environment. The purification process are performed in a closed system where possible.

The manufacturing process for AAV.CB7.CI.hARSAco.rBG was developed and involves transient transfection of human embryonic kidney 293 (HEK293) cells with plasmid DNA. The HEK293 working cell bank (WCB) used in the production was tested and qualified as detailed in FDA and International Council for Harmonisation (ICH) guidelines. To support clinical development, a single batch or multiple batches of the bulk drug substance (BDS) is/are produced by polyethylenimine—(PEI-) mediated triple transfection of HEK293 cells in bioreactors. Harvested AAV material is purified sequentially by clarification, tangential flow filtration (TFF), affinity chromatography, and anion exchange chromatography in disposable, closed bioprocessing systems where possible. The product is formulated in intrathecal final formulation buffer (ITFFB; artificial CSF with 0.001% Pluronic F-68). The BDS batch or batches are frozen, subsequently thawed, pooled if necessary, adjusted to the target concentration, and sterile-filtered through a 0.22 μm filter, and vials are filled.

Two different bioreactors are used: a small or pilot-scale bioreactor and a large-scale bioreactor. The small-scale bioreactor is a linearly scaled bioreactor with equal bed height for cell growth with respect to the large-scale bioreactor. The use of the small-scale bioreactor and the large-scale bioreactor allows for scalable manufacturing with minimal process and material impact. The large-scale bioreactor and/or the small-scale bioreactor is utilized for the production of the toxicology lot(s). The large-scale bioreactor is used for the production of the good manufacturing practice (GMP) drug substance (DS) lot(s) to be utilized in clinical trials and for licensure. Large-scale GMP production batch sizes are generated with multiple batches planned and pooled if necessary to satisfy the needed vector amount for drug product (DP) supply. The manufacturing process for AAV.CB7.CI.hARSAco.rBG remains largely unchanged as the product moves from IND-enabling non-clinical studies to clinical development and through licensure. Process parameters hypothesized to affect product quality are not be modified. Most critical source materials remain the same, including the HEK293 WCB, although the PEI and plasmid DNA utilized for GMP manufacturing is GMP-Source™ or INDReady™ grade materials.

As the scale-up manufacturing process with the large-scale bioreactor is implemented, and based on the combined manufacturing experience in the current bioreactor platform, any potential impact is addressed related to changes in the process through comparability testing to ensure there is no change to identity, purity, potency, and safety of the product. The comparability testing that is conducted to compare a new lot manufactured with an updated procedure or with new material to a previous lot consists of a subset of tests included in the certificate of analysis (COA). The new lot meets the specifications that were previously established, and any tests included in the comparability assessment (the table below) are completed using similar methodologies and, if possible, the same testing sites.

TABLE
Comparability Assessment

| | | | Specification/Acceptance |
|---|---|---|---|
| Comparability | Test | Method | Criteria |
| Potency | In Vitro Potency | Enzyme Activity | Conforms to Reference |
| | | Assay | |
| | GC:IU Ratio | TCID₅₀ | 500-3000 GC:IU |
| Purity | Purity | SDS-PAGE | ≥90% Virion Proteins |
| | Particle Content | AUC | TBD^{a} |
| | Analysis | | |
| Identity | Serotype Identity | MS | Confirmed as AAVhu68 serotype |
| | Molecular | Sanger Sequencing | Conforms to reference sequence, |
| | Identity | or NGS | excluding ITRs |
| Safety | Sterility | USP <71> | No microbial growth |
| | rcAAV | Triple passage, | Not detected |
| | | qPCR | |

^{a}Particle content analysis by AUC is determined upon completion of toxicology lot manufacturing and product establishment manufacturing runs.
AUC, analytical ultracentrifugation;
GC, genome copies;
ITR, inverted terminal repeat;
IU, infectious units;
MS, mass spectrometry;
NGS, next-generation sequencing;
qPCR, quantitative polymerase chain reaction;
rcAAV, replication-competent adeno-associated virus;
SDS-PAGE, sodium dodecyl sulfate polyacrylamide gel electrophoresis;
TBD, to be determined;
TCID₅₀, 50% tissue culture infective dose;
USP, United States Pharmacopeia.

The cell culture and harvest manufacturing process comprise four main manufacturing steps: (a) cell seeding and expansion, (b) transient transfection, (c) vector harvest, and (d) vector clarification. These process setups are depicted in the overview process diagram (FIG. 6). General descriptions of each of these processes are provided below.

### (a) Cell Seeding and Expansion

A fully characterized HEK293 cell line is used for the production process. A WCB has been produced. Cell culture used for vector production is initiated from one or two thawed WCB vials and expanded as per a Master Batch Record (MBR) document. Cells are expanded using tissue culture plastic to allow sufficient cell mass to be generated for seeding in a large-scale bioreactor vessel surface area for vector production per DS batch. Cells are cultivated in medium composed of Dulbecco's Modified Eagle Medium (DMEM) supplemented with 10% gamma irradiated New Zealand-sourced fetal bovine serum (FBS). The cells are anchorage-dependent, and cell disassociation is accomplished using TrypLE™ Select, an animal product-free cell dissociation reagent. Cell seeding is accomplished using sterile, single-use disposable bioprocess bags and tubing sets. The reactor is temperature-, pH-, and dissolved oxygen—(DO-) controlled.

### (b) Transient Transfection

Following approximately 4 days of growth (DMEM media+10% FBS), cell culture media is replaced with fresh, serum-free DMEM media and the cells are transfected with the three production plasmids using a PEI-based transfection method. All plasmids used in the production process are produced in the context of a CMO quality system as described above with infrastructure-utilizing controls to ensure traceability, document control, and materials segregation. Sufficient plasmid DNA transfection complexes are prepared in the BSC to transfect up to 500 m² (per BDS batch). Initially, a DNA/PEI mixture is prepared containing cis (vector genome) plasmid, trans (rep and cap genes) plasmid, and helper plasmid in an optimal ratio with GMP-grade PEI (PEIPro HQ, PolyPlus Transfection SA). This plasmid ratio was determined to be optimal for AAV production in small-scale optimization studies. After mixing well, the solution is allowed to sit at room temperature for up to 25 minutes, then added to serum-free media to quench the reaction, and finally added to the bioreactor. The reactor is temperature- and DO-controlled, and cells are incubated for 5 days.

### (c) Vector Harvesting

Transfected cells and media are harvested from the bioreactor using disposable bioprocess bags by aseptically pumping the medium out of the bioreactor. Following the harvest, detergent, endonuclease, and MgCl₂ (a co-factor for the endonuclease) are added to release vector and digest unpackaged DNA. The product (in a disposable bioprocess bag) is incubated at 37° C. for 2 hours in a temperature-controlled single-use mixer to provide sufficient time for enzymatic digestion of residual cellular and plasmid DNA present in the harvest as a result of the transfection procedure. This step is performed to minimize the amount of residual DNA in the final vector DP. Following incubation, NaCl is added to a final concentration of 500 mM to aid in the recovery of the product during filtration and downstream TFF.

### (d) Vector Clarification

Cells and cellular debris are removed from the product using a pre-filter and depth filter capsule (1.2/0.22 μm) connected in series as a sterile, closed tubing and bag set that is driven by a peristaltic pump. Clarification assures that downstream filters and chromatography columns are protected from fouling, and bioburden reduction filtration ensures that at the end of the filter train, any bioburden potentially introduced during the upstream production process is removed before downstream purification.

The purification process comprises four main manufacturing steps: (a) concentration and buffer exchange by TFF, (b) affinity chromatography, (c) anion exchange chromatography, and (d) concentration and buffer exchange by TFF. These process steps are depicted in the overview process diagram (FIG. 6). General descriptions of each of these processes are provided below.

### Large-Scale Tangential Flow Filtration

Volume reduction (20-fold) of the clarified product is achieved by TFF using a custom sterile, closed bioprocessing tubing, bag, and membrane set. The principle of TFF is to flow a solution under pressure parallel to a membrane of suitable porosity (100 kDa). The pressure differential drives molecules of smaller size through the membrane and effectively into the waste stream while retaining molecules larger than the membrane pores. By recirculating the solution, the parallel flow sweeps the membrane surface, preventing membrane pore fouling and product loss through binding to the membrane. By choosing an appropriate membrane pore size and surface area, a liquid sample may be rapidly reduced in volume while retaining and concentrating the desired molecule. Diafiltration in TFF applications involves addition of a fresh buffer to the recirculating sample at the same rate that liquid is passing through the membrane and to the waste stream. With increasing volumes of diafiltration, increasing amounts of the small molecules are removed from the recirculating sample. This diafiltration results in a modest purification of the clarified product, but also achieves buffer exchange compatible with the subsequent affinity column chromatography step. Accordingly, a 100 kDa, PES (polyethersulfone) membrane for concentration is utilized, which is then diafiltered with a minimum of four diavolumes of a buffer composed of 20 mM Tris pH 7.5 and 400 mM NaCl. The diafiltered product is then further clarified with a 1.2/0.22 μm depth filter capsule to remove any precipitated material.

### Affinity Chromatography

The diafiltered product is applied to a Poros™ Capture-Select™ AAV affinity resin (Life Technologies) that efficiently captures the AAVhu68 serotype. Under these ionic conditions, a significant percentage of residual cellular DNA and proteins flow through the column, while AAV particles are efficiently captured. Following application, the column is treated with volumes of a low-salt endonuclease solution (250 U/mL endonuclease, 20 mM Tris pH 7.5, 40 mM NaCl, and 1.5 mM MgCl₂) to remove any remaining host cells and plasmid nucleic acids. The column is washed to remove additional feed impurities followed by a low pH step elution (400 mM NaCl, 20 mM sodium citrate, pH 2.5) that is immediately neutralized by collection into a 1/10^{th} volume of neutralization buffer (200 mM Bis-Tris propane, pH 10.2).

Anion Exchange Chromatography To achieve further reduction of in-process impurities, including empty AAV particles, the Poros-AAV elution pool is diluted 50-fold (20 mM Bis-Tris propane, 0.001% Pluronic F-68, pH 10.2) to reduce ionic strength and enable binding to a CIMultus™ QA monolith matrix (BIA Separations). Following a low-salt wash, vector product is eluted using a 60 column volume NaCl linear salt gradient (10-180 mM NaCl). This shallow salt gradient effectively separates capsid particles without a vector genome (empty particles) from particles containing vector genome (full particles) and results in a preparation enriched for full particles. The full particle peak eluate is collected and neutralized. The peak area is assessed and compared to previous data for determination of the approximate vector yield.

### Concentration and Buffer Exchange by Hollow Fiber Tangential Flow Filtration

The pooled anion exchange intermediate is concentrated and buffer-exchanged using TFF. In this step, a 100 kDa membrane hollow fiber TFF membrane is used. During this step, the product is brought to a target concentration and then buffer-exchanged into the ITFFB (artificial CSF with 0.001% Pluronic F-68). Samples are removed for testing (FIG. 7). The bulk drug substance (BDS) is sterile-filtered (0.22 μm), stored in sterile containers, and frozen at ≤−60° C. in a quarantine location until release for final fill.

The frozen bulk drug substance are thawed, pooled, and adjusted to the target concentration (dilution or concentrating step via TFF) using the final formulation buffer (FFB). The product is terminally filtered through a 0.22 μm filter and filled into sterile West Pharmaceutical's Crystal Zenith (cyclic olefin polymer) vials with crimp seal stoppers. Labeled vials are stored at ≤−60° C.

Bacterial master cell bank (BMCB) glycerol stocks of the cis, trans and helper plasmids were made by mixing 1 mL from a 1 L overnight culture of transformed Stb12™ E. coli cells with an equal volume of sterile 50% glycerol. Two 0.5 mL aliquots of the BMCB glycerol stocks per construct are prepared from the mixture and stored in Nalgene cryogenic vials at −80° C. To verify BMCB glycerol stocks, amplified plasmid DNA is subjected to in-house structure analysis involving restriction enzyme digestion followed by gel electrophoresis, and full-plasmid sequence analysis by Sanger sequencing at Qiagen. To prepare bacterial working cell bank (BWCB) glycerol stock aliquots for shipping to the plasmid DNA manufacturer, a 3 mL culture is inoculated from a BMCB glycerol stock and grown overnight. Next, 1 mL of the overnight culture is used to prepare BWCB glycerol stock aliquots as described above. New BWCB glycerol stock aliquots are verified by the aforementioned structure analysis on DNA extracted from the remaining 2 mL of overnight bacterial culture. Once received at the plasmid DNA manufacturer, the BWCB glycerol stock is stored in a project-specific location at −80° C. Production cultures are inoculated by scraping the frozen BWCB glycerol stock.

Plasmids used as source material for Good Manufacturing Practice (GMP) vector manufacturing are produced at a facility that is not qualified as a GMP facility; however, plasmids are produced in a manner that is designed to meet the requirements for Current Good Manufacturing Practice (cGMP) intermediates. Plasmid production is conducted on dedicated components and in a dedicated suite. The production procedures and oversight are conducted to ensure a consistent quality product with highly pure DNA, which meets stringent release criteria as captured in the following table. Components used in the production of plasmids are “animal-free” (based on the COAs from each vendor for component products), and all components used in the process (fermentation flasks, containers, membranes, resin, columns, tubing and any component that comes into contact with the plasmid) are dedicated to a single plasmid and are certified TSE-/BSE-free. The PolyFlo® resin, columns and components utilized are procured for the exclusive use in the manufacturing of a single plasmid. The fermentation, lysis and purification of the plasmid occurs in dedicated rooms marked with the designated plasmid name. No other plasmids are processed in those rooms at the same time. The rooms and equipment are cleaned between each plasmid production campaign. Prior to use in the production of recombinant vectors, each manufactured plasmid is fully sequenced using next-generation sequencing (NGS) to rule out contamination by other plasmids, in addition to testing for sterility and the presence of mycoplasma.

All plasmid DNA used in the production of vectors for pharmacology/toxicology are made through Puresyn's Premium-Research Ready Program. Puresyn's Premium-Research Ready Program are produced using cleaning and segregation procedures and single-use components however they are not produced in a dedicated room.

TABLE
Release Specifications for Plasmid Production

| Parameter | Specifications |
|---|---|
| Appearance | Clear, colorless, and no visible particulates |
| A260:280 | 1.7-2.0 |
| Concentration | 1.0-1.1 mg/mL |
| DNA Homogeneity | ≥90% supercoiled |
| Residual RNA | None detected at 1.0 μg load |
| SSDNA, Linear DNA, | None detected at 1.0 μg load |
| Chromosomal DNA | |
| Endotoxin | <30 EU/mg |
| Identity | Consistent with provided sequence and structure |
| | information |
| Protein | For information only |
| Bioburden | No growth after 5 days |
| pH | 7.5-8.5 |
| Formulation | TE (10 mM Tris, 1.0 mM EDTA pH 7.9-8.1) |

HEK293 cells were originally generated by transforming HEK cells with sheared adenovirus type 5 (Ad5) DNA (Graham et al., 1977). The cells express the E1A and E1B gene products required for rAAV production. HEK293 cells are highly transfectable, yielding high levels of rAAV upon plasmid DNA transfection.

### Vector Genome Identity: DNA Sequencing

AAV vector (2.00×10¹¹ GC) is treated with Baseline Zero endonuclease and Plasmid Safe DNAse to eliminate non-encapsulated DNA in the environment and then incubated for 10 min at 95° C. in 1× phosphate-buffered saline (PBS) and 0.5% sodium dodecyl sulfate (SDS) to denature the vector genome. Denatured vector genome is subsequently annealed by slowly cooling the reaction mix to 24° C. at a rate of 0.6° C./minute in a thermocycler, cleaned up using the QIAquick PCR Purification Kit (QIAGEN), and sheared to an average size of 500 bp on a Covaris Ultrasonicator. DNA shearing is evaluated on a 2100 Bioanalyzer with High Sensitivity DNA reagent kit (Agilent). Sheared DNA is prepared into NGS libraries using the NEBNextUltraII library kit according to the manufacturer's protocol, size-selected, and cleaned up by Agencourt AMPure XP beads (Beckman Coulter). Individual NGS libraries are then analyzed on a Bioanalyzer again for fragment size distribution and quantified by a Qubit® 3.0 Fluorometer prior to pooling at equal molarity. The concentration of final pooled library is measured by a Qubit® 3.0 Fluorometer, denatured, and diluted to 8 pM according to Illumina's Miseq System Denature and Dilute Libraries Guide. PhiX control is spiked in the final library at 10%. Sequencing is performed using an Illumina MiSeq Nano Reagent Kit V2 (250 bp paired-end) on a MiSeq sequencer. Data analysis is performed as described above using the NGS alignment approach.

Sequencing reads are automatically de-multiplexed and adapter-trimmed by the MiSeq computer. The trimmed reads for each plasmid are aligned to the corresponding reference sequence, and sequence variants are called using BBTools bioinformatics software suite (sourceforge.net/projects/bbmap). Additionally, BBMap (jgi.doe.gov/data-and-tools/bbtools/) is used to generate VCF and BAM files. VCF files are further parsed by a custom UNIX script to generate simplified tab-delimited tables (retaining only CHROM, REF, ALT, QUAL, TYPE, DEPTH, AF, RAF, SB, DP4 fields). BAM files are visually inspected in IGV Integrated Genomic Viewer software (software.broadinstitute.org/software/igv/) to ensure proper NGS alignments. In parallel with the NGS alignment approach, de novo assembly is conducted to build a long, circularized sequence using NOVOPlasty (github.com/ndierckx/NOVOPlasty). The de novo sequence is aligned against the original vector genome reference sequence to characterize large sequence arrangements that can be overlooked in the alignment approach.

### Vector Capsid Identity: AAV Capsid Mass Spectrometry of VP1

Confirmation of the AAVhu68 serotype of the DP is achieved using trypsin digestion of the VP followed by tandem mass spectrometry (MS) characterization on a Q-Exactive Orbitrap mass spectrometer to sequence the capsid protein peptides. A spectral library from the tandem mass spectra sequenced and a targeted MS method is used to assay for signature peptides that can uniquely identify specific AAV viral particles serotypes. A bank of signature peptides specific for eight serotypes (AAVhu68, AAV1, AAV2, AAV6, AAV8, AAV9, AAVrh10, and AAVhu37) are screened against the tandem mass spectra produced by digestion of the test article. For a positive identification, signature peptide(s) from a single serotype only are detected.

### Genomic Copy Titer

A ddPCR-based technique for determining the GC titer for AAV vectors has been developed (Lock et al., 2014). The reference standard is generated during the pilot runs and is used to qualify the assay. The method is practical, reports equivalent or better titers than qPCR, and does not require a plasmid standard curve. The assay utilized involves digestion with DNase I, followed by ddPCR analysis to measure encapsulated vector GC. DNA detection is accomplished using sequence-specific primers targeting the polyA region in combination with a fluorescently tagged probe hybridizing to this same region. A number of standards, validation samples, and controls (for background and DNA contamination) have been introduced into the assay. This assay is qualified using pilot reference standard. The assay is qualified by establishing and defining assay parameters, including sensitivity, limit of detection (LOD), range of qualification, and intra- and inter-assay precision. An internal AAVhu68 reference lot is established and used to perform the qualification studies.

### Infectious Unit Titer

The infectious unit (IU) assay is used to determine the productive uptake and replication of rAAV vector in RC32 cells (rep2 expressing HeLa cells). A 96-well endpoint format has been employed similar to that previously published. Briefly, RC32 cells are co-infected by serial dilutions of rAAV BDS and a uniform dilution of Ad5 with 12 replicates at each dilution of rAAV. Seventy-two hours after infection, the cells are lysed, and qPCR is performed to detect rAAV vector amplification over input. An endpoint dilution 50% tissue culture infectious dose (TCIDso) calculation (Spearman-Karber) is performed to determine a replicative titer expressed as IU/mL. Since “infectivity” values are dependent on each particle's contact with cells, receptor binding, internalization, transport to the nucleus, and genome replication, they are influenced by assay geometry and the presence of appropriate receptors and post-binding pathways in the cell line used. Receptors and post-binding pathways are not usually maintained in immortalized cell lines, and thus infectivity assay titers are not an absolute measure of the number of “infectious” particles present. However, the ratio of encapsidated GC to “infectious units” (described as GC/IU ratio) can be used as a measure of product consistency from lot to lot.

### Particle Content Analysis

Sedimentation velocity, as measured in an analytical ultracentrifuge (AUC), can detect aggregates, other minor components, as well as provide good quantitation of relative amounts of different particle species based upon their different sedimentation coefficients. This is an absolute method based on fundamental units of length and time, requiring no standard molecules as references. Vector samples are loaded into cells with two-channel charcoal-epon centerpieces with 12 mM optical path length. The supplied dilution buffer is loaded into the reference channel of each cell. The loaded cells are then placed into an AN-60Ti analytical rotor and loaded into a Beckman-Coulter ProteomeLab XL-I analytical ultracentrifuge equipped with both absorbance and RI detectors. After full temperature equilibration at 20° C., the rotor is brought to the final run speed of 12,000 revolutions per minute (RPM). Absorbance at 280 nm scans are recorded approximately every 3 minutes for approximately 5.5 hours (110 total scans for each sample). The raw data is analyzed using the c(s) method and implemented in the analysis program SEDFIT. The resultant size distributions are graphed and the peaks integrated. The percentage values associated with each peak represent the peak area fraction of the total area under all peaks and are based upon the raw data generated at 280 nm. Many labs use these values to calculate full:empty ratios. However, because empty and full particles have different extinction coefficients at this wavelength, the raw data can be adjusted accordingly. The ratio of the empty particle and full monomer peak values both before and after extinction coefficient adjustment is used to determine the full:empty ratio, and both ratios are recorded.

### Host Cell DNA

A qPCR assay is used to detect residual HEK293 DNA. After spiking with a “non-relevant DNA,” total DNA (non-relevant, vector, and residual genomic DNA) is extracted from approximately 1 mL of product. The HCDNA is quantified using qPCR targeting 18S rDNA. The quantities of DNA detected are normalized based on the recovery of the spiked non-relevant DNA. Three different amplicon sizes are tested to establish the size spectrum of residual HCDNA.

### Host Cell Protein

An ELISA is performed to measure levels of contaminating host HEK293 cell proteins. The Cygnus Technologies HEK293 Host Cell Proteins 2nd Generation ELISA kit is used according to the instructions provided by the vendor.

### Replication-Competent AAV Assay

A sample is analyzed for the presence of replication-competent AAV2/hu68 (rcAAV) that could potentially arise during the production process. A three-passage assay has been developed consisting of cell-based amplification and passage followed by detection of rcAAV DNA by real-time qPCR (caphu68 target). The cell-based component consists of inoculating monolayers of HEK293 cells (P1) with dilutions of the test sample and wild type human Ad5. The maximal amount of the product tested is 1.00×10¹⁰ GC of the vector product. Due to the presence of adenovirus, rcAAV amplifies in the cell culture. After 2 days, a cell lysate is generated, and Ad5 is heat-inactivated. The clarified lysate is then passed onto a second round of cells (P2) to enhance sensitivity (again in the presence of Ad5). After 2 days, a cell lysate is generated, and Ad5 is heat-inactivated. The clarified lysate is then passed onto a third round of cells (P3) to maximize sensitivity (again in the presence of Ad5). After 2 days, cells are lysed to release DNA, which is then subjected to qPCR to detect AAVhu68 cap sequences. Amplification of AAVhu68 cap sequences in an Ad5-dependent manner indicates the presence of rcAAV. The use of a AAV2/hu68 surrogate positive control containing AAV2 rep and AAVhu68 cap genes enables the LOD of the assay to be determined (0.1 IU, 1 IU, 10 IU, and 100 IU). Using a serial dilution of rAAV (1.00×10¹⁰ GC, 1.00×10⁹ GC, 1.00×10⁸ GC, and 1.00×10⁹ GC), the approximate quantity of rcAAV present in the test sample can be quantitated. The test method is performed.

### In Vitro Potency

To relate the ddPCR GC titer to gene expression, an in vitro relative potency bioassay is performed. Briefly, cells are plated in a 96-well plate and incubated at 37° C./5% CO₂ overnight. The next day, cells are infected with serially diluted AAV vector and are incubated at 37° C./5% CO₂ for up to 3 days. At the end of the culture period, cell culture media are collected and assayed for ARSA activity based on cleavage of a colorimetric substrate.

### Total Protein, Capsid Protein, Protein Purity, and Capsid Protein Ratio

Vector samples are first quantified for total protein against a bovine serum albumin (BSA) protein standard curve using a bicinchoninic acid (BCA) assay. The determination is made by mixing equal parts of sample with a Micro-BCA reagent provided in the kit. The same procedure is applied to dilutions of a BSA standard. The mixtures are incubated at 60° C. and absorbance measured at 562 nm. A standard curve is generated from the standard absorbance of the known concentrations using a 4-parameter fit. Unknown samples are quantified according to the 4-parameter regression.

To provide a semi-quantitative determination of rAAV purity, the samples are normalized for genome titer, and 5.00×10⁹ GC is separated by sodium dodecyl sulfate polyacrylamide gel electrophoresis (SDS-PAGE) under reducing conditions. The SDS-PAGE gel is then stained with SYPRO Ruby dye. Any impurity bands are quantified by densitometry. Stained bands that appear in addition to the three AAV-specific proteins (VP1, VP2, and VP3) are considered protein impurities. The impurity mass percent as well as approximate molecular weight of contaminant bands are reported. The SDS-PAGE gel is also used to quantify the VP1, VP2, and VP3 proteins and determine their ratio.

### Ratio of Genome Copy to Infectious Unit

The GC/IU ratio is a measure of product consistency. The ddPCR titer (GC/mL) is divided by the “infectious unit” (IU/mL) to give the calculated GC/IU ratio.

### Example 2—Pharmacology and Dose Range Study in Mice

A study was performed to determine the efficacy and dose range of AAVhu68.CB7.CI.hARSAco.rBG (GTP-207), a recombinant adeno-associated viral (AAV) serotype hu68 vector expressing the human arylsulfatase A (ARSA) gene, following intracerebroventricular (ICV) administration in adult male C57BL/6J (wild type) mice.

Adult male C57BL/6J (wild type) mice received a single ICV administration of AAV.CB7.CI.hARSAco.rBG (GTP-207) at a dose of 1.0×10¹⁰ GC (2.5×10¹⁰ GC/brain; low dose) or 1.0×10¹¹ GC (2.5×10¹¹ GC/brain; high dose). Age-matched C57BL6/J mice were administered vehicle (phosphate-buffered saline [PBS]) as a control. Animals were monitored daily for viability. On Day 7 and at necropsy on Day 21, serum was collected for evaluation of transgene product expression (ARSA enzyme activity) and anti-transgene product antibodies (anti-human ARSA antibodies). Brain and liver were also collected at necropsy to evaluate transgene product expression (ARSA enzyme activity).

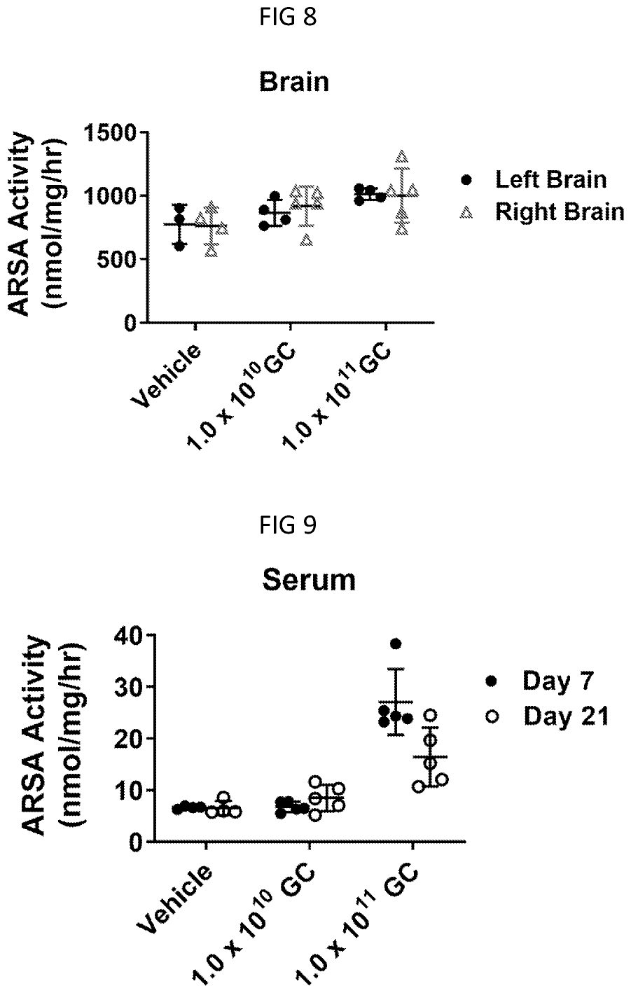

In the brain, ARSA enzyme activity was measured in the left versus right cerebral hemispheres 21 days after AAV.CB7.CI.hARSAco.rBG (GTP-207) administration (FIG. 8). A dose-dependent response was observed, with a 1.2-fold and 1.3-fold increase in ARSA enzyme activity observed in the brains of mice administered the low dose (1.0×10¹⁰ GC) or high dose (1.0×10¹¹ GC) of AAV.CB7.CI.hARSAco.rBG (GTP-207), respectively, compared to vehicle-treated controls. There was no apparent difference in ARSA enzyme activity levels between the right and left hemispheres for AAV.CB7.CI.hARSAco.rBG (GTP-207)-treated animals.

In serum, wild type mice administered the low dose (1.0×10¹⁰ GC) of AAV.CB7.CI.hARSAco.rBG (GTP-207) exhibited transgene product expression (ARSA enzyme activity) levels similar to that of vehicle-treated controls on Day 7, which increased to be 1.3-fold higher than vehicle-treated control levels on Day 21. Wild type mice administered the high dose (1.0×10¹¹ GC) of AAVhu68.CB7.CI.hARSAco.rBG (GTP-207) displayed increased ARSA enzyme activity compared to that of vehicle-treated controls on both Day 7 (4-fold higher) and Day 21 (2.5-fold higher), with slightly higher ARSA enzyme activity levels recorded on Day 7 compared to Day 21. Furthermore, a dose-dependent effect was observed, with wild type mice administered the high dose of AAVhu68.CB7.CI.hARSAco.rBG (GTP-207) (1.0×10¹¹ GC) exhibiting higher ARSA enzyme activity than that of the wild type mice administered the low dose of AAVhu68.CB7.CI.hARSAco.rBG (GTP-207) (1.0×10¹⁰ GC) at both Day 7 (4-fold higher) and Day 21 (2-fold higher) (FIG. 9).

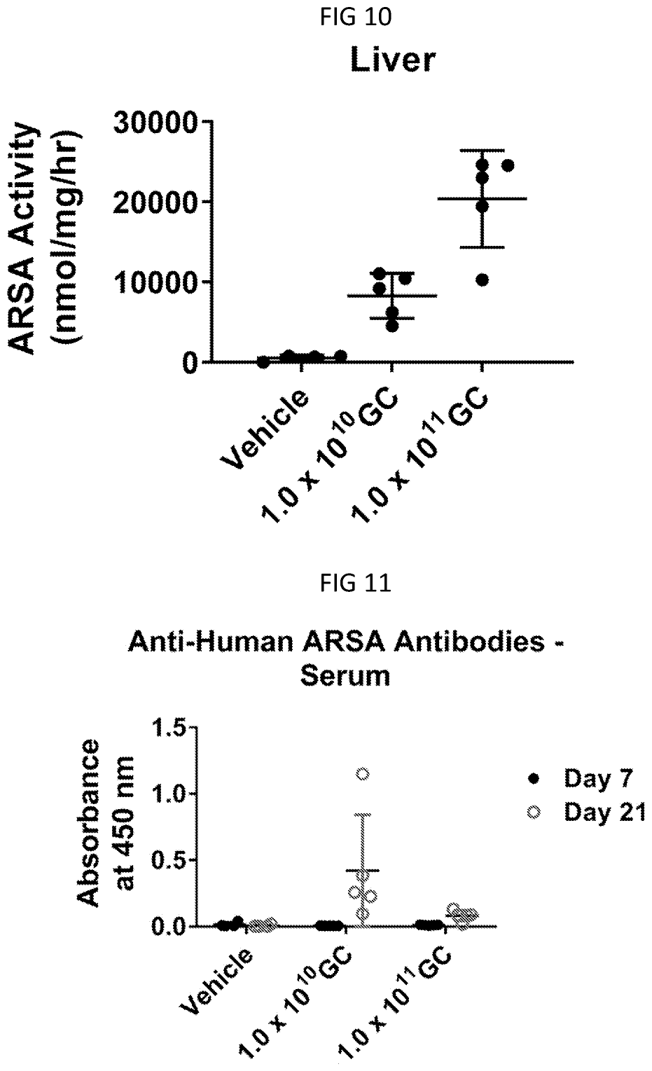

In the liver, a dose-dependent increase in transgene product expression was observed, with a 15-fold and 37-fold increase in ARSA enzyme activity observed in mice administered the low dose (1.0×10¹⁰ GC) or high dose (1.0×10¹¹ GC) of AAV.CB7.CI.hARSAco.rBG (GTP-207), respectively, compared to that of vehicle-treated controls (FIG. 10).

On Day 7, wild type mice administered either the low dose (1.0×10¹⁰ GC) or high dose (1.0×10¹¹ GC) of AAV.CB7.CI.hARSAco.rBG (GTP-207) did not exhibit anti-human ARSA antibody expression in serum above the levels observed in vehicle-treated controls. On Day 21, an increase in anti-human ARSA antibody expression above vehicle-treated control levels was observed in AAV.CB7.CI.hARSAco.rBG (GTP-207)-treated mice, with animals administered the low dose (1.0×10¹⁰ GC) exhibiting higher levels of anti-human ARSA antibodies than animals administered the high dose (1.0×10¹¹ GC) (FIG. 11).

### Summary of Results

A single unilateral ICV injection of AAV.CB7.CI.hARSAco.rBG (GTP-207) led to a dose-dependent increase in transgene product expression (ARSA enzyme activity) in a disease-relevant target organ (brain), with 1.2-fold and 1.3-fold higher levels of ARSA enzyme activity observed in mice administered the low dose (1.0×10¹⁰ GC [2.5×10¹⁰ GC/g brain]) or high dose (1.0×10¹¹ GC [2.5×10¹¹ GC/g brain]) of AAV.CB7.CI.hARSAco.rBG (GTP-207), respectively, compared to vehicle-treated controls.
In the liver, a dose-dependent increase in ARSA enzyme activity was observed, with 15-fold or 37-fold higher expression observed in mice administered the low dose (1.0×10¹⁰ GC [2.5×10¹⁰ GC/g brain]) or high dose (1.0×10¹¹ GC [2.5×10¹¹ GC/g brain]) of AAV.CB7.CI.hARSAco.rBG (GTP-207), respectively, compared to vehicle-treated controls.
In serum, a dose-dependent increase in ARSA enzyme activity was observed. On Day 7, ARSA enzyme activity in wild type mice administered the low dose (1.0×10¹⁰ GC [2.5×10¹⁰ GC/g brain]) or high dose (1.0×10¹¹ GC [2.5×10¹¹ GC/g brain]) of AAV.CB7.CI.hARSAco.rBG (GTP-207) was similar to or 4-fold higher than that of vehicle-treated controls, respectively. By Day 21, ARSA enzyme activity in wild type mice administered the low dose (1.0×10¹⁰ GC [2.5×10¹⁰ GC/g brain]) or high dose (1.0×10¹¹ GC [2.5×10¹¹ GC/g brain]) of AAV.CB7.CI.hARSAco.rBG (GTP-207) was 1.3-fold or 2.5-fold higher than that of vehicle-treated controls, respectively.
Anti-human ARSA antibodies were detectable in serum above vehicle-treated control levels by Day 21. Antibodies are an expected response to expression of a foreign human transgene product in mice. Antibody levels detected by ELISA were inversely correlated with transgene product expression in the liver on Day 21.
Cumulatively, ICV administration of AAV.CB7.CI.hARSAco.rBG (GTP-207) to wild type mice at a dose of 1.0×10¹⁰ GC (2.5×10¹⁰ GC/g brain) or 1.0×10¹¹ GC (2.5×10¹¹ GC/g brain) leads to transgene product expression (ARSA enzyme activity) in a disease-relevant target tissue (the brain) and in the periphery (liver and serum).

### Example 3—Cell Tropism Study in Mice

A study was performed to assess transgene product expression following intracerebroventricular (ICV) administration of AAVhu68.CB7.CI.hARSAco-HA.rBG, a recombinant adeno-associated viral (AAV) serotype hu68 vector expressing the human arylsulfatase A (ARSA) gene, to adult male C57BL/6J (wild type) mice. AAVhu68.CB7.CI.hARSAco-HA.rBG is identical to AAVhu68.CB7.CI.hARSAco.rBG (GTP-207) except that it expresses a hemagglutinin—(HA-) tagged version of human ARSA to enable improved detection in tissues by immunofluorescence (IF).

Adult male C57BL/6J (wild type) mice received a single ICV administration of AAVhu68.CB7.CI.hARSAco-HA.rBG at a dose of 1.0×10¹⁰ GC (2.5×10¹⁰ GC/brain; low dose) or 1.0×10¹¹ GC (2.5×10¹¹ GC/brain; high dose). Age-matched C57BL6/J mice were administered vehicle (phosphate-buffered saline [PBS]) as a control. Animals were monitored daily for viability. On Day 7 and at necropsy on Day 21, serum was collected for evaluation of transgene product expression (ARSA enzyme activity). Brain and liver were also collected at necropsy to evaluate transgene product expression (ARSA enzyme activity or human ARSA immunofluorescence [IF]).

The aim of this study was to assess cellular transgene product expression in disease-relevant target tissues of the CNS (myelin-producing oligodendrocytes) and in the periphery (serum and liver) following ICV administration of an AAV vector similar to AAV.CB7.CI.hARSAco.rBG (GTP-207) to adult C57BL/6J (wild type) mice.

The vector utilized was AAVhu68.CB7.CI.hARSAco-HA.rBG, which is identical to AAV.CB7.CI.hARSAco.rBG (GTP-207) except that it includes a transgene encoding a human codon-optimized ARSA enzyme tagged with a C-terminal hemagglutinin (HA) peptide. The HA-tagged ARSA transgene was preferred for this study because anti-human ARSA primary antibodies used for immunofluorescence (IF) can potentially cross-react with endogenous murine ARSA in wild type animals. The observed ARSA expression profile following ICV administration of this similar AAV vector is expected to be representative of ARSA expression in mice following AAV.CB7.CI.hARSAco.rBG (GTP-207) administration.

On Study Day 0, adult C57BL/6J (wild type) mice received a single ICV administration of either AAVhu68.CB7.CI.hARSAco-HA.rBG at one of two doses (1.0×10¹⁰ GC or 1.0×10¹¹ GC) or control article (PBS [vehicle]). Viability checks were performed daily. On Day 7 and at necropsy on Day 21, serum was collected for evaluation of transgene product expression (ARSA enzyme activity). Brain and liver were also collected at necropsy to evaluate transgene product expression (ARSA enzyme activity). The brain samples collected contained cortex and subcortical white matter to assess transgene product expression (human ARSA IF) in OLIG2-positive oligodendrocytes.

A 21-day study duration was considered sufficient to evaluate transgene product expression (ARSA enzyme activity) during the expected onset, peak, and plateau of transgene expression. The brain was evaluated for transgene product expression because it is an important target organ for the treatment of MLD in humans, and the liver was evaluated because it is a highly perfused organ. Serum was collected to assess the potential for cross-correction in the PNS.

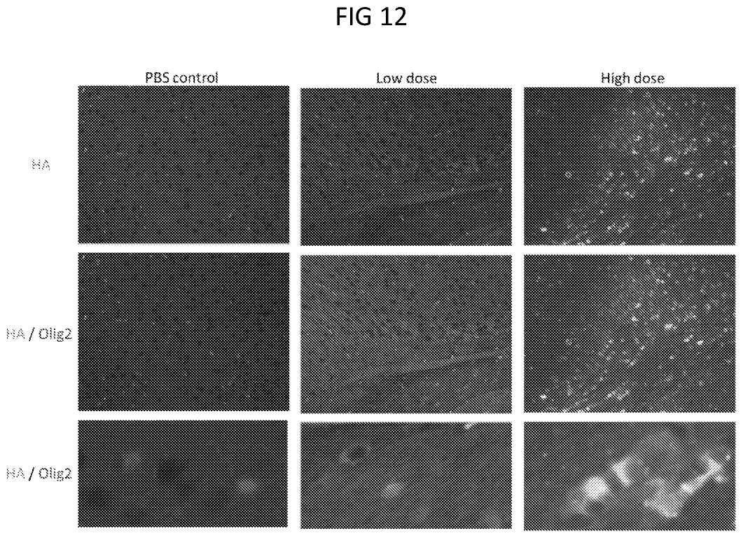

Brain samples containing cortex and subcortical white matter were obtained to assess transgene product expression (HA IF) in OLIG2-positive oligodendrocytes 21 days after AAVhu68.CB7.CI.hARSAco-HA.rBG administration (FIG. 12). Administration of the low dose (1.0×10¹⁰ GC) resulted in a minimal number of human ARSA-expressing cells (detected by the presence of HA positive signal) in the cortex and subcortical white matter. In contrast, animals administered the high dose (1.0×10¹¹ GC) displayed a greater number of cells expressing ARSA in the cortex and subcortical white matter. Moreover, an enrichment of human ARSA-expressing oligodendrocytes (HA-positive, OLIG2-positive cells) was observed in brain regions containing a large number of ARSA-expressing presumptive neurons (HA-positive, OLIG2-negative cells with morphology compatible with a neuron).

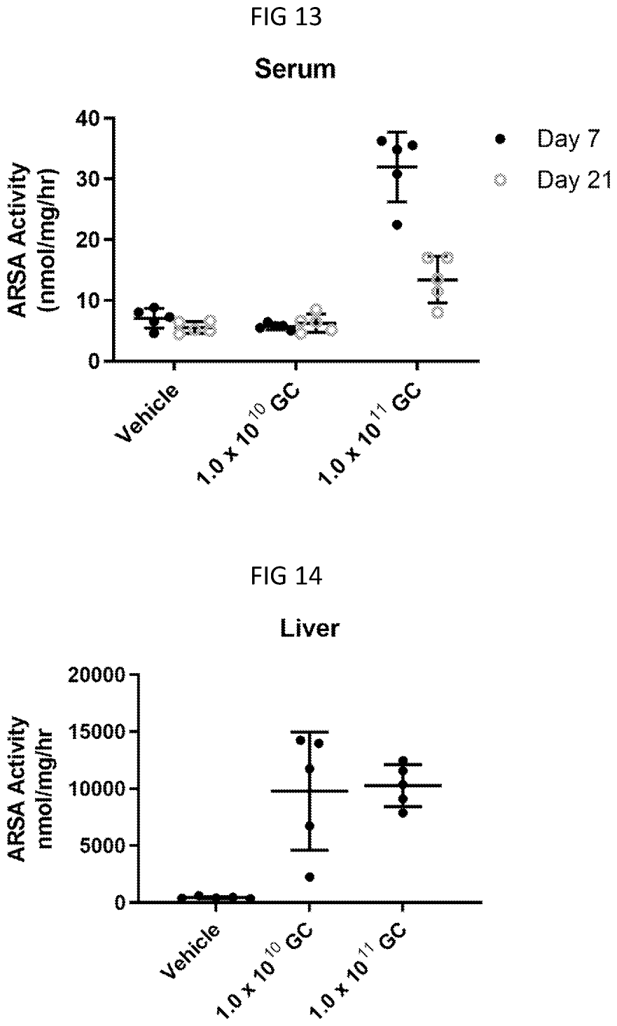

In serum, wild type mice administered the low dose (1.0×10¹⁰ GC) of AAVhu68.CB7.CI.hARSAco-HA.rBG exhibited transgene product expression (ARSA enzyme activity) levels similar to that of vehicle-treated controls on Day 7 and Day 21. Wild type mice administered the high dose (1.0×10¹¹ GC) of AAVhu68.CB7.CI.hARSAco-HA.rBG displayed increased ARSA enzyme activity compared to that of vehicle-treated controls on both Day 7 (5-fold higher) and Day 21 (2-fold higher), with higher ARSA enzyme activity levels observed on Day 7 compared to Day 21. Furthermore, a dose-dependent effect was observed, with wild type mice administered the high dose (1.0×10¹¹ GC) exhibiting higher ARSA enzyme activity than that of the wild type mice administered the low dose (1.0×10¹⁰ GC) at both Day 7 (6-fold higher) and Day 21 (2-fold higher) (FIG. 13).

In the liver, robust transgene product expression was observed at both doses, with a 22-fold and 23-fold increase in ARSA enzyme activity observed in mice administered the low dose (1.0×10¹⁰ GC) or high dose (1.0×10¹¹ GC) of AAVhu68.CB7.CI.hARSAco-HA.rBG, respectively, compared to that of vehicle-treated controls (FIG. 14).

### Summary of Results

A single unilateral ICV injection of AAVhu68.CB7.CI.hARSAco-HA.rBG at a dose of 1.0×10¹⁰ GC (2.5×10¹⁰ GC/g brain) or 1.0×10¹¹ GC (2.5×10¹¹ GC/g brain) led to dose-dependent expression of human ARSA in the cortex and subcortical white matter (HA IF). Human ARSA expression was detectable in both oligodendrocytes (HA-positive, OLIG2-positive cells) and presumptive neurons (HA-positive, OLIG2-negative cells).
In serum, a dose-dependent increase in ARSA enzyme activity was observed. On Day 7, ARSA enzyme activity in wild type mice administered the low dose (1.0×10¹⁰ GC [2.5×10¹⁰ GC/g brain]) or high dose (1.0×10¹¹ GC [2.5×10¹¹ GC/g brain]) of AAVhu68.CB7.CI.hARSAco-HA.rBG was similar to or 5-fold higher than that of vehicle-treated controls, respectively. By Day 21, ARSA enzyme activity in wild type mice administered the low dose (1.0×10¹⁰ GC [2.5×10¹⁰ GC/g brain]) or high dose (1.0×10¹¹ GC [2.5×10¹¹ GC/g brain]) of AAVhu68.CB7.CI.hARSAco-HA.rBG was similar to or 2-fold higher than that of vehicle-treated controls, respectively.
In the liver, a 22-23-fold increase in ARSA enzyme activity was observed in mice administered the low dose (1.0×10¹⁰ GC) or high dose (1.0×10¹¹ GC) of AAVhu68.CB7.CI.hARSAco-HA.rBG compared to that of vehicle-treated controls.
Cumulatively, ICV administration of AAVhu68.CB7.CI.hARSAco-HA.rBG to wild type mice at a dose of 1.0×10¹⁰ GC (2.5×10¹⁰ GC/g brain) or 1.0×10¹¹ GC (2.5×10¹¹ GC/g brain) leads to transgene product expression (ARSA enzyme activity and ARSA protein expression) in a disease-relevant target tissue (myelin-producing oligodendrocytes and presumptive neurons in the brain) and in the periphery (liver and serum), suggesting the possibility for cross-correction in the CNS and PNS.

### Example 4—Transgene Product Expression and Cellular Localization Following Intra-Cistema Magna Administration of AAVhu68.CB7.CI.hARSAco-HA.rBG to Adult Rhesus Macaques

A pharmacology study was performed to evaluate the pharmacodynamic and limited safety profile of AAVhu68.CB7.CI.hARSAco-HA.rBG following intra-cisterna magna (ICM) administration to adult rhesus macaque non-human primates (NHPs). AAVhu68.CB7.CI.hARSAco-HA.rBG is a recombinant adeno-associated viral (AAV) serotype hu68 vector expressing the human arylsulfatase A (ARSA) gene and is identical to AAVhu68.CB7.CI.hARSAco.rBG (GTP-207) except that it expresses a hemagglutinin—(HA-) tagged version of human ARSA to enable improved detection in tissues by immunostaining.

Adult male (N=1) and female (N=1) rhesus macaque non-human primates (NHPs) received a single ICM administration of AAVhu68.CB7.CI.hARSAco-HA.rBG at a dose of 3.0×10¹³ GC [3.3×10¹¹ GC/g brain]). In-life assessments included clinical observations, body weight measurements, clinical pathology of the blood and cerebrospinal fluid (CSF), and evaluation of transgene product expression (ARSA enzyme activity) in serum and CSF. Necropsies were performed on Day 21. At necropsy, tissues of the central nervous system (CNS), peripheral nervous system (PNS), and peripheral organs were collected for evaluation of transgene product expression (ARSA enzyme activity). CNS and PNS tissues were also collected to determine the cellular localization of transgene product expression (ARSA immunohistochemistry [IHC] or ARSA immunofluorescence [IF] using an antibody recognizing the HA tag).

| | | | | | Dose | | | | |
|---|---|---|---|---|---|---|---|---|---|
| | | | | Dose | (GC/g | | Injection | Day of | Day of |
| Group | Treatment | Animal ID | Sex | (GC) | Brain)^{a} | ROA | Volume | Dosing | Necropsy |
| 1 | AAVhu68.CB7.CI.hARSAco- | RA2397 | F | 3.0 × 10¹³ | 3.3 × 10¹¹ | ICM | 1 mL | 0 | 21 ± 1 |
| | HA.rBG | RA2477 | M | | | | | | |

^{a}Dose is scaled based on a brain mass of 90 g for an adult NHP (Herndon et al., 1998).
Abbreviations: F, female; GC, genome copies; ICM, intra-cisterna magna; ID, identification number; M, male; ROA, route of administration.

On Study Day 0, adult rhesus macaque NHPs (5-6 years old) received a single ICM administration of AAVhu68.CB7.CI.hARSAco-HA.rBG at a dose of 3.0×10¹³ GC. In-life assessments included clinical observations, body weight measurements, clinical pathology of the blood and CSF (cell counts, clinical chemistry, and/or total protein), and evaluation of transgene product expression (ARSA enzyme activity) in serum and CSF. Necropsies were performed on Day 21. Tissues of the CNS (brain, spinal cord), PNS (DRG, sciatic nerves), and peripheral organs (pancreas, heart, kidney, and quadriceps muscle) were collected at necropsy for evaluation of transgene product expression (ARSA enzyme activity). CNS tissues (spinal cord) and PNS tissues (DRG, TRG, trigeminal nerve, and peripheral nerves [median, radial, sciatic, peroneal, tibial]) were also collected to determine the cellular localization of transgene product expression (ARSA IHC or ARSA IF using an antibody recognizing the hemagglutinin [HA] tag). Additional tissues were also collected and stored for possible future histopathology and vector biodistribution analysis.

### Results:

### Mortality

Both animals survived to the scheduled necropsy time point.

### Clinical Observations

No treatment-related abnormalities were identified on clinical observations.

### Body Weights

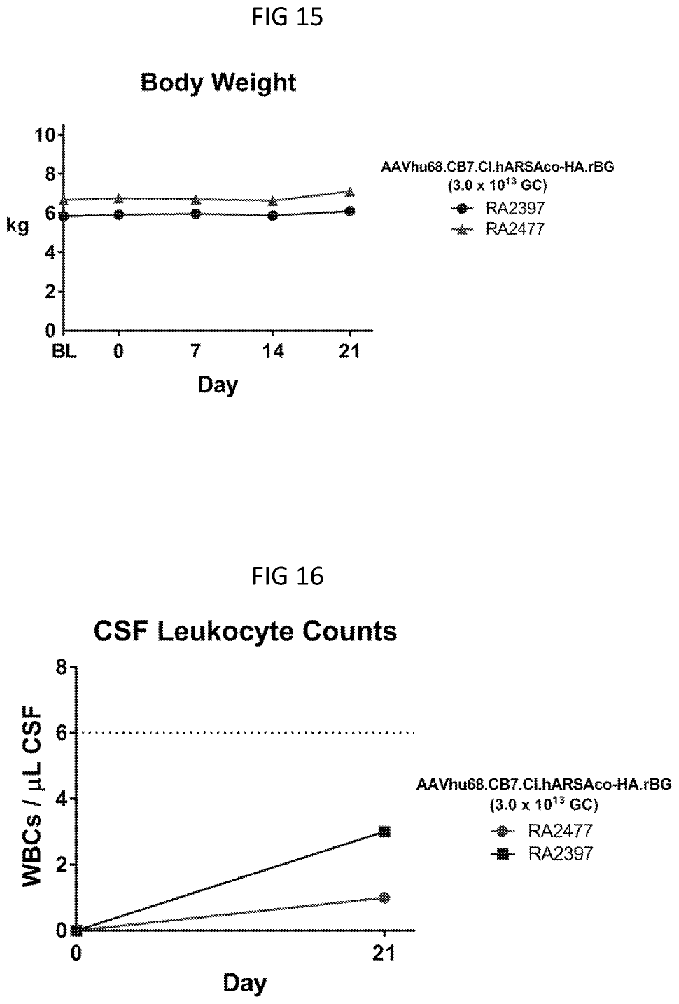

Body weights were stable for both animals throughout the study (FIG. 15).

### Blood

No treatment-related blood clinical pathology abnormalities were identified.

### Cerebrospinal Fluid

On Day 21 (the only time point evaluated post treatment), no test article-related CSF abnormalities were identified, including no evidence of the mild asymptomatic lymphocytic pleocytosis that is frequently observed after ICM administration of AAV vectors (defined as ≥6 leukocytes/μL of CSF) (FIG. 16).

### Cerebrospinal Fluid and Serum

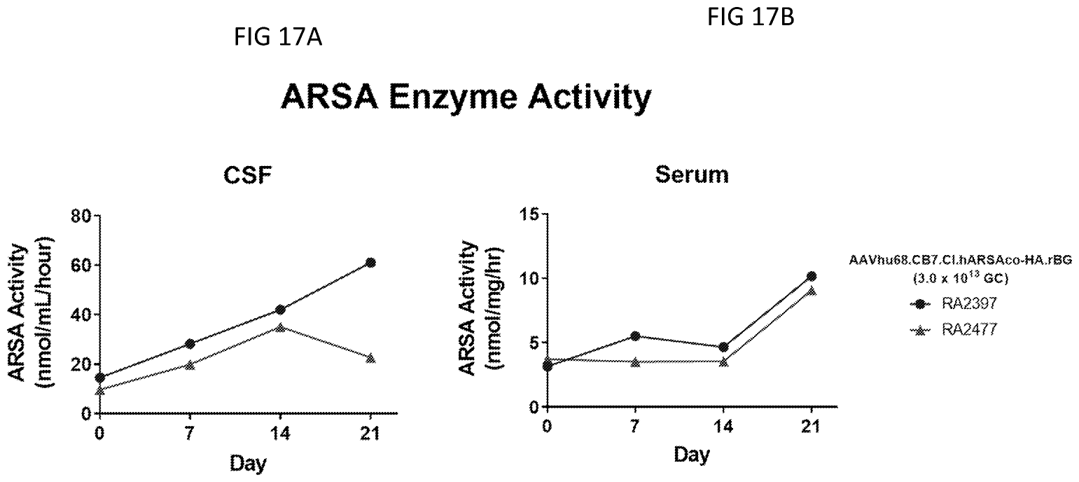

Transgene product expression (ARSA enzyme activity) was evaluated in CSF and serum collected at necropsy on Day 21. However, because this assay could not distinguish between the activity of human ARSA enzyme versus endogenous rhesus ARSA enzyme, the endogenous rhesus ARSA enzyme activity made it difficult to detect enzyme activity increases due to the expression of the human transgene product. For this reason, ARSA enzyme activity was detectable in CSF and serum of both animals at Day 0 prior to AAV administration, and these levels were therefore considered to be baseline levels of endogenous rhesus ARSA enzyme activity for this analysis (FIG. 17).

In CSF, both animals (N=2/2) exhibited an increase in ARSA enzyme activity from baseline levels by Day 7 (the first time point evaluated). For one animal, ARSA enzyme activity levels peaked at Day 14 at 3.6-fold baseline levels, and subsequently decreased to 2.3-fold baseline levels at necropsy on Day 21. For the other animal, ARSA enzyme activity progressively increased between Day 7 through the last time point evaluated on Day 21, peaking at 4.2-fold baseline levels at necropsy on Day 21 (FIG. 17).

In serum, both animals (N=2/2) exhibited a 2.4- to 3.2-fold increase in ARSA enzyme activity from baseline levels at necropsy on Day 21, with minimal (approximately 1.5-fold) to no increase from baseline levels observed at earlier time points on Day 7 or Day 14 (FIG. 17).

### ARSA Enzyme Activity—Cerebrospinal Fluid and Serum

Transgene product expression (ARSA enzyme activity) was evaluated in CSF and serum collected at necropsy on Day 21. However, because this assay could not distinguish between the activity of human ARSA enzyme versus endogenous rhesus ARSA enzyme, the endogenous rhesus ARSA enzyme activity made it difficult to detect enzyme activity increases due to the expression of the human transgene product. For this reason, ARSA enzyme activity was detectable in CSF and serum of both animals at Day 0 prior to AAV administration, and these levels were therefore considered to be baseline levels of endogenous rhesus ARSA enzyme activity for this analysis (FIG. 17).

In CSF, both animals (N=2/2) exhibited an increase in ARSA enzyme activity from baseline levels by Day 7 (the first time point evaluated). For one animal, ARSA enzyme activity levels peaked at Day 14 at 3.6-fold baseline levels, and subsequently decreased to 2.3-fold baseline levels at necropsy on Day 21. For the other animal, ARSA enzyme activity progressively increased between Day 7 through the last time point evaluated on Day 21, peaking at 4.2-fold baseline levels at necropsy on Day 21 (FIG. 17).

In serum, both animals (N=2/2) exhibited a 2.4- to 3.2-fold increase in ARSA enzyme activity from baseline levels at necropsy on Day 21, with minimal (approximately 1.5-fold) to no increase from baseline levels observed at earlier time points on Day 7 or Day 14 (FIG. 17).

### ARSA Enzyme Activity—Tissues

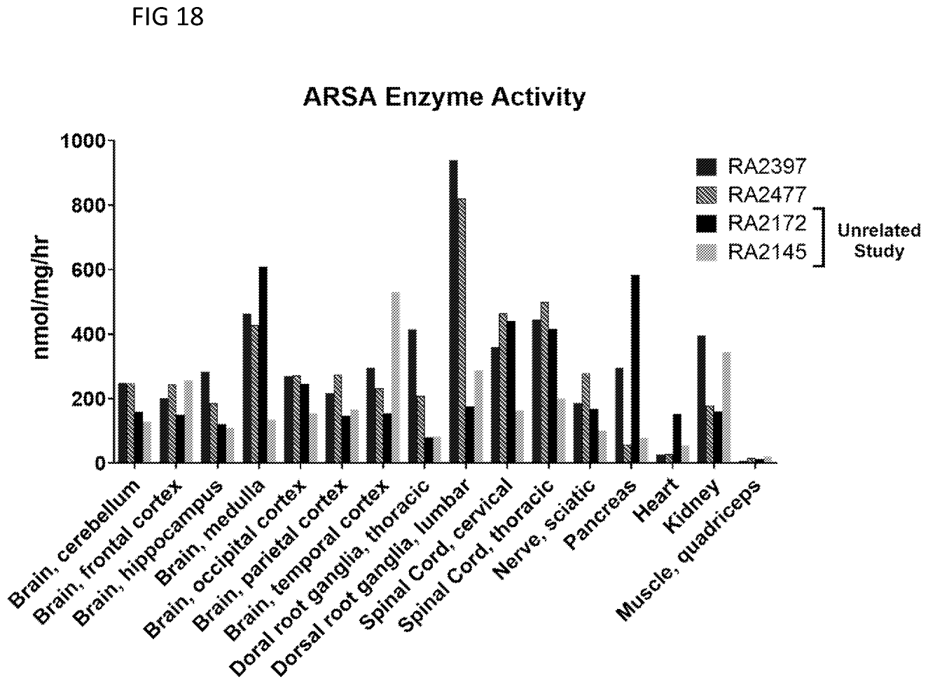

Transgene product expression (ARSA enzyme activity) was evaluated in tissues collected at necropsy on Day 21. However, because this assay could not distinguish between the activity of human ARSA enzyme versus endogenous rhesus ARSA enzyme, endogenous rhesus ARSA enzyme activity made it difficult to detect enzyme activity increases due to the expression of the human transgene product. For this reason, ARSA enzyme activity in tissues from animals in an unrelated study were used to determine background levels of endogenous rhesus ARSA enzyme activity for comparison to the enzyme levels observed in tissues from the AAVhu68.CB7.CI.hARSAco-HA.rBG-treated animals (FIG. 18).

An apparent increase in ARSA enzyme activity above background levels was detected in both animals in some regions of the brain (cerebellum, hippocampus, parietal cortex, occipital cortex), DRG (thoracic and lumbar), and spinal cord (thoracic), in addition to peripheral nerves (sciatic). In contrast, an increase in ARSA enzyme activity above background levels was not apparent in both animals in other regions of the brain (frontal cortex, medulla, temporal cortex) and spinal cord (cervical) or in peripheral organs (pancreas, heart, kidney, quadriceps muscle), although high individual variability in “normal” values from the 2 untreated animals make any interpretation difficult (FIG. 18).

### Human ARSA Immunostaining (HA Tag IHC and IF)

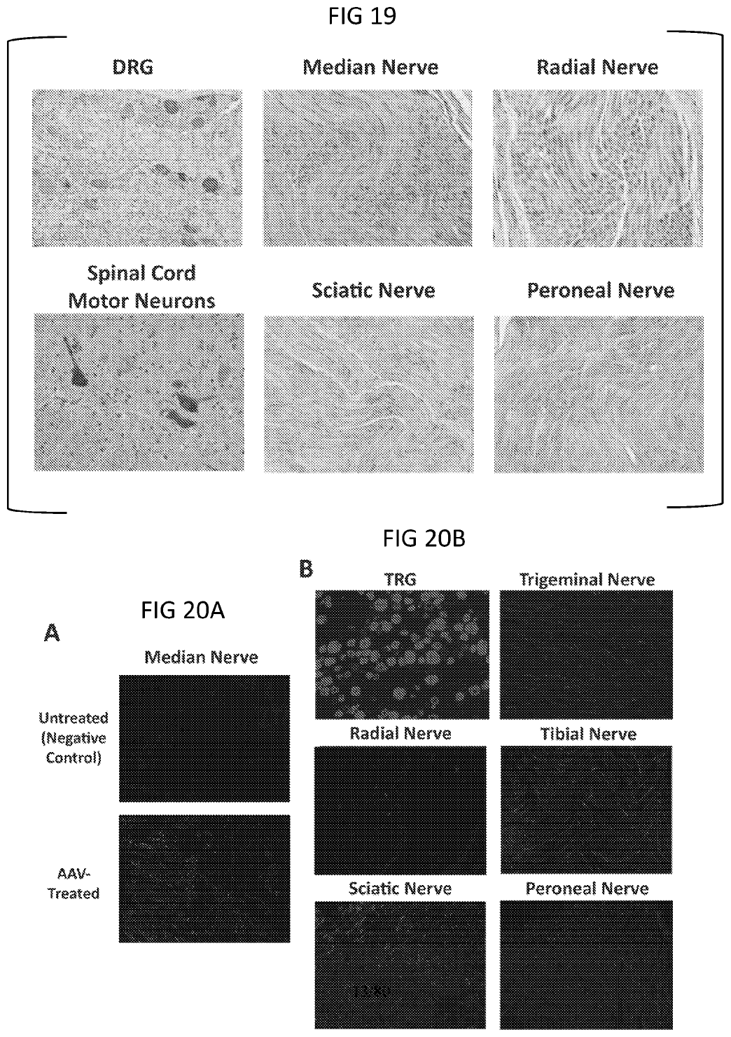

No detectable expression of the transgene product (human ARSA protein, detected via the HA tag immunostaining) was observed in nervous system tissues of untreated control rhesus macaque of comparable age from an unrelated study when analyzed by IHC (FIG. 19) or IF (FIG. 20A), indicating that background signal from endogenous rhesus ARSA protein was not detectable when using an antibody to detect the HA tag incorporated into the ARSA-expressing AAV vector. In contrast, cells expressing human ARSA protein were detected in spinal cord motor neurons (IHC) and cells of the DRG (IHC), TRG (IF), and peripheral nerves (IHC and IF: median, radial, sciatic, and peroneal; IF: tibial and trigeminal) in NHPs administered AAVhu68.CB7.CI.hARSAco-HA.rBG (FIG. 19 and FIG. 20A-FIG. 20B).

### Summary of Results

A single ICM administration of AAVhu68.CB7.CI.hARSAco-HA.rBG at a dose of 3.0×10¹³ GC (3.3×10¹¹ GC/g brain) was well-tolerated, with no test article-related abnormalities noted on clinical observations, body weight measurements, or clinical pathology of the blood or CSF.
AAVhu68.CB7.CI.hARSAco-HA.rBG administration led to detectable transgene product expression (ARSA enzyme activity) in CSF by Day 7 post treatment and serum by Day 21 for both animals (N=2/2). ARSA enzyme activity in CSF peaked at 2.3-4.2-fold baseline levels on Day 14-21. ARSA enzyme activity in serum peaked at 2.4-3.4-fold baseline levels on Day 21, with minimal to no increase from baseline levels observed at earlier time points. Both animals (N=2/2) also exhibited an apparent increase in ARSA enzyme activity above background levels in disease relevant target tissues, including certain brain regions (cerebellum, hippocampus, parietal cortex, occipital cortex), DRG, spinal cord, and peripheral nerves (sciatic). In contrast, an increase in ARSA enzyme activity above background levels was not apparent in peripheral organs (pancreas, heart, kidney, quadriceps muscle) and certain regions of the brain (frontal cortex, medulla, temporal cortex), although high individual variability in “normal” values from the 2 untreated animals make any interpretation difficult.
AAVhu68.CB7.CI.hARSAco-HA.rBG administration led to transgene product expression (ARSA IHC or ARSA IF using an antibody recognizing the HA tag) in spinal cord motor neurons, DRG, TRG, and peripheral nerves (median, radial, sciatic, peroneal, tibial, and trigeminal).
Cumulatively, this study established the potential for intrathecal AAV delivery to achieve therapeutic ARSA expression levels in the CSF and in disease-relevant target tissues of the CNS and PNS for the treatment of MLD.

### Example 5—Dose-Ranging Pharmacodynamic and Pilot Safety Study of AAVhu68.CB7.CI.hARSAco.rBG (GTP-207) Delivered Intrathecally Via the Cistema Magna in Adult Cynomolgus Macaques

A study was performed to evaluate the pharmacodynamic and preliminary safety profile of AAVhu68.CB7.CI.hARSAco.rBG (GTP-207), a recombinant adeno-associated virus (AAV) serotype hu68 vector expressing the human arylsulfatase A (ARSA) gene, following intra-cisterna magna (ICM) administration in adult cynomolgus macaque non-human primates (NHPs).

Adult male and female cynomolgus macaque NHPs received a single ICM administration of AAVHU68.CB7.CI.HARSACO.RBG (GTP-207) at a low dose (3.0×10¹² GC [3.3×10¹⁰ GC/g brain]), mid-dose (1.0×10¹³ GC [1.1×10¹¹ GC/g brain]), or high dose (3.0×10¹³ GC [3.3×10¹¹ GC/g brain]). In-life assessments included daily observations, body weight measurements, clinical pathology of the blood and cerebrospinal fluid (CSF), and evaluation of transgene product expression (ARSA enzyme activity) and anti-transgene product antibodies (anti-human ARSA antibodies) in CSF and serum. Necropsies were performed on Day 42, and the brain, spinal cord, and DRG were evaluated for histopathology and transgene product expression (ARSA immunohistochemistry [IHC]). For histopathology, the spinal cord and DRG were selected for histopathology because previous studies of AAV vectors administered ICM have revealed treatment-related findings in these tissues consisting of asymptomatic minimal to moderate toxicity to DRG sensory neurons and their associated axons. DRG sensory neuron toxicity has been observed with reproducible kinetics, consistently degenerating within 14-21 days after vector administration. Following cell body degeneration, subsequent degeneration of the axons of these cells (axonopathy) in the peripheral nerves and dorsal columns of the spinal cord appears around 30 days after vector administration. The axonal changes continue to be visible in animals sacrificed 90 days after vector administration. Based on these kinetics, we anticipated that the necropsy time points of 42 days would be sufficient to evaluate DRG histological findings and any associated clinical signs.

Following group assignment, each animal received a single ICM injection of AAVhu68.CB7.CI.hARSAco.rBG (GTP-207), the test article, at one of the following doses:
1.) 3.0×10¹² GC (low dose)
2.) 1.0×10¹³ GC (mid-dose)
3.) 3.0×10¹³ GC (high dose)

TABLE
Group Designations, Dose Levels, and Route of Administration

| | | | | | Dose | | | | |
|---|---|---|---|---|---|---|---|---|---|
| | | Animal | | Dose | (GC/g | | Injection | Day of | Day of |
| Group | Treatment | ID | Sex | (GC) | Brain)^{a} | ROA | Volume | Dosing | Necropsy |
| 1 | AAVhu68.CB7.CI.hARSAco.rBG | B5533 | F | 3.0 × 10¹³ | 3.3 × 10¹¹ | ICM | 1 mL | 0 | 42 ± 2 |
| | (GTP-207) | B3081 | M | | | | | | |
| 2 | AAVhu68.CB7.CI.hARSAco.rBG | B4119 | M | 1.0 × 10¹³ | 1.1 × 10¹¹ | ICM | 1 mL | 0 | 42 ± 2 |
| | (GTP-207) | B5012 | M | | | | | | |
| 3 | AAVhu68.CB7.CI.hARSAco.rBG | M00861 | M | 3.0 × 10¹² | 3.3 × 10¹⁰ | ICM | 1 mL | 0 | 42 ± 2 |
| | (GTP-207) | B5573 | F | | | | | | |

^{a}Doses are scaled based on a brain mass of 90 g for an adult NHP.
Abbreviations: F, female; GC, genome copies; ICM, intra-cisterna magna; ID, identification number; M, male; ROA, route of administration.

On Study Day 0, adult cynomolgus macaque NHPs (6-9 years old) received a single ICM administration of AAV.CB7.CI.hARSAco.rBG (GTP-207) at a low dose (3.0×10¹² GC), mid-dose (1.0×10¹³ GC), or high dose (3.0×10¹³ GC). In-life assessments included clinical observations, body weight measurements, clinical pathology of the blood and cerebrospinal fluid (CSF) (cell counts, clinical chemistry, and/or total protein), and evaluation of transgene product expression (ARSA enzyme activity) and anti-transgene product antibodies (anti-human ARSA antibodies) in serum and CSF. Necropsies were performed on Day 42. The brain, spinal cord, and dorsal root ganglion (DRG) were collected for histopathology and/or evaluation of transgene product expression (ARSA immunohistochemistry [IHC]). Additional tissues were collected and stored for possible future histopathological evaluation and vector biodistribution analysis.

### Results

### Clinical Observations

One animal in the mid-dose group (Animal B4119; 1.0×10¹³ GC, Group 2) was noted to be non-weight bearing on its right leg and curling the toes of its right foot on Day 18. This condition persisted throughout the end of the study and was sometimes accompanied by grimacing and face rubbing, although no other deficits were noted. Upon physical exam, the right hind limb palpated normally, had normal range of motion, and displayed symmetrical muscle mass compared to the left limb. Radiographs of pelvis, right leg, and right foot did not show any abnormalities. The animal was treated with meloxicam (0.2 mg/kg) through the end of the study. Necropsy findings revealed mild muscular atrophy of proximal right hindlimb muscles which was normal on histopathological observation. Animal B4119 was also noted to have a small nodule/mass on its ventral neck/right shoulder blade on Day 15 that persisted through the end of the study.

On Day 23, Animal B5012 was observed picking at its tail. Upon closer examination a pink, moist lesion mid-tail about 1 cm in diameter was identified. Despite treatment throughout the study with various medications (meloxicam, triple antibiotic ointment, cephalexin, exceed, silver sulfadiazine, chlorhexidine and saline) the tail lesions persisted through the end of the study.

### Body Weights

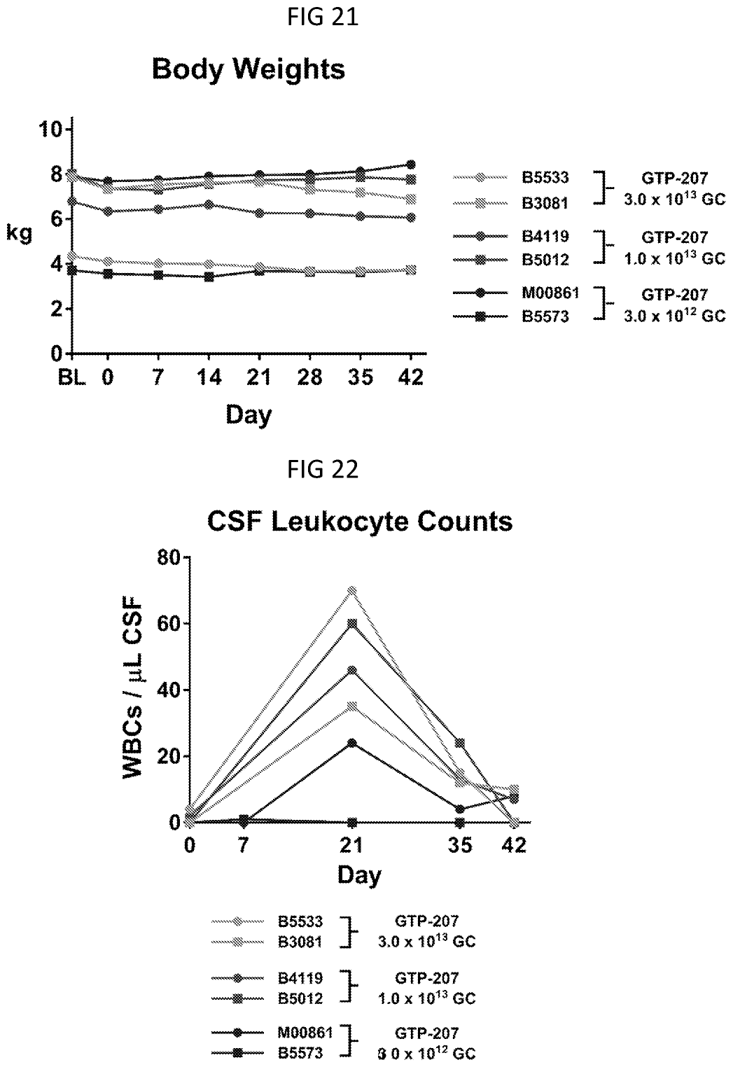

Body weights were stable for all animals throughout the study (FIG. 21).

### Blood

No treatment-related abnormalities were identified.

### Cerebrospinal Fluid

Pleocytosis can be related to hemodilution when red blood cells are observed in CSF samples as a result of blood contamination due to inadvertent contact with a subcutaneous or dural vessel during placement of the spinal needle. Several animals exhibited lymphocytic pleocytosis that was possibly secondary to hemodilution (≥6 leukocytes/μL of CSF with >30 RBCs/μL), including 1/2 animals in the low dose groups (3.0×10¹² GC, Group 3; Animal M00861 [Day 21], 2/2 animals in the mid-dose groups (1.0×10¹³ GC, Group 2; Animal B4119 [Day 35] and Animal B5012 [Days 21 and 35]).

Mild lymphocytic pleocytosis that was not likely attributable to hemodilution (≥6 leukocytes/μL of CSF with ≤20 red blood cells (RBCs)/μL) occurred by Day 21 after AAV administration in 1/2 animals in the low dose group (3.0×10¹² GC, Group 3; Animal M00861 [Day 42]), 1/2 animals in the mid-dose group (1.0×10¹² GC, Group 2; Animal B4119 [Day 42]), and 1/2 animals in the high dose group (3.0×10¹³ GC, Group 1; Animal B3081 [Days 21, 35, and 42]) (FIG. 22). The severity of the pleocytosis appeared dose-dependent, with higher CSF leukocyte counts observed for the mid-dose (1.0×10¹² GC, Group 2) and high dose (3.0×10¹³ GC, Group 1) groups compared to the low dose group (3.0×10¹² GC, Group 3).

For the 5 animals in the study exhibiting elevated leukocyte counts in CSF, a time-dependent response was observed, with CSF leukocyte counts declining from peak levels after Day 21 in all animals without treatment. The pleocytosis fully resolved by Day 42 in 2/2 animals in the mid-dose group (1.0×10¹³ GC, Group 2) and 1/2 animals in the high dose group (3.0×10¹³ GC, Group 1). However, CSF leukocyte counts remained slightly elevated (8-10 WBCs/L CSF) at Day 42 (the last time point evaluated) for the single animal (N=1/1) in the low dose group demonstrating CSF pleocytosis (3.0×10¹² GC, Group 3) and 1/2 animals in the high dose group (3.0×10¹³ GC, Group 1).

### Gross Pathologic Findings

There were no treatment-related gross pathologic findings in any animal under study.

### Histopathologic Findings

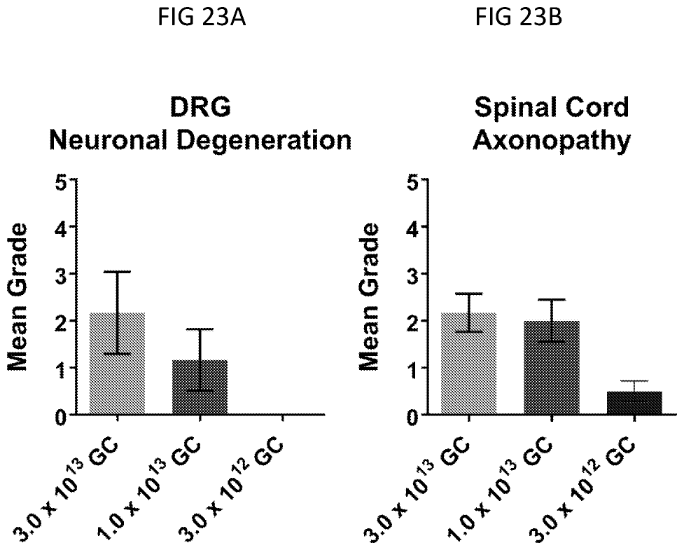

Limited histopathology analysis was performed, focusing on the DRG and their corresponding axons within spinal cord and peripheral nerves as they have been identified as potential targets for AAV-mediated pathology. AAV.CB7.CI.hARSAco.rBG (GTP-207)-related histopathologic findings consisted of degeneration of DRG sensory neurons with a secondary degeneration of the associated central axons in the dorsal white matter tracts of the spinal cord and peripheral nerves (axonopathy), which is consistent with what is usually seen after successful ICM gene transfer. The severity of DRG sensory neuron degeneration and associated axonopathy were dose-dependent (FIG. 23). One cynomolgus animal that was not treated with AAV was included in the histopathology analysis as control to help with determination of test-article versus background lesions as our facility had lesser prior experience with Cynomolgus macaques compared to Rhesus macaques. This animal (M11300) had minimal (grade 1) DRG neuronal degeneration in the lumbar segment, and minimal (grade 1) axonopathy in the dorsal white matter of the spinal cord and in sciatic nerve.

DRG neuronal degeneration. The incidence and severity of DRG neuronal degeneration appeared dose-dependent. The incidence and severity were highest in the high dose group (minimal to severe [Grade 1-5]; 2/2 animals; 4/6 ganglia; 3.0×10¹³ GC, Group 1) followed by the mid-dose group (minimal to marked [Grade 1-4]; 2/2 animals, 3/6 ganglia; 1.0×10¹³ GC, Group 2), while no DRG findings were observed in the low dose group (2/2 animals, 6/6 ganglia; 3.0×10¹² GC, Group 3) (FIG. 23).

Axonopathy in the spinal cord (dorsal white matter tract). The incidence and severity of axonopathy in the dorsal white matter tract of the spinal cord appeared generally dose-dependent. The incidence and severity were highest in the high dose group (minimal to moderate [Grade 1-3]; 2/2 animals, 6/6 spinal cord sections; 3.0×10¹³ GC, Group 1) and the mid-dose group (minimal to marked [Grade 1-4]; 2/2 animals, 6/6 spinal cord sections; 1.0×10¹³ GC, Group 2), and lowest in the low dose group (minimal [Grade 1]; 1/2 animals, 3/3 spinal cord sections; 3.0×10¹² GC, Group 3) (FIG. 23).

Axonopathy in the peripheral nerves. The incidence and severity of axonopathy in the peripheral nerves appeared generally dose-dependent. The incidence and severity were highest in the high dose group (minimal to marked [Grade 1-4]; 2/2 animals, 6/6 nerves; 3.0×10¹³ GC, Group 1) and the mid-dose group (minimal to marked [Grade 1-4]; 2/2 animals, 6/6 nerves; 1.0×10¹³ GC, Group 2), and lowest in the low dose group (minimal to mild [Grade 1-2]; 2/2 animals, 5/6 nerves; 3.0×10¹² GC, Group 3)

### Discussion of Histopathology Findings

The animal with unilateral lameness (B4119) in the mid-dose group presented histopathological findings consistent with AAV-related DRG toxicity with mild to marked dorsal root ganglia (DRG) neuronal degeneration and corresponding minimal (grade 1, median nerve) to moderate (grade 3, sciatic nerve) or marked (grade 4, tibial nerve axonopathy. Peripheral nerve axonopathy with grade 4 severity in tibial and sciatic nerve were also seen in another animal (B5533; 3.0×10¹³ GC) that did not have abnormal clinical sign. The causality between the peripheral nerve findings and the lameness in B4119 could therefore not be determined but cannot be excluded.

### Evaluation of Transgene Product Expression

### Cerebrospinal Fluid and Serum

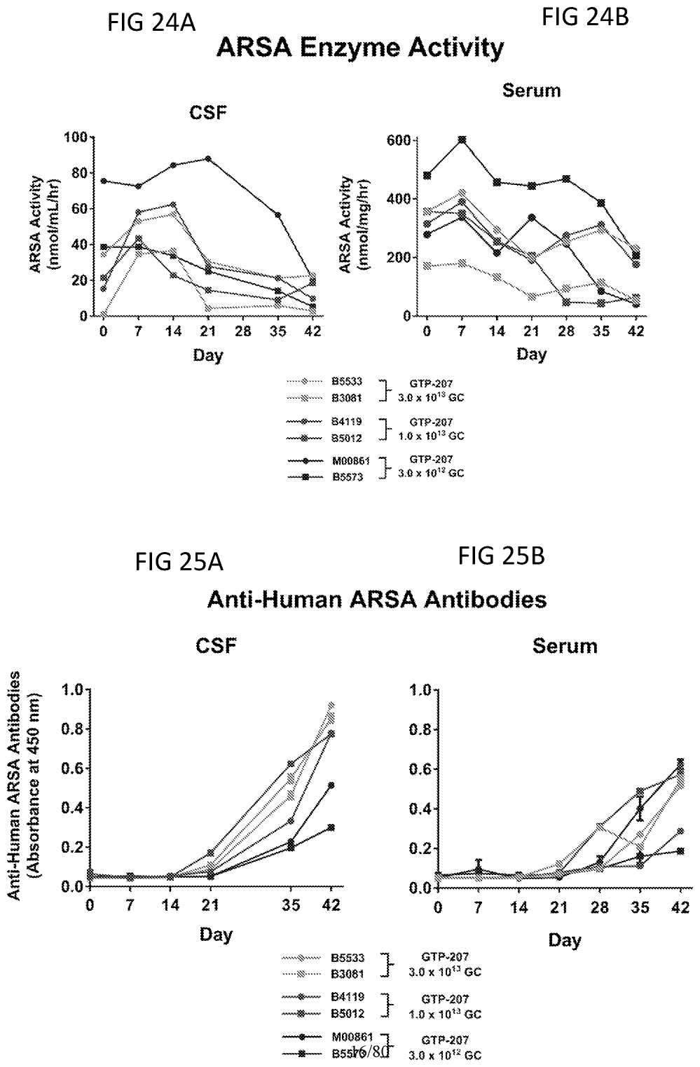

Transgene product expression (ARSA enzyme activity) was evaluated in CSF and serum. However, because the assay could not distinguish between human ARSA enzyme and endogenous Cynomolgus ARSA enzyme, the endogenous ARSA enzyme activity present in normal NHPs made it difficult to detect enzyme activity increases due to expression of the human transgene product. Thus, ARSA enzyme activity was detected in both CSF and serum for all dose groups at Day 0 prior to AAV.CB7.CI.hARSAco.rBG (GTP-207) administration (FIG. 24).

In CSF, ARSA enzyme activity increased from the Day 0 baseline levels for all animals at the high dose (3.0×10¹³ GC, Group 1; N=2/2 animals) and mid-dose (1.0×10¹³ GC, Group 2; N=2/2) and 1/2 animals at the low dose (3.0×10¹² GC; Group 3) by Day 7-14. For these animals, ARSA enzyme activity levels peaked between Day 7-21. ARSA enzyme activity appeared dose-dependent, with the mid-dose and high dose groups exhibiting a greater increase in expression from baseline levels (approximately 2-4-fold and 1.6-40-fold higher, respectively) compared to the low dose group (approximately 1.1-fold higher) (FIG. 24). As expected, ARSA enzyme activity in CSF declined to levels near or below baseline values by Day 42, which correlated with the onset of anti-human ARSA antibody expression around Day 21-35 in CSF and serum (FIG. 25).

In serum, ARSA enzyme activity increased from the Day 0 baseline levels for all animals with the exception of one animal in the mid-dose group (1.0×10¹³ GC, Group 2; Animal B5012), with peak levels observed by Day 7 (FIG. 24). The increase in ARSA enzyme activity did not appear to be dose-dependent. As expected, ARSA enzyme activity in serum declined to levels near or below baseline values by Day 42, correlated with the onset of anti-human ARSA antibody expression in serum and CSF (FIG. 25).

### Brain and Spinal Cord

Brain and spinal cord tissues were harvested from NHPs necropsied 42 days after treatment for a comprehensive histological evaluation of human ARSA expression by IHC.

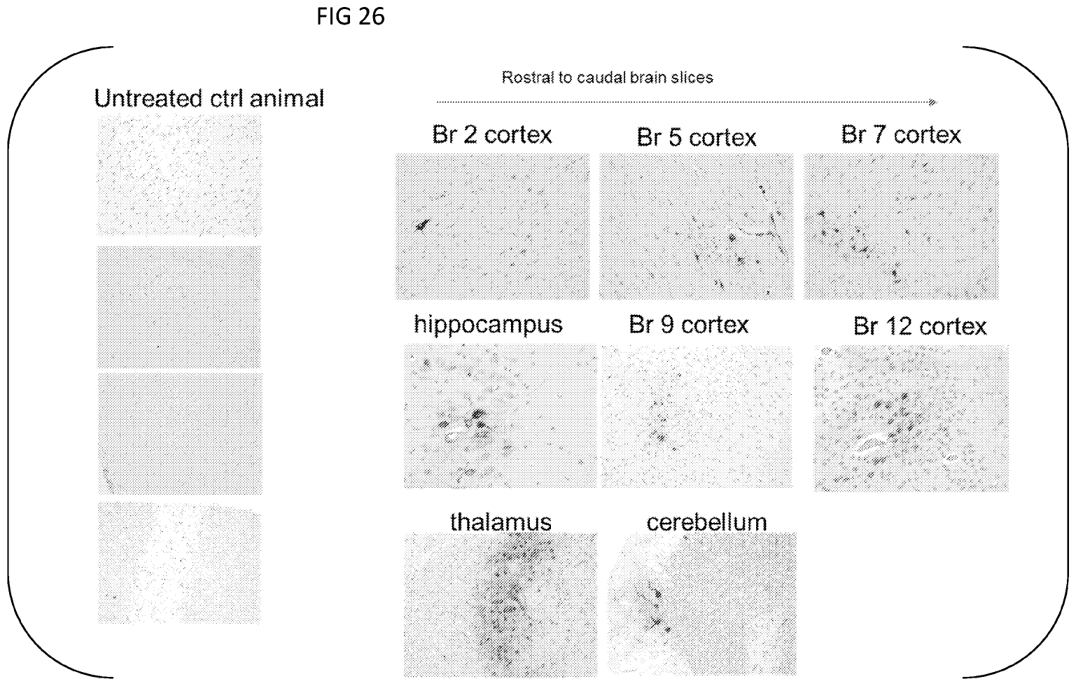

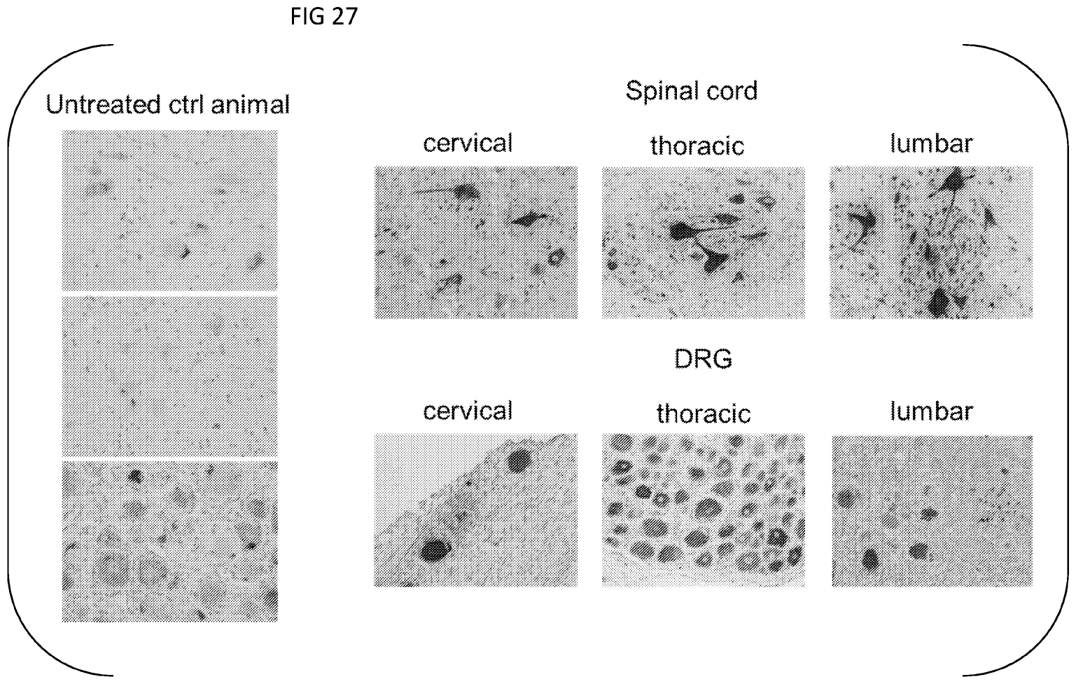

In NHPs administered the high dose of AAVHU68.CB7.CI.HARSACO.RBG (GTP-207) (3.0×10¹³ GC, Animals B5533 and B3081), transduced cells expressing human ARSA enzyme were detected throughout the brain, including the cortex, hippocampus, thalamus, and cerebellum (FIG. 26). Cells of the cervical, thoracic, and lumbar spinal cord along with cervical, thoracic, and lumbar DRG also expressed human ARSA enzyme (FIG. 27).

### Summary of Results

AAV.CB7.CI.hARSAco.rBG (GTP-207) was well-tolerated, although one mid dose animal demonstrated non-weight bearing unilateral lameness that may be test-article related although the relationship with histopathological changes was not conclusive. Clinical pathology changes included lymphocytic pleocytosis beginning on Day 21. CSF leukocyte counts declined from peak levels after Day 21 without treatment but remained slightly elevated at necropsy on Day 42 for some animals.
Transgene product expression (ARSA enzyme activity) was detectable in CSF and serum of most animals by Day 7-14 post treatment. Peak expression was observed by Day 7-14 in CSF and Day 7 in serum. CSF ARSA enzyme activity appeared dose-dependent, with the mid-dose (1.0×10¹³ GC) and high dose (3.0×10¹³ GC) groups exhibiting a greater increase in expression from baseline levels (approximately 2-4-fold and 1.6-40-fold higher, respectively) compared to the low dose group (approximately 1.1-fold higher; 3.0×10¹² GC). In contrast, ARSA enzyme activity levels in serum did not appear dose-dependent. As expected, ARSA enzyme activity in both CSF and serum declined to levels near or below baseline values by Day 42, correlating with the onset of a humoral response to the foreign human transgene product (anti-human ARSA antibodies) in CSF and serum around Day 21-35.
AAV.CB7.CI.hARSAco.rBG (GTP-207)-treated NHPs demonstrated transgene product expression (human ARSA IHC) in key target tissues for the treatment of MLD (brain and DRG). This result indicates that despite a humoral immune response to the foreign human transgene product, transduced cells still persisted within the target tissues for at least 42 days post treatment, producing ARSA where it would be needed to correct neurons and myelin-producing cells.
Test article-related histopathologic findings on Day 42 consisted of an asymptomatic degeneration of DRG sensory neurons with a secondary degeneration of the associated central axons in the spinal cord and peripheral nerves (axonopathy). The sensory neuron findings were minimal to severe (Grade 1-5) in severity, and the incidence and severity of findings were generally dose-dependent, with some Animals from the mid- and high dose groups demonstrating marked (Grade 4) or severe (Grade 5) DRG neuronal degeneration, respectively.
Cumulatively, this study established the potential for intrathecal AAV delivery to achieve therapeutic ARSA expression levels in the CSF of a large animal model. AAV.CB7.CI.hARSAco.rBG (GTP-207) demonstrated delivery of ARSA to deficient neurons and myelin-producing cells in the CNS and PNS. The treatment was well tolerated although one mid-dose animal demonstrated non-weight bearing unilateral lameness. Histopathology findings in the spinal cord and DRG were consistent with similar findings reported in NHPs after ICM administration of AAV vectors.

### Example 6—Arsa^{-/-} Mouse Model

The purpose of this natural history study was to characterize the phenotype of a novel Arsa−/− mouse model of metachromatic leukodystrophy (MLD) created using clustered regularly interspaced short palindromic repeats-(CRISPR)-associated protein 9 (CRISPR-Cas9) gene editing technology.

On Study Day 0, two mouse models of MLD derived from the same line (Line 407047) were enrolled in the study. The models were 1) untreated Arsa−/− mice and 2) Arsa−/− mice administered an AAV vector expressing GAL3ST1 to increase sulfatide storage in an attempt to create an aggravated model of MLD (referred to hereafter as AAV-GAL3ST1-treated Arsa−/− mice). For the MLD mouse models, adult mice were enrolled at ˜3 months of age and age-matched C57BL/6J wild type mice were included as controls.

In-life assessments included survival monitoring, body weight measurements, clinical scoring assessments, and evaluation of neuromotor function (ledge test, RotaRod assay, and CatWalk gait analysis). Necropsies were performed at approximately 9 and 15 months of age. Sulfatide storage was assessed in plasma (liquid chromatography/mass spectrometry [LC/MS]), peripheral organs, and target tissues relevant for the treatment of the neurological features of MLD (central nervous system [CNS] and peripheral nervous system [PNS]) (Alcian blue staining and LC/MS). Lysosomal storage lesions (lysosomal-associated membrane protein 1 [LAMP-1] immunohistochemistry [IHC]) and astrogliosis/neuroinflammation (glial fibrillary acidic protein [GFAP] IHC) were quantified in the CNS. Residual endogenous ARSA enzyme activity was also assessed in the CNS, peripheral organs, and serum to evaluate the extent of ARSA knockdown in the mouse models.

No naturally occurring animal model of MLD has been reported in the literature. There are two laboratory-generated Arsa knockout mouse models of MLD, both of which were created by Volkmar Gieselmann's group in Germany (Hess et al., 1996; Ramakrishnan et al., 2007). Both existing mouse models of MLD exhibit a normal lifespan. However, they do display some features observed in patients with MLD, including neurological symptoms and accumulation of the toxic sulfatides in cells of the CNS (oligodendrocytes, microglia, certain types of neurons) and PNS (Schwann cells and macrophages), with or without associated demyelination in the CNS and PNS.

TABLE
Previously Published Mouse Models of Metachromatic Leukodystrophy

| | | Sulfatide | | Neurological | |
|---|---|---|---|---|---|
| Genotype | Description | Accumulation | Demyelination | Symptoms | Lifespan |
| Arsa−/− | Arsa knockout | By 6 months | None | Gait pattern | Normal |
| (Single | (homologous | (lipid | | abnormalities and | |
| Mutant) (Hess | recombination) | analysis) | | reduced rotarod | |
| et al., 1996) | | By 10-12 | | performance by 6- | |
| | | months | | 10 months | |
| | | (histology) | | | |
| tg/Arsa−/− | Arsa knockout | By 6 months | By | Hind limb | Normal |
| (Double | (homologous | (lipid | 17-18 months | grasping upon tail | |
| Mutant) | recombination) | analysis) | (PNS > CNS) | suspension in mice | |
| (Ramakrishnan | Gal3stl | By 17 months | | >16 months old | |
| et | transgene under the | (histology) | | leading to | |
| al., 2007) | control of the Plpl | | | progressive hind | |
| | promoter (driving | | | limb paralysis and | |
| | overexpression in | | | inability to stay on | |
| | myelinating cells) | | | rotarod by | |
| | | | | 21-24 months | |
| | | | | Reduced NCV | |
| | | | | by 22-24 months | |

Abbreviations:
Arsa, arylsulfatase A (gene, mouse);
CNS, central nervous system;
Gal3stl, galactose-3-O-sulfotransferase-1 (gene, mouse);
GAL3STI, galactose-3-O-sulfotransferase-1 (protein);
NCV, nerve conduction velocity;
PNS, peripheral nervous system;
tg, transgene.

The new Arsa−/− mouse line was created using embryonic microinjection of CRISPR/Cas9. This genetic engineering strategy targeted the mouse Arsa gene located on chromosome 15 using several guide RNAs to facilitate the targeted deletion of exon 2 through exon 4. While the classic Arsa−/− model employed homologous recombination of a neomycin cassette to produce a null allele, CRISPR/Cas9 gene editing was anticipated to produce a complete knock out with a comparable phenotype in less time than previous gene targeting methods. Using this approach, four founders were produced that had deletions of exons 2-4 of the mouse Arsa gene, ranging from 1105 base pairs (bp) to 1133 bp in length. All four founders were bred once with C57BL/6J wild type (WT) mice and successfully transmitted the deleted allele to the F1 generation. F1 generation carriers were crossbred once more into a C57BL6/J background to further dilute any unwanted off-target editing. All four lines produced F2 generation carriers, which were bred to generate and characterize four Arsa−/− mouse lines (Lines 407046, 407047, 407048, and 407049).

This study characterized the phenotype of the Line 407047 Arsa−/− mouse model, along with additional Line 407047 Arsa−/− mice administered a single dose of an AAV vector expressing human galactose-3-O-sulfotransferase 1 (GAL3ST1) (AAV9-PHP.B.CB7.hGal3ST1co.rBG). The GAL3ST1 enzyme catalyzes the sulfation of membrane glycolipids, including the final step in the synthesis of sulfatide, a major lipid component of the myelin sheath. Administration of AAV9-PHP.B.CB7.hGal3STlco.rBG was hypothesized to increase sulfatide storage in an attempt to create an aggravated (i.e., more severe) mouse model of MLD. Details about the AAV9-PHP.B.CB7.hGal3ST1co.rBG vector are presented in Table 3.

Study animals were not randomized. Group designations, dose levels, and the route of administration (ROA) are presented in the table below.

TABLE
Group Designations, Dose Levels, and Route of Administration

| | | | | | Dose | | | Necropsy | Necropsy |
|---|---|---|---|---|---|---|---|---|---|
| | N and | | | Dose | Volume | | Dosing | Study | Age |
| Group | Sex | Genotype | Treatment | (GC/Animal) | (μL) | ROA | Day | Day | (Months) |
| 1 | SM | Arsa−/− | None | N/A | N/A | N/A | N/A | 360 ± 8 | 15 |
| | SF | | | | | | | | |
| 2 | 7M | C57BL/6 | None | N/A | N/A | N/A | N/A | 360 ± 8 | 14-15 |
| | 6F | J (WT) | | | | | | | |
| 3 | 4M | Arsa−/− | None | N/A | N/A | N/A | N/A | 180 ± 12b | 8-9 |
| | 4F | | | | | | | | |
| 4 | 4M | C57BL/6J | None | N/A | N/A | N/A | N/A | J80 ± 12b | 9 |
| | 4F | (WT) | | | | | | | |
| 5 | SM | Arsa−/− | AAV9- | 1.0 × 10¹² | 100 | IV | 0 | 180 ± 12 | 9 |
| | OF | | PHP.B.CB7.hGal3STlco.rBG | | | | | | |
| 6 | 6M | C57BL/6J | AAV9- | 1.0 × 10¹² | 100 | IV | 0 | 180 ± 12 | 8-10 |
| | OF | (WT) | PHP.B.CB7.hGal3STlco.rBG | | | | | | |

Two animals in this group were necropsied early on Day 128 to assess AR_SA expression at 4 months of age (IM, IF); the remaining animals were necropsied on Day 180±12.

Abbreviations: Arsa, arylsulfatase A (gene, mouse); F. female; GC., genome copies; ID, identification number; IV, intravenous: M, male; N, number of animals; NIA. not applicable; ROA, route of administration; WT, wild type.

On Study Day 0, untreated adult (˜3 months of age) Arsa^{-/-} mice and age-matched C57BL/6J wild type controls were enrolled in the study. Additionally, AAV-GAL3ST1-treated adult (˜2 months of age) Arsa^{-/-} mice and C57BL/6J wild type controls were enrolled in the study to attempt to produce an aggravated mouse model of MLD.

In-life assessments included viability checks, body weight measurements, clinical scoring assessments, and evaluation of neuromotor function (ledge test, RotaRod assay, and CatWalk gait analysis) at various time points. Necropsies were performed on Day 180 (˜9 months of age) and Day 360 (˜15 months of age). Sulfatide storage was assessed in plasma (LC/MS), brain, spinal cord, sciatic nerve, liver, spleen, kidney, heart, and quadriceps muscle (Alcian blue staining and LC/MS). Lysosomal storage lesions (LAMP1 IHC) and astrogliosis/neuroinflammation (GFAP IHC) were quantified in the brain and spinal cord. ARSA enzyme activity was also assessed in the brain, spinal cord, liver, spleen, kidney, and serum.

The study included necropsy time points at Day 180 and Day 360. The Day 180 time point, which corresponded to ˜9 months of age, was chosen for Groups 3 and 4 to evaluate an early stage of the disease phenotype when sulfatide storage and neurological abnormalities have been observed in previously generated mouse models of MLD. Additionally, the Day 180 necropsy time point was chosen for Groups 5 and 6 because it was hypothesized that successful aggravation of sulfatide storage through treatment with AAV9-PHP.B.CB7.hGal3STlco.rBG would lead to earlier phenotype development. The Day 360 time point, which corresponds to ˜15 months of age, was selected for Groups 1 and 2 to evaluate the long-term phenotype progression and possible late-onset demyelination in the CNS and PNS, which has been observed in a previously generated mouse model of MLD. For two animals in Groups 3 and 4 each, an early necropsy was performed on Day 128 (4 months of age). This earlier necropsy time point was selected for this subset of animals to obtain an early readout regarding the extent of knockdown of ARSA expression in the Arsa^{-/-} mouse model.

All in-life assessments were performed at frequent intervals during the expected onset and progression of the disease phenotype. Body weights were acquired monthly to monitor for weight loss (i.e., body wasting), which could be expected with deteriorating neuromotor function and is similarly observed in patients with MLD. Clinical scoring assessments of tremors, gait and coordination, clasping reflex, posture, and fur quality were performed every other week and were based on the known phenotype of previously generated mouse models of ataxia. Clinical scoring enabled evaluation of disease progression, including the development of ataxia, which is similarly observed in MLD patients (Guyenet et al., 2010); higher clinical scores would indicate a more severe phenotype. Neuromotor function was evaluated using the ledge test, RotaRod assay, and CatWalk gait analysis. The ledge test was performed every other week and consisted of evaluating the animal's ability to balance and walk on the ledge of its cage. The RotaRod assay was performed monthly and evaluated coordination and balance by measuring the time to fall for mice running on a spinning rod that progressively accelerates. The CatWalk assay was performed every 2 months and consisted of a system that tracked the footprints of mice as they walked across a glass plate to quantify the animal's speed and various aspects of gait. These assays were selected to assess coordination and balance (ledge test, RotaRod assay) and gait (CatWalk) because progressive motor phenotypes have been observed in previous MLD mouse models that are reminiscent of those observed in human patients, including ataxia, hindleg weakness, and paralysis. A decrease in neuromotor function would be expected to result in increased scores on the ledge test or a decrease in fall latencies on the RotaRod test. A decrease in neuromotor function could also be expected to result in gait and/or walking speed abnormalities as assessed by the CatWalk assay when compared to healthy controls, including abnormalities in animals' base of support, print positions, cadence, step sequence regularity, average body speed, and/or stride length.

At necropsy, key target tissues for the treatment of MLD (CNS [brain, spinal cord] and PNS [sciatic nerve]), along with peripheral organs (liver, spleen, kidney, heart, quadriceps muscle) and plasma were collected to evaluate sulfatide storage (Alcian blue staining and quantification by LC/MS) because sulfatides are the toxic substrates that accumulate in the absence of functional ARSA enzyme in both mice and humans with MLD. Lysosomal storage lesions (LAMP1 IHC) and astrogliosis/neuroinflammation (GFAP IHC) were also evaluated in the brain and spinal cord because these are neuropathologic hallmarks of MLD in mice and humans, which increase over time as the disease progresses. Additionally, residual endogenous ARSA enzyme activity was assessed in a subset of mice at 4 months of age to evaluate the knockdown of ARSA expression in Arsa^{-/-} mice. Residual ARSA enzyme activity was measured in target tissues relevant for the treatment of the neurological features of MLD (brain, spinal cord), along with peripheral organs (liver, kidney, spleen) and systemically in serum.

### Clinical Scoring Assessment

Clinical signs were scored by two research staff members using an
assessment of clasping ability, gait, tremor, kyphosis, and fur quality.
These measures were chosen to assess clinical status based on
the symptoms typically exhibited by Arsa−/− mice. Scores above 0
indicated clinical deterioration. Table. Clinical Scoring Assessment

| Assessment | | |
|---|---|---|
| Category | Observation | Score |
| Fur Quality | Shiny coat, smooth fur, well groomed, clean coat | 0 |
| | Not well groomed, slightly oily or rough coat | 1 |
| | Rough Hair coat-hair coat is oily, dirty, stands on end | 2 |
| | Very rough hair coat, dehydration | 3 |
| Gait | Active, energetic. Body weight supported on all limbs. | 0 |
| | Even hindlimbs, abdomen raised | |
| | Able to run, but sluggish. Limp when walking. | 1 |
| | Reluctant to move, severe limp, lowered pelvis, feet | 2 |
| | point away from body | |
| | Moves only when manually prodded & minimally, | 3 |
| | drags abdomen | |
| Tremor | No sign of tremor | 0 |
| | Very slight, impermanent | 1 |
| | Mild, visible upon close observation | 2 |
| | Moderate, visible immediately | 3 |
| | Severe, impacts mobility | 4 |
| Clasping | Hindlimbs consistently splayed outward from abdomen | 0 |
| | 1 hindlimb is retracted toward abdomen for >50% time | 1 |
| | Both limbs partially retracted for >50% of time | 2 |
| | Hindlimbs entirely retracted and touching abdomen | 3 |
| | for >50% time | |
| | Permanent full grasping of 4 limbs | 4 |
| Posture | Normal, flat back-easily straightens spine as it walks | 0 |
| | Transitory hunching, evident only when resting-mild | 1 |
| | but able to straighten spine | |
| | Rests upright with back hunched, unable to straighten | 2 |
| | completely when moving but mild | |
| | Pronounced and maintained kyphosis while walking | 3 |
| | and sitting | |

### Neuromotor Function Assessments

### Ledge Test

The ledge test measures coordination, which is impaired in neurodegenerative diseases associated with ataxia, such as MLD. Mice were evaluated for phenotypic progression through conducting the ledge test according to the published protocol (Guyenet et al., 2010). Briefly, the animal was lifted from its cage and placed on the cage's ledge. The mouse was observed and assigned a score based on its ability to navigate along the ledge and get itself back into its cage. Scores above 0 indicated a decrease in neuromotor function.

TABLE
Ledge Test Scoring

| Score | Observation |
|---|---|
| 0 | Mouse walks along the ledge without losing its balance, and lowers |
| | itself back into its cage using its paws gracefully and without |
| | losing balance |
| 1 | Mouse loses its footing while walking along the ledge, but |
| | otherwise appears coordinated. |
| 2 | Mouse does not effectively use its hind legs or lands on its head |
| | rather than its paws when descending into the cage |
| 3 | Mouse nearly or completely falls off the ledge while walking or |
| | attempting to lower itself, or shakes and refuses to move at all |
| | despite encouragement. |

Abbreviations:
N/A, not applicable.

### RotaRod

Coordination and balance were measured using the RotaRod test (Ugo Basile; Gemonio, Italy). Briefly, mice were first habituated to the RotaRod by placing up to five mice per trial in a lane of the RotaRod device facing the wall. Mice were allowed to stabilize themselves on the fixed (non-rotating) rod for 2 minutes. Two habituation trials were then performed with the rod rotating for 1 minute at a constant speed of 5 revolutions per minute (RPM). Between each habituation trial, mice were allowed to rest in the RotaRod collecting box for approximately 1 minute. If a mouse fell during the habituation phase, it was immediately placed back on the rod.

Immediately following habituation, testing trials were performed to measure how long each mouse could remain on the rotating rod while it was accelerating. The mice were placed in a lane of the RotaRod device facing the wall and allowed to equilibrate on the fixed (non-rotating) rod to establish a firm grip. The rod was then set to spin at a constant speed of 5 RPM for a few seconds to allow the mice to equilibrate. Once equilibrated, the rod was set to accelerate from 5 RPM to 40 RPM over 300 seconds. For each animal, the testing trial was considered terminated when the mouse fell off the rod, completed two passive revolutions, or 300 seconds had elapsed. The fall latency (defined as the time between the initiation of rod acceleration and trial termination) was recorded. A total of three sequential test replicates were performed for the mice in each trial, with a 1-3 minute pause in between runs to allow the animals to rest in the collecting box.

### CatWalk Gait Analysis

Gait and walking speed were assessed using the CatWalk XT gait analysis system (Noldus Information Technology, Wageningen, The Netherlands). The CatWalk XT tracks the footprints of mice as they walk across a glass plate. The system quantifies the dimensions of each paw print and statistically analyzes the animal's speed and other features of gait.

To perform this assessment, the Catwalk XT was calibrated, with the appropriate width of the walkway set, prior to the start of the test. All experiment settings were entered into the Catwalk XT software, including animal type, time point, and run criteria. Animals were brought into the room and allowed to acclimate in darkness for at least 30 minutes prior to running on the Catwalk XT. Once acclimation was complete, an animal was selected and placed at the entrance of the walkway. The researcher started the acquisition software and allowed the animal to walk down the walkway. The animal's home cage was placed at the end of the walkway for encouragement. The run was complete when the animal had successfully walked to the end of the catwalk within the allotted time limit, otherwise the run was repeated. Animals ran three trials with a minimum duration of 0.50 seconds and a maximum duration of 5.00 seconds. Three successful runs were needed for the trial to be considered complete. If an animal failed to complete three runs after 10 minutes of testing, only the completed runs were used for analysis. Animals were tested twice on two consecutive days. The first day of testing was used to habituate animals to the testing apparatus, and the second day of testing was scored. Runs were auto-classified using the Catwalk XT software, after which footprints were checked for accuracy and proper labeling. Any non-footprint data were manually removed. All data were exported into Microsoft Excel and GraphPad Prism 7.0 for analysis.

### Catwalk Gait Analysis Parameters Evaluated

Parameters automatically measured by the Catwalk XT system included base of support, print positions, cadence, step sequence regularity, average body speed, and stride length as described below. Mean values were calculated and analyzed for each group.

Base of support was determined by the Catwalk XT system as the average width between either the front paws or the hind paws. Print positions were determined by the Catwalk XT system as the distance between the position of the hind paw and the position of the previously placed front paw on the same side of the body (ipsilateral) and in the same Step Cycle. The animal's cadence was determined by the Catwalk XT system as steps per second. The step sequence was evaluated by the Catwalk XT system by determining the percent of steps that falls into one of six regular patterns typically observed in healthy mice. The average body speed was determined by the Catwalk XT system based on the step cycle of a specific paw by dividing the distance that the animal's body traveled from one initial contact of that paw to the next by the time to travel that distance. The stride length was determined by the Catwalk XT system based on the distance (in Distance Units) between successive placements of the same paw. Contact area was determined by the Catwalk XT system based on Illuminated Footprints™ technology where paws are captured by a high-speed video camera that is positioned underneath the walkway. Print width and print length were determined by the Catwalk XT system from the video images with paw prints used in the footprint classification. Once classification was done, the CatWalk software automatically calculated parameters related to individual footprints.

### Histological Processing and Evaluation

### LAMP-1 IHC (Evaluating Lysosomal Storage Lesions)

LAMP-1 immunohistochemical staining was performed on deparaffinized paraffin sections. Briefly, antigen retrieval was performed by boiling slides at 100° C. for 6 minutes in 10 mM citrate buffer (pH 6.0). Slides were then incubated with 2% hydrogen peroxide for 15 minutes, blocked using avidin/biotin reagents for 15 minutes each (Vector Laboratory; Catalog number: SP-2001), and incubated with 1% donkey serum in phosphate-buffered saline (PBS) with 0.2% Triton-X for 10 minutes at room temperature. Slides were then incubated with a rat anti-mouse LAMP-1 primary antibody (Abcam, Catalog #Ab25245) at 37° C. for 1 hour. Slides were washed and then incubated with a biotinylated donkey anti-rabbit IgG secondary antibody (Jackson; Catalog number: 711-065-152) for 45 minutes at room temperature. Slides were washed and then incubated with Vectastain ABC reagent (Vector Laboratories; Catalog number: PK-6100). Colorimetric development was performed using a 3,3′-Diaminobenzidine (DAB) kit (Vector Laboratories; Catalog number: SK-4100) followed by counterstaining with hematoxylin and coverslipping for evaluation.

### GFAP IHC (Evaluating Astrogliosis/Neuroinflammation)

GFAP immunohistochemical staining was performed on deparaffinized paraffin sections. Briefly, antigen retrieval was performed by boiling slides at 100° C. for 6 minutes in 10 mM citrate buffer (pH 6.0). Slides were then incubated with 2% hydrogen peroxide for 15 minutes, blocked using avidin/biotin reagents for 15 minutes each (Vector Laboratory; Catalog number: SP-2001), and incubated with 1% donkey serum in PBS with 0.2% Triton-X for 10 minutes at room temperature. Slides were then incubated with a rabbit anti-mouse GFAP primary antibody (Abcam, Catalog #ab7260) at 37° C. for one hour. Slides were washed and then incubated with a biotinylated donkey anti-rabbit IgG secondary antibody (Jackson; Catalog number: 711-065-152) for 45 minutes at room temperature. Slides were washed and then incubated with Vectastain ABC reagent (Vector Laboratories; Catalog number: PK-6100). Colorimetric development was performed using a DAB kit (Vector Laboratories; Catalog number: SK-4100) followed by counterstaining with hematoxylin and coverslipping for evaluation.

### Alcian Blue Staining (Evaluating Sulfatide Storage)

Alcian Blue staining was performed on deparaffinized paraffin sections. Briefly, slides were stained in Alcian Blue (1 g of Alcian Blue, 90 mL H2O, 10 mL 1N HCl; pH 1.0) for 15 minutes. Slides were then removed from the stain, washed under running tap water for 1 minute, and counterstained in Nuclear Fast Red for 2-3 minutes. The slides were dehydrated in ethanol followed by xylene and coverslipped for evaluation.

### Histopathological Evaluation

LAMP-1 and GFAP IHC staining was quantified from whole-slide scanned digital images (scanner Aperio AT2) and positive surface divided by the whole tissue surface present on the slide using VisioPharm image analysis software. Alcian Blue staining was not quantified. Briefly, well-stained and intact regions of sections of the brain and spinal cord were manually outlined using VIS version 2019.07.0.6328 (Visiopharm, Hoersholm, Denmark). For the brain, LAMP-1 positive area was quantitated via thresholding using the IHS-S (Intensity, Hue, Saturation model) classification feature. The LAMP-1-negative area was quantified via thresholding using the HDAB-Hematoxylin classification feature, and the LAMP-1-positive and LAMP-1-negative area classifications were used to generate the percentage of the outlined section that was LAMP-1 positive, the number of LAMP-1 positive objects, and the average size of all LAMP-1 objects identified in the section. For the spinal cord, LAMP-1 positive and LAMP-1 negative areas were quantified via thresholding using the HDAB-DAB classification feature, and the LAMP-1-positive and LAMP-1-negative area classifications were used to generate the percentage of the outlined section that was IBA1-positive, the number of LAMP-1-positive objects, and the average size of all LAMP-1 objects identified in the section.

### Quantification of Sulfatide Storage by LC/MS

Thawed tissues were lyophilized overnight and ground to a fine powder in 2.0 mL polypropylene tubes with ceramic beads using a Precellys bead-beating homogenizer (Bertin Technologies, Rockville, MD) at 4° C. Aliquots of powder (˜2.5-5.0 mg) were weighed on an analytical balance followed by homogenization in 500 μL of 80% methanol in the Precellys homogenizer at 4° C. A 100 μL aliquot of homogenate was then spiked with 10 μL of a d₃-C18-sulfatide internal standard (25 μM) and extracted with 400 μL of ice cold methanol in a 2.0 mL Eppendorf tube. The sample was centrifuged for 5 minutes at 14,000×g at 4° C. Aliquots (400 μL) of methanolic supernatants were dried under nitrogen in a 96-well plate at 45° C. and reconstituted in 150 μL of methanol for LC/MS analysis.

Calibration samples of sulfatide standards were prepared. Standard powders of sulfatides (lysosulfatide and C16, C18, d₃-C18, and C24:1; Matreya, State College, PA) were weighed on an analytical balance, and individual stock solutions (1 mM) were prepared in 2:1 methyl tert-butyl ether/methanol. The d₃-C18 sulfatide internal standard stock solution was diluted in methanol to give a 25 μM spiking internal standard solution. Aliquots of the individual stock solutions were combined to make a high calibration spiking solution of 50 μM lysosulfatide, 50 μM C16, 250 μM C18, and 250 μM C24:1 sulfatide in methanol. This high calibration spiking solution was serially diluted in methanol to make 0.1, 0.25, 0.5, 1, 5, 10, 25, and 50 μM calibration curve spiking solutions for lysosulfatide and C16 sulfatide, along with 0.5, 1.25, 2.5, 5, 25, 50, 125, and 250 μM calibration curve spiking solutions for C18 and C24:1 sulfatide. Calibration curve solutions for LC/MS analysis were created by pipetting 10 μL of each spiking solution and 10 μL of the d₃-C18-sulfatide internal standard (25 μM) into 100 μL of 80% methanol, resulting in LC/MS calibration curves of 0.01, 0.025, 0.05, 0.1, 0.5, 1, 2.5, and 5 μM for lysosulfatide and C16 sulfatide, along with LC/MS calibration curves of 0.05, 0.125, 0.25, 0.5, 2.5, 5, 12.5, and 25 μM for C18 and C24:1 sulfatide. A 400 μL aliquot of methanol was added to each solution. The sample was vortexed, and 400 μL was dried under nitrogen in a 96-well plate at 45° C. and reconstituted in 150 μL of methanol for LC/MS analysis.

Sulfatides were quantified with an Agilent 1290 Infinity UHPLC/6495B triple quadrupole mass spectrometer. Biological extracts and calibration solutions in 96-well plates were injected (5 μL) and separated on the UHPLC. Sulfides were eluted by gradient elution on a Waters Acquity BEH C18 2×100 mm, 1.7 μM column at a flow rate of 0.4 mL/minute at 45° C. A 7.5 minute gradient was used beginning with 35% solvent A (70/30 deionized water/acetonitrile/0.1% formic acid) and 65% solvent B (50/50 acetonitrile/isopropanol/0.1% formic acid) held for 0.5 minutes and increased to 100% solvent B over 5.5 minutes, held at 100% solvent B until 7.5 minutes followed by reequilibration back to starting conditions from 7.6 to 10 minutes. The HPLC flow was diverted to waste for the first 0.5 minute then directed to the electrospray ionization source. Sulfatides were ionized by electrospray ionization in the positive ionization mode on the mass spectrometer. The Agilent Jet Stream electrospray ionization source was operated with a nitrogen gas temperature of 250° C., gas flow of 14 L/minute, nebulizer of 45 psi, sheath gas temperature of 325° C., sheath gas flow of 12 L/minute, capillary voltage of 3500 V, and nozzle voltage of 500 V. Multiple reaction monitoring (MRM) was used to quantitate sulfatides with a peak width of 0.7 Da and an electron multiplier voltage of 400 V in the positive ion mode. As an example, a primary transition for C16 sulfatide of m/z 780.57→264.2 was used for the quantitation of C16 by monitoring m/z 264.2, the while the secondary transition m/z 780.57→682.6, generated by neutral loss of H₂SO₄ from the parent ion, was used to confirm the primary transition as an authentic sulfatide. Agilent MassHunter software was used to generate linear or quadratic calibration curves (1/x or 1/x² weighting and R² 0.99 or better) to quantify sulfatides in biological samples.

### Measuring ARSA Enzyme Activity

ARSA enzyme activity was measured in dialyzed serum or tissues samples using a p-nitrochatechol assay. Briefly, dialyzed serum (diluted 1:5, 1 part serum+4 parts diluent; or tissues (diluted 0.3 mg/mL) were diluted into a base buffer (0.5 M sodium acetate buffer, pH 5.0; 10% sodium chloride; 0.5 mM sodium pyrophosphate) with or without 125 μM silver nitrate, and L of the diluted sample was loaded into eight wells (four duplicates) into a 96-well plate. Next, 30 μL of substrate (10 mM 4-nitrocatechol sulfate) was added. The reaction was stopped by immediately adding 90 μL 1N NaOH (stop solution) in two of the duplicates (4 wells) and the rest of the samples were incubated at 37° C. for 1 hour. The reaction was stopped by adding 90 μL 1N NaOH (stop solution). The absorbance was measured by reading the plate at 515 nm using a plate reader. The aborbance at 60 minutes minus the absorbance at 0 minute was calculated for with and without silver nitrate wells. The value obtained with silver nitrate was subtracted from values obtained without silver nitrate. ARSA-specific activity was determined by multiplying the final absorbance value with the extinction coefficient of 4-nitrocathecol at 515 nm. The results were expressed as ARSA activity per milligram of protein per hour.

### Results

### Animal Disposition

All animals survived to the scheduled necropsy except one female Arsa−/− mouse (Animal 1301, Group 1) that was euthanized on Day 157 (33 weeks of age, 8 months of age) due to a skin condition unrelated to phenotype progression.

### Body Weight

Untreated female Arsa−/− mice exhibited weight gain similar to that of age-matched female wild type controls until approximately 7 months of age, when weight gain patterns for Arsa−/− and wild type mice began to diverge. After this time point, weight generally plateaued for untreated female Arsa−/− mice, and by 9 months of age, female Arsa−/− mice exhibited significantly lower body weights than that of female wild type controls at most time points evaluated, while wild type controls continued to gain weight through 15 months of age (the last time point evaluated).

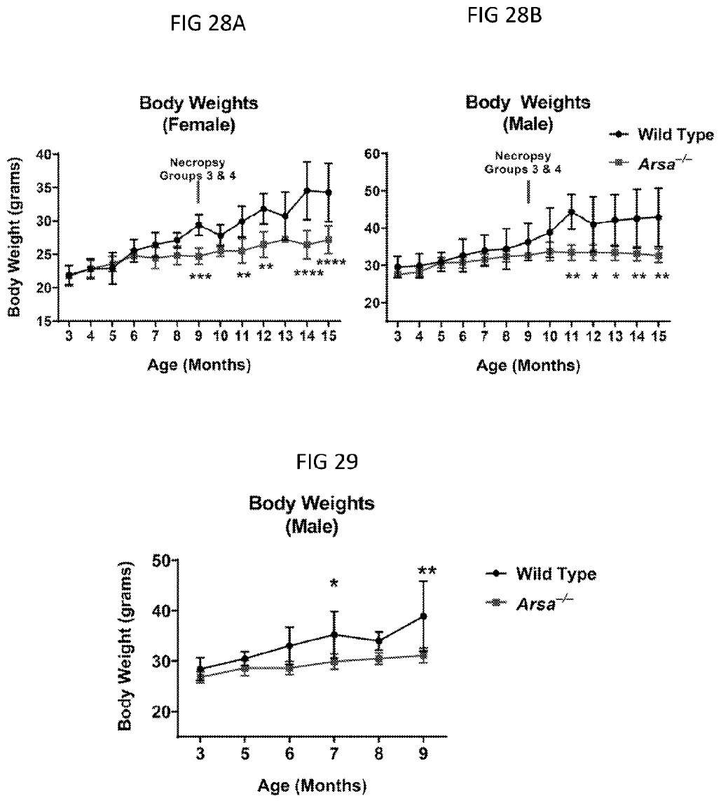

Untreated male Arsa−/− mice exhibited weight gain similar to age-matched male wild type controls through 10 months of age. By 11 months of age, untreated male Arsa−/− mice exhibited significantly lower body weights than that of male wild type controls at most time points evaluated, while the wild type controls continued to gain weight through 15 months of age (the last time point evaluated) (FIG. 28).

AAV-GAL3ST1-treated male Arsa−/− mice exhibited significantly lower body weights than age-matched male wild type controls by 9 months of age. (FIG. 29). AAV-GAL3ST1-treated mice were not evaluated for a longer period as it was hypothesized that aggravation of sulfatide storage might lead to earlier phenotype development. It is therefore unknown how they may have progressed beyond 9 months of age.

### Clinical Scoring Assessments

Clinical scoring was used to assess the clinical status of mice, with scores above 0 indicating clinical deterioration.

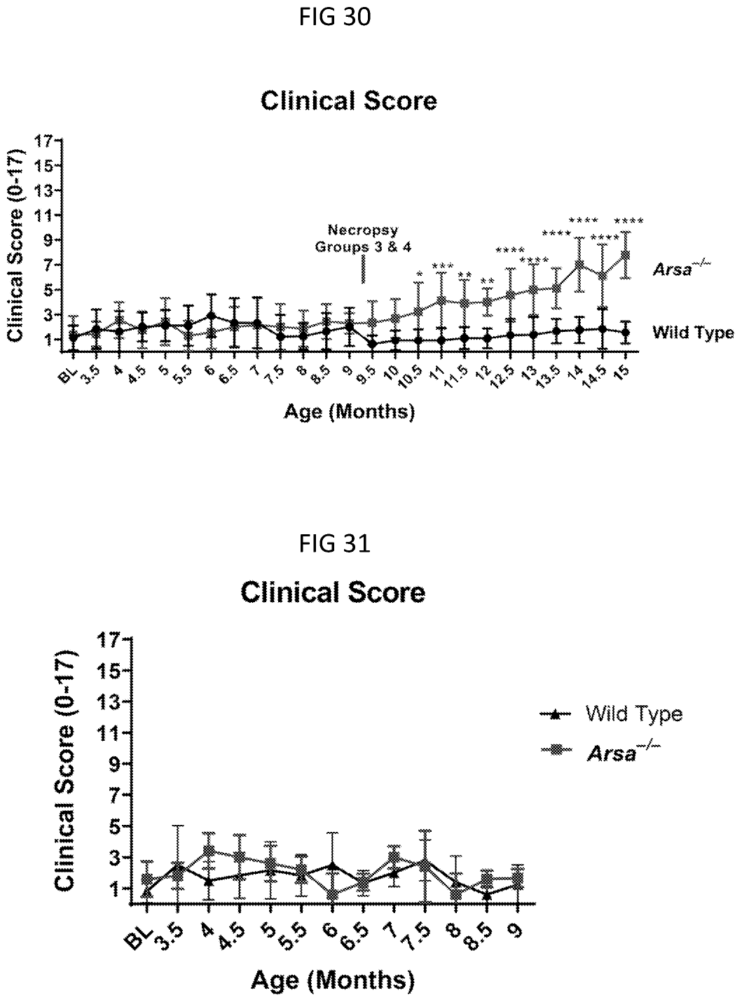

Beginning at Week 32 (˜10 months of age), untreated Arsa−/− mice exhibited significantly higher clinical scores than that of age-matched wild type controls at all time points evaluated, with clinical scores progressively increasing throughout the study to the last time point evaluated at Week 52 (˜15 months of age). This result indicates a progressive worsening of clinical status for untreated Arsa−/− mice (FIG. 30).

Similar to what was observed in untreated Arsa−/− mice, there was no difference between the clinical scores of AAV-GAL3ST1-treated Arsa−/− mice and age-matched male wild type controls through Study Week 26 (the last time point evaluated, ˜9 months of age). This result indicates that treatment with AAV.GAL3ST1 did not produce an earlier phenotype compared to untreated Arsa−/− mice (FIG. 31).

### Neuromotor Function

### Ledge Test

The ledge test measured coordination, which is impaired in neurodegenerative diseases associated with ataxia, such as MLD. Mice were assigned a score from 0 to 3, with higher scores indicating reduced coordination.

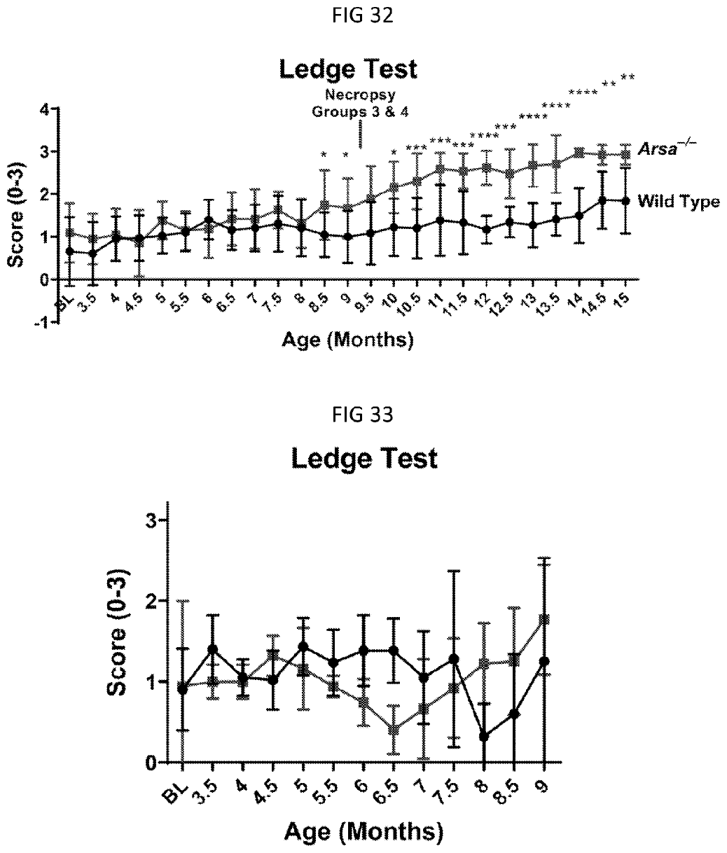

Beginning at Week 24 (˜8 months of age), untreated Arsa−/− mice exhibited significantly higher average ledge test severity scores than that of age-matched wild type controls at most time points evaluated, with severity scores progressively increasing throughout the study to the last time point evaluated at Week 52 (˜15 months of age). This result indicates a progressive worsening of neuromotor function for untreated Arsa−/− mice (FIG. 32).

Similar to what was observed in untreated Arsa−/− mice, there was no difference between the ledge test severity scores of AAV-GAL3ST1-treated Arsa−/− mice and age-matched male wild type controls until Week 26 (˜9 months of age), when AAV-GAL3ST1-treated Arsa−/− mice demonstrated a statistically significant increase in average ledge test severity scores compared to age-matched wild type controls. This result indicates that AAV.GAL3ST1 did not produce an earlier phenotype compared to untreated Arsa−/− mice (FIG. 33).

### RotaRod

Neuromotor function was assessed by the RotaRod test, which evaluates coordination and balance by measuring the time to fall for mice running on a spinning rod that progressively accelerates. A decrease in fall latency indicates neuromotor impairment, while an increase in fall latency indicates improved neuromotor function.

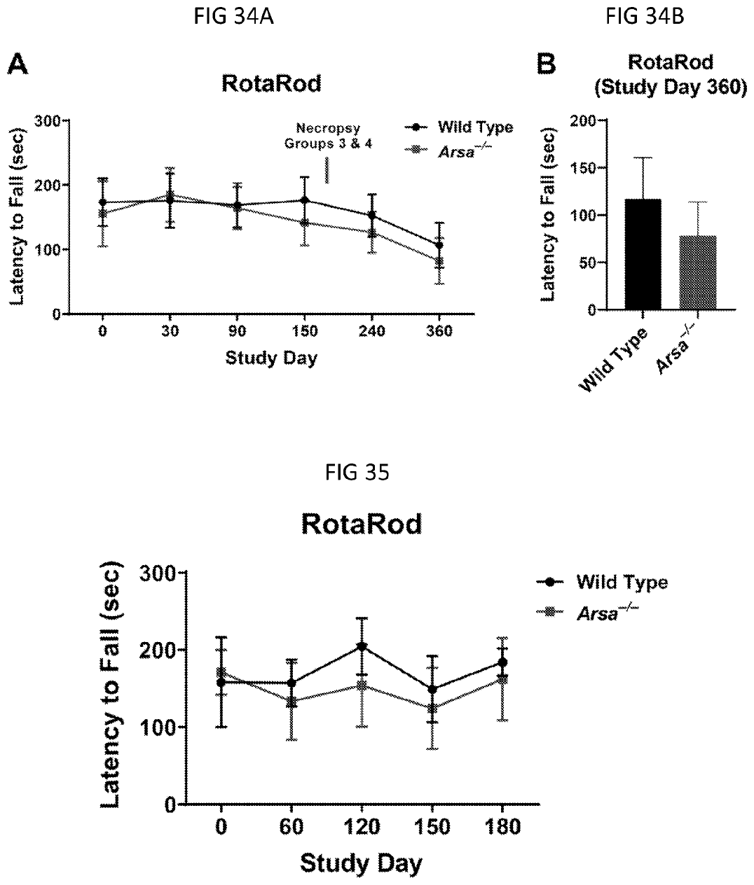

Although untreated Arsa−/− mice showed a trend suggesting a slight reduction in average latency to fall compared to wild type controls at Study Day 360 (˜15 months of age), the results were not statistically different between Arsa−/− mice and wild type controls at any time point evaluated (FIG. 34).

No difference was seen in RotaRod performance between AAV-GAL3ST1-treated Arsa−/− mice and wild type controls through Study Day 180 (˜9 months of age) (FIG. 35).

### CatWalk Gait Analysis

Neuromotor function was assessed using the CatWalk XT gait analysis system through measuring base of support, print positions, cadence, step sequence regularity, average body speed, stride length, contact area, print width, and print length. Neuromotor function abnormalities would be expected to result in gait and/or walking speed abnormalities in Arsa−/− mice when compared to wild type controls.

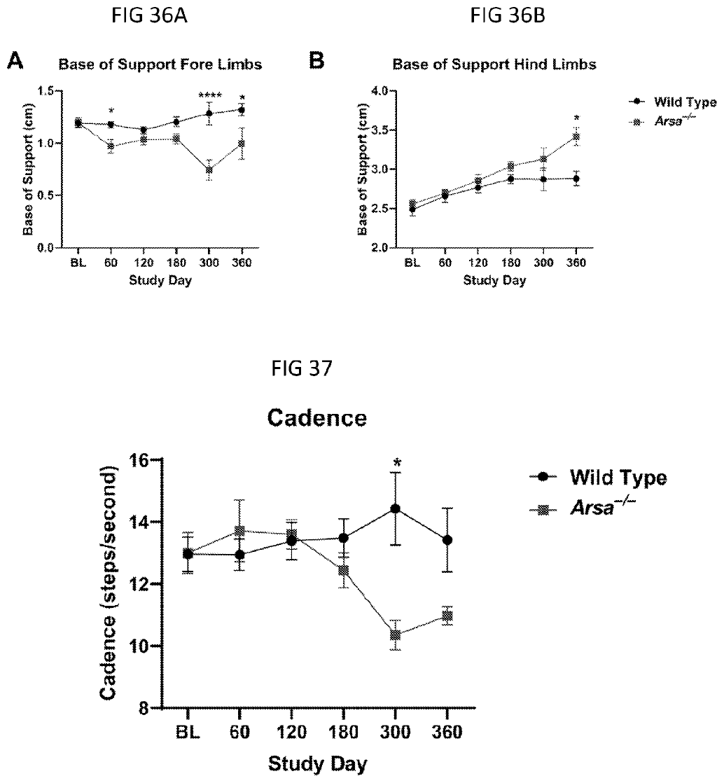

By Day 300 (˜13 months of age), untreated Arsa−/− mice exhibited a statistically significant reduction in the fore limb base of support compared to wild type controls, which persisted until the last time point evaluated on Day 360 (˜15 months of age; FIG. 36A). Untreated Arsa−/− mice also exhibited a statistically significant increase in hind limb base of support compared to wild type controls on Day 360 (˜15 months of age; FIG. 36B).

No statistically significant difference was observed in print positions on the left or right sides in untreated Arsa−/− mice compared to wild type controls (data not shown).

A statistically significant reduction in cadence was observed on Day 300 (˜13 months of age) in untreated Arsa−/− mice compared to wild type controls (FIG. 37), but this difference may have been due to assay variability since a statistically significant reduction in cadence was not observed at the last time point evaluated on Day 360 (˜15 months of age).

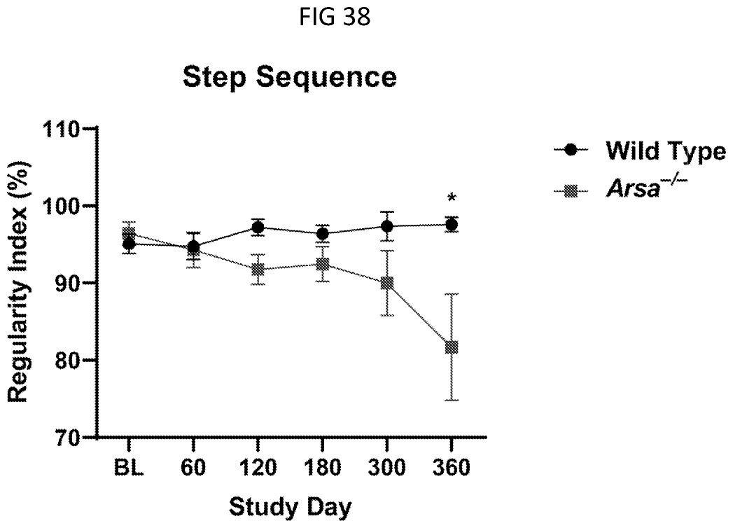

By Day 360 (˜15 months of age), untreated Arsa−/− mice exhibited a significant reduction in step sequence regularity compared to wild type controls (FIG. 38).

No statistically significant difference was observed in average speed in untreated Arsa−/− mice compared to wild type controls (data not shown).

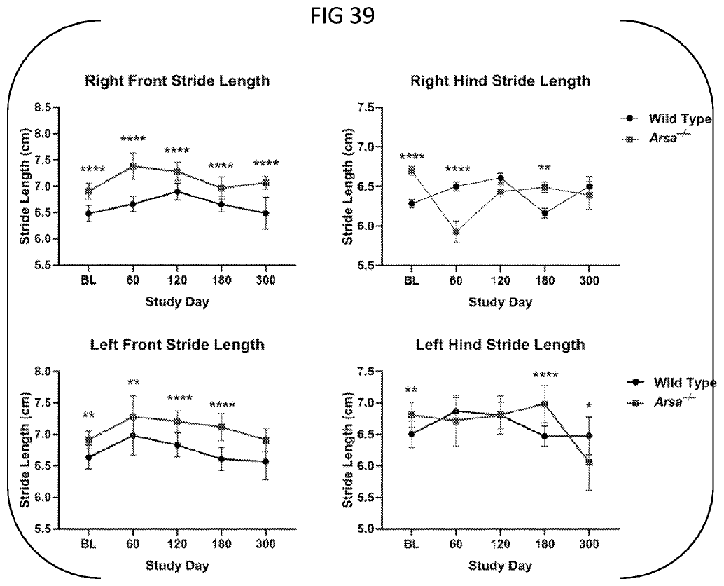

Right and left front stride length was significantly longer in untreated Arsa−/− mice compared to wild type controls at all time points measured, except for the left side on Day 300 (˜13 months of age) (FIG. 39). There was more variation in the right and left hind stride length. The right hind stride length was significantly longer in untreated Arsa−/− mice compared to wild type controls at baseline (˜3 months of age) and Day 180 (˜9 months of age), and significantly shorter in untreated Arsa−/− mice compared to wild type controls on Day 60 (˜4 months of age). Left hind stride length was significantly longer in untreated Arsa−/− mice compared to wild type controls at baseline (˜3 months of age) and on Day 180 (˜9 months of age), and significantly shorter in untreated Arsa−/− mice compared to wild type controls on Day 300 (˜13 months of age).

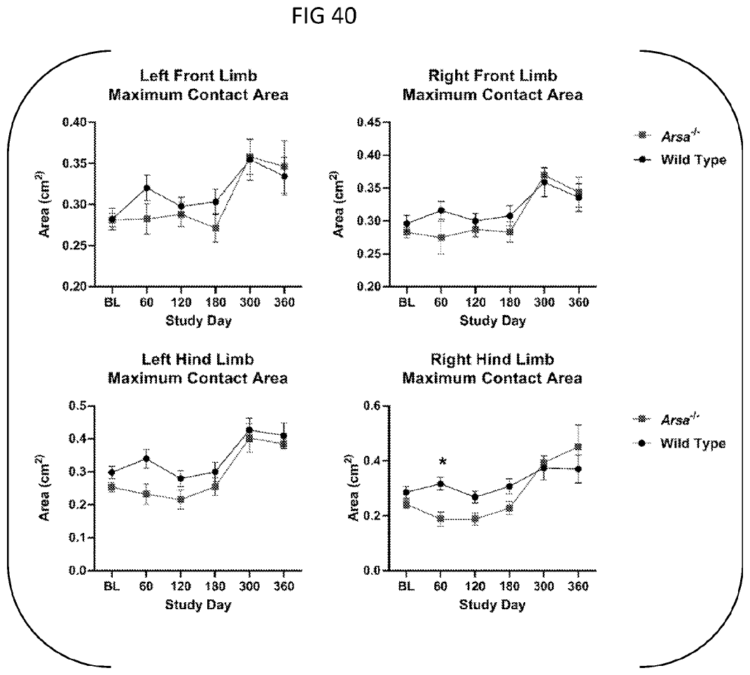

Analysis of footprint area parameters revealed no statistically significant difference between untreated Arsa−/− mice compared to wild type controls in print width and print length measurements (data not shown), except for a statistically significant reduction in right hind limb footprint contact area in Arsa−/− mice compared to wild type controls on Day 60 (˜4 months of age (FIG. 40).

CatWalk gait analysis did not reveal any significant differences between AAV-GAL3ST1-treated Arsa−/− mice and wild type controls (data not shown).

Taken together, the differences between the various CatWalk measurements recorded in untreated Arsa−/− mice compared to wild type controls suggest the emergence of a mild gait abnormality and reduced neuromotor function in the untreated Arsa−/− mice over the course of this study.

### Histological Findings

### LAMP-1 IHC (Evaluating Lysosomal Storage Lesions)

LAMP-1 IHC was performed to evaluate lysosomal storage lesions in the brain and spinal cord of untreated Arsa−/− mice and wild type controls. An increase in LAMP-1-positive area would indicate an increase in lysosomal storage.

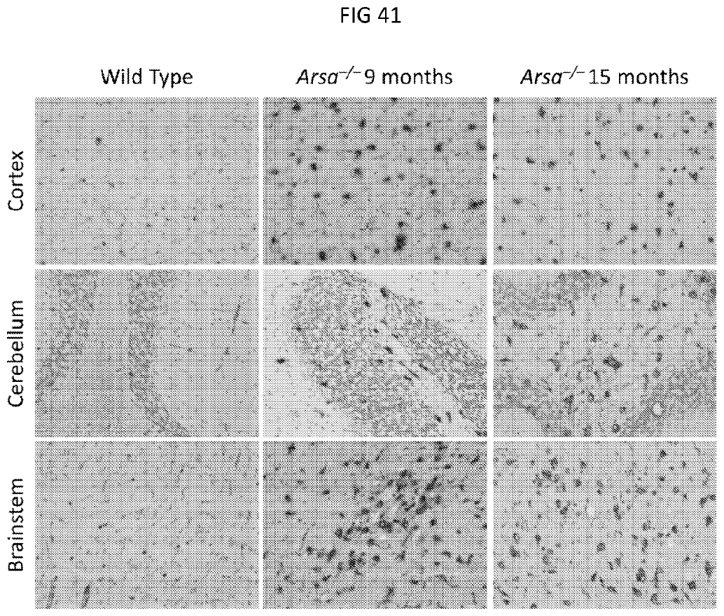

At Week 27 (˜9 months of age) and Week 52 (˜15 months of age), untreated Arsa−/− mice demonstrated increased LAMP-1 staining in the cortex, cerebellum, and brainstem compared to age-matched wild type controls (FIG. 41).

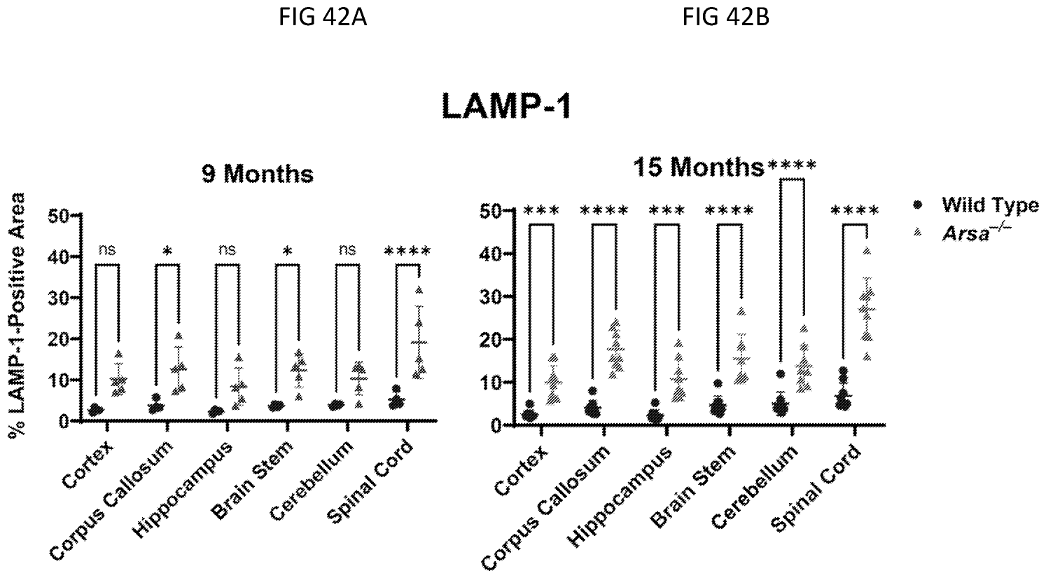

Quantification of LAMP-1 IHC staining using image analysis software confirmed that untreated Arsa−/− mice exhibited more LAMP-1-positive staining (indicated by a larger average LAMP-1-positive area) throughout the brain (cortex, corpus callosum, hippocampus, cerebellum, brainstem) and the spinal cord compared to wild type controls at both Week 27 (˜9 months of age) and Week 52 (˜15 months of age) (FIG. 42). Arsa−/− mice also exhibited a time-dependent increase in LAMP-1-positive staining (indicated by an increase in average LAMP-1-positive area) from Week 27 (˜9 months of age) to Week 52 (˜15 months of age) in the spinal cord and all brain regions evaluated (cortex, corpus callosum, hippocampus, cerebellum, brainstem), with the greatest increase observed in the spinal cord.

LAMP-1 IHC analyses were not conducted on AAV-GAL3ST1-treated Arsa−/− mice (Groups 5 and 6), as they did not show the expected more pronounced or earlier phenotype.

### FAP IHC (Evaluating Astrogliosis/Neuroinflammation)

GFAP IHC was performed to visualize reactive astrocytes and assess astrogliosis and neuroinflammation in the brain and spinal cord. An increase in GFAP-positive area indicates an increase in astrogliosis and neuroinflammation.

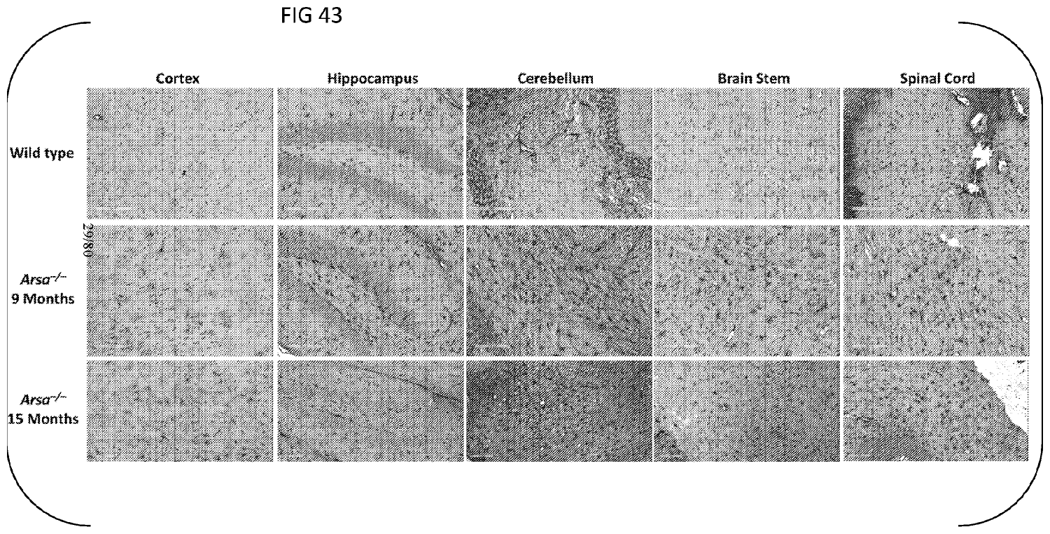

At Week 27 (˜9 months of age) and Week 52 (˜15 months of age), untreated Arsa−/− mice showed an increase in GFAP IHC staining in the brain (cortex, hippocampus, cerebellum, brainstem) and spinal cord compared to age-matched wild type controls (FIG. 43).

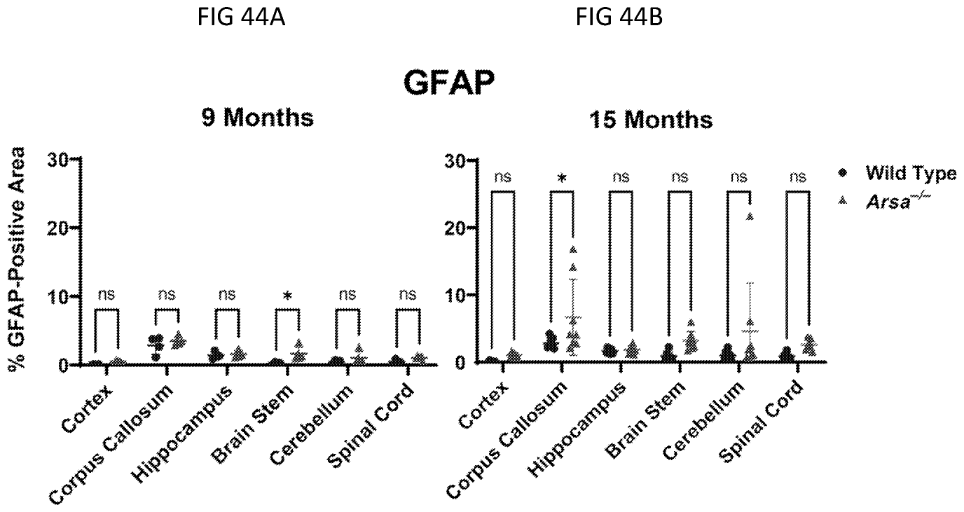

Quantification of GFAP IHC using image analysis software confirmed that untreated Arsa−/− mice exhibited a trend towards more GFAP-positive staining (indicated by a larger average GFAP-positive area) in the spinal cord and all brain regions (cortex, corpus callosum, brainstem, cerebellum) with only brainstem and corpus callosum reaching statistical significance when compared to wild type controls at Week 27 (˜9 months of age) and Week 52 (˜15 months of age) (FIG. 44). Arsa−/− mice also exhibited a time-dependent increase in GFAP-positive staining (indicated by an increase in average GFAP-positive area) Week 27 (˜9 months of age) to Week 52 (˜15 months of age) in the spinal cord and all brain regions evaluated except hippocampus (cortex, corpus callosum, brainstem, cerebellum), indicating a progression of astrogliosis/neuroinflammation over time.

GFAP IHC analyses were not conducted on AAV-GAL3ST1-treated Arsa−/− mice (Groups 5 and 6).

### Alcian Blue Staining (Evaluating Sulfatide Storage)

Sulfatide storage in the brain, kidneys, lung, sciatic nerve, and spinal cord were evaluated by Alcian Blue staining. An increase in Alcian blue staining intensity indicates an increase in sulfatide storage (i.e., the toxic substrate of ARSA enzyme).

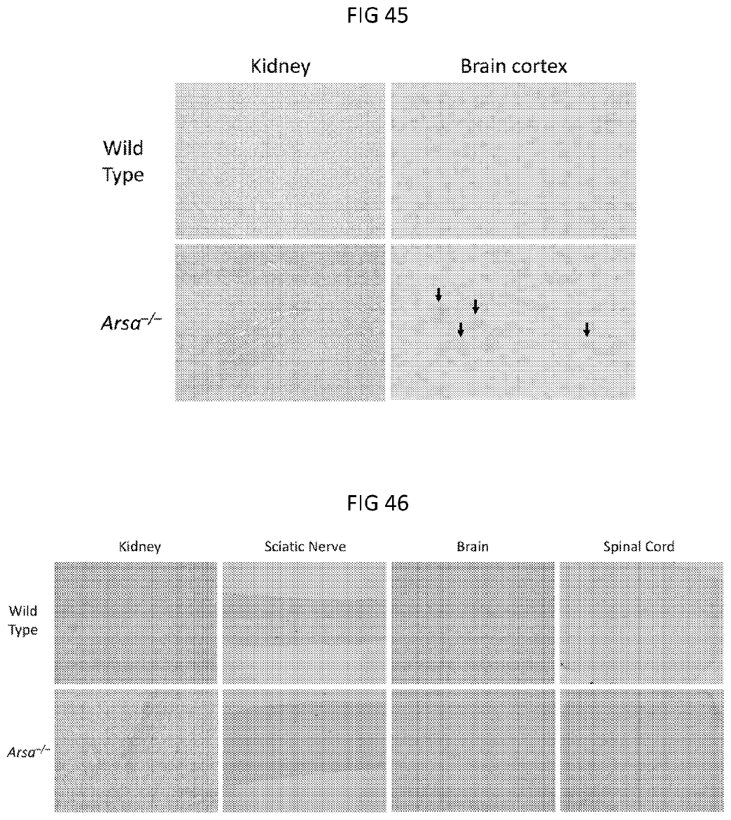

At Week 52 (˜15 months of age), the last time point evaluated, untreated Arsa−/− mice exhibited little to no Alcian blue staining (sulfatide storage) in the spinal cord and peripheral nerves, similar to that of wild type controls (data not shown). However, untreated Arsa−/− mice did exhibit a substantial increase in Alcian blue staining in the kidney and foci of Alcian blue staining in neurons in the compared to wild type controls, indicating substantial kidney sulfatide storage and minimal brain neuronal sulfatide storage based on the colorimetric detection method (FIG. 45).

At ˜9 months of age (Study Week 27), AAV-GAL3ST1-treated Arsa−/− mice exhibited minimal to no Alcian blue staining (sulfatide storage) in the brain, sciatic nerve, and spinal cord, similar to that of wild type controls. However, AAV-GAL3ST1-treated Arsa−/− mice did demonstrate increased Alcian blue staining in the kidney compared to wild type controls, indicating increased kidney sulfatide storage (FIG. 46).

### LC/MS (Quantifying Sulfatide Storage)

LC/MS analysis was performed to quantify sulfatide storage in the brain, spinal cord, sciatic nerve, liver, spleen, kidney, heart, quadriceps, and plasma at ˜9 months of age (Study Week 27) in untreated Arsa−/− mice and AAV-GAL3ST1-treated Arsa−/− mice. Untreated Arsa−/− mice were also evaluated at ˜15 months of age (Study Week 52) to assess progression of sulfatide storage over time.

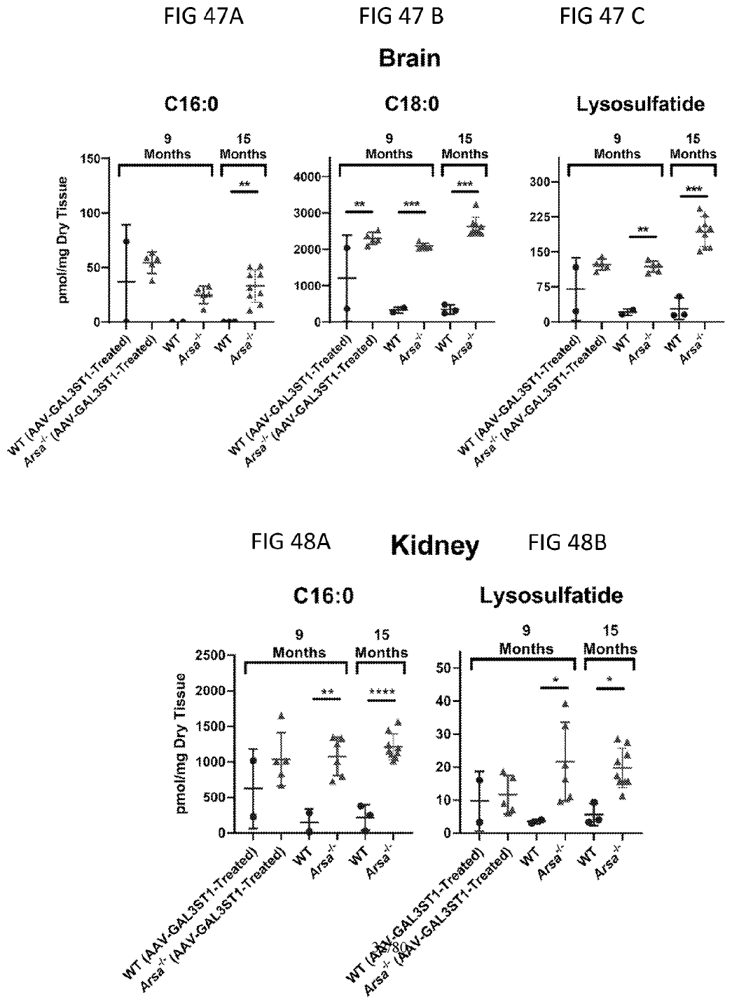

In the brain, a significant increase in levels of C16:0 sulfatides, C18:0 sulfatide, and lysosulfatide species was observed in untreated Arsa−/− mice at both ˜9 months and ˜15 months of age when compared to age-matched WT controls. Storage levels were comparable in AAV-GAL3ST1-treated Arsa−/− mice when compared to untreated Arsa−/− mice (FIG. 47).

In the kidney, a significant increase in the levels of C16:0 sulfatide species and lysosulfatide was observed in untreated Arsa−/− mice at both ˜9 months and ˜15 months of age when compared to the levels observed in wild type controls. In contrast, there was no significant difference in the levels of C16:0 sulfatide species or lyosulfatide between AAV-GAL3ST1-treated Arsa−/− mice and wild type controls at ˜9 months of age (FIG. 48).

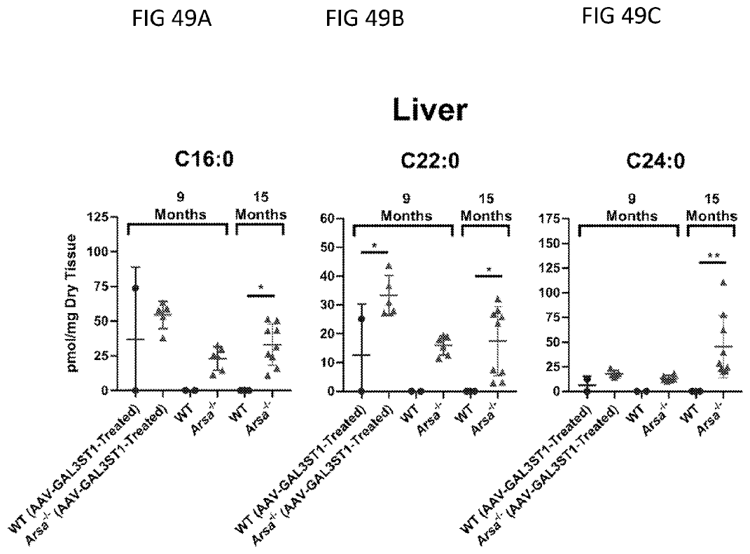

In the liver, untreated Arsa−/− mice exhibited significantly higher levels of C16:0, C:22:0, and C24:0 sulfatide species compared to wild type controls at ˜15 months of age, but not ˜9 months of age, indicating a progression of sulfatide storage over time. In AAV-GAL3ST1-treated Arsa−/− mice, levels of C22:0 sulfatide species were significantly higher than the levels observed in wild type controls at ˜9 months of age; however, no significant difference in levels of C16:0 and C24:0 sulfatide species was observed between AAV-GAL3ST1-treated Arsa−/− mice and wild type controls at this age (FIG. 49).

In the quadriceps muscle, untreated Arsa−/− mice exhibited similar and 3-fold higher levels of C16:0 and C:18:0 sulfatide species, respectively, at ˜9 months of age. These differences grew such that 2-fold and 14-fold higher levels of C16:0 and C:18:0 sulfatide species, respectively, were observed the quadriceps of ˜15 month old Arsa−/− mice compared to wild type controls, indicating a progression of sulfatide storage over time. In the sciatic nerve, ˜9 and ˜15-month-old Arsa−/− mice exhibited 2-fold and 3-fold higher levels of C16:0 sulfatide species, respectively, compared to wild type controls. By ˜15 months of age, Arsa−/− mice had 16-fold higher levels of lysosulfatide in the sciatic nerve compared to wild type controls. In the spinal cord at both ˜9 and ˜15 months of age, Arsa−/− mice exhibited 3-fold higher levels of C16:0 sulfatide species compared to wild type controls. Spinal cord lyosulfatide levels were 2-fold and 3-fold higher in Arsa−/− mice ˜9 and ˜15 months of age, respectively, compared to wild type controls. In the heart, Arsa−/− mice exhibited 12-fold and 10-fold higher levels of C16:0 sulfatide species at ˜9 and ˜15 months of age, respectively, compared to wild type controls. In the spleen, levels of C16:0 sulfatide species were 22-fold and 14-fold higher in Arsa−/— mice at ˜9 and ˜15 months of age, respectively, compared to wild type controls. Finally, plasma levels of C16:0 sulfatide species were 5-fold and 6-fold higher in Arsa−/− mice at ˜9 and ˜15 months of age, respectively, compared to wild type controls.

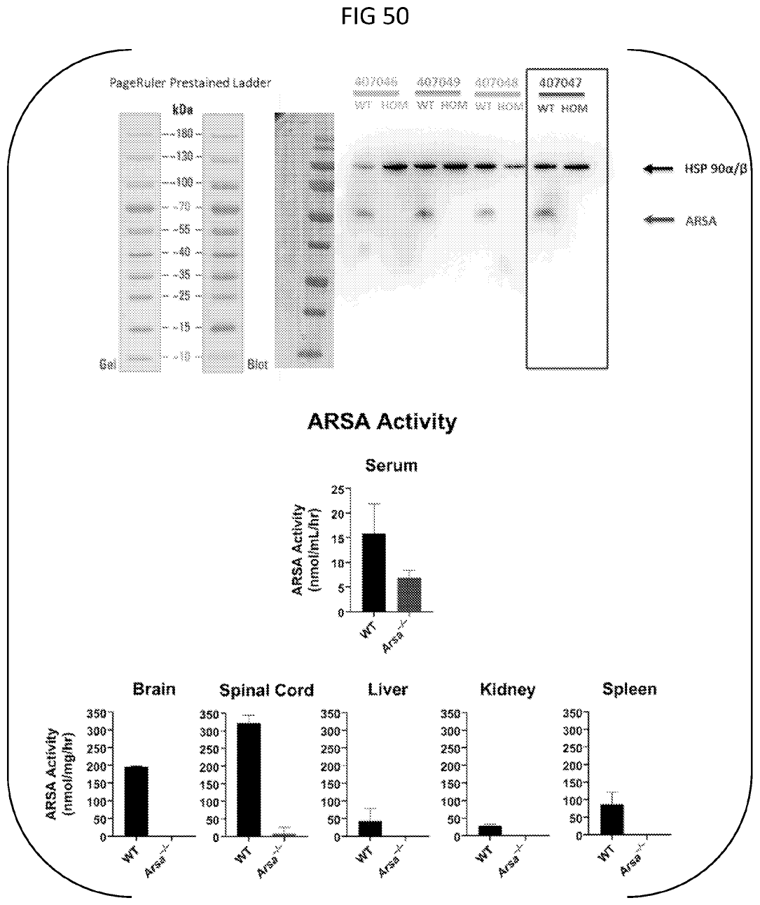

Endogenous ARSA Enzyme Activity Endogenous ARSA enzyme activity was assessed in the serum and tissues (brain, spinal cord, liver, kidney, spleen) of a subset of wild type and untreated Arsa−/− mice. The untreated Arsa−/− mice included in this analysis exhibited minimal non-specific enzyme activity in serum, with average levels lower than that of wild type mice. Untreated Arsa−/− mice also demonstrated minimal to no residual ARSA enzymatic activity in the brain, spinal cord, liver, kidney, and spleen when using a p-nitrocatechol based assay subtracting values obtained with ARSA inhibitor silver nitrate (nonspecific activity) to values obtained without inhibitors (total sulfatases activity). This aligns with the absence of band on a western blot using an anti-ARSA antibody (FIG. 50). These results confirm the successful knockdown of ARSA expression in Arsa−/− mice.

### Summary of Results

Arsa−/− mice exhibited a normal lifespan up to the last time point evaluated (˜15 months of age).
Untreated Arsa−/− mice exhibited various clinical, behavioral, biochemical, and histological phenotypes reminiscent of MLD.
Untreated Arsa−/− mice exhibited significantly reduced body weight gain beginning at 9 months of age for females and 11 months of age for males when compared to age-matched wild type controls.
Untreated Arsa−/− mice exhibited significant progressive clinical decline on clinical scoring assessments beginning ˜10 months of age when compared to wild type controls.
Various neuromotor deficits were noted in untreated Arsa−/− mice, including significantly reduced ledge test performance beginning ˜8 months of age and mild gait abnormalities observable by ˜13 months of age on CatWalk gait assessment. No abnormalities were observed for untreated Arsa−/− mice on the RotaRod assessment at up to ˜15 months of age (the last time point evaluated).
Untreated Arsa−/− mice exhibited a progressive increase in storage lesions (LAMP-1 IHC) and astrogliosis/neuroinflammation (GFAP IHC) throughout the brain (cortex, corpus callosum, cerebellum, brainstem) and in the spinal cord beginning by ˜9 months of age.
Compared to wild type controls, untreated Arsa−/− mice demonstrated significantly increased sulfatide storage by LC/MS analysis in the brain, spinal cord, sciatic nerve, heart, quadriceps, kidney, liver, spleen and plasma.
Untreated Arsa−/− mice demonstrated minimal to no residual ARSA enzymatic activity at 16 weeks of age (4 months of age) in brain, spinal cord, liver, kidney, and spleen. Some non-specific residual enzyme activity was detected in serum, with levels lower than that of wild type controls.
AAV-GAL3ST1-treated Arsa−/− mice exhibited a normal lifespan and similar phenotype severity and progression as that of untreated Arsa−/− mice, indicating that the attempt to produce an earlier and/or more pronounced phenotype with increased sulfatide storage in AAV-GAL3ST1-treated Arsa−/− mice was not successful. Untreated Arsa−/− mice were therefore selected for future pharmacology studies.

### Example 7—Evaluating Human Arylsulfatase a (ARSA) Expression and Efficacy Following Intracerebroventricular Administration of AAVhu68.CB7.CI.hARSAco.rBG (GTP-207) to Arsa^{-/-} Mice

A study was performed to evaluate the short-term effects of AAVhu68.CB7.CI.hARSAco.rBG (GTP-207) following intracerebroventricular (ICV) administration to adult Arsa^{-/-} mice and to optimize biomarker analyses for future pharmacology studies. AAVhu68.CB7.CI.hARSAco.rBG (GTP-207) is a recombinant adeno-associated viral (AAV) serotype hu68 vector expressing the human arylsulfatase A (ARSA) gene.

Adult (6-7 months old) male Arsa^{-/-} mice received a single ICV administration of AAVhu68.CB7.CI.hARSAco.rBG (GTP-207) at a dose of 4.5×10¹⁰ GC (1.1×10¹¹ GC/g brain, N=2). Age-matched Arsa or C57BL6/J (wild type) mice were administered vehicle (phosphate-buffered saline [PBS]) as a control (N=1 and N=2 respectively. In this pilot study, a low number of animals were enrolled based on availability in the colony (N=1-2 per group). The purpose was to optimize biomarkers and conduct a qualitative assessment of the short-term pharmacological effect of AAVhu68.CB7.CI.hARSAco.rBG (GTP-207) in preparation of contemporaneous larger pharmacology studies. In-life assessments included viability checks performed daily and collection of serum for evaluation of transgene product expression (ARSA enzyme activity). Necropsies were performed on Day 30 post treatment. Sulfatide storage was assessed in plasma, peripheral organs, and target tissues relevant for the treatment of MLD (central nervous system [CNS] and peripheral nervous system [PNS]) (liquid chromatography/mass spectrometry [LC/MS]). Lysosomal storage lesions (lysosomal-associated membrane protein 1 [LAMP-1] immunohistochemistry [IHC]) and astrogliosis/neuroinflammation (glial fibrillary acidic protein [GFAP] IHC) were analyzed in the CNS. Transgene product expression (ARSA IHC and/or ARSA enzyme activity) was also evaluated in CNS and peripheral tissues.

### Group Designations, Dose Levels, and Route of Administration

Study animals were not randomized. Group designations, dose levels, and the route of administration (ROA) are presented in the table below.

TABLE
Group Designations, Dose Levels, and Route of Administration

| | | | | | | Dose | Dose | | | Age at |
|---|---|---|---|---|---|---|---|---|---|---|
| | | | | | Dose | (GC/g | Volume | | Dosing | Necropsy |
| Group | N | Sex | Genotype | Treatment | (GC/Animal) | Brain)^{a} | (μL) | ROA | Day | (months) |
| 1 | 1 | M | Arsa^{-/-} | None | N/A | N/A | N/A | N/A | N/A | 9^{b} |
| 2 | 2 | M | C57BL/ | PBS | N/A | N/A | 5.0 | ICV | 0 | 8 |
| | | | 6J (WT) | | | | | | | |
| 3 | 1 | M | Arsa^{-/-} | PBS | N/A | N/A | 5.0 | ICV | 0 | 7 |
| 4 | 2 | M | Arsa^{-/-} | AAVhu68.CB7.CI.hARSAco.rBG | 4.5 × 10¹⁰ | 1.1 × 10¹¹ | 5.0 | ICV | 0 | 7 |
| | | | | (GTP-207) | | | | | | |

^{a}Values were calculated using 0.4 g brain mass for an adult mouse (Gu et al., 2012).
^{b}This animal was enrolled in Nonclinical Study 5 (W2977). Only data for sulfatide quantification by LC/MS
are included for this animal in the present study.
Abbreviations: F, female; GC, genome copies; ICV, intracerebroventricular; ID, identification number; LC/MS, liquid chromatography/mass spectrometry; N, number of animals; N/A, not applicable; PBS, phosphate-buffered saline; ROA, route of administration; WT, wild type.

On Study Day 0, adult C57BL/6J (wild type) mice received a single ICV administration of either AAVhu68.CB7.CI.hARSAco.rBG (GTP-207)(4.5×10¹⁰ GC) or control article (PBS [vehicle]). Viability checks were performed daily. On Day 7 and at necropsy on Day30, serum was collected for evaluation of transgene product expression (ARSA enzyme activity). At necropsy, brain, spinal cord, liver, kidney, heart, and spleen were collected for evaluation of transgene product expression (ARSA enzyme activity assay and/or ARSA IHC). Brain, spinal cord, liver, kidney, heart, spleen, sciatic nerve, quadriceps muscle, and plasma were collected to assess sulfatide storage (LC/MS). Lysosomal storage lesions (LAMP1 IHC) and astrogliosis/neuroinflammation (GFAP IHC) were assessed in the CNS (brain and spinal cord).

### Histological Processing and Evaluation

### LAMP-1 IHC (Evaluating Lysosomal Storage Lesions)

LAMP-1 immunohistochemical staining was performed on deparaffinized paraffin sections. Briefly, antigen retrieval was performed by boiling slides at 100° C. for 6 minutes in 10 mM citrate buffer (pH 6.0). Slides were then incubated with 2% hydrogen peroxide for 15 minutes, blocked using avidin/biotin reagents for 15 minutes each (Vector Laboratory; Catalog number: SP-2001), and incubated with 1% donkey serum in phosphate-buffered saline (PBS) with 0.2% Triton-X for 10 minutes at room temperature. Slides were then incubated with a rat anti-mouse LAMP-1 primary antibody (Abcam, Catalog #Ab25245) at 37° C. for 1 hour. Slides were washed and then incubated with a biotinylated donkey anti-rabbit IgG secondary antibody (Jackson; Catalog number: 711-065-152) for 45 minutes at room temperature. Slides were washed and then incubated with Vectastain ABC reagent (Vector Laboratories; Catalog number: PK-6100). Colorimetric development was performed using a 3,3′-Diaminobenzidine (DAB) kit (Vector Laboratories; Catalog number: SK-4100) followed by counterstaining with hematoxylin and coverslipping for evaluation.

### GFAP IHC (Evaluating Astrogliosis/Neuroinflammation)

GFAP immunohistochemical staining was performed on deparaffinized paraffin sections. Briefly, antigen retrieval was performed by boiling slides at 100° C. for 6 minutes in 10 mM citrate buffer (pH 6.0). Slides were then incubated with 2% hydrogen peroxide for 15 minutes, blocked using avidin/biotin reagents for 15 minutes each (Vector Laboratory; Catalog number: SP-2001), and incubated with 1% donkey serum in PBS with 0.2% Triton-X for 10 minutes at room temperature. Slides were then incubated with a rabbit anti-mouse GFAP primary antibody (Abcam, Catalog #ab7260) at 37° C. for one hour. Slides were washed and then incubated with a biotinylated donkey anti-rabbit IgG secondary antibody (Jackson; Catalog number: 711-065-152) for 45 minutes at room temperature. Slides were washed and then incubated with Vectastain ABC reagent (Vector Laboratories; Catalog number: PK-6100). Colorimetric development was performed using a DAB kit (Vector Laboratories; Catalog number: SK-4100) followed by counterstaining with hematoxylin and coverslipping for evaluation.

### ARSA Immunohistochemistry (IHC)

Following deparaffinization, IHC for human ARSA protein was performed. Briefly, antigen retrieval was performed in a pressure cooker at 100° C. for 20 minutes using a citric acid-based antigen unmasking solution (Vector Laboratories; Catalog number: H-3300). Slides were incubated with 3% hydrogen peroxide for 10 minutes, blocked using avidin/biotin reagents for 15 minutes each (Vector Laboratory; Catalog number: SP-2001), and incubated with 1% donkey serum with 0.2% Triton-X for 15 minutes at room temperature. Slides were then incubated with a rabbit ARSA primary antibody (Sigma; Catalog number: HPA005554) diluted 1:500 at 4° C. overnight. Slides were incubated with a biotinylated donkey anti-rabbit IgG secondary antibody (Jackson; Catalog number: 711-065-152) at a dilution of 1:500 for 30 minutes at room temperature. Slides were washed and then incubated with Vectastain ABC reagent (Vector Laboratories; Catalog number: PK-6100). Colorimetric development was performed using a DAB kit (Vector Laboratories; Catalog number: SK-4100) followed by counterstaining with hematoxylin and coverslipping.

### Quantification of Sulfatide Storage by LC/MS

Thawed tissues were lyophilized overnight and ground to a fine powder in 2.0 mL polypropylene tubes with ceramic beads using a Precellys bead-beating homogenizer (Bertin Technologies, Rockville, MD) at 4° C. Aliquots of powder (˜2.5-5.0 mg) were weighed on an analytical balance followed by homogenization in 500 μL of 80% methanol in the Precellys homogenizer at 4° C. A 100 μL aliquot of homogenate was then spiked with 10 μL of a C18:0-CD3-sulfatide internal standard (N-omega-CD3-Octadecanoyl-sulfatide Matreya State College, PA, catalog #1536; 25 μM) and extracted with 400 μL of ice cold methanol in a 2.0 mL Eppendorf tube. The sample was centrifuged for 5 minutes at 14,000×g at 4° C. Aliquots (400 μL) of methanolic supernatants were dried under nitrogen in a 96-well plate at 45° C. and reconstituted in 150 μL of methanol for LC/MS analysis.

Calibration samples of sulfatide standards were prepared. Standard powders of sulfatides (lysosulfatide catalog #1904; C16:0 catalogue #1875, C18:0 catalogue #1932, C18:0-CD3 catalogue #1536, and C24:1 catalogue #1931; Matreya, State College, PA) were weighed on an analytical balance, and individual stock solutions (1 mM) were prepared in 2:1 methyl tert-butyl ether/methanol. The C18:0-CD3sulfatide internal standard stock solution was diluted in methanol to give a 25 μM spiking internal standard solution. Aliquots of the individual stock solutions were combined to make a high calibration spiking solution of 50 μM lysosulfatide, 50 μM C16:0, 250 μM C18:0, and 250 μM C24:1 sulfatide in methanol. This high calibration spiking solution was serially diluted in methanol to make 0.1, 0.25, 0.5, 1, 5, 10, 25, and 50 μM calibration curve spiking solutions for lysosulfatide and C16:0 sulfatide, along with 0.5, 1.25, 2.5, 5, 25, 50, 125, and 250 μM calibration curve spiking solutions for C18:0 and C24:1 sulfatide. Calibration curve solutions for LC/MS analysis were created by pipetting 10 μL of each spiking solution and 10 μL of the C18:0-CD3-sulfatide internal standard (25 μM) into 100 μL of 80% methanol, resulting in LC/MS calibration curves of 0.01, 0.025, 0.05, 0.1, 0.5, 1, 2.5, and 5 μM for lysosulfatide and C16 sulfatide, along with LC/MS calibration curves of 0.05, 0.125, 0.25, 0.5, 2.5, 5, 12.5, and 25 μM for C18:0 and C24:1 sulfatide. A 400 μL aliquot of methanol was added to each solution. The sample was vortexed, and 400 μL was dried under nitrogen in a 96-well plate at 45° C. and reconstituted in 150 μL of methanol for LC/MS analysis.

Sulfatides were quantified with an Agilent 1290 Infinity UHPLC/6495B triple quadrupole mass spectrometer. Biological extracts and calibration solutions in 96-well plates were injected (5 μL) and separated on the UHPLC. Sulfides were eluted by gradient elution on a Waters Acquity BEH C18 2×100 mm, 1.7 μM column at a flow rate of 0.4 mL/minute at 45° C. A 7.5 minute gradient was used beginning with 35% solvent A (70/30 deionized water/acetonitrile/0.1% formic acid) and 65% solvent B (50/50 acetonitrile/isopropanol/0.1% formic acid) held for 0.5 minutes and increased to 100% solvent B over 5.5 minutes, held at 100% solvent B until 7.5 minutes followed by re-equilibration back to starting conditions from 7.6 to 10 minutes. The HPLC flow was diverted to waste for the first 0.5 minute then directed to the electrospray ionization source. Sulfatides were ionized by electrospray ionization in the positive ionization mode on the mass spectrometer. The Agilent Jet Stream electrospray ionization source was operated with a nitrogen gas temperature of 250° C., gas flow of 14 L/minute, nebulizer of 45 psi, sheath gas temperature of 325° C., sheath gas flow of 12 L/minute, capillary voltage of 3500 V, and nozzle voltage of 500 V. Multiple reaction monitoring (MRM) was used to quantitate sulfatides with a peak width of 0.7 Da and an electron multiplier voltage of 400 V in the positive ion mode. As an example, a primary transition for C16:0 sulfatide of m/z 780.57→264.2 was used for the quantitation of C16:0 by monitoring m/z 264.2, the while the secondary transition m/z 780.57→682.6, generated by neutral loss of H₂SO₄ from the parent ion, was used to confirm the primary transition as an authentic sulfatide. Agilent MassHunter software was used to generate linear or quadratic calibration curves (1/x or 1/x2 weighting and R2 0.99 or better) to quantify sulfatides in biological samples.

### Measuring ARSA Enzyme Activity

ARSA enzyme activity was measured in dialyzed serum or tissues samples using a p-nitrochatechol assay. Briefly, dialyzed serum (diluted 1:5, 1 part serum+4 parts diluent) or tissues were diluted into a base buffer (0.5 M sodium acetate buffer, pH 5.0; 10% sodium chloride; 0.5 mM sodium pyrophosphate) and 40 μL diluted sample was loaded in four wells (2 duplicates) into a 96-well plate. Next, 40 μL of substrate (10 mM 4-nitrocatechol sulfate in base buffer) was added to the samples, and the reaction was stopped by immediately adding 120 μL 1N NaOH (stop solution) in two of the four wells. The plate was then incubated at 37° C. for 5 hours. The reaction was stopped by adding 120 μL 1N NaOH (stop solution). The absorbance was measured by reading the plate at 515 nm using a plate reader. ARSA-specific activity was determined by multiplying the absorbance obtained at five hours minus the absorbance at 0 minute with the extinction coefficient of a 4-nitrocathecol standard curve at 515 nm and by dividing by the amount of protein in the well (mg) as measured by BCA assay. The results for ARSA activity were expressed in nmol per milligram of protein per five hours (nmol/mg/5 hr).

### Animal Disposition

All animals survived to the scheduled necropsy on Day 30.

### Histological Findings

### LAMP-1 IHC (Evaluating Lysosomal Storage Lesions)

LAMP-1 IHC was performed to evaluate lysosomal storage lesions in the brain and spinal cord of Arsa^{-/-} mice and wild type controls. An increase in LAMP-1-positive area would indicate an increase in lysosomal storage. Data collected from brain tissues are presented.

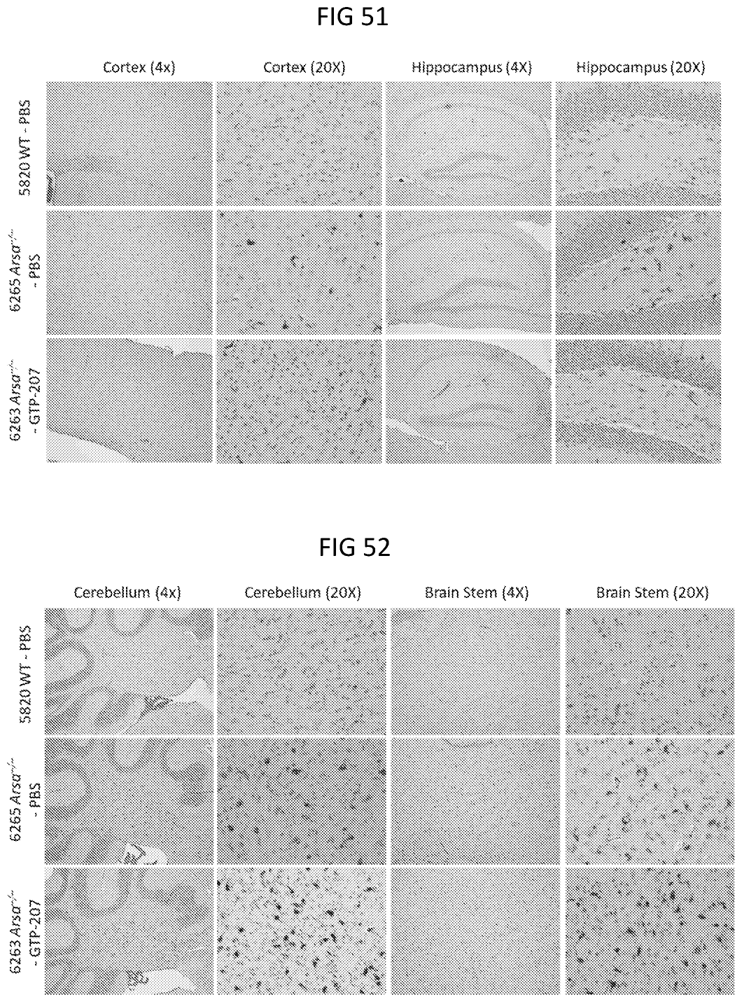

On Day 30, the vehicle-treated Arsa^{-/-} mouse demonstrated increased LAMP-1 staining in the cortex, hippocampus, cerebellum, and brainstem compared to the age-matched wild type control mouse (FIG. 51 and FIG. 52). The Arsa^{-/-} mouse administered AAVhu68.CB7.CI.hARSAco.rBG (GTP-207) demonstrated diminished LAMP-1 staining in the cortex and hippocampus compared to the vehicle-treated Arsa^{-/-} mouse. However, no differences in LAMP-1 staining was seen in the cerebellum, brain stem (FIG. 52), and spinal cord (data not shown) of the AAVhu68.CB7.CI.hARSAco.rBG (GTP-207)-treated Arsa mouse.

### GFAP IHC (Evaluating Astrogliosis/Neuroinflammation)

GFAP IHC was performed to visualize reactive astrocytes and assess astrogliosis and neuroinflammation in the brain and spinal cord. An increase in GFAP-positive area indicates an increase in astrogliosis and neuroinflammation. Data collected from brain tissues are presented.

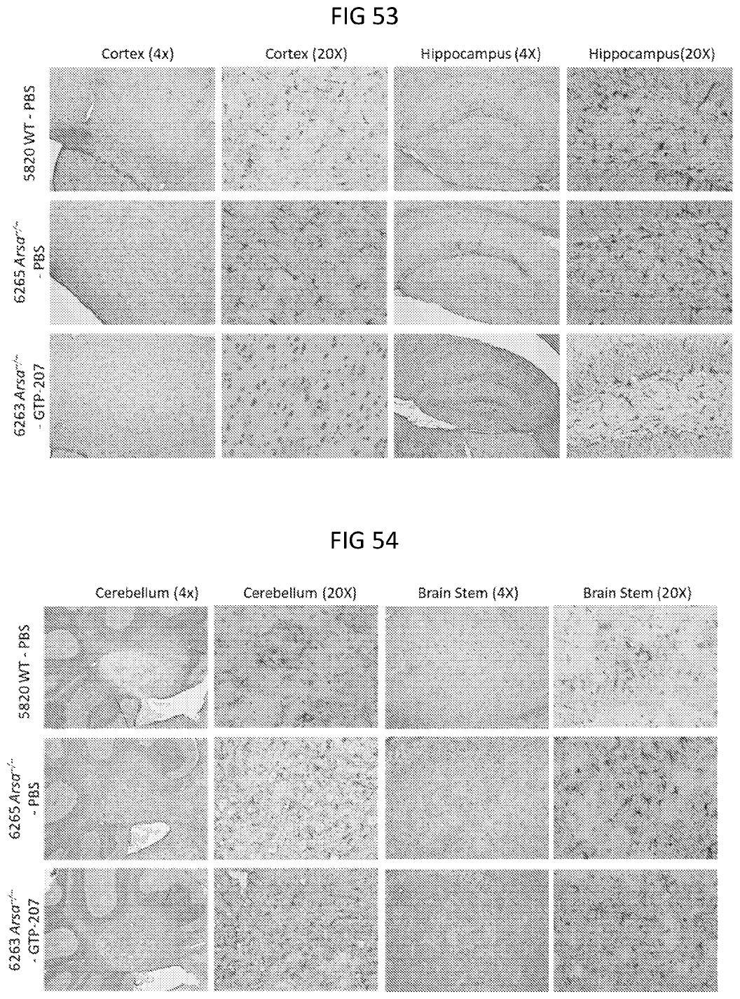

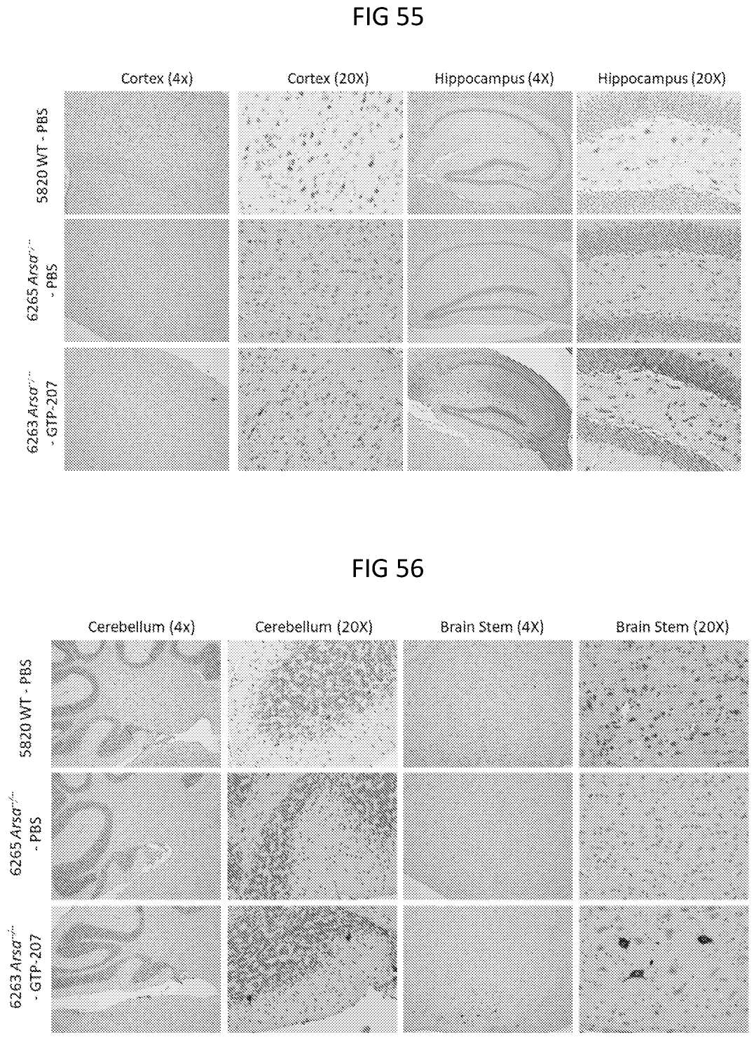

On Day 30, the vehicle-treated Arsa^{-/-} mouse demonstrated increased GFAP staining in the cortex, cerebellum, and brainstem compared to the age-matched wild type control mouse (FIG. 53 and FIG. 54). The Arsa mouse administered AAVhu68.CB7.CI.hARSAco.rBG (GTP-207) demonstrated diminished GFAP staining in the cortex and hippocampus compared to the vehicle-treated Arsa^{-/-} mouse. However, no differences in GFAP staining were seen in the cerebellum, brainstem (FIG. 54), or spinal cord (data not shown) of the AAVhu68.CB7.CI.hARSAco.rBG (GTP-207) treated Arsa^{-/-} mouse.

### ARSA IHC (Transgene Product Expression)

Several CNS and PNS tissues were harvested at necropsy for a comprehensive histological evaluation of human ARSA expression by IHC. Data collected from brain tissues are presented.

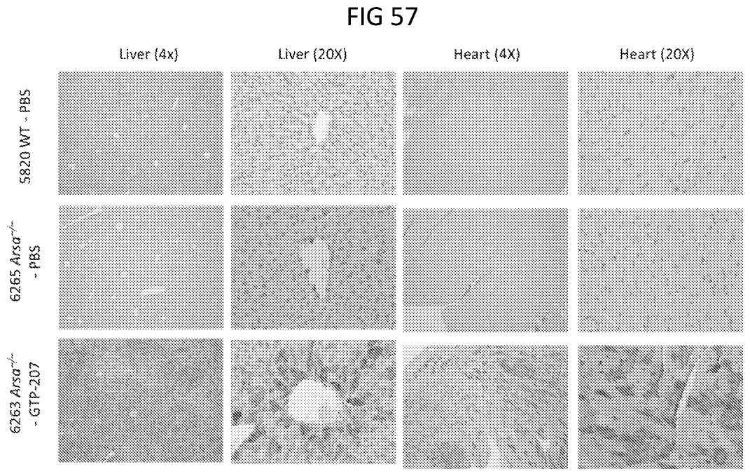

At necropsy on Day 30, the vehicle-treated Arsa mouse demonstrated no ARSA protein expression in cells of the cortex, hippocampus, cerebellum, and brainstem (FIG. 55 and FIG. 56). In contrast, the Arsa^{-/-} mouse administered AAVhu68.CB7.CI.hARSAco.rBG (GTP-207) demonstrated ARSA protein expression in each of these tissues, and ARSA-positive cells were more abundant in the cortex and hippocampus compared to the cerebellum and brainstem. ARSA expression was not seen in the spinal cord of the AAVhu68.CB7.CI.hARSAco.rBG (GTP-207)-treated mouse (data not shown). In peripheral tissues, only liver and heart demonstrated strong expression of ARSA in the Arsa^{-/-} mouse administered AAVhu68.CB7.CI.hARSAco.rBG (GTP-207) (FIG. 57) ARSA expression was not seen in the spleen, kidney, quadriceps, or lung (data not shown).

### LC/MS (Quantifying Sulfatide Storage)

LC/MS analysis was performed to quantify sulfatide storage in the brain, sciatic nerve, liver, spleen, kidney, heart, quadriceps muscle, and plasma at necropsy on Day 30. Only the sulfatide species that could be detected in the tissue tested are presented below.

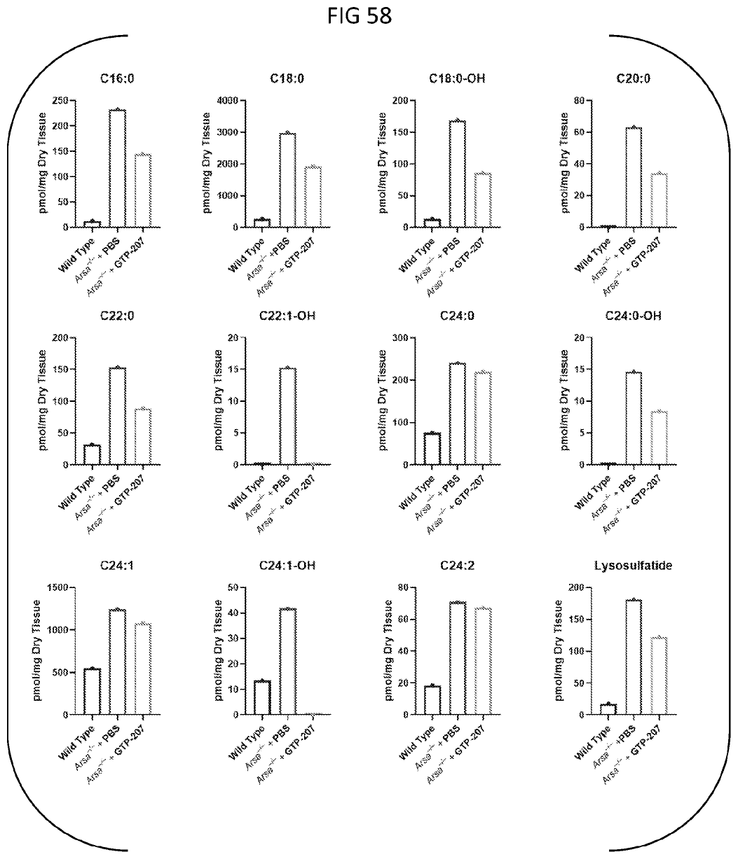

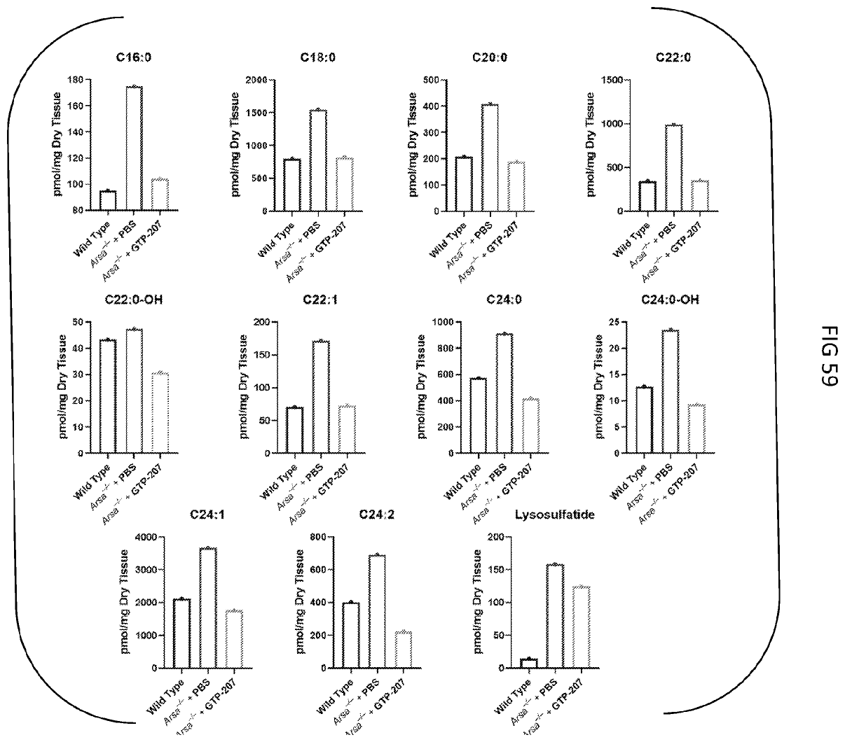

In the brain, all sulfatide species analyzed were higher in the vehicle-treated Arsa mouse compared to the age-matched wild type control mouse. The Arsa^{-/-} mouse administered AAVhu68.CB7.CI.hARSAco.rBG (GTP-207) demonstrated correction of the sulfatide storage, with reduction in the levels of several species (C16, C18, C20, C22 and lysosulfatide) compared to the vehicle-treated Arsa^{-/-} mouse (FIG. 58).

In the sciatic nerve, the highest levels of all detectable sulfatide species analyzed were detected in the vehicle-treated Arsa^{-/-} mouse (FIG. 59). Overall, Arsa mouse administered AAVhu68.CB7.CI.hARSAco.rBG (GTP-207) showed correction of sulfatide accumulation, with levels similar to or lower than those measured in the age-matched wild type control mouse.

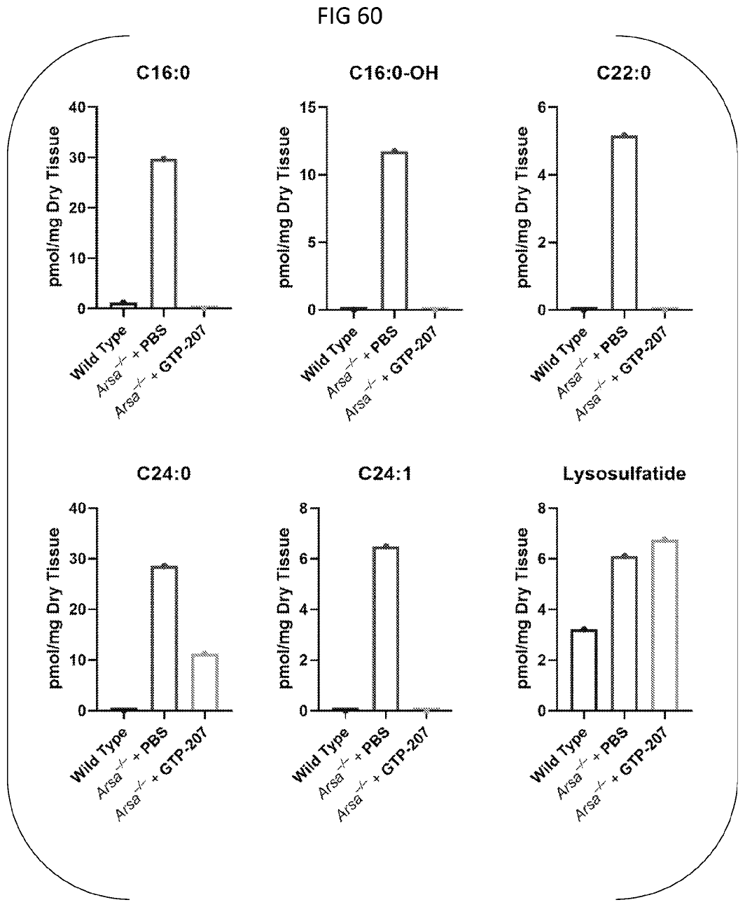

In the liver, all detectable sulfatide species analyzed were higher in the vehicle-treated Arsa mouse than the age-matched wild type control mouse (FIG. 60). The Arsa mouse administered AAVhu68.CB7.CI.hARSAco.rBG (GTP-207) demonstrated reduced levels of all sulfatide species except lyosulfatide compared to the vehicle-treated Arsa^{-/-} mouse, with similar or lower levels of C16:0, C16:0-OH, C22:0, and C24:1 sulfatide species compared to the age-matched wild type control mouse.

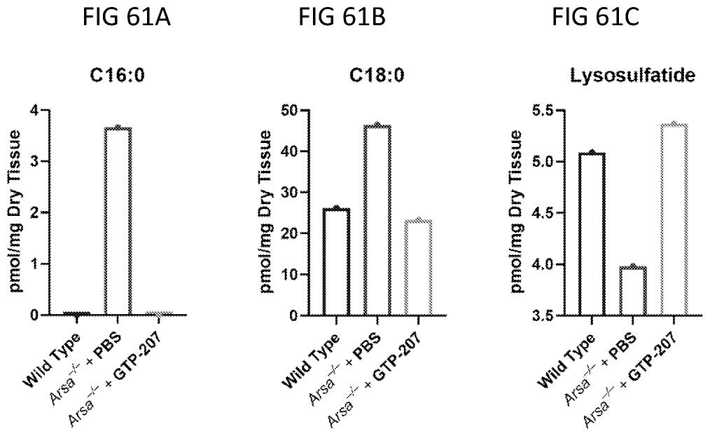

In the spleen, levels of C16:0 and C18:0 sulfatide species were highest in the vehicle-treated Arsa^{-/-} mouse and similar between the Arsa^{-/-} mouse administered AAVhu68.CB7.CI.hARSAco.rBG (GTP-207) and the age-matched wild type control mouse (FIG. 61). The concentration of lysosulfatide was highest in the Arsa mouse administered AAVhu68.CB7.CI.hARSAco.rBG (GTP-207), and similar to the WT control.

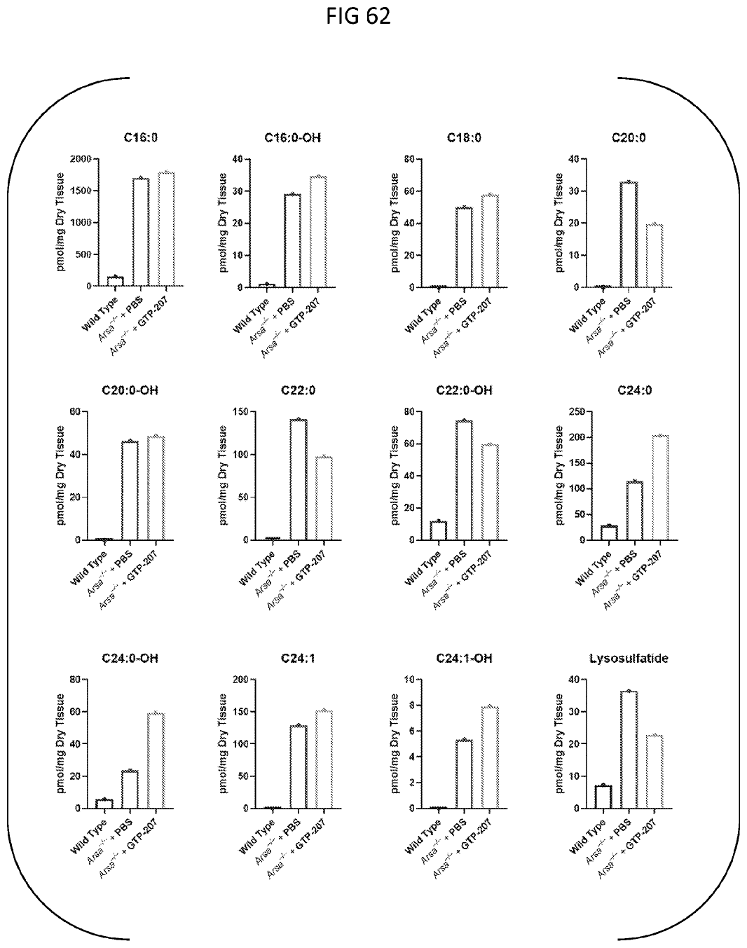

In the kidney, all sulfatide species analyzed were higher in the vehicle-treated Arsa mouse compared to the age-matched wild type control mouse (FIG. 62). Some sulfatide species showed decreased levels in AAVhu68.CB7.CI.hARSAco.rBG (GTP-207) treated Arsa^{-/-} mice, while remaining higher than WT control (C20:0, C22:0, C22-0-OH).

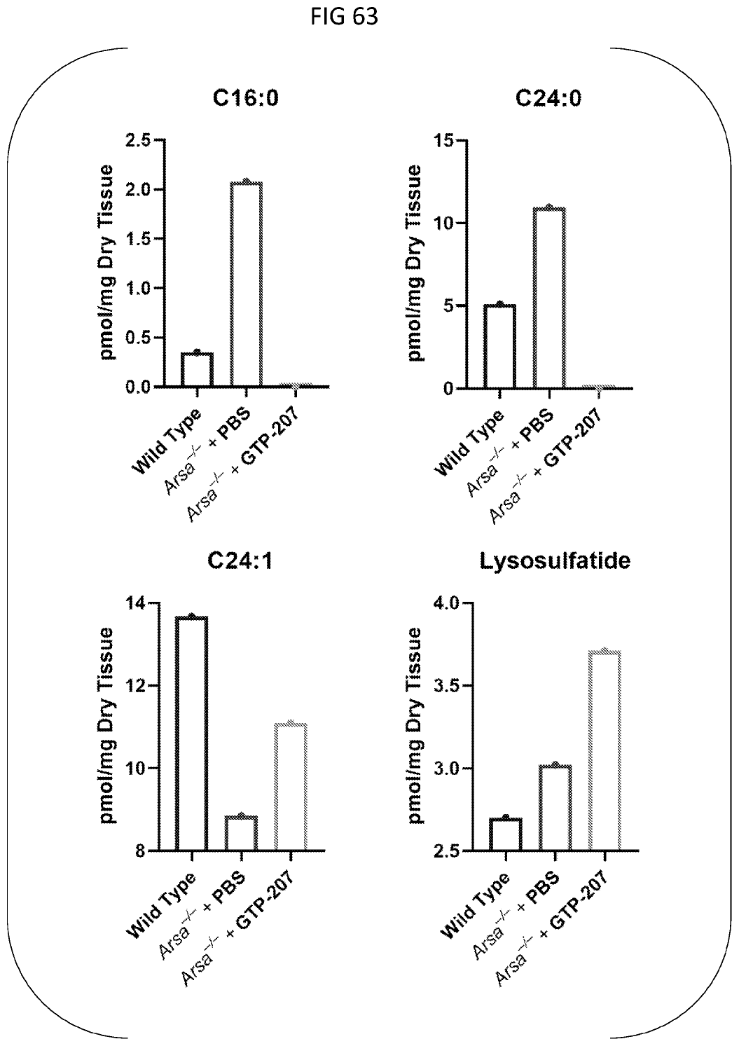

In the heart, levels of C16:0 and C24:0 sulfatide species were highest in the vehicle-treated Arsa^{-/-} mouse and was corrected in the Arsa mouse administered AAVhu68.CB7.CI.hARSAco.rBG (GTP-207) (FIG. 63). However, AAVhu68.CB7.CI.hARSAco.rBG (GTP-207) administration did not correct the levels of C24:1 and lysosulfatide in the Arsa mouse.

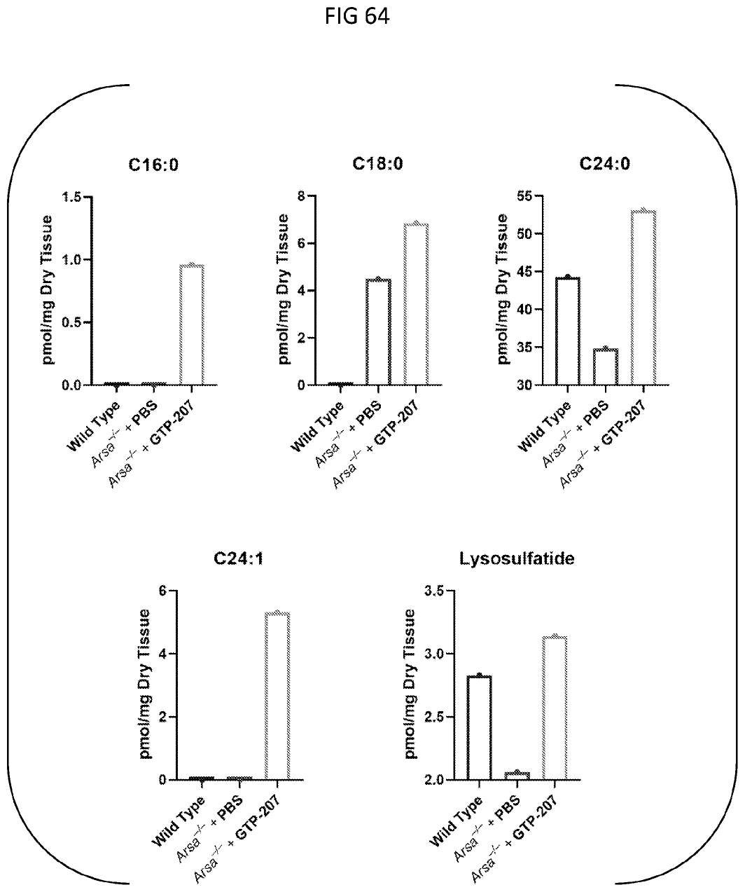

In the quadriceps muscle, administration AAVhu68.CB7.CI.hARSAco.rBG (GTP-207) did not correct the accumulation of sulfatides (FIG. 64).

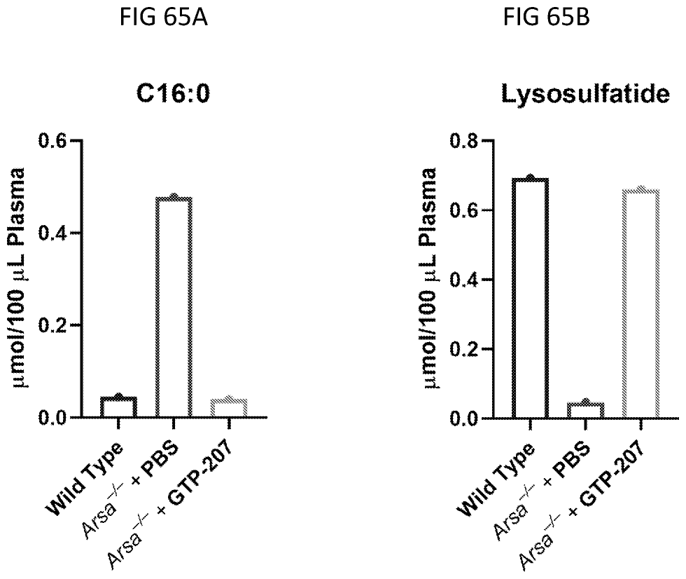

In plasma, administration of AAVhu68.CB7.CI.hARSAco.rBG (GTP-207) corrected the levels of C16:0 and lysosulfatide in the Arsa^{-/-} mouse administered AAVhu68.CB7.CI.hARSAco.rBG (GTP-207), and levels were similar to the age-matched wild type control mouse (FIG. 65).

### ARSA Enzyme Activity (Evaluating Transgene Product Expression)

ARSA enzyme activity was assessed in the serum and tissues (brain, heart, spinal cord, liver, kidney, spleen). For measuring ARSA enzyme activity, three different protein concentrations were tested to determine the optimum protein loading for the assay (FIG. 66). In brain and spinal cord, protein concentrations of 0.3 mg/mL (12 μg per well) appeared optimal for the tissues tested. However, the results showed similar ARSA enzyme activity between AAVhu68.CB7.CI.hARSAco.rBG (GTP-207) and vehicle-treated animals in brain. Potential explanations for this result are the method of tissue collection and/or the low sensitivity and/or specificity of the assay. For this study, the entire right sagittal half of the brain was collected and processed. Given that the expression of the transgene product is highest at the site of injection (human ARSA IHC) and gradually declines further away from the site of injection, assaying activity in the hemi-brain may have resulted in dilution of ARSA enzyme activity in the sample tested from the AAVhu68.CB7.CI.hARSAco.rBG (GTP-207)-treated animal. Moreover, the substrate used in these assay is not specific to ARSA and other sulfatases like ARSB, ARSK, C2 sulfatase, which can cleave the sulfate group from 4-nitrocatechol (Benitez and Halver, 1982; Lubke and Damme, 2020). As a result, there is a background non-specific activity due to the activity of other hydrolases, which explains a positive activity close to wild type levels in the vehicle-treated Arsa^{-/-} mouse. In peripheral tissues, the protein loading concentration of the sample did not affect ARSA enzyme activity. Interestingly we detected a strong increase in the liver and spleen of the AAVhu68.CB7.CI.hARSAco.rBG (GTP-207)-treated animals compared to vehicle-treated Arsa^{-/-} mice and wild type controls.

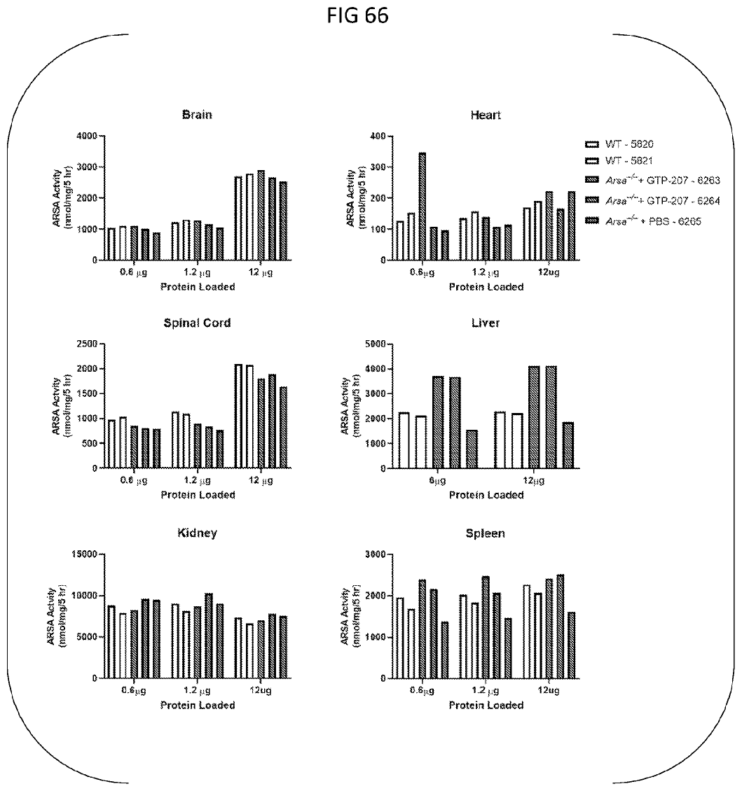

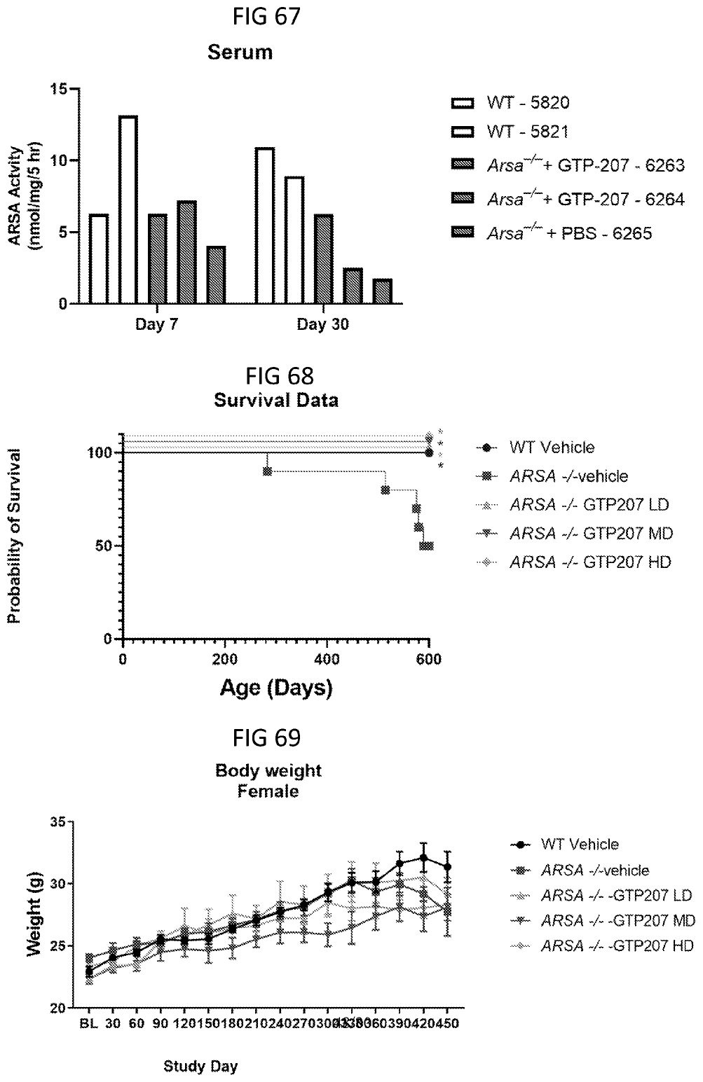

On Day 7, ARSA enzyme activity levels were generally similar between vehicle-treated wild type control mice and Arsa^{-/-} mice administered AAVhu68.CB7.CI.hARSAco.rBG (GTP-207), and ARSA enzyme activity levels were increased compared with Arsa^{-/-} controls (FIG. 67). At necropsy on Day 30, vehicle-treated wild type control mice had similar ARSA enzyme activity levels as those measured on Day 7. However, Arsa^{-/-} mice administered AAVhu68.CB7.CI.hARSAco.rBG (GTP-207) demonstrated a slight reduction in ARSA enzyme activity compared to Day 7.

### Summary of Results

The Arsa^{-/-} mouse administered AAVhu68.CB7.CI.hARSAco.rBG (GTP-207) ARSA protein expression by immunohistochemistry in cells of the cerebral cortex, hippocampus, cerebellum, and brain stem, with ARSA positive cells more abundant in the cortex and hippocampus.
Treatment of the Arsa mouse with AAVhu68.CB7.CI.hARSAco.rBG (GTP-207) resulted in a reduction in lysosomal pathology (measured by LAMP-1 IHC) and astrogliosis/neuroinflammation (GFAP IHC) in the cerebral cortex and hippocampus compared to the vehicle-treated Arsa^{-/-} mouse. No differences were noted in the cerebellum, brain stem and spinal cord. This corresponds to better correction in regions where more abundant ARSA expression is observed by IHC.
The Arsa^{-/-} mice administered AAVhu68.CB7.CI.hARSAco.rBG (GTP-207) had reduced levels of several sulfatide species in the brain, liver, spleen, heart, sciatic nerve, and plasma compared to levels measured in the vehicle-treated Arsa^{-/-} mouse. In the brain, AAVhu68.CB7.CI.hARSAco.rBG (GTP-207) administration demonstrated correction of the sulfatides with reduction in the levels of C16, C18, C20, C22 and lysosulfatide compared to the vehicle-treated Arsa^{-/-} mouse. Similar results were also observed in the heart, liver, spleen, and in plasma levels of C16. In the kidney, whilst some sulfatides were lower in treated mice versus control, the increased levels of sulfatide did not correct to wild type levels.
Analysis of different protein concentrations indicated that a loading concentration of 0.3 mg/mL (12 μg per reaction) is optimum for ARSA enzyme activity in brain and spinal cord. No difference was observed in the brain ARSA enzyme activity of AAVhu68.CB7.CI.hARSAco.rBG (GTP-207)-treated and vehicle-treated treated Arsa mice. Assaying ARSA activity from the whole sagittal hemi-brain may have resulted in dilution of the enzyme activity as transduction is more robust in rostral and periventricular region at the dose that was tested.
The Arsa^{-/-} mice administered AAVhu68.CB7.CI.hARSAco.rBG (GTP-207) demonstrated increased ARSA enzyme activity in liver, spleen, and serum.

### Example 8—Study Evaluating the Efficacy and Optimal Dose of AAVhu68.CB7.CI.hARSAco.rBG (GTP-207) Following Intracerebroventricular Administration to Arsc^{-/-} Mice

An efficacy and dose range study was performed to characterize the long-term effect of AAVhu68.CB7.CI.hARSAco.rBG (GTP-207), a recombinant adeno-associated viral (AAV) serotype hu68 vector expressing the human arylsulfatase A (ARSA) gene following intracerebroventricular (ICV) administration in the novel Arsa^{-/-} mouse model of metachromatic leukodystrophy (MLD).

On Study Day 0, adult (4-5 months old) Arsa^{-/-} mice received a single ICV administration of AAVhu68.CB7.CI.hARSAco.rBG (GTP-207) at one of three doses (1.3×10¹⁰ GC [3.3×10¹⁰ GC/g brain], 4.5×10¹⁰ GC [1.1×10¹¹ GC/g brain], or 1.3×10¹¹ GC [3.3×10¹¹ GC/g brain]). Additional Arsa^{-/-} mice and wild type C57BL6/J mice administered vehicle (phosphate-buffered saline [PBS]) were included as controls. Ten animals per group (5 males and females) were evaluated.

In-life assessments included survival monitoring, body weight measurements, clinical scoring assessments, and evaluation of neuromotor function (ledge test, RotaRod assay, and CatWalk gait analysis). Necropsies were performed at approximately 19-20 months of age. ARSA enzyme activity was assessed in the CNS, peripheral organs, and serum. Sulfatide storage was assessed in plasma (liquid chromatography/mass spectrometry [LC/MS]), peripheral organs, and target tissues relevant for the treatment of the neurological features of MLD (central nervous system [CNS] and peripheral nervous system [PNS]) by LC/MS. Histological markers of disease burden, lysosomal storage lesions (lysosomal-associated membrane protein 1 [LAMP-1] immunohistochemistry [IHC]) and astrogliosis/neuroinflammation (glial fibrillary acidic protein [GFAP] IHC) were quantified in the CNS.

The aim of this study was to characterize the long-term efficacy, including impact on neurobehavioral function and survival, of a dose range of AAVhu68.CB7.CI.hARSAco.rBG (GTP-207) after ICV administration to adult (4-5 months old) Arsa^{-/-} mice.

Group designations, dose levels, and the route of administration (ROA) are presented in the table below.

TABLE
Group Designations, Dose Levels, and Route of Administration

| | N | | | (GC/Animal | Dose | Dose | | | Necropsy | Necropsy |
|---|---|---|---|---|---|---|---|---|---|---|
| | and | | | Dose | (GC/g | Volume | | Dosing | Study | Age |
| Group | Sex | Genotype | Treatment | ) | brain)^{a} | (μL) | ROA | Day | Day | (Months) |
| 1 | 5 M | C57BL/6J | PBS | N/A | N/A | 5 | ICV | 0 | 450 | 19-20 |
| | 5 F | (WT) | | | | | | | | |
| 2 | 5 M | Arsa^{-/-} | PBS | N/A | N/A | 5 | ICV | 0 | 144-450^{b} | 9-20 |
| | 5 F | | | | | | | | | |
| 3 | 6 M | Arsa^{-/-} | GTP-207 | 1.3 × 10¹¹ | 3.3 × 10¹¹ | 5 | ICV | 0 | 450 | 19-20 |
| | 5 Fc | | | | | | | | | |
| 4 | 5 M | Arsa^{-/-} | GTP-207 | 4.5 × 10¹⁰ | 1.1 × 10¹¹ | 5 | ICV | 0 | 450 | 19-20 |
| | 5 F | | | | | | | | | |
| 5 | 5 M | Arsa^{-/-} | GTP-207 | 1.3 × 10¹⁰ | 3.3 × 10¹⁰ | 5 | ICV | 0 | 450 | 19-20 |
| | 5 F | | | | | | | | | |

^{a}Values were calculated using 0.4 g brain mass for an adult mouse (Gu et al., 2012).
bThree animals in this group were found dead on Study Days 433 (N = 1) and 443 (N = 2), two animals had emergency necropsy per veterinary recommendation due to humane endpoint on Study Days 144 and 375.
^{c}One mouse was euthanized per veterinary recommendation for reaching humane endpoint 4 days post ICV dosing.
The animal exhibited increased respiratory effort and poor body condition. Therefore, 11 mice were injected with vector at 1.3 × 1011 GC. Abbreviations: Arsa, arylsulfatase A (gene, mouse); F, female; GC, genome copies; ID, identification number; IV, intravenous; M, male; N, number of animals; N/A, not applicable; ROA, route of administration; WT, wild type.

On Study Day 0, adult (4-5 months of age) Arsa^{-/-} mice and age-matched C57BL/6J wild type controls received a single ICV administration of either AAVhu68.CB7.CI.hARSAco.rBG (GTP-207) at one of three doses (1.1×10¹¹ GC or 4.5×10¹⁰ GC, or 1.1×10¹⁰ GC) or control article (PBS [vehicle]).

In-life assessments included viability checks, veterinary clinical observation, body weight measurements, clinical scoring assessments, and evaluation of neuromotor function (ledge test, RotaRod assay, and CatWalk gait analysis) at various time points. Necropsies were performed on Day 450 (19-20 months of age) or when mice reached a humane endpoint based on Veterinary clinical observations. Sulfatide storage was assessed in plasma, brain, spinal cord, sciatic nerve, liver, and kidney (by LC/MS). Lysosomal storage lesions (LAMP-1 IHC) and astrogliosis/neuroinflammation (GFAP IHC) were quantified in the brain and spinal cord. ARSA enzyme activity was also assessed in the brain (disease relevant target), liver, heart (major peripheral organs transduced after an ICV dosing), and serum.

### Clinical Observations

### Clinical Scoring Assessment

Clinical signs were scored by two research staff members using an unpublished assessment of clasping ability, gait, tremor, kyphosis, and fur quality. These measures were chosen to assess clinical status based on the symptoms typically exhibited by Arsa^{-/-} mice. Scores above 0 indicated clinical deterioration. Operators were blinded to the treatment and genotype when collecting and recording scores.

| Assessment | | |
|---|---|---|
| Category | Observation | Score |
| Fur Quality | Shiny coat, smooth fur, well groomed, clean coat | 0 |
| | Not well groomed, slightly oily or rough coat | 1 |
| | Rough Hair coat-hair coat is oily, dirty, stands on end | 2 |
| | Very rough hair coat, dehydration | 3 |
| Gait | Active, energetic. Body weight supported on all limbs. Even hindlimbs, | 0 |
| | abdomen raised | |
| | Able to run, but sluggish. Limp when walking. | 1 |
| | Reluctant to move, severe limp, lowered pelvis, feet point away from body | 2 |
| | Moves only when manually prodded & minimally, drags abdomen | 3 |
| Tremor | No sign of tremor | 0 |
| | Very slight, impermanent | 1 |
| | Mild, visible upon close observation | 2 |
| | Moderate, visible immediately | 3 |
| | Severe, impacts mobility | 4 |
| Clasping | Hindlimbs consistently splayed outward from abdomen | 0 |
| | 1 hindlimb is retracted toward abdomen for >50% time | 1 |
| | Both limbs partially retracted for >50% of time | 2 |
| | Hindlimbs entirely retracted and touching abdomen for >50% time | 3 |
| | Permanent full grasping of 4 limbs | 4 |
| Posture | Normal, flat back-easily straightens spine as it walks | 0 |
| | Transitory hunching, evident only when resting-mild but able to | 1 |
| | straighten spine | |
| | Rests upright with back hunched, unable to straighten completely when | 2 |
| | moving but mild | |
| | Pronounced and maintained kyphosis while walking and sitting | 3 |

### Neuromotor Function Assessments

### Ledge Test

The ledge test measures coordination, which is impaired in neurodegenerative diseases associated with ataxia, such as MLD. Mice were evaluated for phenotypic progression through conducting the ledge test according to the published protocol (Guyenet et al., 2010). Briefly, the animal was lifted from its cage and placed on the cage's ledge. The mouse was observed and assigned a score based on its ability to navigate along the ledge and get itself back into its cage. Scores above 0 indicated a decrease in neuromotor function.

| Score | Observation |
|---|---|
| 0 | Mouse walks along the ledge without losing its balance, and lowers itself back into its |
| | cage using its paws gracefully and without losing balance |
| 1 | Mouse loses its footing while walking along the ledge, but otherwise appears |
| | coordinated. |
| 2 | Mouse does not effectively use its hind legs or lands on its head rather than its paws |
| | when descending into the cage |
| 3 | Mouse nearly or completely falls off the ledge while walking or attempting to lower |
| | itself or shakes and refuses to move at all despite encouragement. |

Abbreviations:
N/A, not applicable.

### RotaRod

Coordination and balance were measured using the RotaRod test (Ugo Basile; Gemonio, Italy). Briefly, mice were first habituated to the RotaRod by placing up to five mice per trial in a lane of the RotaRod device facing the wall. Mice were allowed to stabilize themselves on the fixed (non-rotating) rod for 2 minutes. Two habituation trials were then performed with the rod rotating for 1 minute at a constant speed of 5 revolutions per minute (RPM). Between each habituation trial, mice were allowed to rest in the RotaRod collecting box for approximately 1 minute. If a mouse fell during the habituation phase, it was immediately placed back on the rod.

Immediately following habituation, testing trials were performed to measure how long each mouse could remain on the rotating rod while it was accelerating. The mice were placed in a lane of the RotaRod device facing the wall and allowed to equilibrate on the fixed (non-rotating) rod to establish a firm grip. The rod was then set to spin at a constant speed of 5 RPM for a few seconds to allow the mice to equilibrate. Once equilibrated, the rod was set to accelerate from RPM to 40 RPM over 120 seconds. For each animal, the testing trial was considered terminated when the mouse fell off the rod, completed two passive revolutions, or 120 seconds had elapsed. The fall latency (defined as the time between the initiation of rod acceleration and trial termination) was recorded. A total of three sequential test replicates were performed for the mice in each trial, with a 1-3 minute pause in between runs to allow the animals to rest in the collecting box.

### CatWalk Gait Analysis

Gait and walking speed were assessed using the CatWalk XT gait analysis system (Noldus Information Technology, Wageningen, The Netherlands). The CatWalk XT tracks the footprints of mice as they walk across a glass plate. The system quantifies the dimensions of each paw print and statistically analyzes the animal's speed and other features of gait.

To perform this assessment, the Catwalk XT was calibrated, with the appropriate width of the walkway set, prior to the start of the test. All experiment settings were entered into the Catwalk XT software, including animal type, time point, and run criteria. Animals were brought into the room and allowed to acclimate in darkness for at least 30 minutes prior to running on the Catwalk XT. Once acclimation was complete, an animal was selected and placed at the entrance of the walkway. The researcher started the acquisition software and allowed the animal to walk down the walkway. The animal's home cage was placed at the end of the walkway for encouragement. The run was complete when the animal had successfully walked to the end of the catwalk within the allotted time limit, otherwise the run was repeated. Animals ran three trials with a minimum duration of 0.50 seconds and a maximum duration of 5.00 seconds. Three successful runs were needed for the trial to be considered complete. If an animal failed to complete three runs after 10 minutes of testing, only the completed runs were used for analysis. Animals were tested twice on two consecutive days. The first day of testing was used to habituate animals to the testing apparatus, and the second day of testing was scored. Runs were auto-classified using the Catwalk XT software, after which footprints were checked for accuracy and proper labeling. Any non-footprint data were manually removed. All data were exported into Microsoft Excel and GraphPad Prism 7.0 for analysis.

### Catwalk Gait Analysis Parameters Evaluated

Parameters automatically measured by the Catwalk XT system included base of support, print positions, cadence, step sequence regularity, average body speed, and stride length as described below. Mean values were calculated and analyzed for each group.

Base of support was determined by the Catwalk XT system as the average width between either the front paws or the hind paws. Print positions were determined by the Catwalk XT system as the distance between the position of the hind paw and the position of the previously placed front paw on the same side of the body (ipsilateral) and in the same Step Cycle. The animal's cadence was determined by the Catwalk XT system as steps per second. The step sequence was evaluated by the Catwalk XT system by determining the percent of steps that falls into one of six regular patterns typically observed in healthy mice. The average body speed was determined by the Catwalk XT system based on the step cycle of a specific paw by dividing the distance that the animal's body traveled from one initial contact of that paw to the next by the time to travel that distance. The stride length was determined by the Catwalk XT system based on the distance (in Distance Units) between successive placements of the same paw. Contact area was determined by the Catwalk XT system based on Illuminated Footprints™ technology where paws are captured by a high-speed video camera that is positioned underneath the walkway. Print width and print length were determined by the Catwalk XT system from the video images with paw prints used in the footprint classification. Once classification was done, the CatWalk software automatically calculated parameters related to individual footprints.

### LAMP-1 IHC (Evaluating Lysosomal Storage Lesions) and GFAP IHC (Evaluating Astrogliosis/Neuroinflammation)

IHC was performed on sections from FFPE tissues on a Leica Bond Rx autostainer following a standard immunohistochemistry (IHC) protocol with the Bond polymer detection system (Leica Biosystems, DS9800) and DAB as chromogen. For all stains citrate buffer pH6 was used for antigen retrieval (20 min). LAMP1 was detected with monoclonal rat antibody 1D4B (Abcam ab25245, diluted 1:50), GFAP with a rabbit antibody from Abcam (ab7260, diluted 1:4000), and ARSA with a rabbit antibody from Sigma (HPA005554, diluted 1:200). Incubation time for all primary antibodies was set to 30 min. Ready to use secondary polymer antibodies were either from Vector Laboratories (anti-rat for LAMP1, MP-7444, incubation time 20 min) or from Leica (for GFAP and ARSA rabbit antibodies, BOND Polymer Refine Detection DS9800, incubation time 8 min). After staining slides were dehydrated through ethanol and xylene and coverslipped.

The LAMP-1 and GFAP IHC were quantified using image analysis software. Briefly, well-stained and intact regions of sections of the brain, spinal cord, and sciatic nerve were manually outlined using VIS version 2019.07.0.6328 (Visiopharm, Hoersholm, Denmark). For the brain, LAMP-1 positive area was quantitated via thresholding using the IHS-S (Intensity, Hue, Saturation model) classification feature. The LAMP-1-negative area was quantified via thresholding using the HDAB-Hematoxylin classification feature, and the LAMP-1-positive and LAMP-1-negative area classifications were used to generate the percentage of the outlined section that was LAMP-1 positive, the number of LAMP-1 positive objects, and the average size of all LAMP-1 objects identified in the section. For the spinal cord, LAMP-1 positive and LAMP-1 negative areas were quantified via thresholding using the HDAB-DAB classification feature, and the LAMP-1-positive and LAMP-1-negative area classifications were used to generate the percentage of the outlined section that was IBA1-positive, the number of LAMP-1-positive objects, and the average size of all LAMP-1 objects identified in the section. For the sciatic nerve, the LAMP-1-positive area was quantitated via thresholding using the HDAB-DAB classification feature. The LAMP-1 negative area and empty space induced by processing were quantified via thresholding using the HDAB-Hematoxylin classification feature, and the LAMP-1 positive and LAMP-1 negative area (but not the empty space) classifications were used to generate the percentage of the outlined section that was LAMP1-positive, the number of LAMP-1 positive objects, and the average size of all LAMP-1 objects identified in the section.

### Quantification of Sulfatide Storage by LC/MS

Thawed tissues were lyophilized overnight and ground to a fine powder in 2.0 mL polypropylene tubes with ceramic beads using a Precellys bead-beating homogenizer (Bertin Technologies, Rockville, MD) at 4° C. Aliquots of powder (˜2.5-5.0 mg) were weighed on an analytical balance followed by homogenization in 500 μL of 80% methanol in the Precellys homogenizer at 4° C. A 100 μL aliquot of homogenate was then spiked with 10 μL of a C18:0-CD3-sulfatide internal standard (N-omega-CD3-Octadecanoyl-sulfatide Matreya State College, PA, catalog #1536; 25 μM) and extracted with 400 μL of ice cold methanol in a 2.0 mL Eppendorf tube. The sample was centrifuged for 5 minutes at 14,000×g at 4° C. Aliquots (400 μL) of methanolic supernatants were dried under nitrogen in a 96-well plate at 45° C. and reconstituted in 150 μL of methanol for LC/MS analysis.

Calibration samples of sulfatide standards were prepared. Standard powders of sulfatides (lysosulfatide catalog #1904; C16:0 catalogue #1875, C18:0 catalogue #1932, C18:0-CD3 catalogue #1536, and C24:1 catalogue #1931; Matreya, State College, PA) were weighed on an analytical balance, and individual stock solutions (1 mM) were prepared in 2:1 methyl tert-butyl ether/methanol. The C18:0-CD3sulfatide internal standard stock solution was diluted in methanol to give a 25 μM spiking internal standard solution. Aliquots of the individual stock solutions were combined to make a high calibration spiking solution of 50 μM lysosulfatide, 50 μM C16:0, 250 μM C18:0, and 250 μM C24:1 sulfatide in methanol. This high calibration spiking solution was serially diluted in methanol to make 0.1, 0.25, 0.5, 1, 5, 10, 25, and 50 μM calibration curve spiking solutions for lysosulfatide and C16:0 sulfatide, along with 0.5, 1.25, 2.5, 5, 25, 50, 125, and 250 μM calibration curve spiking solutions for C18:0 and C24:1 sulfatide. Calibration curve solutions for LC/MS analysis were created by pipetting 10 μL of each spiking solution and 10 μL of the C18:0-CD3-sulfatide internal standard (25 μM) into 100 μL of 80% methanol, resulting in LC/MS calibration curves of 0.01, 0.025, 0.05, 0.1, 0.5, 1, 2.5, and 5 μM for lysosulfatide and C16 sulfatide, along with LC/MS calibration curves of 0.05, 0.125, 0.25, 0.5, 2.5, 5, 12.5, and 25 μM for C18:0 and C24:1 sulfatide. A 400 μL aliquot of methanol was added to each solution. The sample was vortexed, and 400 μL was dried under nitrogen in a 96-well plate at 45° C. and reconstituted in 150 μL of methanol for LC/MS analysis.

Sulfatides were quantified with an Agilent 1290 Infinity UHPLC/6495B triple quadrupole mass spectrometer. Biological extracts and calibration solutions in 96-well plates were injected (5 μL) and separated on the UHPLC. Sulfides were eluted by gradient elution on a Waters Acquity BEH C18 2×100 mm, 1.7 μM column at a flow rate of 0.4 mL/minute at 45° C. A 7.5 minute gradient was used beginning with 35% solvent A (70/30 deionized water/acetonitrile/0.1% formic acid) and 65% solvent B (50/50 acetonitrile/isopropanol/0.1% formic acid) held for 0.5 minutes and increased to 100% solvent B over 5.5 minutes, held at 100% solvent B until 7.5 minutes followed by re-equilibration back to starting conditions from 7.6 to 10 minutes. The HPLC flow was diverted to waste for the first 0.5 minute then directed to the electrospray ionization source. Sulfatides were ionized by electrospray ionization in the positive ionization mode on the mass spectrometer. The Agilent Jet Stream electrospray ionization source was operated with a nitrogen gas temperature of 250° C., gas flow of 14 L/minute, nebulizer of 45 psi, sheath gas temperature of 325° C., sheath gas flow of 12 L/minute, capillary voltage of 3500 V, and nozzle voltage of 500 V. Multiple reaction monitoring (MRM) was used to quantitate sulfatides with a peak width of 0.7 Da and an electron multiplier voltage of 400 V in the positive ion mode. The MRM table of parent to product ion transitions with collision energies is shown below (100). As an example, a primary transition for C16:0 sulfatide of m/z 780.57-264.2 was used for the quantitation of C16:0 by monitoring m/z 264.2, the while the secondary transition m/z 780.57-682.6, generated by neutral loss of H₂SO₄ from the parent ion, was used to confirm the primary transition as an authentic sulfatide. Agilent MassHunter software was used to generate linear or quadratic calibration curves (1/x or 1/x2 weighting and R2 0.99 or better) to quantify sulfatides in biological samples.

### Measuring ARSA Enzyme Activity

ARSA enzyme activity was measured in dialyzed tissues samples using a p-nitrochatechol assay. Briefly, dialyzed tissues (brain=0.6 mg/mL; liver 0.025-0.3 mg/mL; heart=0.3 mg/mL) were diluted into a base buffer (0.5 M sodium acetate buffer, pH 5.0; 10% sodium chloride; 0.5 mM sodium pyrophosphate) and 40 μL of diluted sample was loaded into four wells (2 duplicates) of a 96-well plate. Next, 40 μL of substrate (10 mM 4-nitrocatechol sulfate in base buffer) was added to the samples, and the reaction was stopped immediately by adding 120 μL 1N NaOH (stop solution) in two of the four wells. The plate was then incubated at 37° C. for 5 hours. The reaction was stopped by adding 120 μL 1N NaOH (stop solution) in the remaining wells. The absorbance was measured by reading the plate at 515 nm using a plate reader. ARSA-specific activity was determined by multiplying the absorbance obtained at five hours minus the absorbance at 0 minute with the extinction coefficient of a 4-nitrocathecol (4-NC) standard curve at 515 nm and by dividing by the amount of protein in the well (mg) as measured by BCA assay. The results for ARSA activity were expressed as nmol 4-NC generated per milligram tissue per five hours.

### Results

Vehicle-treated Arsa^{-/-} control mice had a shortened lifespan, with 50% of mice alive at Study Day 450 (median survival age: 594.5 days) compared to 100% of the vehicle-treated wild type controls (median survival undefined). All AAVhu68.CB7.CJ.hARSAco.rBG (GTP-207)-treated Arsa animals survived to the scheduled necropsy except one male Arsa mouse (Animal 1139, Group 3) that was euthanized on Study Day 4 (4.8 months of age). The animal exhibited increased respiratory rate and effort on examination with a poor body condition. The clinical signs were likely attributable to the injection procedure, and the animal was therefore excluded from the study and survival analysis. This result indicates that AAVhu68.CB7.CI.hARSAco.rBG (GTP-207) ICV administration provided a survival benefit at all doses and ameliorated the shortened lifespan seen in Arsa^{-/-} mice (FIG. 68).

### Body Weight

Untreated female Arsa r mice exhibited weight gain similar to that of age-matched female wild type controls until approximately 15-16 months of age (Day 330), when weight gain patterns for Arsa- and wild type mice began to diverge. After this time point, weight generally plateaued and then decreased for untreated female Arsa^{-/-} mice, although the difference was not statistically different due to inter-animal variability. None of the AAVhu68.CB7.CJ.hARSAco.rBG (GTP-207)-treated Arsa^{-/-} female mice displayed a significantly different body weight than that of the vehicle-treated wild type mice, although the decline observed between Day 390 and Day 450 seemed prevented by AAVhu68.CB7.CJ.hARSAco.rBG (GTP-207) treatment at both the mid-dose (4.5×10¹⁰ GC) and high dose (1.3×10¹¹ GC). Treated Arsa^{-/-} mice from all groups started with a lower baseline body weight than that of vehicle-treated controls (p<0.05* for low dose and mid dose groups, not significant for high dose group based on mixed effect model with multiple comparison Dunnett's test) for unknown reasons. As this was prior to AAVhu68.CB7.CJ.hARSAco.rBG (GTP-207) administration, this lower baseline body weight was not considered treatment-related (FIG. 69).

Untreated male Arsa^{-/-} mice exhibited weight gain similar to that of age-matched male wild type controls until approximately 14-15 months of age (Day 300), when weight gain patterns for Arsa^{-/-} mice and wild type mice began to diverge. After this time point, weight generally plateaued and then decreased for untreated female Arsa^{-/-} mice, although the difference was not statistically different due to inter-animal variability. None of the AAVhu68.CB7.CJ.hARSAco.rBG (GTP-207)-treated Arsa^{-/-} male mice displayed a significantly different body weight than that of untreated mice. Arsa^{-/-} mice administered the mid-dose of AAVhu68.CB7.CJ.hARSAco.rBG (GTP-207) (4.5×10¹⁰ GC) started with a lower baseline body weight than vehicle-treated controls (p<0.05*, based on a mixed effect model with multiple comparison Dunnett's test) for unknown reasons. As this was prior to AAVhu68.CB7.CI.hARSAco.rBG (GTP-207) administration, this lower baseline body weight was not considered treatment-related (FIG. 69).

### Clinical Scoring Assessments

Clinical scoring was used to assess the clinical status of mice using a compound scoring adapted from ataxia evaluation scores assessing general health and neurological parameters: fur quality, tremors, gait, kyphosis, and clasping reflex, with scores above 0 indicating clinical deterioration and a maximal theoretical score of 17.

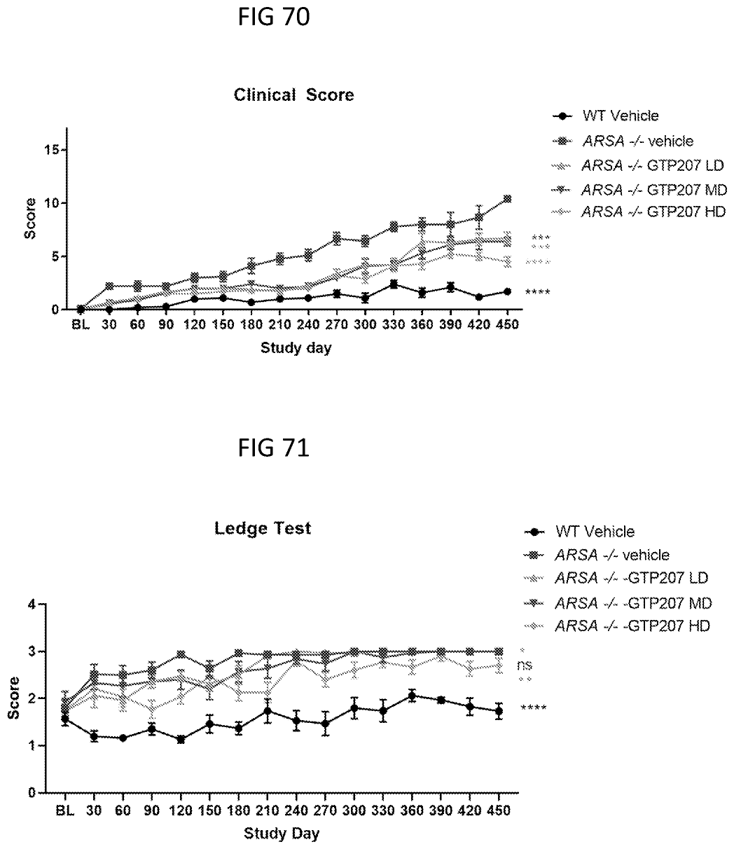

Vehicle-treated Arsa^{-/-} mice exhibited significantly higher clinical scores than that of age-matched wild type controls, with clinical scores progressively increasing throughout the study to the last time point evaluated at Day 450 (19-20 months of age). AAVhu68.CB7.CI.hARSAco.rBG (GTP-207)-treated Arsa^{-/-} mice (low dose [1.3×10¹⁰ GC], mid-dose [4.5×10¹⁰ GC], and high dose [1.3×10¹¹ GC]) displayed significantly lower clinical scores than that of age-matched vehicle-treated control Arsa^{-/-} mice. This result indicates that AAVhu68.CB7.CI.hARSAco.rBG (GTP-207) administration prevented the progressive worsening of clinical status seen in Arsa^{-/-} mice at all doses (FIG. 70).

### Neuromotor Function

### Ledge Test

The ledge test measured coordination, which is impaired in neurodegenerative diseases associated with ataxia, such as MLD. Mice were assigned a score from 0 to 3, with higher scores indicating reduced coordination.

Vehicle-treated Arsa^{-/-} mice exhibited significantly higher ledge test scores than that of age-matched wild type controls, with progressive increase throughout the study and maximal scores observed by Study Day 180 (10-11 months of age). Arsa^{-/-} mice administered the low dose of AAVhu68.CB7.CI.hARSAco.rBG (GTP-207) (1.3×10¹⁰ GC) displayed statistically significantly lower ledge test scores than that of age-matched vehicle-treated Arsa^{-/-} mice until Day 120 but were similar to controls thereafter. Arsa^{-/-} mice administered the high dose of AAVhu68.CB7.CI.hARSAco.rBG (GTP-207) (1.3×10¹¹ GC) displayed significantly lower clinical score than that of age-matched vehicle controls and remained lower until the final time point at Day 450 (19-20 months of age). This result indicates that AAVhu68.CB7.CI.hARSAco.rBG (GTP-207) administration ameliorated the progressive worsening of coordination seen in Arsa^{-/-} mice, although only the highest dose (1.3×10¹¹ GC) showed a sustained effect (FIG. 71).

### RotaRod

Neuromotor function was assessed by the RotaRod test, which evaluates coordination and balance by measuring the time to fall for mice running on a spinning rod that progressively accelerates. A decrease in fall latency indicates neuromotor impairment, while an increase in fall latency indicates improved neuromotor function.

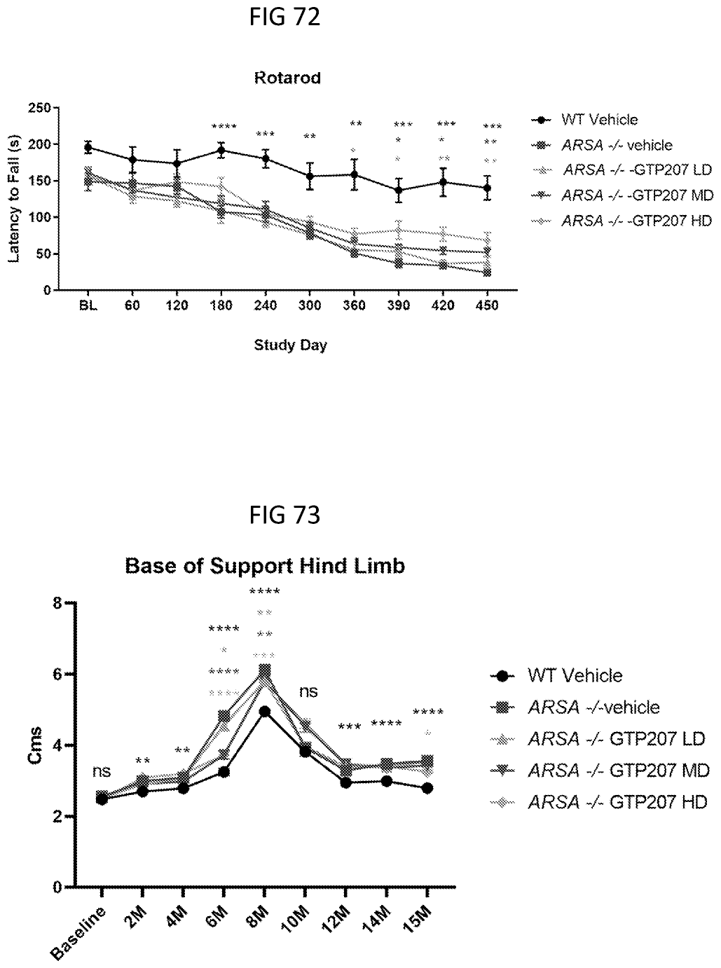

Vehicle-treated Arsa^{-/-} mice exhibited a significantly shorter fall latency than that of age-matched wild type controls, with progressive worsening from Day 180 (10-11 months of age) to Day 450 (19-20 months of age). Arsa^{-/-} mice administered the high dose of AAVhu68.CB7.CI.hARSAco.rBG (GTP-207) (1.3×10¹¹ GC) and high dose of AAVhu68.CB7.CI.hARSAco.rBG (GTP-207) (4.5×10¹⁰ GC) displayed a significantly increased latency to fall than that of age-matched vehicle-treated Arsa^{-/-} mice from Day 360-450 and from D390-450 respectively. This result indicates that AAVhu68.CB7.CI.hARSAco.rBG (GTP-207) administration ameliorated the progressive worsening of neuromotor function seen in Arsa mice, at the two highest doses (1.3×10¹¹ GC and 4.5×10¹⁰ GC) (FIG. 72).

### CatWalk Gait Analysis

Neuromotor function was assessed using the CatWalk XT gait analysis system, which measures a variety of parameters. Neuromotor function abnormalities would be expected to result in gait and/or walking speed abnormalities in Arsa^{-/-} mice when compared to wild type controls.

Base of support (distance between the 2 hind paws) was progressively increased in the hind limbs of vehicle-treated Arsa^{-/-} mice compared to age-matched vehicle-treated wild type mice from 2 months to 8 months post-dosing (6-7 months of age to 12-13 months of age). There was a subsequent loss of phenotype and apparent normalization with similar values in vehicle-treated Arsa^{-/-} mice compared to age-matched vehicle-treated wild type mice at 10 months (14-months of age). Arsa^{-/-} mice administered the low dose (1.3×10¹⁰ GC), mid-dose (4.5×10¹⁰ GC), or high-dose (1.3×10¹¹ GC) of AAVhu68.CB7.CI.hARSAco.rBG (GTP-207) displayed significantly improved base of support of hind limbs at 6 months (10-11 months of age) and 8 months (12-13 months of age); the reason for the apparent normalization in all groups in subsequent time points is unknown.

This result indicates that AAVhu68.CB7.CI.hARSAco.rBG (GTP-207) administration ameliorated the base of support at all doses (low dose [1.3×10¹⁰ GC], mid-dose [4.5×10¹⁰ GC], and high dose [1.3×10¹¹ GC]) up to 12-13 months of age, with limitations due to apparent inconsistency of this readout over time in later time points (FIG. 73). The base of support for the fore limb was inconsistent with the natural history study, with an apparent reversal of phenotype around 12 months (16-17 months of age), rendering any treatment effect uninterpretable.

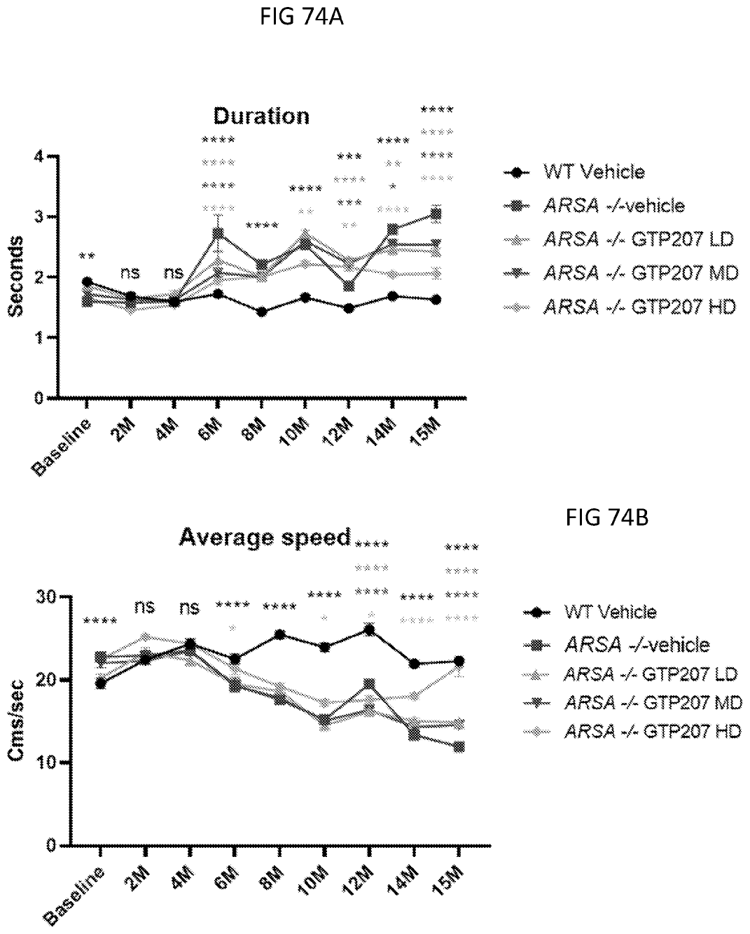

Duration and average speed, two indicators of how rapidly mice cross the catwalk ramp, showed that Arsa^{-/-} mice exhibit a significantly slower gait compared to that of age-matched wild type controls from 6 months (10-11 months of age) to the final 15-month time point (19-20 months of age). Arsa^{-/-} mice administered the low dose (1.3×10¹⁰ GC), mid-dose (4.5×10¹⁰ GC), or high-dose (1.3×10¹¹ GC) of AAVhu68.CB7.CI.hARSAco.rBG (GTP-207) displayed significantly shorter duration at 14 and 15 months (18-20 months of age), while speed was higher than that of Arsa^{-/-} controls at 15 months (FIG. 74). This result indicates that AAVhu68.CB7.CI.hARSAco.rBG (GTP-207) administration ameliorated the mobility and gait of the mice at all doses. Statistical significances prior to the two final time points are not interpretable due to high variability between measurements, likely due to milder phenotype (see apparent amelioration at 12 months in FIG. 74).

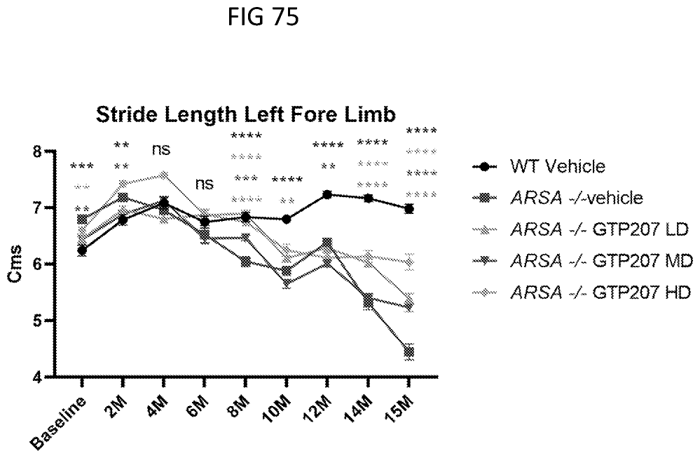

Stride length, the distance one paw travels during one step, showed a progressive decrease of movement amplitude in Arsa^{-/-} mice compared to age-matched wild type controls from 8 months (12-13 months of age) to the final 15 month time point (19-20 months of age). Arsa^{-/-} mice administered the low dose (1.3×10¹⁰ GC), mid-dose (4.5×10¹⁰ GC), or high-dose (1.3×10¹¹ GC) of AAVhu68.CB7.CI.hARSAco.rBG (GTP-207) displayed significantly increased movement amplitude when compared to that of Arsa^{-/-} controls in at least one paw at 14 and 15 months (18-20 months of age) (FIG. 75) This result indicates that AAVhu68.CB7.CI.hARSAco.rBG (GTP-207) administration ameliorated the mobility and gait of the mice at all doses. Statistical significances prior to the two final time points are not interpretable due to high variability between measurements, likely due to milder phenotype (see apparent amelioration at 12 months in FIG. 74).

### ARSA Enzyme Activity

ARSA enzyme activity was measured using a colorimetric assay that measures the release of a colored product (p-nitrocatechol) from p-nitrocatechol sulfate artificial substrate. This assay is not specific to ARSA, as the substrate can be cleaved by other sulfatases, such as ARSB, which is hypothesized to explain the positive values (i.e., non-specific enzyme activity) measured in vehicle-treated Arsa^{-/-} mice. Increases in ARSA activity AAVhu68.CB7.CI.hARSAco.rBG (GTP-207)-treated mice compared to vehicle-treated mice reflect expression of the human ARSA transgene product, as other sulfatases are not expected to increase following treatment.

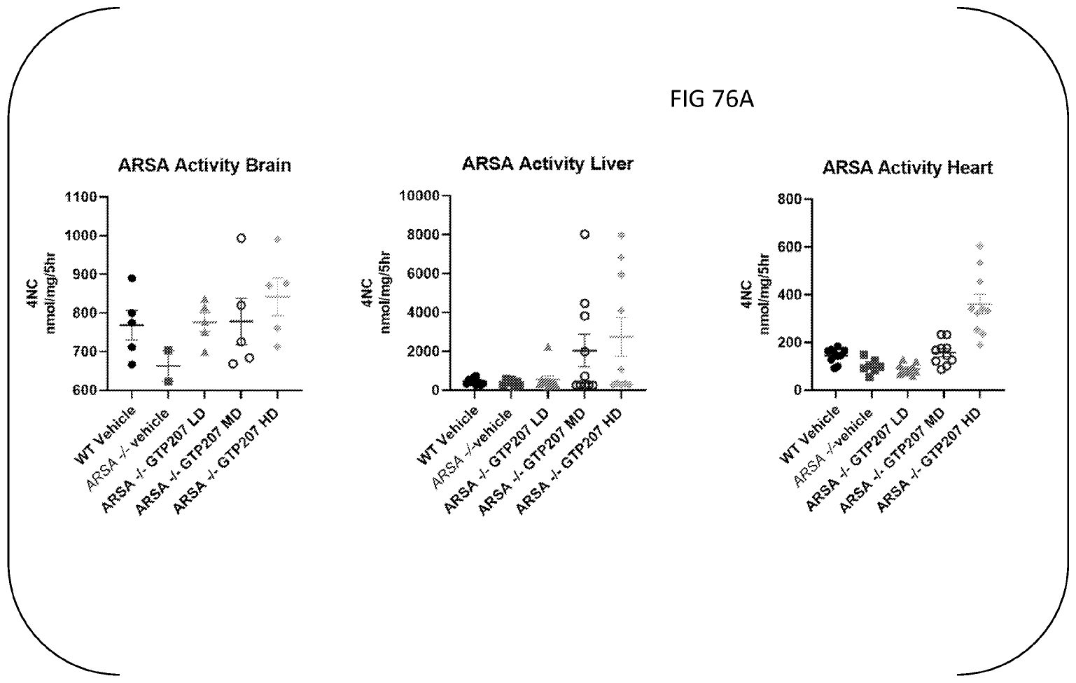

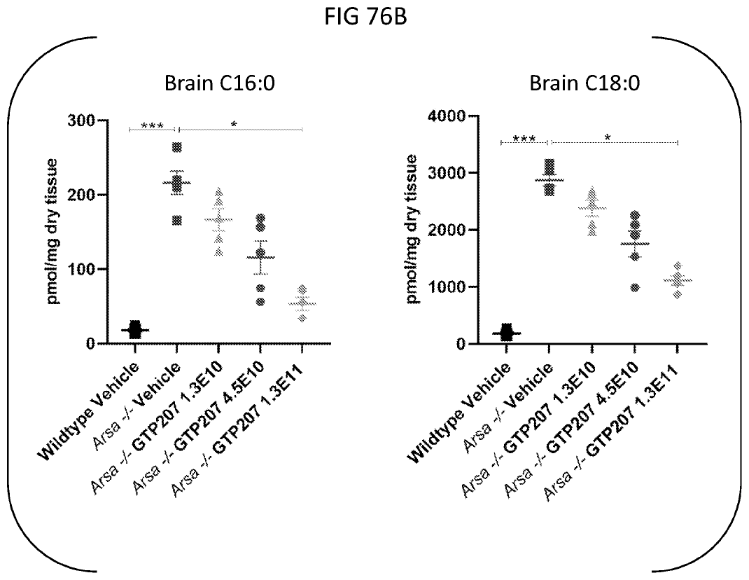

ARSA enzyme activity was increased 15 months after AAVhu68.CB7.CI.hARSAco.rBG (GTP-207) administration in all organs evaluated (liver, brain, and heart), and the ARSA enzyme activity levels observed were equivalent to or higher than that of vehicle-treated wild type animals. In the brain, the ARSA enzyme activity increase compared to vehicle-treated controls was 1.2-fold higher at the low dose (1.3×10¹⁰ GC) and mid-dose (4.5×10¹⁰ GC), and 1.3-fold higher at the high dose (1.3×10¹¹ GC). In the liver, the ARSA enzyme activity increase compared to vehicle-treated controls was 1.4-fold higher at the low dose (1.3×10¹⁰ GC), 5.3-fold higher at the mid-dose (4.5×10¹⁰ GC), and 7.1-fold higher at the high dose (1.3×10¹¹ GC). In heart, the ARSA enzyme activity increase compared to vehicle controls was 1.6-fold higher at the mid-dose (4.5×10¹⁰ GC), and 3.6-fold higher at the high dose (1.3×10¹¹ GC), while no increase in ARSA enzyme activity in the heart was observed at the low dose (1.3×10¹⁰ GC) (FIG. 76A).

Liquid chromatography-mass spectrometry (LC-MS) analysis was performed to quantify sulfatide storage in brain. Arsa^{-/-} mice demonstrated a significant increase in C16 and C18 sulfatide species, which was corrected with AAVhu68.CB7.CI.hARSAco.rBG (GTP-207) treatment (FIG. 76B).

### Summary of Results

Arsa^{-/-} mice exhibited a shortened lifespan, which was rescued by AAVhu68.CB7.CI.hARSAco.rBG (GTP-207) treatment at all doses (1.3×10¹⁰ GC [3.3×10¹⁰ GC/g brain], 4.5×10¹⁰ GC [1.1×10¹¹ GC/g brain], or 1.3×10¹¹ GC [3.3×10¹¹ GC/g brain]).
There were no statistical differences in body weights among groups.
Arsa^{-/-} mice exhibited a progressive neurological impairment measured by a compound clinical score that measure neurological and general health parameters. The neurological deterioration was significantly slowed in AAVhu68.CB7.CI.hARSAco.rBG (GTP-207) treated mice at all doses (1.3×10¹⁰ GC [3.3×10¹⁰ GC/g brain], 4.5×10¹⁰ GC [1.1×10¹¹ GC/g brain], or 1.3×10¹¹ GC [3.3×10¹¹ GC/g brain]).
Arsa^{-/-} mice exhibited progressive coordination impairment measured by the ledge test, which was partly ameliorated by AAVhu68.CB7.CI.hARSAco.rBG (GTP-207) treatment at the highest dose of 1.3×10¹¹ GC (3.3×10¹¹ GC/g brain).
Arsa^{-/-} mice exhibited progressive neuromotor impairment measured by the RotaRod assay, which was partly ameliorated by AAVhu68.CB7.CI.hARSAco.rBG (GTP-207) treatment at the at the mid-dose (4.5×10¹⁰ GC [1.1×10¹¹ GC/g brain]) and high dose (1.3×10¹¹ GC [3.3×10¹¹ GC/g brain]).
Arsa^{-/-} mice exhibited progressive gait impairment measured by the CatWalk system consisting of abnormal base of support, slower speed of movement, and decreased amplitude of paw movements (stride length), which were ameliorated by AAVhu68.CB7.CI.hARSAco.rBG (GTP-207) treatment at all doses: (1.3×10¹⁰ GC [3.3×10¹⁰ GC/g brain], 4.5×10¹⁰ GC [1.1×10¹¹ GC/g brain], or 1.3×10¹¹ GC [3.3×10¹¹ GC/g brain]).
AAVhu68.CB7.CI.hARSAco.rBG (GTP-207) administration increased the levels of ARSA enzyme activity to higher than or equivalent to wild type levels at all doses in brain and liver (1.3×10¹⁰ GC [3.3×10¹⁰ GC/g brain], 4.5×10¹⁰ GC [1.1×10¹¹ GC/g brain], or 1.3×10¹¹ GC [3.3×10¹¹ GC/g brain]) and in the heart at the mid-dose (4.5×10¹⁰ GC [1.1×10¹¹ GC/g brain]) and high dose (1.3×10¹¹ GC [3.3×10¹¹ GC/g brain]).
The lowest dose tested in this study (1.3×10¹¹ GC [3.3×10¹¹ GC/g brain]) demonstrated significant efficacy based on survival rescue, compound clinical scoring amelioration, gait improvement, and ARSA enzyme activity levels in brain and liver.

### Example 9—Efficacy of AAVhu68.CB7.CI.hARSAco.rBG (GTP-207) Following Intracerebroventricular Administration in ARSA^{-/-} Mice to Determine the Minimum Effective Dose

A study was performed to determine the minimum effective dose (MED) and transgene expression levels in a mouse model of infantile Metachromatic Leukodystrophy (MLD) disease following intracerebroventricular (ICV) administration of AAVhu68.CB7.CI.hARSAco.rBG (GTP-207).

Four-month-old Arsa^{-/-} mice received a single administration of AAVhu68.CB7.CI.hARSAco.rBG (GTP-207) at one of four dose levels (1.3×10¹¹ genome copies [GC][3.3×10¹¹ GC/g brain], 4.5×10¹⁰ GC [1.1×10¹¹ GC/g brain], 1.3×10¹⁰ GC [3.3×10¹⁰ GC/g brain], or 4.5×10⁹ GC [1.1×10¹⁰ GC/g brain]). Additional Arsa^{-/-} mice and wild type mice were administered either vehicle (intrathecal final formulation buffer [ITFFB]) or remained untreated baseline controls. The age of the animals was selected to model the disease stage of early symptomatic patients.

Group designations, dose levels, and the route of administration (ROA) are presented in the table below.

TABLE

| | | | | | | | Sacrifice: |
|---|---|---|---|---|---|---|---|
| | | | | | | | 180 + /− |
| | | | Affected | WT | | | 5 Days |
| | | Dose | Mice | Mice | | | Post |
| Group | Treatment^{b} | Volume | (ARSA−/−) | ( + / + ) | In-Life | Sacrifice: | Injection |
| # | (Dose) | (mL) | (N) | (N) | Evaluation | baseline | (N) |
| 1 | None | N/A | — | 10 (5M, | None | 10 (WT) | N/A |
| | | | | 5F) | | 10 | |
| 2 | None | N/A | 10 (5M, | | — | (ARSA−/−) | |
| | | | 5F) | | | | |
| 3 | Vehicle | 0.005 | — | 20 (10M, | Daily Viability | N/A | 20 |
| | (Artificial | | | 10F) | check | | |
| | CSF ITFFB) | | | | Weekly Clinical | | |
| 4^{c} | Vehicle | 0.005 | 20 (10M, | — | Observation | N/A | 20 |
| | (Artificial | | 10F) | | Weekly body | | |
| | CSF ITFFB | | | | weight | | |
| 5^{d} | GTP-207 | 0.005 | 20 (10M, | — | Blood biomarker | N/A | 20 |
| | 1.3 × 10¹¹ GC | | 10F) | | evaluations | | |
| 6 | GTP-207 | 0.005 | 20 (10M, | — | (ARSA enzyme | N/A | 20 |
| | 4.5 × 10¹⁰ GC | | 10F) | | activity and | | |
| 7 | GTP-207 | 0.005 | 20 (10M, | — | sulfatide | N/A | 20 |
| | 1.3 × 10¹⁰ GC | | 10F) | | quantification) | | |
| 8 | GTP-207 | 0.005 | 20 (10M, | — | Neurological | N/A | 20 |
| | 4.5 × 10⁹ GC | | 10F) | | score | | |

Values were calculated using 0.4 g brain mass for an adult mouse. One animal 1351 (Vehicle, ITFFB; Group 4) was found dead on study day 144. No clinical abnormalities were noted, and the animal was recorded as alive during previous viability check. One mouse, 5988 (GTP-207, 1.3×10¹¹ GC; Group 5) was euthanized per veterinary recommendation on study day 60 following a procedure related complication, the animal exhibited a decreased respiratory rate with transient increased respiratory effort. The ears, front paws, and muzzle were pale. One animal 8566 (GTP-207, 1.3×10¹¹ GC; Group 5) was found dead on study day 112. No clinical abnormalities were noted, and the animal was recorded as alive during previous viability check. Abbreviations: Arsa, arylsulfatase A (gene, mouse); F, female; GC, genome copies; ID, identification number; IV, intravenous; M, male; N, number of animals; N/A, not applicable; ROA, route of administration; WT, wild type; ml, milliliter; ITFFB, Intrathecal Final Formulation Buffer.

This study employed four dose levels of AAVhu68.CB7.CI.hARSAco.rBG (GTP-207) to identify the MED following ICV administration. The highest dose of 1.3×10¹¹ genome copies [GC][3.3×10¹¹ GC/g brain] was selected because it is near the maximum feasible dose in a mouse, which is limited by volume constraints (preferably 5.0 μL or less for ICV administration in adult mice) and by the expected vector titers. The maximum feasible dose and three lower doses of 4.5×10¹⁰ GC [1.1×10¹¹ GC/g brain], 1.3×10¹⁰ GC [3.3×10¹⁰ GC/g brain], or 4.5×10⁰⁹ GC [1.1×10¹⁰ GC/g brain]) were selected to bracket the dose range planned for toxicology study in non-human primates.

4-month-old Arsa^{-/-} mice received a single administration of AAVhu68.CB7.CI.hARSAco.rBG (GTP-207) at one of four dose levels (1.3×10¹¹ , 4.5×10¹⁰ GC, 1.3×10¹⁰ GC, 4.5×10⁰⁹ GC). Additional Arsa^{-/-} mice and wild type mice were administered either vehicle (intrathecal final formulation buffer [ITFFB]) or remained untreated as controls.

In-life assessments included daily viability checks, body weight measurements, neurological exams (baseline, day 90 and day 180), evaluation of serum transgene expression (ARSA enzyme activity) and quantification of sulfatide levels in plasma (day 170). Necropsies were performed on the day of dosing (age 4 months [untreated mice, baseline cohort]) and day 180 post dosing. At necropsy, a comprehensive list of tissues was collected for histopathological evaluation. Samples of the brain, spinal cord, and sciatic nerves were collected for evaluation of lysosomal dysfunction (LAMP-1 staining) along with neuroinflammation (only in brain and spinal cord) (Glial fibrillary acidic protein [GFAP] immunohistochemistry [IHC]). Rostral brain (for subgroups 1 & 3), liver and heart were collected for transgene expression assay (ARSA enzyme activity). Rostral Brain (for subgroup 2 & 4), spinal cord, sciatic nerve, liver, and kidney were collected for sulfatide analysis using liquid chromatography mass spectrometry (LC-MS). Blood was collected for complete blood counts (CBCs) with differentials and serum clinical chemistry analysis.

Viability was evaluated twice daily, and body weights were measured once a week to monitor for weight loss (i.e., body wasting or cessation of weight gain), which could be expected with deteriorating neuromotor function and is similarly observed in patients with MLD. Clinical scoring assessments of tremors, gait and coordination, clasping reflex, posture, and fur quality were performed monthly and were based on the known phenotype of previously generated mouse models of ataxia. Clinical scoring enabled evaluation of disease progression, including the development of ataxia, which is similarly observed in MLD patients (Guyenet et al., 2010); higher clinical scores would indicate a more severe phenotype.

To evaluate whether AAVhu68.CB7.CI.hARSAco.rBG (GTP-207) administration increased transgene product expression (ARSA enzyme) in Arsa^{-/-} mice, ARSA activity was measured in serum, and in disease-relevant tissues (brain) and in tissues known to be transduced after AAV ICV administration (brain, liver, heart) to assess pharmacology of AAVhu68.CB7.CI.hARSAco.rBG (GTP-207).

In addition, key target tissues for the treatment of MLD (CNS [brain, spinal cord] and PNS [sciatic nerve]), along with peripheral organs (liver, kidney) and plasma were collected to evaluate sulfatide storage (LC/MS) because sulfatides are the toxic substrates that accumulate in the absence of functional Arsa enzyme and those tissues are disease-relevant targets in both mice and humans with MLD. Lysosomal storage lesions (LAMP1 IHC) and astrogliosis/neuroinflammation (GFAP IHC) were also evaluated in the brain and spinal cord because these are neuropathologic hallmarks of MLD in mice and humans, which increase over time as the disease progresses.

### Clinical Scoring Assessment

Neurological examination and scoring of the mice were performed at baseline, Day 90 (±3 days), and Day 180 (±5 days) using an unpublished assessment of clasping ability, gait, tremor, kyphosis, and fur quality as detailed in the table below. These measures effectively assessed the clinical status of Arsa^{-/-} mice based upon the symptoms they typically present. Scores above 0 indicated clinical deterioration. The evaluator was blinded for the group information of the animals during the scoring procedure.

TABLE
Clinical Scoring Assessment

| Assessment | | |
|---|---|---|
| Category | Observation | Score |
| Fur Quality | Shiny coat, smooth fur, well groomed, clean coat | 0 |
| | Not well groomed, slightly oily or rough coat | 1 |
| | Rough Hair coat-hair coat is oily, dirty, stands on end | 2 |
| | Very rough hair coat, dehydration | 3 |
| Gait | Active, energetic. Body weight supported on all limbs. Even hindlimbs, | 0 |
| | abdomen raised | |
| | Able to run, but sluggish. Limp when walking. | 1 |
| | Reluctant to move, severe limp, lowered pelvis, feet point away from body | 2 |
| | Moves only when manually prodded & minimally, drags abdomen | 3 |
| Tremor | No sign of tremor | 0 |
| | Very slight, impermanent | 1 |
| | Mild, visible upon close observation | 2 |
| | Moderate, visible immediately | 3 |
| | Severe, impacts mobility | 4 |
| Clasping | Hindlimbs consistently splayed outward from abdomen | 0 |
| | 1 hindlimb is retracted toward abdomen for >50% time | 1 |
| | Both limbs partially retracted for >50% of time | 2 |
| | Hindlimbs entirely retracted and touching abdomen for >50% time | 3 |
| | Permanent full grasping of 4 limbs | 4 |
| Posture | Normal, flat back-easily straightens spine as it walks | 0 |
| | Transitory hunching, evident only when resting-mild but able to straighten | 1 |
| | spine | |
| | Rests upright with back hunched, unable to straighten completely when | 2 |
| | moving but mild | |
| | Pronounced and maintained kyphosis while walking and sitting | 3 |

### Histological Processing and Evaluation

### LFB/PAS Staining (Evaluating Myelination)

Following deparaffinization, sections of the brain, spinal cord, and sciatic nerve were stained with LFB/PAS staining. Briefly, slides were incubated with LFB solution (SLMP, LLC; Catalog number: STLFBPT) overnight at 65° C. Sections were differentiated in a 0.05% lithium carbonate solution and 70% ethanol and monitored under the microscope until differentiation was completed. Slides were then placed in 0.5% periodic acid for 5 minutes (Sigma; Catalog number: 395B-1Kit). After washing with running tap water, the slides were transferred into Schiff's reagent (Sigma; Catalog number: 395B-1Kit) for 15 minutes. Slides were washed with running tap water for 5 minutes, counterstained briefly with hematoxylin for nuclei identification, and coverslipped. Histopathological evaluation was performed.

### Immunohistochemistry Stainings (LAMP-1, GFAP, ARSA)

IHC was performed on sections from FFPE tissues on a Leica Bond Rx autostainer following a standard immunohistochemistry (IHC) protocol with the Bond polymer detection system (Leica Biosystems, DS9800) and DAB as chromogen. For all stains citrate buffer pH6 was used for antigen retrieval (20 min). LAMP1 was detected with monoclonal rat antibody 1D4B (Abcam ab25245, diluted 1:50), GFAP with a rabbit antibody from Abcam (ab7260, diluted 1:4000), and ARSA with a rabbit antibody from Sigma (HPA005554, diluted 1:200). Incubation time for all primary antibodies was set to 30 min. Ready to use secondary polymer antibodies were either from Vector Laboratories (anti-rat for LAMP1, MP-7444, incubation time 20 min) or from Leica (for GFAP and ARSA rabbit antibodies, BOND Polymer Refine Detection DS9800, incubation time 8 min). After staining slides were dehydrated through ethanol and xylene and coverslipped.

The LAMP-1 and GFAP IHC were quantified using image analysis software. Briefly, well-stained and intact regions of sections of the brain, spinal cord, and sciatic nerve were manually outlined using VIS version 2019.07.0.6328 (Visiopharm, Hoersholm, Denmark). For the brain, LAMP-1 positive area was quantitated via thresholding using the IHS-S (Intensity, Hue, Saturation model) classification feature. The LAMP-1-negative area was quantified via thresholding using the HDAB-Hematoxylin classification feature, and the LAMP-1-positive and LAMP-1-negative area classifications were used to generate the percentage of the outlined section that was LAMP-1 positive, the number of LAMP-1 positive objects, and the average size of all LAMP-1 objects identified in the section. For the spinal cord, LAMP-1 positive and LAMP-1 negative areas were quantified via thresholding using the HDAB-DAB classification feature, and the LAMP-1-positive and LAMP-1-negative area classifications were used to generate the percentage of the outlined section that was IBA1-positive, the number of LAMP-1 positive objects, and the average size of all LAMP-1 objects identified in the section. For the sciatic nerve, the LAMP-1-positive area was quantitated via thresholding using the HDAB-DAB classification feature. The LAMP-1 negative area and empty space induced by processing were quantified via thresholding using the HDAB-Hematoxylin classification feature, and the LAMP-1 positive and LAMP-1 negative area (but not the empty space) classifications were used to generate the percentage of the outlined section that was LAMP1-positive, the number of LAMP-1 positive objects, and the average size of all LAMP-1 objects identified in the section.

### Quantification of Sulfatide Storage by LC/MS

Thawed tissues were lyophilized overnight and ground to a fine powder in 2.0 mL polypropylene tubes with ceramic beads using a Precellys bead-beating homogenizer (Bertin Technologies, Rockville, MD) at 4° C. Aliquots of powder (˜2.5-5.0 mg) were weighed on an analytical balance followed by homogenization in 500 μL of 80% methanol in the Precellys homogenizer at 4° C. A 100 μL aliquot of homogenate was then spiked with 10 μL of a C18:0-CD3-sulfatide internal standard (N-omega-CD3-Octadecanoyl-sulfatide Matreya State College, PA, catalog #1536; 25 μM) and extracted with 400 μL of ice cold methanol in a 2.0 mL Eppendorf tube. The sample was centrifuged for 5 minutes at 14,000×g at 4° C. Aliquots (400 μL) of methanolic supernatants were dried under nitrogen in a 96-well plate at 45° C. and reconstituted in 150 μL of methanol for LC/MS analysis.

Calibration samples of sulfatide standards were prepared. Standard powders of sulfatides (lysosulfatide catalog #1904; C16:0 catalogue #1875, C18:0 catalogue #1932, C18:0-CD3 catalogue #1536, and C24:1 catalogue #1931; Matreya, State College, PA) were weighed on an analytical balance, and individual stock solutions (1 mM) were prepared in 2:1 methyl tert-butyl ether/methanol. The C18:0-CD3sulfatide internal standard stock solution was diluted in methanol to give a 25 μM spiking internal standard solution. Aliquots of the individual stock solutions were combined to make a high calibration spiking solution of 50 μM lysosulfatide, 50 μM C16:0, 250 μM C18:0, and 250 μM C24:1 sulfatide in methanol. This high calibration spiking solution was serially diluted in methanol to make 0.1, 0.25, 0.5, 1, 5, 10, 25, and 50 μM calibration curve spiking solutions for lysosulfatide and C16:0 sulfatide, along with 0.5, 1.25, 2.5, 5, 25, 50, 125, and 250 μM calibration curve spiking solutions for C18:0 and C24:1 sulfatide. Calibration curve solutions for LC/MS analysis were created by pipetting 10 μL of each spiking solution and 10 μL of the C18:0-CD3-sulfatide internal standard (25 μM) into 100 μL of 80% methanol, resulting in LC/MS calibration curves of 0.01, 0.025, 0.05, 0.1, 0.5, 1, 2.5, and 5 μM for lysosulfatide and C16 sulfatide, along with LC/MS calibration curves of 0.05, 0.125, 0.25, 0.5, 2.5, 5, 12.5, and 25 μM for C18:0 and C24:1 sulfatide. A 400 μL aliquot of methanol was added to each solution. The sample was vortexed, and 400 μL was dried under nitrogen in a 96-well plate at 45° C. and reconstituted in 150 μL of methanol for LC/MS analysis.

Sulfatides were quantified with an Agilent 1290 Infinity UHPLC/6495B triple quadrupole mass spectrometer. Biological extracts and calibration solutions in 96-well plates were injected (5 μL) and separated on the UHPLC. Sulfides were eluted by gradient elution on a Waters Acquity BEH C18 2×100 mm, 1.7 μM column at a flow rate of 0.4 mL/minute at 45° C. A 7.5 minute gradient was used beginning with 35% solvent A (70/30 deionized water/acetonitrile/0.1% formic acid) and 65% solvent B (50/50 acetonitrile/isopropanol/0.1% formic acid) held for 0.5 minutes and increased to 100% solvent B over 5.5 minutes, held at 100% solvent B until 7.5 minutes followed by re-equilibration back to starting conditions from 7.6 to 10 minutes. The HPLC flow was diverted to waste for the first 0.5 minute then directed to the electrospray ionization source. Sulfatides were ionized by electrospray ionization in the positive ionization mode on the mass spectrometer. The Agilent Jet Stream electrospray ionization source was operated with a nitrogen gas temperature of 250° C., gas flow of 14 L/minute, nebulizer of 45 psi, sheath gas temperature of 325° C., sheath gas flow of 12 L/minute, capillary voltage of 3500 V, and nozzle voltage of 500 V. Multiple reaction monitoring (MRM) was used to quantitate sulfatides with a peak width of 0.7 Da and an electron multiplier voltage of 400 V in the positive ion mode. As an example, a primary transition for C16:0 sulfatide of m/z 780.57-264.2 was used for the quantitation of C16:0 by monitoring m/z 264.2, the while the secondary transition m/z 780.57-682.6, generated by neutral loss of H₂SO₄ from the parent ion, was used to confirm the primary transition as an authentic sulfatide. Agilent MassHunter software was used to generate linear or quadratic calibration curves (1/x or 1/x2 weighting and R2 0.99 or better) to quantify sulfatides in biological samples.

### Measuring ARSA Activity

ARSA enzyme activity was measured in dialyzed serum or tissues samples using a p-nitrocatechol assay. Briefly, dialyzed serum (diluted 1:5, 1 part serum+4 parts diluent) or tissues were diluted into a base buffer (0.5 M sodium acetate buffer, pH 5.0; 10% sodium chloride; 0.5 mM sodium pyrophosphate) and 40 μL of diluted sample was loaded into four wells (2 duplicates) of a 96-well plate. Next, 40 μL of substrate (10 mM 4-nitrocatechol sulfate in base buffer) was added to the samples, and the reaction was stopped immediately by adding 120 μL 1N NaOH (stop solution) in two of the four wells. The plate was then incubated at 37° C. for 5 hours. The reaction was stopped by adding 120 μL 1N NaOH (stop solution) in the remaining wells. The absorbance was measured by reading the plate at 515 nm using a plate reader. ARSA-specific activity was determined by multiplying the absorbance obtained at five hours minus the absorbance at 0 minute with the extinction coefficient of a 4-nitrocathecol (4-NC) standard curve at 515 nm and by dividing by the amount of protein in the well (mg) as measured by BCA assay. The results for ARSA activity were expressed as nmol 4-NC generated per milligram tissue or per milliliter serum per five hours.

### Results

### Clinical Observations

No clinical abnormalities related to AAVhu68.CB7.CI.hARSAco.rBG (GTP-207) were noted during the study.

### Body Weights

Vehicle treated Arsa^{-/-} mice started the study with lower body weights on average than WT mice and demonstrated statistically significantly lower body weights beginning at Study Day 21 in males and Study Day 84 in females compared to age matched vehicle-treated WT controls.

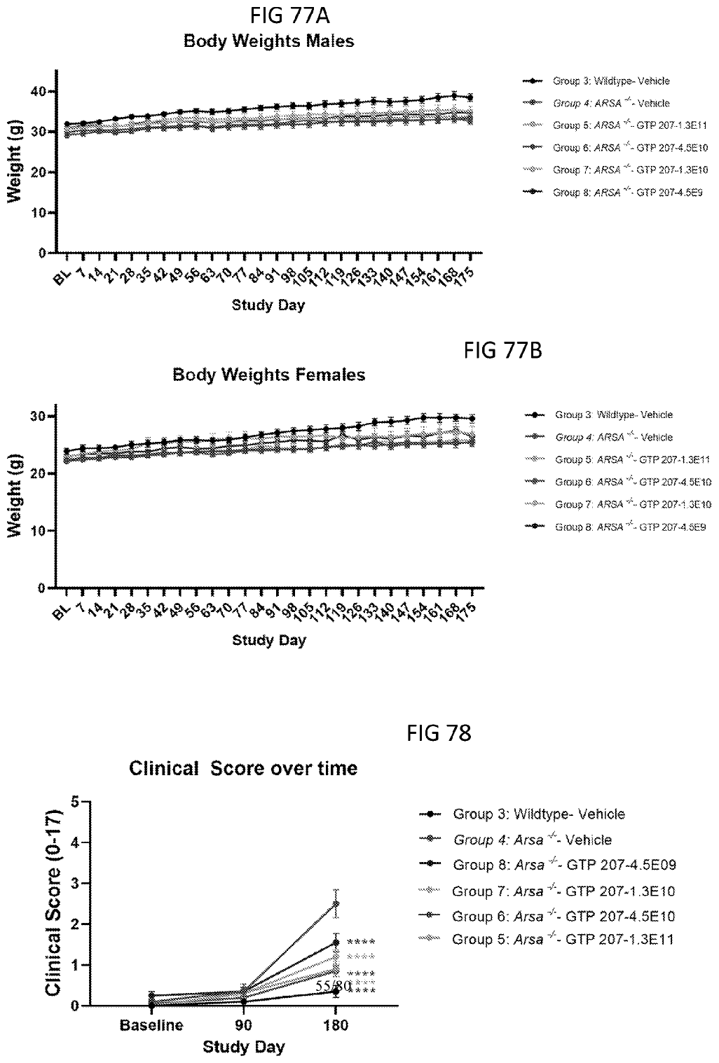

Body weights were stable throughout the study in all groups and administration of AAVhu68.CB7.CI.hARSAco.rBG (GTP-207) at the four doses (1.3×10¹¹ GC, 4.5×10¹⁰ GC, or 1.3×10¹⁰ GC, 4.5×10⁹ GC) did not lead to any significant fluctuation in body weights in either male or female Arsa^{-/-} mice (FIG. 77).

### Clinical Scoring Assessments

Clinical scoring was used to assess the clinical status of mice, with scores above 0 indicating clinical deterioration.

As observed in a natural history study of our Arsa^{-/-} model, a compound clinical scoring blinded assessment measuring clasping reflex, gait, tremors, kyphosis, and fur quality, showed clinical deterioration of Arsa^{-/-} mice compared to WT controls from Study Day 180 (10 months of age). Administration of AAVhu68.CB7.CI.hARSAco.rBG (GTP-207) at all doses (1.3×10¹¹ GC, 4.5×10¹⁰ GC, 1.3×10¹⁰ GC, 4.5×10⁹ GC) to Arsa^{-/-} mice led to a significant reduction in severity scores compared to that of age-matched vehicle-treated Arsa^{-/-} mice. This result indicates that AAVhu68.CB7.CI.hARSAco.rBG (GTP-207) administration led to a dose-dependent reduction of phenotype in a mildly symptomatic Arsa^{-/-} mouse model (FIG. 78).

### Transgene Expression and Anti-Transgene Antibodies (ELISA)

Transgene expression was measured by enzyme activity assays in blood serum and tissue lysates (brain, liver, heart) as well as by immunostaining (IHC) in brain formalin fixed paraffin embedded (FFPE) sections.

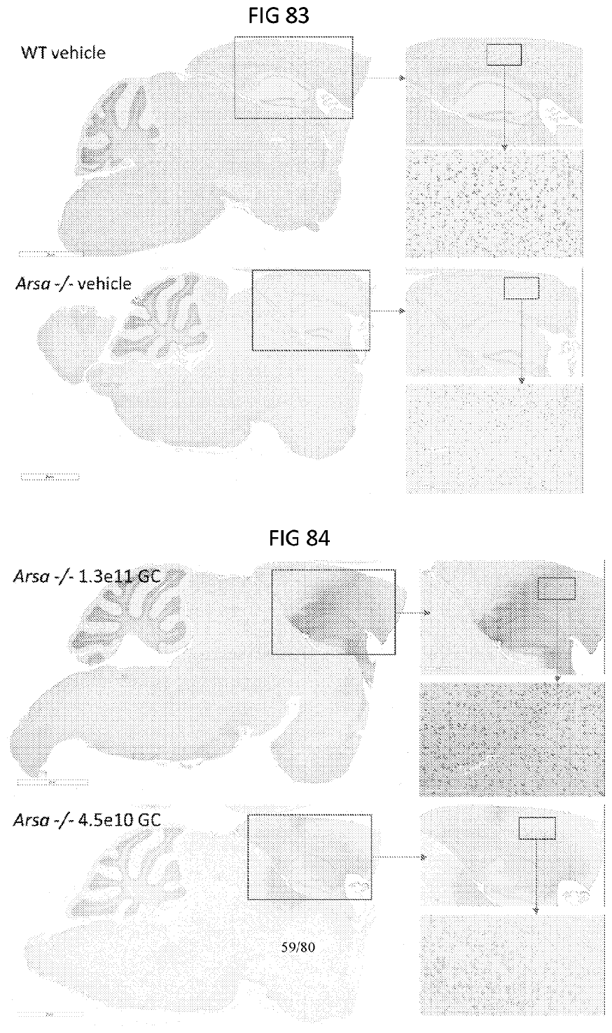

No detectable ARSA protein was measured by immunostaining in Arsa^{-/-} vehicle controls (FIG. 83). Although Arsa^{-/-} animals have measurable enzyme activity levels due to nonspecific activity of other sulfatases towards the artificial substrate, any increase over Arsa^{-/-} vehicle controls can be attributed to AAVhu68.CB7.CI.hARSAco.rBG (GTP-207) administration and hARSA transgene expression as other sulfatases are not expected to be modified by the treatment.

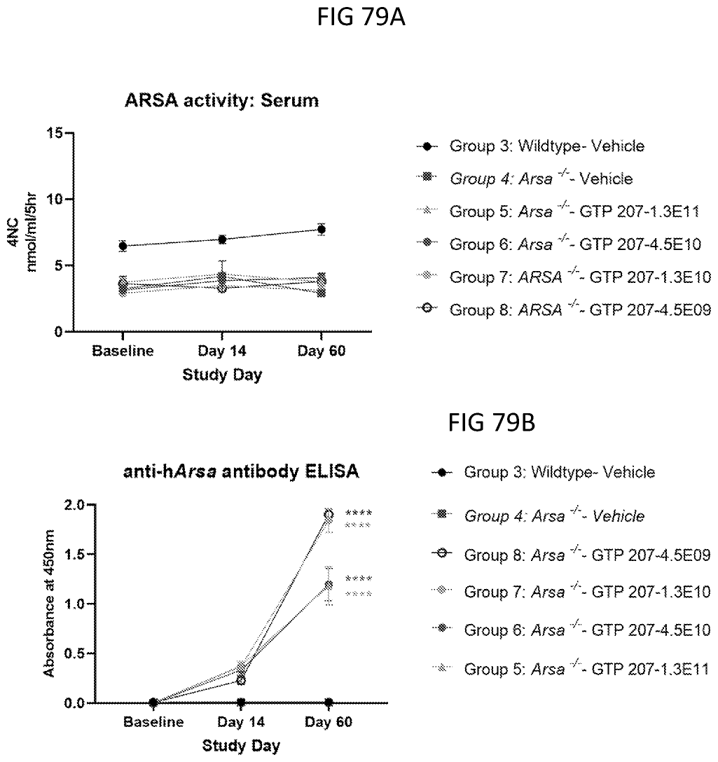

Administration of AAVhu68.CB7.CI.hARSAco.rBG (GTP-207), regardless of dose, did not lead to significant increases in ARSA serum enzyme activity on Study Day 14 and Study Day 60 as compared with Baseline measurements (FIG. 79). It is hypothesized that this may be due to the presence of anti-transgene antibodies since circulating anti-hARSA antibodies were detected as early as Study Day 14 (FIG. 79).

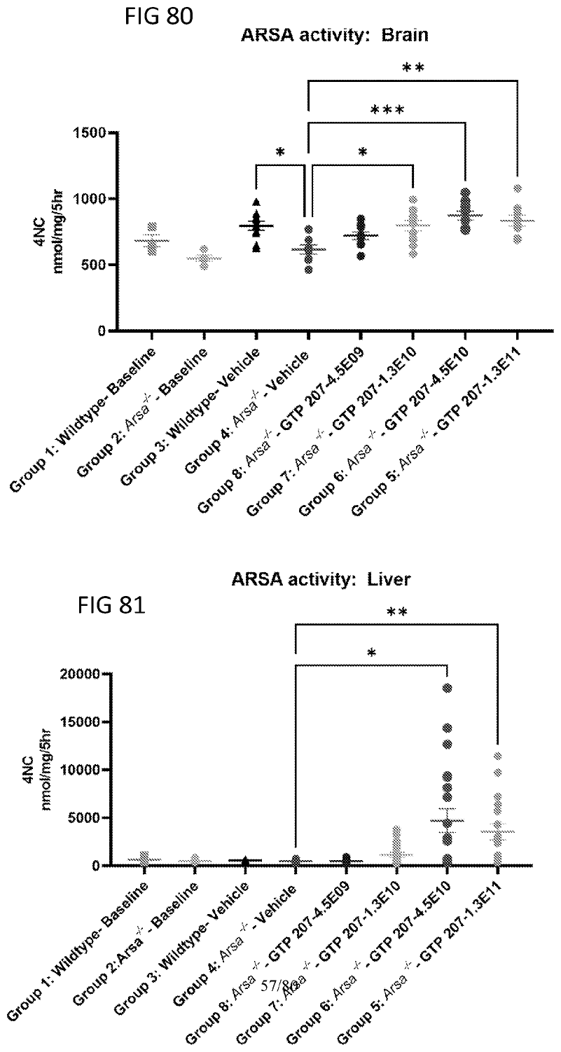

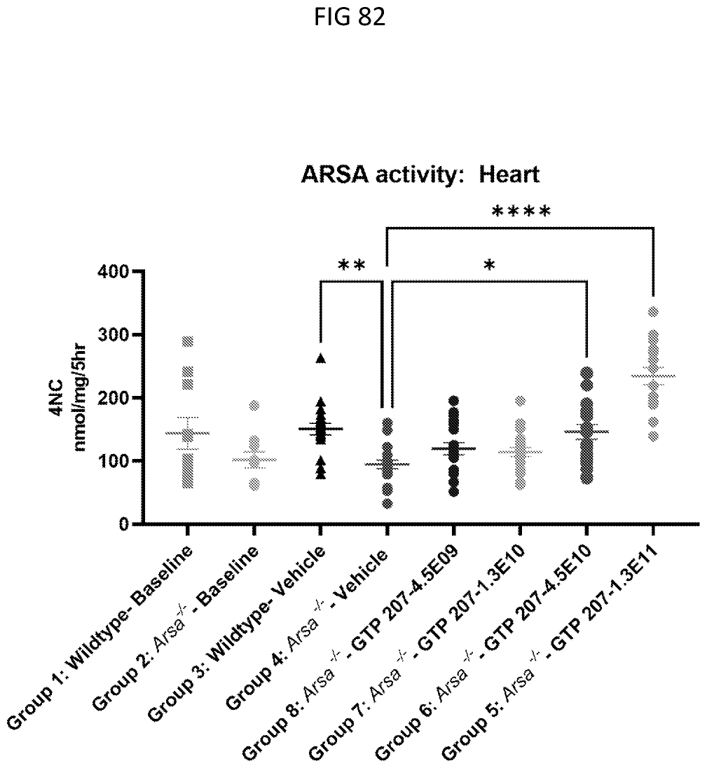

In the brain, administration of AAVhu68.CB7.CI.hARSAco.rBG (GTP-207) at the three highest doses (1.3×10¹¹ GC, 4.5×10¹⁰ GC, or 1.3×10¹⁰ GC) to Arsa^{-/-} mice resulted in a significant dose-dependent increase in ARSA enzyme activity compared to that of vehicle-treated Arsa^{-/-} mice. Furthermore, these doses of AAVhu68.CB7.CI.hARSAco.rBG (GTP-207) (1.3×10¹¹ GC, 4.5×10¹⁰ GC, or 1.3×10¹⁰ GC) increased average ARSA enzyme activity in Arsa^{-/-} mice to levels comparable to vehicle-treated wild type controls (FIG. 80). In liver and heart, administration of AAVhu68.CB7.CI.hARSAco.rBG (GTP-207) at the two highest doses (1.3×10¹¹ GC, 4.5×10¹⁰ GC) resulted in a significant dose-dependent increase in ARSA enzyme activity compared to that of vehicle-treated Arsa^{-/-} mice (FIG. 81 and FIG. 82). Levels in liver were higher in males compared to females, as expected, due to androgen-dependent enhancement of AAV-mediated liver transduction in male mice. This phenomenon has not been reported in other species.

hARSA IHC was performed on a subset of animals from each group (N=2/group) in order to analyze qualitatively the profile of expression of hARSA protein in the brain following the administration of AAVhu68.CB7.CI.hARSAco.rBG (GTP-207) or vehicle. In WT animals, ARSA IHC showed a generalized ARSA expression throughout the entire brain due to cross reactivity of the antibody with murine ARSA (FIG. 83). Under higher magnification, the ARSA protein signal appeared punctate in the cytoplasm, consistent with a lysosomal localization. In contrast, the vehicle treated Arsa^{-/-} mouse did not show any positive ARSA staining in any region of the brain, showing the specificity of the immunostaining for ARSA (FIG. 83).

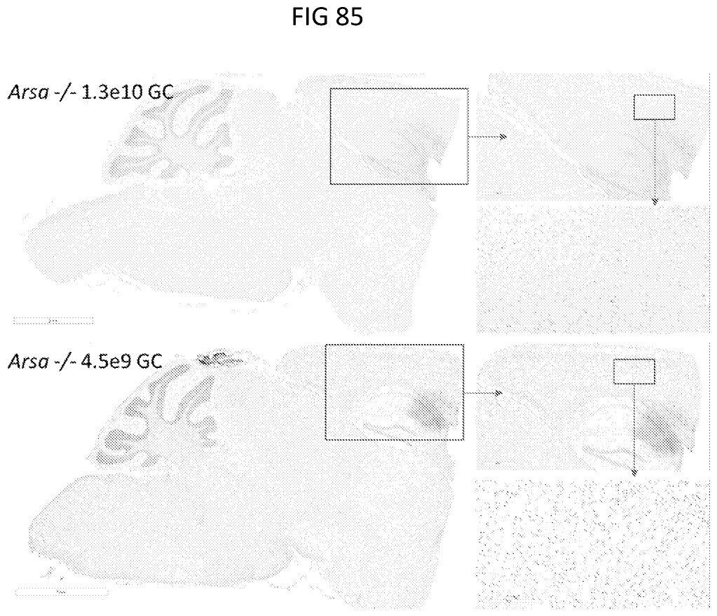

AAVhu68.CB7.CI.hARSAco.rBG (GTP-207) administration resulted in a dose-dependent increase in hARSA IHC signal. The hARSA staining was particularly strong near the injection site (lateral ventricle) and was seen in cerebral cortex, hippocampus, and corpus callosum in all the treated cohorts. In the two highest dose groups (1.3×10¹¹ GC and 4.5×10¹⁰ GC; FIG. 84), the distribution of hARSA positive staining was more extensive than in the two lower-dose groups (FIG. 85), with hARSA staining in cells in the mid brain and brain stem in addition to cerebral cortex, hippocampus, and corpus callosum. In the two lowest dose groups (1.3×10¹⁰ GC and 4.5×10⁹ GC; FIG. 85), the distribution of hARSA positive staining was restricted to smaller areas largely in hippocampus and cerebral cortex. Under higher magnification, hARSA protein expression appeared punctate in the cytoplasm and similar in distribution to the cellular localization seen in WT mice. Staining was also seen in the neuropil (FIG. 84 and FIG. 85).

### Clinical Pathology (Clinical Chemistry and Hematology)

### Serum Clinical Chemistries

No toxicity associated with AAVhu68.CB7.CI.hARSAco.rBG (GTP-207) treatment was observed in the serum clinical chemistry parameters evaluated.

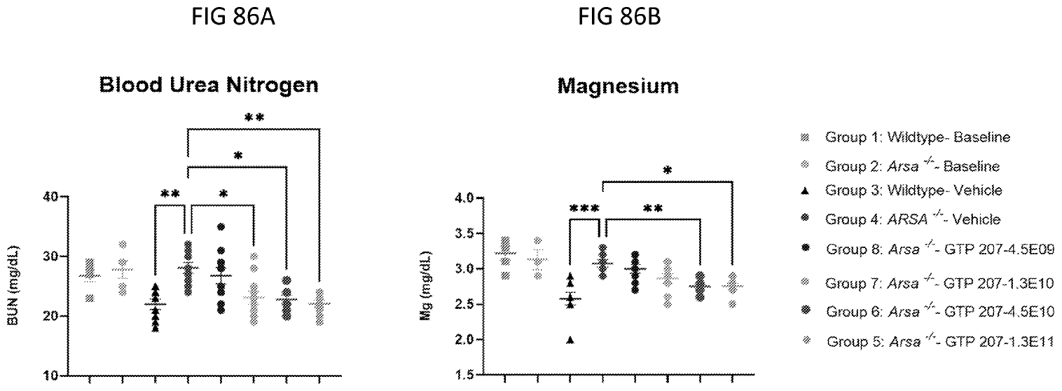

Some genotype-related abnormalities in Arsa^{-/-} mice were observed in kidney parameters that were rescued by AAV treatment. At Study Day 180 serum chemistry showed significant increases in blood urea nitrogen (BUN) and in magnesium levels in the vehicle-treated Arsa^{-/-} mice when compared to levels in vehicle-treated wild type controls. Administration of AAVhu68.CB7.CI.hARSAco.rBG (GTP-207) at the three highest doses (1.3×10¹¹ GC, 4.5×10¹⁰ GC, and 1.3×10¹⁰ GC) significantly improved BUN levels which were restored to wild-type levels, and the two highest doses (1.3×10¹¹ GC and, 4.5×10¹⁰ GC) significantly improved magnesium levels in Arsa^{-/-} mice (FIG. 86).

Total protein and globulin levels were slightly elevated in Arsa^{-/-} mice compared to age-matched WT controls (***p<0.001); no treatment effect was observed for those parameters.

The rest of the serum chemistry parameters evaluated did not show any statistically significant differences between vehicle treated Arsa^{-/-} mice and wild type controls.

### Hematology

No toxicity associated with AAVhu68.CB7.CI.hARSAco.rBG (GTP-207) treatment was observed in the hematology parameters evaluated.

### Histology

### Histopathology, Safety Readout

The purpose of the pathology portion of study was to evaluate the toxicity of intracerebroventricular (ICV) delivery of AAVhu68.CB7.CI.hARSAco.rBG (GTP-207) in ARSA^{-/-} (ARSA KO) mice. AAVhu68.CB7.CI.hARSAco.rBG (GTP-207)-injected mice were evaluated at Study Day 180 following administration of one of four doses on Study Day 0: 1.3×10¹¹ GC (high; Group 5), 4.5×10¹⁰ GC (Group 6), 1.3×10¹⁰ GC (Group 7), 4.5×10⁹ GC (low; Group 8). To evaluate the presence or absence of test-article related toxicity, first all organs from high-dose treated mice were evaluated. For animals that received the three lower doses only organs with suspected findings at high dose were evaluated histopathologically. Baseline (Groups 1-2) and Day 180 (Groups 3-4) WT and ARSA KO mice served as controls.

There were no test-article related necropsy findings of gross tissue changes in any AAVhu68.CB7.CI.hARSAco.rBG (GTP-207)-treated Arsa^{-/-} mice. Microscopic changes were identified in some animals, the majority of which were considered incidental or related to the ARSA KO mouse phenotype. AAVhu68.CB7.CI.hARSAco.rBG (GTP-207)-treatment resulted in an increased incidence of minimal (grade 1) to mild (grade 2) sciatic nerve degeneration (incidence 34/39 grade 1, 1/39 grade 2) compared to WT (incidence 3/15, grade 1) and ARSA KO control mice (incidence 2/9, grade 1). The cause of this finding and its definitive relationship to AAVhu68.CB7.CI.hARSAco.rBG (GTP-207) is not clear. There were no microscopic abnormalities found in the DRG of GTP-207-treated mice, and rare degeneration of individual axons within the dorsal nerve roots was observed (incidence 5/39, grade 1).

### Histopathology, Efficacy Readout

The Arsa^{-/-} mouse histopathological phenotype consisted of cytoplasmic accumulation of storage materials in neurons and oligodendrocytes in the brain and spinal cord. The white matter tracts of the brain and spinal cord also exhibited vacuolation which was often associated with secondary axonal degeneration. These findings were most prominent in the white matter and nuclei of the cerebellum and brainstem, along with the white matter tracts of the frontal cortex. Vacuolation with evidence of axonal degeneration was observed in all white matter tracts throughout the spinal cord and were most prominent in the lateral and ventral tracts.

Periodic acid-Schiff (PAS) stain for polysaccharides was used to investigate further the storage material observed in CNS cells from Arsa^{-/-} mice, however sections only stained lightly with the PAS, suggesting the material was not consistent with polysaccharides nor glycolipids. Luxol fast blue (LFB) staining for myelin confirmed the presence of dilated myelin sheaths with axonal degeneration; however, no significant demyelination was observed. While efficacy was not the primary focus of this evaluation, AAVhu68.CB7.CI.hARSAco.rBG (GTP-207)-treated mice at the three highest doses (1.3×10¹⁰ GC, 4.5×10¹⁰ GC, or 1.3×10¹¹ GC) had reduced incidence and severity of white matter vacuolation and storage material in spinal cord (all dose groups were evaluated to investigate presence or absence of test-article related DRG toxicity. In brain, for which only the highest dose was evaluated (1.3×10¹¹ GC), there was also a reduced incidence and severity of phenotype-related white matter findings.

### Lysosomal Compartment Immunostaining (LAMP-1) and Astrogliosis

The Arsa^{-/-} mouse phenotype consisted of lysosomal compartment expansion as measured by LAMP-1 immunostaining and automated whole-slide quantification of the positive immunohistochemical signal in brain, spinal cord, and sciatic nerve.

At 4 months of age (baseline), untreated Arsa^{-/-} mice demonstrated lysosomal expansion as shown by the increase in LAMP-1 staining area ratio in the brain (cortex, hippocampus, corpus callosum, cerebellum and brain stem), spinal cord, and sciatic nerve when compared to untreated wild type controls. This shows that disease-related pathology was already present in Arsa^{-/-} mice at the time of AAVhu68.CB7.CI.hARSAco.rBG (GTP-207) administration.

At Study Day 180 (10 months of age), AAVhu68.CB7.CI.hARSAco.rBG (GTP-207)-treated Arsa^{-/-} mice exhibited improvements in lysosomal dysfunction indicated by a decrease in LAMP-1 staining when compared to that of vehicle-treated Arsa^{-/-} controls in several neuroanatomical areas examined, including brain [cortex, hippocampus, corpus callosum] and spinal cord [lumbar].

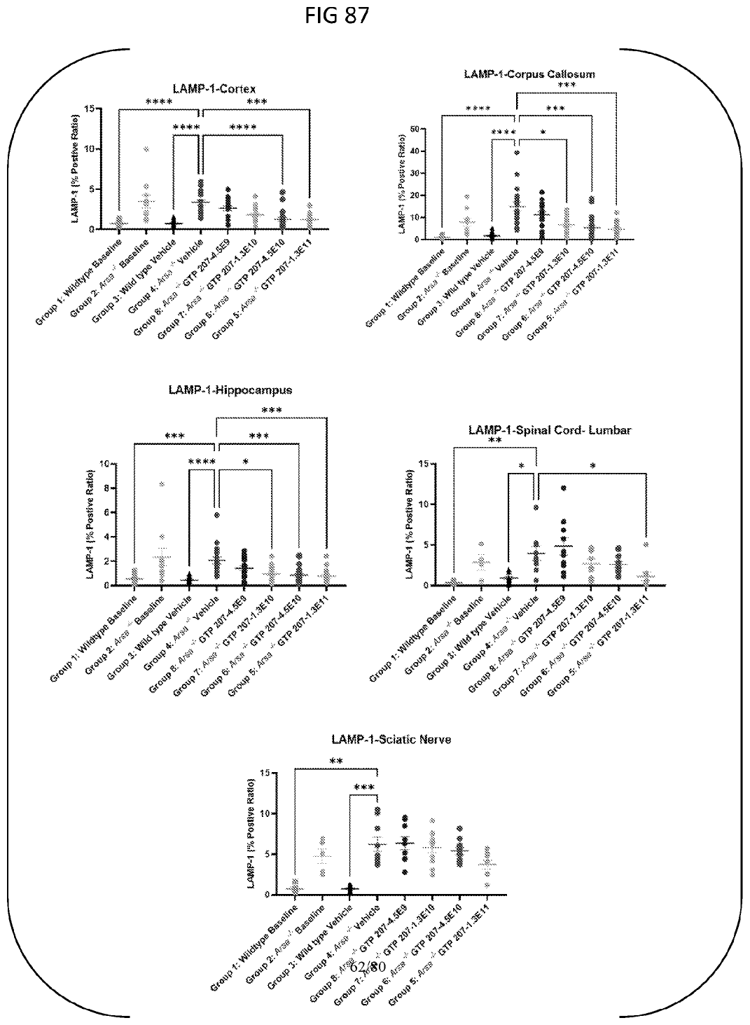

Administration of AAVhu68.CB7.CI.hARSAco.rBG (GTP-207) at the three highest doses (1.3×10¹⁰ GC, 4.5×10¹⁰ GC, or 1.3×10¹¹ GC) to Arsa^{-/-} mice led to a significant amelioration of lysosomal pathology (LAMP-1 immunostaining) in brain hippocampus and in the corpus callosum (white matter) when compared to aged-matched vehicle-treated controls (FIG. 87). This result suggests therapeutic benefit in the CNS at doses as low as 1.3×10¹⁰ GC in regions that were robustly transduced (as inferred by ARSA immunostaining. Importantly, LAMP-1 staining was already increased at baseline in Arsa^{-/-} mice, indicating a reversal of pathology after treatment with AAVhu68.CB7.CI.hARSAco.rBG (GTP-207).

In CNS regions with lower levels of Arsa transduction, the two highest doses of AAVhu68.CB7.CI.hARSAco.rBG (GTP-207) (4.5×10¹⁰ GC or 1.3×10¹¹ GC) significantly ameliorated lysosomal pathology (normalized LAMP-1 immunostaining) in cerebral cortex; and the high dose (1.3×10¹¹ GC) reduced LAMP-1 pathology in lumbar spinal cord (FIG. 87). CNS regions distal to the ICV delivery site that showed low to no apparent Arsa transduction (based on ARSA immunostaining), did not demonstrate decreases in LAMP-1 staining following AAVhu68.CB7.CI.hARSAco.rBG (GTP-207) treatment. These regions included cerebellum, brain stem, and rostral regions of the spinal cord (cervical and thoracic segments). In sciatic nerve, the highest doses of AAVhu68.CB7.CI.hARSAco.rBG (GTP-207) (1.3×10¹¹ GC) demonstrated a trend towards decrease in lysosomal dysfunction however the difference was not statistically significant).

### Astrocyte Immunostaining (GFAP)

The Arsa^{-/-} mouse phenotype consisted of cerebral and spinal cord astrogliosis as measured by GFAP immunostaining and automated whole-slide quantification of the immunohistochemical positive signal.

At 4 months of age (baseline, Group 2) untreated Arsa^{-/-} mice exhibited astrogliosis as indicated by the increased GFAP positive area ratio staining in some regions of the brain (cerebral cortex) and in the spinal cord. This shows that disease-related pathology was already present in Arsa^{-/-} mice at the time of AAVhu68.CB7.CI.hARSAco.rBG (GTP-207) administration. Corpus callosum, hippocampus, and cerebellum did not have significant astrogliosis in Arsa^{-/-} mice compared to WT mice.

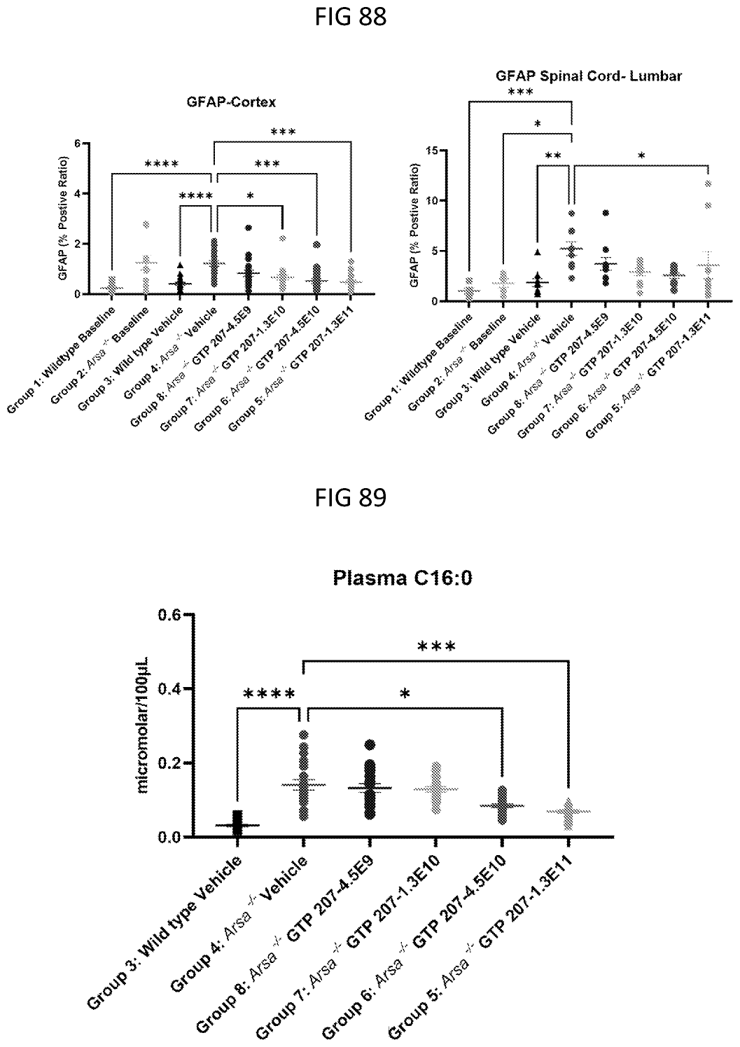

At Study Day 180 (10 months of age), AAVhu68.CB7.CI.hARSAco.rBG (GTP-207)-treated Arsa^{-/-} mice exhibited significant reductions in astrogliosis when compared to vehicle-treated Arsa^{-/-} controls. In the cortex, the three highest doses of AAVhu68.CB7.CI.hARSAco.rBG (GTP-207) (1.3×10¹¹ GC, 4.5×10¹⁰ GC, and 1.3×10¹⁰GC) showed a significant reduction in the percentage of evaluated tissue showing GFAP-positive staining when compared to that of vehicle-treated Arsa^{-/-} controls, while the highest dose (1.3×10¹¹ GC) led to a statistically significant reduction in GFAP staining area in lumbar spinal cord sections (FIG. 88).

### Sulfatide Analysis Using LC-MS

Liquid chromatography-mass spectrometry (LC-MS) analysis was performed to quantify sulfatide storage in plasma (collected on Study Day 170) and in tissues collected at necropsy (baseline and Study Day 180).

On Study Day 170, plasma samples were collected to determine the effect of AAVhu68.CB7.CI.hARSAco.rBG (GTP-207) on circulating levels of sulfatides. Vehicle-treated Arsa^{-/-} mice showed a significantly higher level of C16:0 (the only sulfatide detectible in plasma) compared to levels in age-matched vehicle-treated wild type controls. Administration of AAVhu68.CB7.CI.hARSAco.rBG (GTP-207) at the two highest doses (1.3×10¹¹ GC, 4.5×10¹⁰ GC) led to dose-dependent decreases of C16:0 levels in the plasma when compared to the vehicle-treated Arsa^{-/-} mice (FIG. 89).

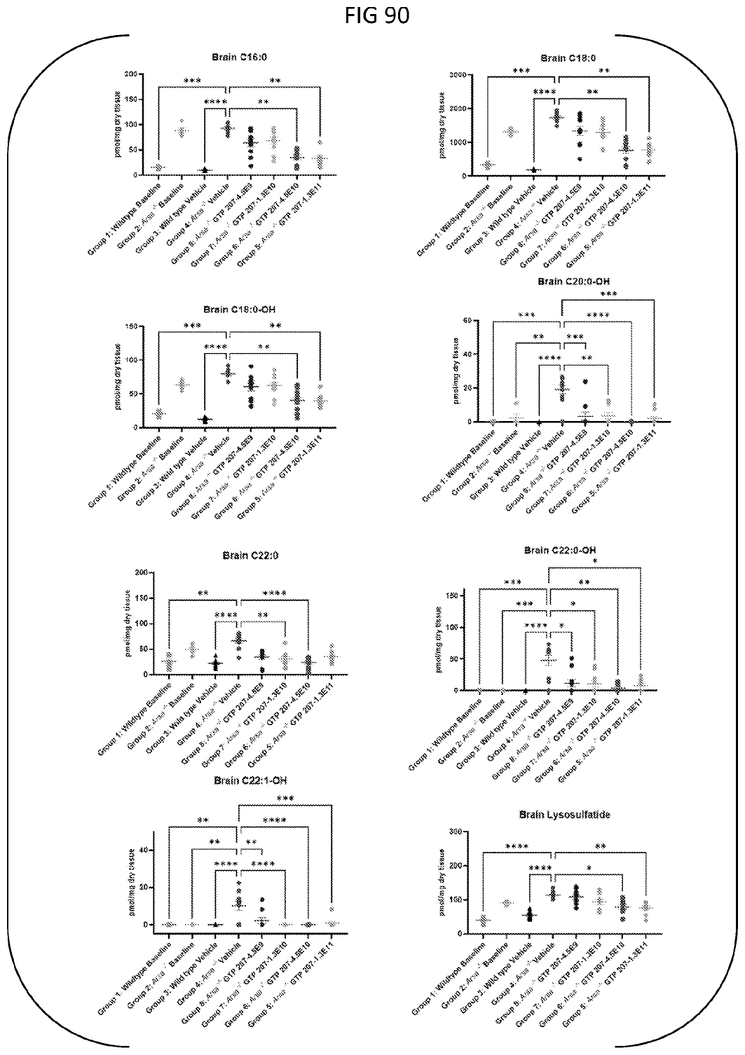

In the brain the majority of the sulfatide species analyzed were higher in vehicle-treated Arsa^{-/-} mice compared to levels in age-matched vehicle-treated wildtype controls both at baseline prior to AAV administration) and at Study Day 180. This shows that sulfatide storage pathology was present in the CNS at the time of AAVhu68.CB7.CI.hARSAco.rBG (GTP-207) treatment in four month-old Arsa^{-/-} mice administration of AAVhu68.CB7.CI.hARSAco.rBG (GTP-207) at the two highest doses (1.3×10¹¹ GC, 4.5×10¹⁰ GC) led to a significant correction of sulfatide storage, with reductions in the levels of several species (C16:0, C18:0, C18:0-OH, C20:0-OH, C22, C22:0-OH, C22:1-OH and lysosulfatide) compared to levels in the vehicle-treated Arsa mice. In addition, levels of C20:0-OH, C22:0-OH, and C22:1-OH species of sulfatides were significantly corrected following all four doses (1.3×10¹¹ GC, 4.5×10¹⁰ GC, 1.3×10¹⁰ GC, or 4.5×10⁹ GC) when compared to the vehicle-treated Arsa^{-/-} mice (FIG. 90).

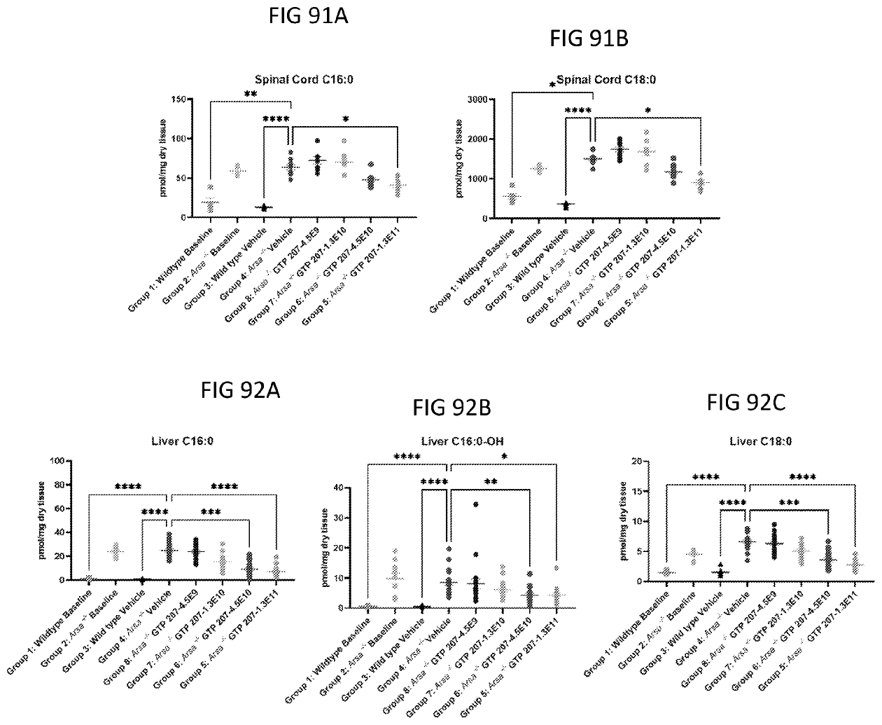

In the spinal cord, several sulfatide species analyzed were higher in the vehicle-treated Arsa^{-/-} mice compared to the age-matched vehicle-treated wildtype controls at baseline, prior to AAV administration, and at the study conclusion (Day 180). This shows that storage pathology was present in the spinal cord at the time of AAVhu68.CB7.CI.hARSAco.rBG (GTP-207) treatment. Administration of AAVhu68.CB7.CI.hARSAco.rBG (GTP-207) at the highest doses (1.3×10¹¹ GC) led to a significant correction of sulfatide storage with reductions in the levels of C16:0 and C18:0 sulfatides compared to levels in vehicle-treated Arsa^{-/-} mice. The second highest dose (4.5×10¹⁰ GC) also showed trends to decrease sulfatide levels although changes did not reach statistical significance (FIG. 91).

In liver the number of sulfatide species detected were comparatively fewer than those in the brain. Nonetheless, liver sulfatide levels were significantly higher in the vehicle-treated Arsa mice compared to the age-matched vehicle-treated wildtype controls at baseline (prior to AAV administration) and at Study Day 180. Administration of AAVhu68.CB7.CI.hARSAco.rBG (GTP-207) at the two highest doses (1.3×10¹¹ GC, 4.5×10¹⁰ GC) resulted in correction of sulfatide storage, with statistically significant reductions in the levels of several species (C16:0, C18:0, C16:0-OH,) compared to levels in vehicle-treated Arsa^{-/-} mice (FIG. 92).

In sciatic nerve all the sulfatide species that were detected were significantly higher in the vehicle-treated Arsa^{-/-} mice compared to the age-matched vehicle-treated wildtype controls (data not shown). Administration of AAVhu68.CB7.CI.hARSAco.rBG (GTP-207) at all the doses tested (1.3×10¹¹ GC, 4.5×10¹⁰ GC, 1.3×10¹⁰ GC, 4.5×10⁹ GC) showed a trend towards correction of sulfatide storage however the differences were not statistically significant when compared to levels in vehicle-treated Arsa^{-/-} mice.

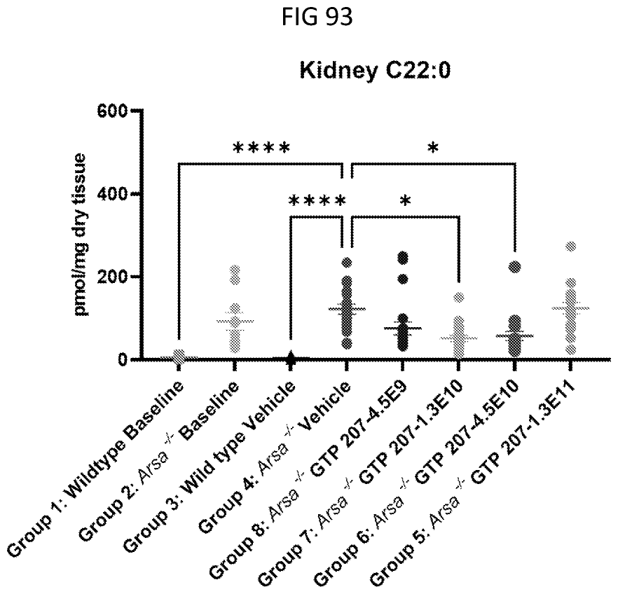

In kidney all the sulfatide species that were detected were significantly higher in the vehicle-treated Arsa^{-/-} mice compared to the age-matched vehicle-treated wildtype controls at baseline (prior to AAV administration) and at Study Day 180 (FIG. 93). Administration of AAVhu68.CB7.CI.hARSAco.rBG (GTP-207) resulted in trends towards correction of C22:0 sulfatide storage after administration of 1.3×10¹¹ GC and 4.5×10⁹ doses, however only 4.5×10¹⁰ GC and 1.3×10¹⁰ GC doses showed statistically significant reductions when compared to levels in vehicle-treated Arsa^{-/-} mice).

### Summary of Results

Administration of AAVhu68.CB7.CI.hARSAco.rBG (GTP-207) was well tolerated at all doses tested: 1.3×10¹¹ GC, 4.5×10¹⁰ GC, 1.3×10¹⁰ GC, or 4.5×10⁹ GC via ICV delivery to 4-month-old Arsa^{-/-} mice. There were no adverse effects on body weight gain, clinical observations, bloodwork, survival, and no test-article related adverse finding on histopathology.
The study's final timepoint Study Day 180 (10 months of age) coincided with the onset of a mild phenotype in our novel Arsa^{-/-} model as determined by a compound score performed in a blinded manner that comprises neurologic and general health parameters. Administration of AAVhu68.CB7.CI.hARSAco.rBG (GTP-207) at all doses (4.5×10⁹ GC, 1.3×10¹⁰ GC, 4.5×10¹⁰ GC, or 1.3×10¹¹ GC) led to a significant reduction in phenotype in Arsa^{-/-} mice when compared to aged-matched vehicle-treated controls. This suggests a potential for therapeutic benefit in terms of general health and neurologic function at doses as low as 4.5×10⁹ GC (1.1×10¹⁰ GC/g brain).
Administration of AAVhu68.CB7.CI.hARSAco.rBG (GTP-207) at the three highest doses (1.3×10¹⁰ GC, 4.5×10¹⁰ GC, or 1.3×10¹¹ GC) led to statistically significant dose-dependent increases in ARSA activity in the brain of Arsa^{-/-} mice when compared to aged-matched vehicle-treated controls.
Administration of AAVhu68.CB7.CI.hARSAco.rBG (GTP-207) at all doses (4.5×10⁹ GC, 1.3×10¹⁰ GC, 4.5×10¹⁰ GC, or 1.3×10¹¹ GC) induced hARSA protein expression in the brains of Arsa^{-/-} mice, as observed by IHC, with highest levels of expression nearest to the injection site including in the hippocampus, corpus callosum, and caudal cerebral cortex, while expression was low to undetected regions distal to the ICV injection site including in cerebellum and brainstem. This profile of expression is expected after an ICV administration into an adult mouse brain.
Myelin pathology, characterized by white matter vacuolation and axonal degeneration, was observed in the brain and spinal cord of Arsa^{-/-} mice at Study Day 180 (10 months of age), and in brain prior to treatment (at baseline, 4 months of age). There was a lower incidence, and reduced severity, of white matter findings in the spinal cord in AAVhu68.CB7.CI.hARSAco.rBG (GTP-207) treated mice at the three highest doses tested (1.3×10¹⁰ GC, 4.5×10¹⁰ GC, or 1.3×10¹¹ GC), and in brain at the highest dose (1.3×10¹¹ GC; noting that for brain only the highest dose was evaluated since the primary purpose of the analysis was to detect potential AAV-associated toxicity. Since no adverse findings were observed after the highest dose of AAVhu68.CB7.CJ.hARSAco.rBG (GTP-207), animals that received lower doses were not examined).
Administration of AAVhu68.CB7.CI.hARSAco.rBG (GTP-207) at the three highest doses (1.3×10¹⁰ GC, 4.5×10¹⁰ GC, or 1.3×10¹¹ GC) led to a significant amelioration of lysosomal pathology (normalization of elevated LAMP-1 immunostaining) in the hippocampus and in the corpus callosum (white matter) of Arsa^{-/-} mice when compared to aged-matched vehicle-treated controls. This observation suggests the potential for therapeutic benefit at doses as low as 1.3×10¹⁰ GC (3.3×10¹⁰ GC/g brain) in regions that are robustly transduced following AAV delivery. Importantly, LAMP-1 immunostaining was already increased at baseline, indicating a reversal of existing lysosomal enlargement after treatment with AAVhu68.CB7.CJ.hARSAco.rBG (GTP-207).
Regions of the CNS more distal to the injection site with lower transduction levels such as the spinal cord and caudal cerebral cortex showed evidence of LAMP-1 normalization after the highest treatment dose (1.3×10¹¹ GC or 3.25×10¹¹ GC/g brain), and two highest doses (1.3×10¹¹ GC [3.25×10¹¹ GC/g brain], and 4.5×10¹⁰ GC [1.1×10¹¹ GC g brain]).
Administration of AAVhu68.CB7.CI.hARSAco.rBG (GTP-207) at the three highest doses (1.3×10¹⁰ GC, 4.5×10¹⁰ GC, or 1.3×10¹¹ GC) led to a significant amelioration of neuroinflammation (as detected by GFAP immunostaining for astrogliosis) in the cerebral cortex of Arsa^{-/-} mice when compared to aged-matched vehicle controls. This suggests a potential therapeutic benefit in terms of reduced neuroinflammation at doses as low as 1.3×10¹⁰ GC (3.3×10¹⁰ GC/g brain). Importantly, GFAP increase was already present at baseline, indicating a reversal of the pathology after treatment with AAVhu68.CB7.CI.hARSAco.rBG (GTP-207). The other brain regions analyzed did not demonstrate significant astrogliosis in Arsa^{-/-} mice compared to WT controls.
ICV administration of AAVhu68.CB7.CI.hARSAco.rBG (GTP-207) at all doses (4.5×10⁹ GC, 1.3×10¹⁰ GC, 4.5×10¹⁰ GC, or 1.3×10¹¹ GC) led to significant reductions of several sulfatide species in the brains of Arsa^{-/-} mice as measured by LC/MS, demonstrating reduction of the storage material, and potential therapeutic benefit at doses as low as 4.5×10⁹ GC (1.1×10¹⁰ GC/g brain). Sulfatide storage was also significantly reduced in the spinal cord after administration of the highest dose (1.3×10¹¹ GC [3.3×10¹¹ GC/g brain]).
In peripheral tissues, administration of AAVhu68.CB7.CI.hARSAco.rBG (GTP-207) at the two highest doses (4.5×10¹⁰ GC, or 1.3×10¹¹ GC) led to a significant dose-dependent increase of ARSA activity levels in the liver and the heart of Arsa^{-/-} mice when compared to aged-matched vehicle-treated controls. The third highest dose (1.3×10¹⁰ GC [3.3×10¹⁰ GC/g brain]) induced trends towards increases in ARSA activity in the liver, which was more pronounced in females than in males. Changes in the combined gender group did not reach significance, however, putatively due to the reported gender-related variability of liver transduction by AAVs in rodents. This suggests the potential for meaningful transduction of peripheral organs following administration of AAVhu68.CB7.CI.hARSAco.rBG (GTP-207) into cerebrospinal fluid (CSF), and the potential to provide a peripheral source of ARSA enzyme for cross-correction of cells at doses as low as 1.3×10¹⁰ GC after ICV administration (3.3×10¹⁰ GC/g brain). Increased ARSA activity in peripheral organs led to significantly reduced sulfatide levels in liver and plasma after the two highest doses tested (4.5×10¹⁰ GC, and 1.3×10¹¹ GC), and sulfatide reduction in kidney at the 2 middle doses (1.3×10¹⁰ GC, 4.5×10¹⁰ GC). Impairments in kidney function in Arsa^{-/-} mice, as measured by elevations in BUN levels were ameliorated dose-dependently by treatment with AAVhu68.CB7.CI.hARSAco.rBG (GTP-207).
In summary, the minimum effective dose (MED) after ICV administration of AAVhu68.CB7.CI.hARSAco.rBG (GTP-207) to Arsa^{-/-} mice was determined to be the lowest dose of 4.5×10⁹ GC (equivalent to 1.1×10¹⁰ GC/g brain weight) because this dose significantly ameliorated the phenotype of Arsa^{-/-} mice as assessed by a compound clinical score, produced detectable levels of hARSA transgene in the brain as detected by immunostaining, and significantly reduced several sulfatide species in the brain. A 3-fold higher dose (1.3×10¹⁰ GC [3.3×10¹⁰ GC/g brain]) demonstrated a broader pharmacological effect with significant reductions in neuroinflammation and lysosomal pathology in brain (as measured by GFAP and LAMP-1 immunostaining, respectively), significant increase in brain ARSA activity and significant reductions in additional sulfatide species in the brain and in the kidney.

### Example 10—Toxicology Study in Nonhuman Primates

A toxicology study was performed to assess the safety and tolerability of AAVhu68.CB7.CI.hARSAco.rBG (GTP-207), a recombinant adeno-associated virus (AAV) serotype hu68 vector expressing human arylsulfatase A (ARSA), following intra-cisterna magna (ICM) administration in juvenile non-human primates (NHPs).

Juvenile male and female rhesus macaques received a single ICM administration of vehicle (intrathecal final formulation buffer [ITFFB]) or AAVhu68.CB7.CI.hARSAco.rBG (GTP-207) at a dose of 3.0×10¹² genome copies (GC) (low dose; 3.3×10¹⁰ GC/g brain), 1.0×10¹³ GC (mid-dose; 1.1×10¹¹ GC/g brain), or 3.0×10¹³ GC (high dose; 3.3×10¹¹ GC/g brain). Animals from each cohort were euthanized either 90 or 180 days following administration.

In-life evaluations included clinical observations performed daily, physical exams, standardized neurological monitoring, sensory nerve conduction studies (NCS), body weights, clinical pathology of the blood and cerebrospinal fluid (CSF), evaluation of serum-circulating neutralizing antibodies (NAbs), assessment of vector pharmacokinetics and vector excretion, and evaluation of transgene product expression (ARSA enzyme activity) and antibodies against the transgene product (anti-human ARSA antibodies) in CSF and serum. Animals were necropsied, and tissues were harvested for a comprehensive histopathological examination and measurement of T cell responses to the vector capsid and transgene product.

Following Group assignment, each animal received a single ICM administration of one of the following treatments consisting of either control article (ITFFB) or AAVhu68.CB7.CI.hARSAco.rBG (GTP-207):
1.) ITFFB (control article)
2.) a low dose of GTP-207 (3.0×10¹² GC; test article)
3.) a mid-dose of GTP-207 (1.0×10¹³ GC; test article)
4.) a high dose of GTP-207 (3.0×10¹³ GC; test article)

The day of dose administration (Day 0) was staggered with animals representing as many study groups as possible across administration dates. The study design is summarized in the table below.

TABLE
Group Designations, Dose Levels, and Route of Administration

| | | | | | | | Administration | | |
|---|---|---|---|---|---|---|---|---|---|
| | | Dose | Dose | Animal | | | Volume | Day of | Day of |
| Group | Treatment | (GC) | (GC/g Brain)^{a} | ID | Sex | ROA | (mL) | Dosing | Necropsy |
| 1 | ITFFB | N/A | N/A | 19-030 | M | ICM | 1.0 | 0 | 90 ± 4 |
| 2 | GTP-207 | 3.0 × 10¹² | 3.3 × 10¹⁰ | 18-223 | F | | | | |
| | (Low | | | 18-226 | F | | | | |
| | Dose) | | | 19-033 | M | | | | |
| 3 | GTP-207 | 1.0 × 10¹³ | 1.1 × 10¹¹ | 18-205 | M | | | | |
| | (Mid- | | | 18-207 | M | | | | |
| | Dose) | | | 19-024 | F | | | | |
| 4 | GTP-207 | 3.0 × 10¹³ | 3.3 × 10¹¹ | 18-225 | F | | | | |
| | (High | | | 18-228 | F | | | | |
| | Dose) | | | 19-023 | M | | | | |
| 5 | ITFFB | N/A | N/A | 19-034 | F | ICM | 1.0 | 0 | 180 ± 5 |
| 6 | GTP-207 | 3.0 × 10¹² | 3.3 × 10¹⁰ | 18-218 | F | | | | |
| | (Low | | | 18-220 | F | | | | |
| | Dose) | | | 18-222 | F | | | | |
| 7 | GTP-207 | 1.0 × 10¹³ | 1.1 × 10¹¹ | 18-206 | M | | | | |
| | (Mid- | | | 18-219 | F | | | | |
| | Dose) | | | 19-014 | F | | | | |
| 8 | GTP-207 | 3.0 × 10¹³ | 3.3 × 10¹¹ | 18-215 | F | | | | |
| | (High | | | 19-015 | M | | | | |
| | Dose) | | | 19-028 | M | | | | |

^{a}Doses are scaled based on a brain mass of 90 g for a juvenile NHP (Herndon et al., 1998).
Abbreviations: GC, genome copies; ICM, intra-cisterna magna; ID, identification number; ITFFB, intrathecal final formulation buffer; N/A, not applicable; NHP, non-human primate; ROA, route of administration.

### Test Article and Control Article Administration

### Administration Procedure

On Study Day 0, animals were sedated prior to dosing. Prior to control article or test article administration, animals were weighed, and vital signs were recorded. Analgesics were provided to animals.

Animals were then dosed with a single injection of either control article (ITFFB) or AAVhu68.CB7.CI.hARSAco.rBG (GTP-207) via sub-occipital puncture into the cisterna magna (ICM administration) with fluoroscopic confirmation. Briefly, anaesthetized macaques were transferred from the animal-holding space and placed on an X-ray table in the lateral decubitus position with the head flexed forward for CSF collection and dosing into the cisterna magna. The site of injection was aseptically prepared. Using aseptic technique, a 1.0-3.5-inch 18-23 gauge Quincke spinal needle was advanced into the sub-occipital space until the flow of CSF was observed. Up to 1.0 mL of CSF was collected for baseline analysis prior to dosing. The needle was directed at the wider superior gap of the cisterna magna to avoid blood contamination and potential brainstem injury. After CSF collection, a small-bore T extension catheter was connected to the spinal needle to facilitate dosing of contrast media followed by either control article or test article. Up to 1.0 mL of contrast media was administered via the catheter and spinal needle. After verifying needle placement via CSF return and visualization of the needle by fluoroscopy, a syringe containing either the control article or test article (volume equivalent to 1.0 mL plus the volume of syringe and linker dead space) was connected to the flexible linker and injected over 30±5 seconds. After administration, the needle was removed, and direct pressure was applied to the puncture site.

### Observations

Viability Assessments (in-Cage)

Animals were observed daily visually for general appearance or signs of toxicity, which included but were not limited to neurologic signs or lethargy, distress, and changes in behavior by following SOP 7404. The NHP Daily Observation Sheet Form 7404-F1 was completed for the duration of the study. The Clinical Veterinarian or designee and the Study Director was notified of any unusual conditions. Treatment was conducted only after approval by the Clinical Veterinarian or designee and Study Director, except in cases of emergency imperiling the NHP or for humanely euthanizing the NHP if the Clinical Veterinarian and/or the Study Director could not be contacted promptly.

### In-Life Examinations

### Neurological Monitoring

Animals underwent neurological monitoring at various time points. Briefly, the assessment was divided into five sections evaluating the following: mentation, posture and gait, proprioception, cranial nerves, and spinal reflexes. The tests for each assessment were performed in the same order each time. Assessors were not formally blinded to the treatment group; however, assessors typically remained unaware of treatment Group at the time of assessment. Numerical scores were given for each assessment category as applicable and recorded (normal: 1; abnormal: 2; decreased: 3; increased: 4; none: 5; N/A: not applicable).

### Mentation

To assess mentation, NHPs were assessed cage-side prior to manipulation by the examiner by noting how the animal interacted with the examiner and the environment. Any changes, such as depressed, dull, disoriented, or comatose behavior, were recorded in addition to respiratory character and effort and any excessive lacrimation or salivation.

### Posture and Gait

To assess posture and gait, NHPs were assessed cage-side prior to manipulation by the examiner by observing how the animal moved around in the cage. Any impairments, such as ataxia, paresis, paralysis, or stumbling/falling/tremors/convulsions/uncoordinated movement were recorded. The examiner also observed the animal's posture, head position (head tilt, head or neck turn), wide-based stance, ability to perch, tremors, or unintentional movements, and any abnormalities were recorded.

### Proprioception

Proprioceptive assessments were optional as they can only be performed on a restrained animal. Proprioceptive positioning was assessed by standing the animal on a flat surface, such as a tabletop, and flipping the dorsal aspect of each of the hind feet (one at a time) onto the tabletop. The animal should correct the placement of the foot immediately, and any delayed responses and/or failures to correct placement of the foot were recorded. Visual placing was assessed by slowly moving the primate towards a flat surface with a ledge (such as a tabletop) and allowing the dorsal aspect of the hind feet to touch the surface. The primate should respond by placing the plantar aspect of both feet on the tabletop. Tactile placing was assessed in the same way as visual placing except that the primate's eyes were covered by the examiner's hand.

### Cranial Nerves

The cranial nerve assessment was performed in the cage by utilizing the squeeze-back mechanism or outside the cage in a chair while restrained by noting facial/head symmetry as well as facial and cranial muscle tone. Any abnormalities were recorded. The menace reflex was assessed by advancing the hand of the handler toward each eye of the primate, using caution so as not to create an air current or touch any part of the primate's face. The menace reflex test determined whether the animal blinked each eye as the examiner's hand approached the face, and any abnormalities were recorded. Each eye was examined for symmetry (placement, pupil size and shape). Pupillary light reflex was assessed in both eyes using either a trans-illuminator or pen light by covering the eyes with both hands for 5 seconds, removing hand, and then shining light directly into the eye to assess pupillary constriction. The symmetry of pupillary response (speed of contraction and overall degree of constriction) was noted. The palpebral reflex was assessed in both eyes by touching a cotton tip applicator to the lateral canthus followed by the medial canthus of the eye. The animal should blink with each touch, and any abnormalities were recorded. Sensation of the nasal septum was assessed by pinching the nasal septum with forceps and determining whether the animal reacted to the noxious stimulus. Eye positioning was assessed in chaired or in manually restrained animals in which the nose can be elevated while the eyes stayed in a normal position. When the head was gently moved side to side, the eyes should have followed the movement of the head (nystagmus), and any abnormalities were recorded.

### Spinal Reflex

The spinal nerves/spinal reflexes were assessed by evaluating the primate's muscle strength. If manually restrained, muscle strength was assessed by the handler holding both hind limbs (one in each hand) to assess the primate's ability to resist manipulation of the limbs. If assessed cage-side, muscle strength was assessed by handing the animal a toy or other appropriate object to grasp in its hand while the examiner continued to hold the object while pulling back. In each case, the animal's ability to resist the examiner's action was recorded. The withdrawal reflex was assessed by pinching each of the hind feet with a hemostat and determining whether the animal quickly flexed the knee and drew its limb up toward the body. The response was recorded. The perineal reflex was assessed by gently stroking the skin around the anus with a cotton-tipped applicator and assessing whether the animal contracted the external sphincter muscles indicated by a puckering of the surrounding skin.

### Sensory Nerve Conduction Study

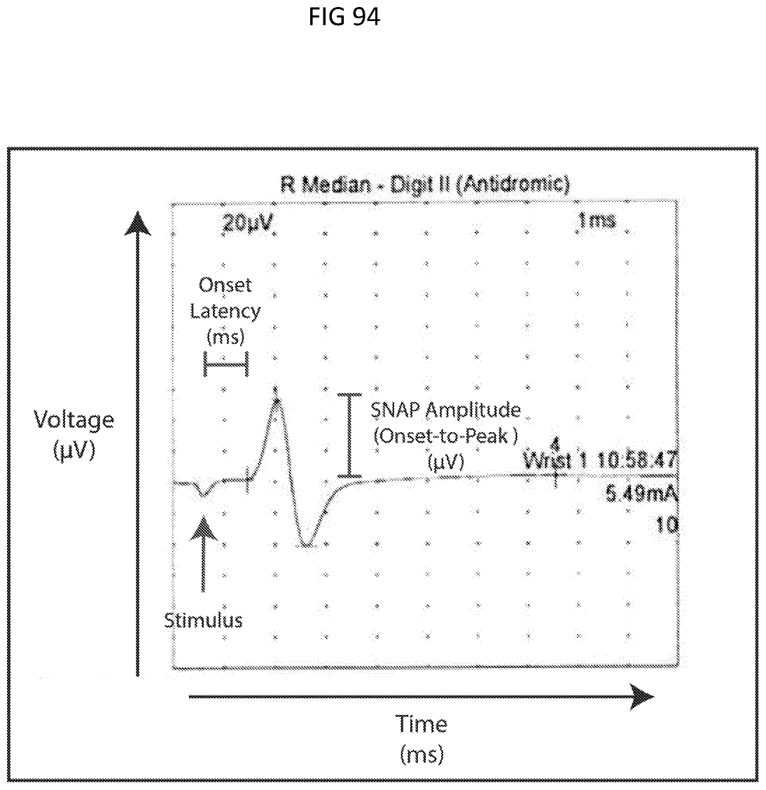

Nerve conduction studies (NCS), also referred to as sensory nerve conduction velocity (NCV) tests in the study protocol. Briefly, NCS were performed on the left and right median nerves in accordance with SOP 7807 using the Nicolet EDX® system (Natus Neurology) and Viking® analysis software to measure SNAP amplitudes and conduction velocities. Briefly, animals were sedated with a combination of ketamine/dexmedetomidine. Sedated animals were placed in lateral or dorsal recumbency on a procedure table with heat packs to maintain body temperature. Electronic warming devices were not used due to the potential for interference with electrical signal acquisition. The stimulator probe was positioned over the median nerve with the cathode closest to the recording site. Two needle electrodes were inserted subcutaneously on digit II at the level of the distal phalanx (reference electrode) and proximal phalanx (recording electrode), while the ground electrode was placed proximal to the stimulating probe (cathode). A WR50 Comfort Plus Probe pediatric stimulator (Natus Neurology) was used. The elicited responses were differentially amplified and displayed on the monitor. The initial acquisition stimulus strength was set to 0.0 mA in order to confirm a lack of background electrical signal. In order to find the optimal stimulus location, the stimulus strength was increased up to 10.0 mA, and a train of stimuli were generated while the probe was moved along the median nerve until the optimal location was found as determined by a maximal definitive waveform. Keeping the probe at the optimal location, the stimulus strength was progressively increased up to 10.0 mA in a stepwise fashion until the peak amplitude response no longer increased. The last thirty stimulus responses were recorded and saved in the software. Up to 10 maximal stimuli responses were averaged and reported for the median nerve. The distance (cm) from the recording site to the stimulation cathode was measured and entered into the software. The conduction velocity was calculated using the onset latency of the response and the distance (cm). Both the conduction velocity and the average of the SNAP amplitude were reported (FIG. 94). The median nerve was tested bilaterally. All raw data generated by the instrument were retained as part of the study file.

### Evaluation of Transgene Product Expression (ARSA Enzyme Activity)

Human ARSA enzyme activity was measured in CSF and serum using a p-nitrochatechol assay. Briefly, dialyzed serum (diluted 1:5, 1 part serum+4 parts diluent; Section 4.4.3.1) or undialyzed CSF (diluted 1:2, 1 part of CSF+1 part of diluent) were diluted into a base buffer (0.5 M sodium acetate buffer, pH 5.0; 10% sodium chloride; 0.5 mM sodium pyrophosphate), and L diluted sample was loaded in four wells (2 duplicates) into a 96-well plate. Next, 40 μL of substrate (10 mM 4-nitrocatechol sulfate in base buffer) was added to the samples, and the reaction was stopped by immediately adding 120 μL 1N NaOH (stop solution) in two of the four wells. The plate was then incubated at 37° C. for 5 hours. The reaction was stopped by adding 120 μL 1N NaOH (stop solution) in the remaining wells. The absorbance was measured by reading the plate at 515 nm using a plate reader. ARSA-specific activity was determined by multiplying the absorbance obtained at five hours minus the absorbance at 0 minute with the extinction coefficient of a 4-nitrocathecol standard curve at 515 nm and by dividing by the amount of protein in the well (mg) as measured by BCA assay (serum only). The results for ARSA activity were expressed as nmol per milligram protein per five hours (serum) or nmol per milliliter per five hours (CSF).

### Evaluation of Antibodies Against the Transgene Product (Anti-Human ARSA Antibodies)

Immunoglobulin G (IgG) antibodies against human ARSA protein were measured in CSF and serum by an indirect ELISA according to SOP 7009. Briefly, a high-binding polystyrene ELISA plate was coated overnight at 4° C. with ≥1 μg/mL recombinant human ARSA protein diluted in Acidic DPBS. After washing, the plate was blocked with 2% bovine serum albumin (BSA) in Acidic DPBS for 2 hours at room temperature, followed by sample incubation for 1.5 hours at room temperature. CSF samples were diluted 1:20 in DPBS, and serum samples were diluted 1:1000 in DPBS. After washing, bound anti-human ARSA antibody was detected with a biotinylated goat anti-human IgG antibody and a streptavidin-conjugated HRP. Plates were developed using the TMB substrate for 20 minutes. The reaction was then stopped with 2 N sulfuric acid, and absorbance was measured at 450 nm.

### Results

### Mortality

All animals survived to the scheduled necropsy time point.

### Clinical Observations

Animals were monitored daily throughout the study. There were no clinical abnormalities attributable to test article administration. Several abnormalities unrelated to test article administration were noted. These observations and associated treatment, if needed, did not impact the study because symptoms fully resolved over time and none of the observations were suspected to be test article-related based on the symptoms and diagnostics. None of the observations impacted the in-life endpoints, with the exception of animal 18-219 (1.0×10¹³ GC, Group 7) that exhibited an injury to digit II of its left hand at the Day 28 NCS, which may have contributed to the reduction in median nerve SNAP amplitudes on its left side.

### Neurological Examinations

Standardized neurological examinations were performed at baseline and on Days 14, 28, 60, 90, 120, 150, and 180 after administration. Animals were occasionally uncooperative with the exam, precluding some assessments. However, all required components of the exam were assessed at most time points for each animal. No abnormal neurologic signs attributed to the test article were noted.

A number of animals in the Day 90 and Day 180 cohorts occasionally exhibited a decreased, absent, or abnormal (delayed or increased) withdrawal reflex at some time points. This observation was attributed to either anxiety or habituation to the procedure because no other abnormalities related to grasping ability and/or other testing parameters were noted during the neurological examination in most cases, and there was no clear association with test-article or dose dependance. A decreased, absent, or abnormal (delayed or increased) withdrawal reflex was observed on Day 14 for Animal 18-228 (GTP-207; 3.0×10¹³ GC; Group 4); on Days 14, 28, 60, and 90 for Animal 18-205 (GTP-207; 1.0×10¹³ GC; Group 3); on Days 14 and 120 for Animal 19-015 (GTP-207; 3.0×10¹³ GC; Group 8); on Day 28 for Animal 18-207 (GTP-207; 1.0×10¹³ GC; Group 3); on Days 28 and 60 for Animal 19-024 (GTP-207; 1.0×10¹³ GC; Group 3); on Days 28, 60, 150, and 180 for Animal 18-206 (GTP-207; 1.0×10¹³ GC; Group 7); on Day 60 for Animal 18-225 (GTP-207; 3.0×10¹³; Group 4); on Days 60 and 90 for Animal 19-034 (ITFFB; Group 5) and Animal 18-218 (GTP-207; 3.0×10¹² GC; Group 6); on Day 120 for Animal 19-028 (GTP-207; 3.0×10¹³ GC; Group 8); and on Day 150 for Animal 18-220 (GTP-207; 3.0×10¹² GC; Group 6).

Additionally, several animals in the Day 90 and Day 180 cohorts exhibited decreased or no response to the nasal septum test at some of the time points assessed. These atypical responses were attributed to either anxiety or habituation to the exam since no other abnormalities were noted in the neurological exam and/or all facial movements were within normal limits. A decreased or absent response to the nasal septum test was observed on Day 14 for Animal 18-228 (GTP-207; 3.0×10¹³ GC; Group 4); on Day 14, 60, and 90 for Animal 18-205 (GTP-207; 1.0×10¹³ GC; Group 3); on Day 28 for Animal 18-225 (GTP-207; 3.0×10¹³ GC; Group 4); Animal 18-218 (GTP-207; 3.0×10¹² GC; Group 6), and Animal 18-222 (GTP-207; 3.0×10¹² GC; Group 6); on Days 60 and 90 for Animal 19-034 (ITFFB; Group 5); on Days 60, 150, and 180 for Animal 18-206 (GTP-207; 1.0×10¹³ GC; Group 7); on Day 120 for Animal 19-028 (GTP-207; 3.0×10¹³ GC; Group 8); and on Day 180 for Animal 18-215 (GTP-207; 3.0×10¹³ GC; Group 8).

### Sensory Nerve Conduction Studies

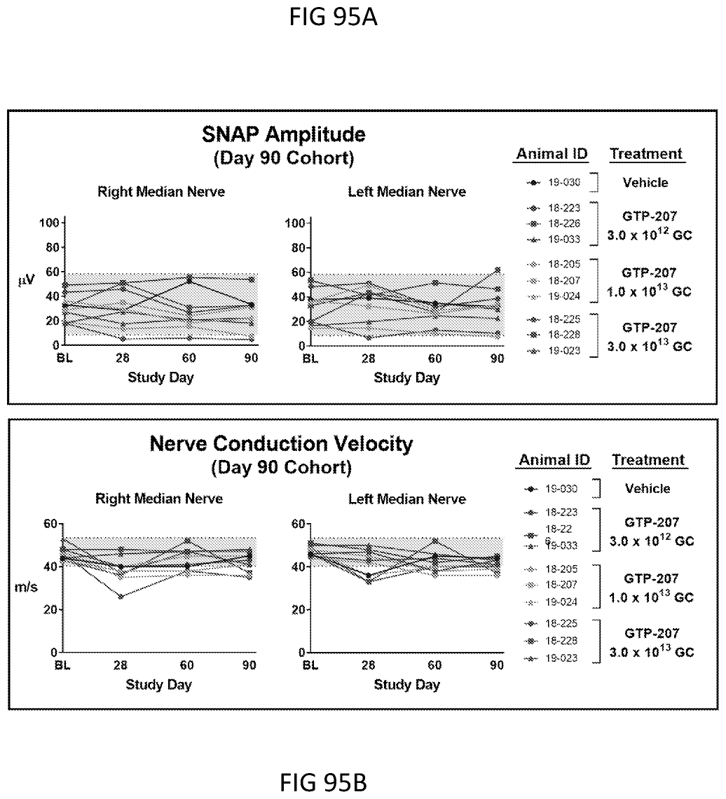

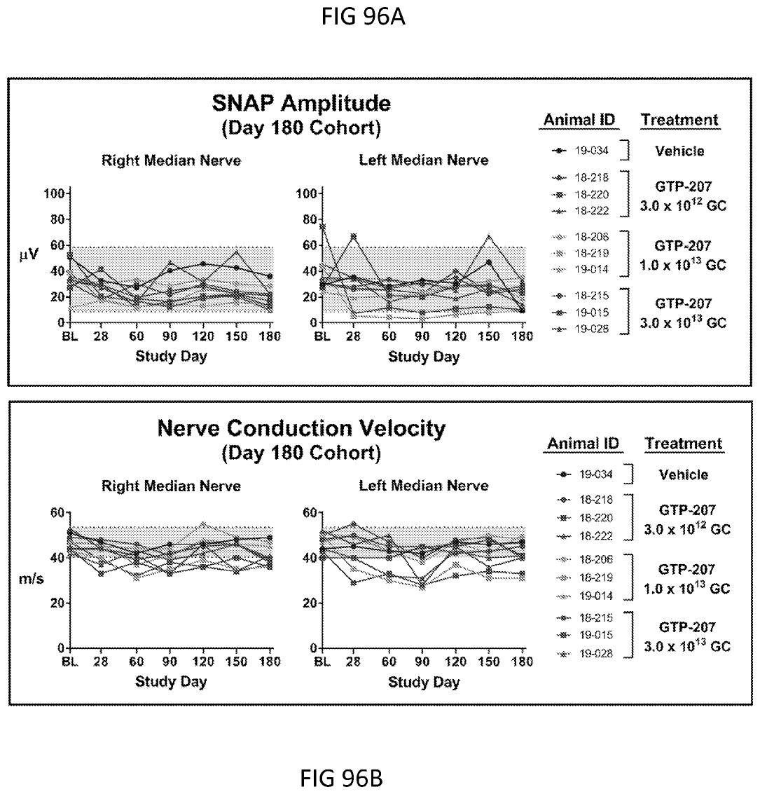

Sensory nerve conduction studies (NCS) were performed for all animals at baseline and monthly thereafter to measure bilateral median nerve sensory nerve action potential (SNAP) amplitudes and conduction velocities (FIG. 95 and FIG. 96).

A bilateral or unilateral reduction in median nerve SNAP amplitudes from baseline levels that exceeded normal individual animal variability with values below 2 standard deviations of baseline average was observed in 2/6 animals from the mid-dose groups (1.0×10¹³ GC; Animal 18-205, Group 3, Day 90 [bilateral]; Animal 18-219, Group 7, Days 28, 60, 90, 120, and 150 [unilateral, left side]) and 2/6 animals from the high dose groups (3.0×10¹³ GC; Animal 18-225, Group 4, Days 28 [bilateral], 60, and 90 [unilateral, right side]; Animal 19-015, Group 8, Days 28 and 90 [unilateral, left side]; FIG. 95 and FIG. 96). All of these animals displayed minimal or mild neuronal degradation/necrosis in at least two of the three segments of the dorsal root ganglia, and the severity of spinal cord dorsal axonopathy ranged from mild (grade 2) to moderate (grade 3) and marked (grade 4) on tissues analyzed postmortem. For the two Day 90 cohort animals, there was a correlation between abnormal low SNAP amplitude and severity of histopathology findings, with Animal 18-205 (1.0×10¹³ GC, Group 3) and Animal 18-225 (3.0×10¹³ GC, Group 4) demonstrating the most spinal cord dorsal axonopathy (up to grade 3, and grade 4 respectively), axonal degradation and endoneurial fibrosis in the nerve root of the DRG and mild (grade 2) endoneurial fibrosis in the median nerves. A correlation between abnormal low SNAP amplitude and severity of histopathology findings was less clear in the Day 180 cohort animals, although Animal 19-015 (3.0×10¹³ GC, Group 8) had grade 1) endoneurial fibrosis in the median nerves. The two animals in the Day 180 cohort with abnormal low SNAP amplitudes from Study Day 28 and longer follow-up demonstrated a trend to time-dependent amelioration, with a value back within the normal range by Day 120 (Animal 19-015, 3.0×10¹³ GC, Group 8) or Day 180 (Animal 18-219, 1.0×10¹³ GC, Group 7), although they were still markedly decreased compared to the baseline. Animal 18-219 (1.0×10¹³ GC, Group 7) also exhibited an injury to digit II of its left hand at the Day 28 NCS evaluation, which may have contributed to the reduction in median nerve SNAP amplitudes on its left side while this injury was healing.

Taken together, the decreased SNAP amplitudes, when correlated with histopathology findings are likely attributable to test article-related sensory neuron toxicity. Inter- and intra-animal variability in SNAP amplitudes were apparent throughout the study, and the position of the recording needle relative to the nerve can impact SNAP amplitude. Variations in SNAP amplitudes are frequently observed in longitudinal studies, especially in juvenile animals that are still growing and may therefore have slightly modified anatomical landmarks from one time point to another, which is why definitive interpretation needs to be made in correlation (or lack thereof) with histopathology data. Overall, 3/4 animals with decreased SNAP amplitudes had correlation with worse axonopathy (Animals 18-205 and 18-225) or endoneurial fibrosis (Animal 19-015), while 1/4 had low values that can be attributed to a non-test-article related injury (Animal 18-219).

Nerve conduction velocities are typically less affected by the electrode positioning and were therefore expected to display less variation for most animals (FIG. 95 and FIG. 96). However, a reduction in nerve conduction velocities from baseline levels that exceeded normal individual animal variability was observed at more than one time point in 1/2 vehicle-treated animals (ITFFB, Animal 19-030, Group 1, Days 28 and 60), 5/6 animals in the low dose groups (3.0×10¹² GC; Animal 18-223, Day 28; Animal 19-033, Day 60 [Group 2]; Animal 18-218, Days 60 and 180; Animal 18-220, Days 28-180; Animal 18-222, Days 90, 150, and 180 [Group 6]), 6/6 animals in the mid-dose groups (1.0×10¹³ GC; Animal 18-205 and Animal 18-207, Days 28, 60, and 90; Animal 19-024, Days 28 and 60 [Group 3]; Animal 18-219, Days 28-180; Animal 19-014, Day 60; Animal 18-206, Day 90 [Group 7]), and 6/6 animals in the high dose groups (3.0×10¹³ GC; Animal 19-023, Day 28; Animal 18-225, Days 28, 60, and 90; Animal 18-228, Days 28 and 90 [Group 4]; Animal 18-215, Days 90 and 180; Animal 19-015, Group 8, Days 28-180; Animal 19-028, Days 28, 60, 90, 150, and 180 [Group 8]). Since reduced nerve conduction velocities were observed across groups, including in one animal from the vehicle-treated control group, and there was no clear correlation between reduced nerve conduction velocities and dose group, it is unlikely that these findings are test article-related.

### Body Weights

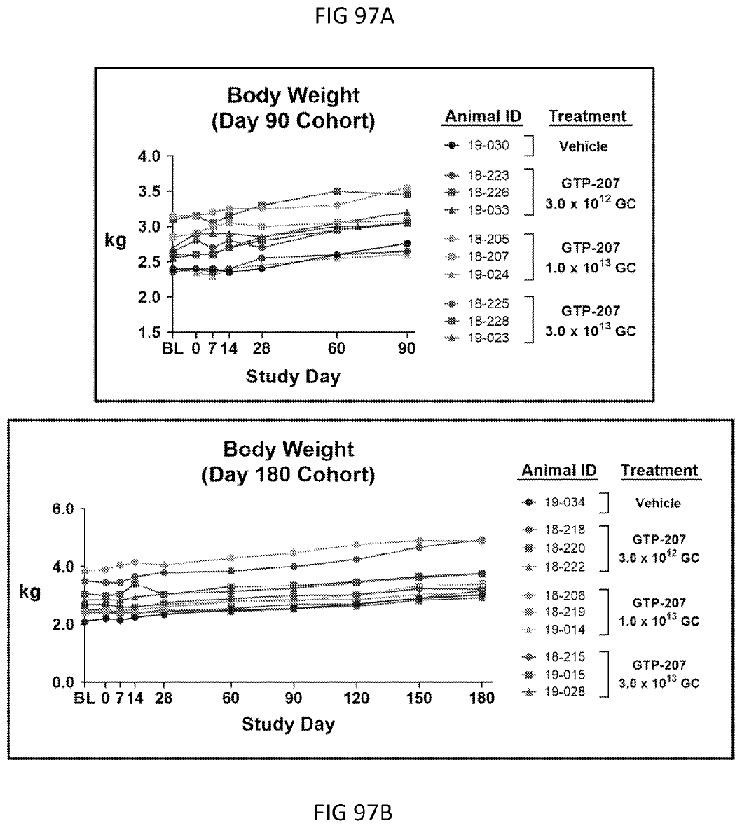

Animals in both the Day 90 and Day 180 cohorts exhibited weight gain after test article administration and throughout the study (FIG. 97). A transient body weight loss was observed at no more than two time points in some animals, including 1/2 vehicle-treated controls and 7/18 AAVhu68.CB7.CI.hARSAco.rBG (GTP-207)-treated NHPs on Day 7, 1/2 vehicle-treated controls on Day 14, 5/18 GTP-207-treated NHPs on Day 28, 1/18 GTP-207-treated NHPs on Day 60, 1/18 AAVhu68.CB7.CI.hARSAco.rBG (GTP-207)-treated NHPs on Day 90, and 2/9 AAVhu68.CB7.CI.hARSAco.rBG (GTP-207)-treated NHPs on Day 180. Additionally, 4/18 GTP-207-treated NHPs exhibited body weight loss at two time points, including 1/18 animals on Days 7 and 28, 1/18 on Days 7 and 90, 1/18 animals on Days 7 and 180, and 1/18 on Days 28 and 180. The observed transient weight loss was considered unrelated to the test article.

On Day 7, 8/18 animals experienced body weight loss from the previously recorded weight, including 1/2 vehicle-treated control (ITFFB; Animal 19-034 [−2.27%, Group 5]), 2/6 animals administered the low dose of AAVhu68.CB7.CI.hARSAco.rBG (GTP-207) (3.0×10¹² GC; Animal 18-223 [−2.08%; Group 2] and Animal 18-218 [−3.70%, Group 6]), 2/6 animals administered the mid-dose (1.0×10¹³ GC; Animal 19-024 [−2.13%; Group 3] and Animal 19-014 [−2.00%, Group 7]), and 3/6 animals administered the high dose (3.0×10¹³ GC; Animal 18-225 [−3.57%; Group 4], Animal 18-228 [−3.17%; Group 4], and Animal 19-015 [−5.88%, Group 8]). For some animals, this weight loss could be accounted for by observations of reduced appetite (Animal 18-223, Animal 19-024, Animal 19-015, and Animal 19-014), intermittent soft stool (Animal 19-014), or skin irritation (Animal 18-228) during the first 7 days post treatment. For the remaining animals (Animals 19-034, 18-218, and 18-225), no clinical symptoms were noted that accounted for the observed temporary weight loss.

On Day 14, 1/2 vehicle-treated controls (ITFFB; Animal 19-030 [−2.08%; Group 1]) exhibited weight loss from the previously recorded weight, and no clinical symptoms were noted that accounted for the observed temporary weight loss. The animal continued to gain weight throughout the remainder of the study on supplemental feeding.

On Day 28, 5/18 animals experienced body weight loss from the previously recorded weight, including 2/6 animals administered the low dose of AAVhu68.CB7.CI.hARSAco.rBG (GTP-207) (3.0×10¹² GC; Animal 19-033 [−1.72%; Group 2] and Animal 18-220 [−10.29%; Group 6]), 2/6 animals administered the mid-dose (1.0×10¹³ GC; Animal 18-207 [−1.64%; Group 3] and Animal 18-206 [−2.41%; Group 7]), and 1/6 animals administered the high dose (3.0×10¹³ GC; Animal 18-225 [−3.57%; Group 4]. The weight loss of −10.29% for Animal 18-220 on Day 28 was noted to be an outlier by the Study Veterinarian and possibly the result of a clerical error at the time of recording, as the animal's weight on Day 14 was similar to that on Day 60. Animal 18-207 was observed to be going through a growth spurt at this time, which may be associated with the transient weight loss observed. Animal 18-206 got its right foot stuck in its cage bars on Day 22, causing significant swelling and several superficial abrasions that may or may not be related to the subsequent reduction in weight observed on Day 28. For Animals 19-033 and 18-225, no clinical symptoms were noted that accounted for the observed temporary weight loss.

On Day 60, 1/6 animals administered the high dose of AAVhu68.CB7.CI.hARSAco.rBG (GTP-207) (3.0×10¹³ GC; Animal 19-028 [−2.00%; Group 8]) demonstrated body weight loss from the previously recorded weight that was not attributed to any clinical symptoms.

On Day 90, 1/6 animals administered the high dose of AAVhu68.CB7.CI.hARSAco.rBG (GTP-207) (3.0×10¹³ GC; Animal 18-228 [−1.43%; Group 4]) demonstrated body weight loss from the previously recorded weight that was not attributed to any clinical symptoms.

On Day 180, 1/6 animals administered the low dose of AAVhu68.CB7.CI.hARSAco.rBG (GTP-207) (3.0×10¹² GC; Animal 18-218 [−0.31%; Group 6]) and 1/6 animals administered the mid-dose (1.0×10¹³ GC; Animal 18-206 [−0.61%; Group 7]) exhibited body weight loss from the previously recorded weight that was not attributed to any clinical symptoms.

Despite the transient reductions in weight discussed above, all animals continued to gain and/or maintain weight at all subsequent time points for the duration of the study as applicable.

### Hematology, Coagulation, and Clinical Chemistry (Blood and Cerebrospinal Fluid) Blood

No test article-related abnormalities were noted on blood CBCs, coagulation studies, or serum chemistry panels. Several minor abnormalities unrelated to test article administration were observed that were considered clinically insignificant.

### Hematology (Complete Blood Counts)

Based on analysis of leukogram data, some animals presented with leukocytosis during the study. Animals 18-219, 18-207, and 18-206 presented with a leukocytosis at various time points throughout the study that were not test-article related and that were attributed to various wounds (18-219, 18-207, 18-206) and/or to menstruation (18-219). Animal 18-223 presented with leukocytosis on baseline and study days 7, 15, 28 and 62, and the majority of these time-points were classified as a lymphocytic leukocytosis. In this case, the leukocytosis was mild and most likely due to individual variation as it was first noted at baseline. Animals 18-226, 18-205, 18-225, 18-228, 18-218, 18-222, 19-014, and 18-215 presented with a leukocytosis at various time points that did not correspond with clinical signs cage-side. All leukogram changes were mild in nature and likely clinically insignificant. Bloodwork abnormalities did not appear to correlate with dose group.

Analysis of hemogram data revealed that a subset of animals presented with mild relative erythrocytosis or thrombocytosis that may have been secondary to hemoconcentration due to loss of intravascular fluid or epinephrine-mediated splenic contraction. Additionally, Animals 18-223, 18-225 and 19-030 presented with a mildly low mean corpuscular hemoglobin at all time points, which was likely due to individual variation as it was first noted at baseline. A subset of animals presented with hypochromic red blood cells (decreased mean corpuscular hemoglobin concentration) slightly out of reference range, which was likely related to individual variation or secondary to a regenerative process. Finally, Animal 18-220 presented with intermittent minor thrombocytopenia, starting from Day 0 prior to test-article administration, that was not associated with any clinical signs of impaired hemostasis and could have been related to individual variation, artifact (i.e., clot in the collection tube), or splenic sequestration. Overall, all abnormalities noted from hemogram analysis highlighted above were minor and considered clinically insignificant.

### Serum Clinical Chemistry

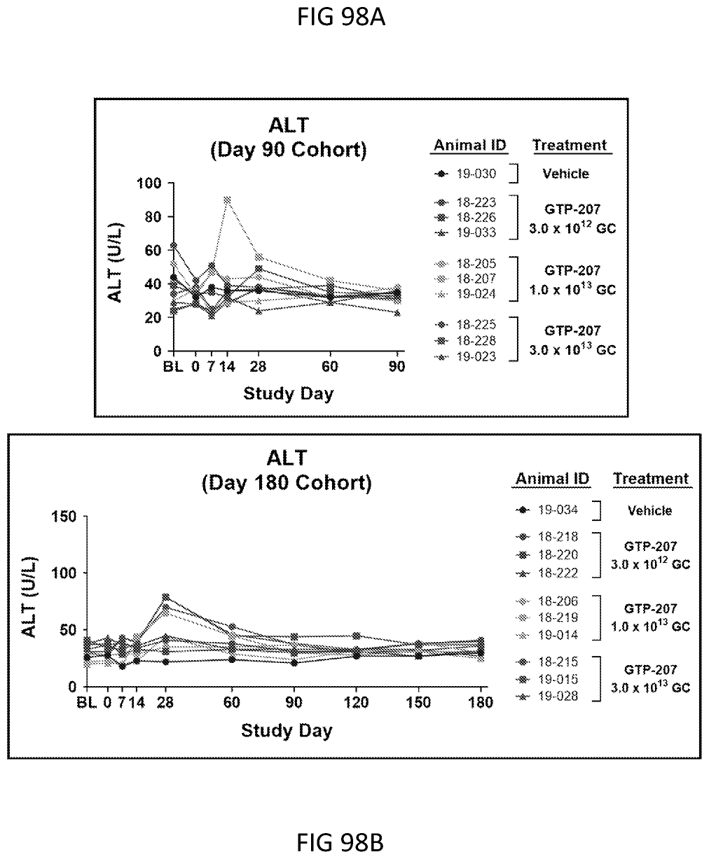

Overall, AST (liver enzyme) was unremarkable for all animals in this study. The animals showed no clinical signs of hepatotoxicity and overall, any elevations in ALT were very mild and many were either at baseline or Day 0 (FIG. 98). The cause of transient elevations cannot be determined definitively, but differentials include mild hepatocellular injury secondary to inflammation (bacterial, viral or fungal), congenital conditions (i.e. microvascular dysplasia), muscle trauma or test article administration. The mild self-limited ALT elevation noted on Day 28 in the Day 180 cohort is possibly test-article related as it was observed in 3/3 animals administered the high-dose (3.0×10¹³ GC, Animals 18-215, 19-015, and 19-028) and 1/3 animals administered mid-dose (1.0×10¹³ GC, Animal 18-219) and this timepoint coincides with possible adaptive T cell immune response to the non-self transgene and/or AAV capsid. The 4 animals with mild transient ALT elevation on Day 28 had positive T cell responses to the non-self transgene product as shown by ELIPSOT. The two Animals in the mid-dose group (1.0×10¹³ GC, Animals 18-206 and 19-014) that did not have elevated ALT values noted on Day 28 did not have an IFN-γ T cell response to hARSA at this timepoint. All Day 180 cohort Animals administered the low dose (3.0×10¹² GC, Animals 18-218, 18-220, and 18-222) did not have elevated ALT values on Day 28; while these Animals had an IFN-γ T cell response to hARSA on Day 28, the average of the highest response (83.7 spot forming units) for these Animals at this timepoint was much lower than the corresponding value (317.3 spot forming units) for the four Animals from the mid- and high-dose groups that had elevated ALT values on Day 28.

Transient elevations in blood glucose were observed in a subset of animals that were likely not associated with test or control article administration as many of these abnormalities occurred at baseline or Day 0 and may have been associated with alpha-2 adrenergic drugs used to sedate the animals that are also known to incite hyperglycemia. Other animals presented with intermittent hypoglycemia, several of which at baseline or Day 0, which indicates that this is not test article related. Those that were hyperglycemic at other time points may have been due to juvenile hypoglycemia, which is thought to occur to due hepatic immaturity and is often observed in young animals with low body mass.

Many animals presented with increases in serum alkaline phosphatase (ALP) throughout the course of this study. ALP has multiple isoforms including liver, kidney, and bone. While increases in ALP due to hepatobiliary or intestinal disease cannot be ruled out, these changes are most likely physiologic in nature due to the age of the test system. In young, growing animals ALP values may be up to 10 times higher than adults due high levels of the bone isoform of the enzyme. Many animals also had elevations in phosphorous that were also likely due to release from bone secondary to bone growth.

Multiple animals had elevations in CPK at various time points. Elevations involved the skeletal muscle isoform of CPK and could have been secondary to mild muscle trauma during sedation or venipuncture.

All abnormalities in the chemistry panels highlighted above, including those not explicitly discussed, are minor, transient, and considered clinically insignificant and likely a result of normal individual variation, however mild changes secondary to test article administration cannot be definitively ruled out.

### Coagulation

The coagulation profile data collected from this study revealed that there was evidence of hemolysis, and in some cases serum turbidity, in a subset of the samples, which may have contributed to the elevation in D-dimers.

Animals 19-033 and 19-024 had prolonged PT values on study days 28 and 60, respectively. For animal 19-033, this time point also had abnormalities in fibrinogen, D-Dimer, and FDP values, and the sample was noted to be hemolyzed. There were no clinically relevant abnormalities at any other time points for this animal, and the noted elevations were likely due to hemolysis of the sample. Animal 18-215 also had a mild increase in APTT at study day 185. None of these animals showed evidence of any clinical signs associated with coagulopathy, and the clotting times returned to within the normal reference ranges by the next study time point.

Animal 18-228 had elevated fibrinogen, D-Dimer and FDP values on the second baseline time point, and the sample was reported as hemolyzed. There were no clinically relevant abnormalities at any other time points for this animal and the noted elevations were likely due to hemolysis of the sample.

### Cerebrospinal Fluid

No test article-related abnormalities were noted on CSF clinical pathology with the exception of an asymptomatic mild transient increase in CSF leukocytes in some animals.

### Hematology (Cell Counts)

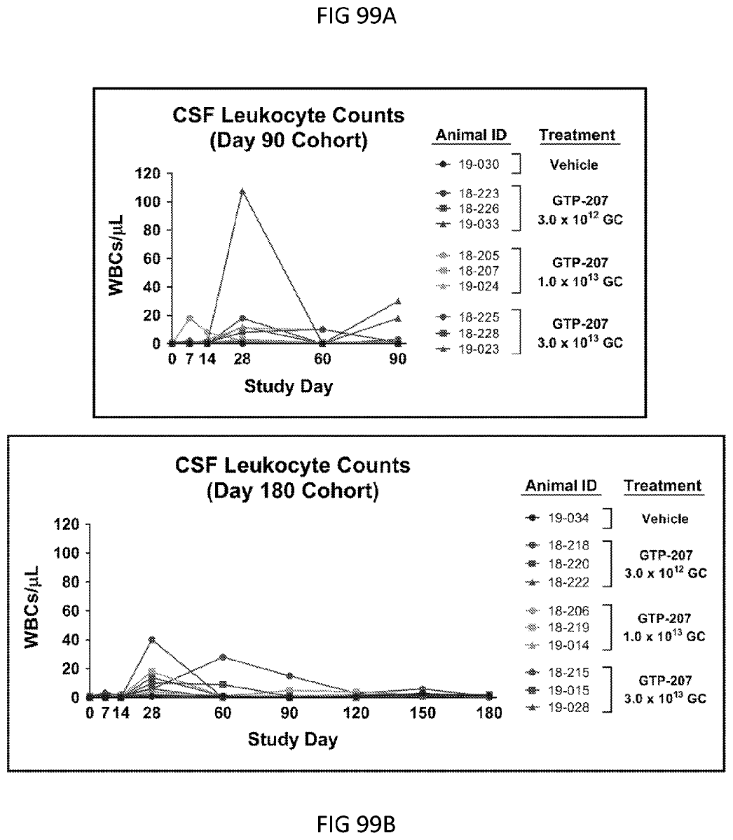

Pleocytosis can be related to hemodilution when >20 red blood cells (RBCs)/L are observed in CSF samples as a result of blood contamination due to inadvertent contact with a subcutaneous or dural vessel during placement of the spinal needle, and when the ratio of WBC per RBC is lesser in CSF than in blood. Of note, 4/18 GTP-207-treated animals exhibited pleocytosis consisting primarily of lymphocytes that was likely secondary to hemodilution (≥6 leukocytes/μL of CSF with >20 RBCs/μL and WBC to RBC ratio lesser in CSF than in blood), including 2/6 animals in the low dose groups (3.0×10¹² GC; Animal 18-220 [Group 6, Days 60] and Animal 19-033 [Group 2, Days 28]), 1/6 animals in the mid-dose groups (1.0×10¹³ GC; Animal 18-205 [Group 3, Days 7]), and 1/6 animals in the high dose groups (Animal 19-023 [Group 4, Day 90]). No pleocytosis was observed in the 2/2 vehicle-treated controls at any time point evaluated (FIG. 99).

Mild pleocytosis consisting primarily of lymphocytes that was not attributable to hemodilution (≥6 leukocytes/μL of CSF with ≤20 RBCs/μL, or with more than 20 RBCs with a WBC to RBC ratio higher in CSF than in blood) occurred in 12/18 AAVhu68.CB7.CI.hARSAco.rBG (GTP-207)-treated animals, including 4/6 animals in the low dose groups (3.0×10¹² GC; Animal 19-033 [Group 2, Day 90], Animal 18-223 [Group 2, Days 28 and 60], Animal 18-220 [Group 6, Day 28] and Animal 18-218 [Group 6, Day 28]), 3/6 animals in the mid-dose groups (1.0×10¹³ GC; Animal 19-024 [Group 3, Days 28 and 60], Animal 18-205 [Group 3, Day 14], and Animal 18-219 [Group 7, Day 28]), and 5/6 animals in the high dose groups (3.0×10¹³ GC; Animal 18-225 [Group 4, Day 28], Animal 19-023 [Group 4, Day 28], Animal 18-215 [Group 8, Days 28, 60, 90, 150], Animal 19-015 [Group 8, Day 28], and Animal 19-028 [Group 8, Day 28]). Among these AAVhu68.CB7.CI.hARSAco.rBG (GTP-207)-treated animals, peak CSF leukocyte counts not attributable to hemodilution ranged from 6-40 cells/μL, with peak leukocyte counts observed 28-60 days after AAVhu68.CB7.CI.hARSAco.rBG (GTP-207) administration. Since no pleocytosis was observed in the 2/2 vehicle-treated controls at any time point evaluated, mild CSF pleocytosis not attributable to hemodilution was therefore considered test article-related. In all cases, CSF pleocytosis was self-limited and not associated with clinical sequelae. Furthermore, there did not appear to be any correlation between dose and pleocytosis not attributable to blood contamination. These observations are consistent with the mild transient increases in CSF leukocyte counts historically observed in NHPs administered vector intrathecally, which have not been observed to have adverse effects 0.

### Clinical Chemistry

No abnormalities of CSF total protein or glucose were observed in any animals during the study.

### Presence of Neutralizing Antibodies Against AAVhu68 Capsid

At baseline, pre-existing NAbs against the AAVhu68 capsid were detectable in the serum of 4/20 animals in the study, including 1/2 vehicle-treated controls and 3/18 AAVhu68.CB7.CI.hARSAco.rBG (GTP-207)-treated NHPs.

Group 1 control Animal 19-030 (ITFFB) had a NAb titer of 160 at baseline as did Group 2 Animal 19-033 (low dose, 3.0×10¹² GC). Group 3 Animal 19-024 (mid dose, 1.0×10¹³ GC) and Group 6 Animal 18-222 (low dose, 3.0×10¹² GC) both had a baseline NAb titer of 40. The remaining animals on the study (16/20, 80%) had baseline NAb titers below the detection limit of the assay (<5).

Among the vehicle-treated controls, minimal to no change in baseline NAb titers were observed throughout the study. The animal that was negative for pre-existing AAVhu68 NAbs at baseline (Animal 19-034, Group 5, ITFFB) remained negative for AAVhu68 NAbs throughout the study until necropsy on Day 180. The NAb titer of the vehicle-treated control that was positive for pre-existing AAVhu68 NAbs (Animal 19-030, Group 1, ITFFB) decreased from a titer of 160 at baseline to a titer of 40 on Day 28 and remained at that level at necropsy on Day 90.

Among the AAVhu68.CB7.CI.hARSAco.rBG (GTP-207)-treated animals, NAb responses to the AAVhu68 capsid were observed in 18/18 animals by Day 28. AAVhu68 NAbs were detected through necropsy on either Day 90 or Day 180 for all animals administered GTP-207, with the peak response in the majority of animals occurring on either Day 28 or Day 90, followed by a reduction or maintenance of the NAb titer until necropsy. In general, the magnitude and kinetics of the NAb response were similar across all dose groups, with a two sample t-test with unequal variances demonstrating that there were no statistically significant differences in NAb titers among any of the dose groups at any of the study days evaluated (p≥0.05). The presence of pre-existing AAVhu68 NAbs prior to vector administration may have had an impact in animals administered the low dose of AAVhu68.CB7.CI.hARSAco.rBG (GTP-207) (3.0×10¹² GC; Animal 19-033, Group 2; Animal 18-222, Group 6), resulting in a higher magnitude and/or more rapid kinetics of the NAb response when compared to animals without pre-existing NAb that received the same vector dose.

NAb responses to the AAVhu68 capsid were not associated with abnormal clinical observations or changes in hematology, coagulation, and serum chemistry parameters. NAb responses to the AAVhu68 capsid in serum collected throughout the study.

### T Cell Responses to the AAV Capsid and Transgene Product

4/20 NHPs exhibited no IFN-γ T cell response to either the capsid (AAVhu68) or the transgene product (human ARSA) during the study. These non-responders included 2/2 vehicle-treated controls and 2/18 AAVhu68.CB7.CI.hARSAco.rBG (GTP-207)-treated animals. Both vehicle-treated non-responders (ITFFB; Animal 19-030, Group 1 and Animal 19-034, Group 5) remained negative for T cell responses through necropsy on Day 90 and Day 180, respectively. Among AAVhu68.CB7.CI.hARSAco.rBG (GTP-207)-treated animals, 1/6 non-responders in the mid-dose group (1.0×10¹³ GC; Animal 18-206, Group 7) remained negative for T cell responses through necropsy on Day 180, and 1/6 non-responders in the high dose group (3.0×10¹³ GC; Animal 19-023, Group 4) remained negative through Day 90.

16/20 NHPs exhibited IFN-γ T cell responses to the capsid and/or the transgene product during the study. All of these responders were AAVhu68.CB7.CI.hARSAco.rBG (GTP-207)-treated animals, including 6/6 animals in the low dose groups (3.0×10¹² GC, Groups 2 and 6), 5/6 animals in the mid-dose group (1.0×10¹³ GC, Groups 3 and 7), and 5/6 animals in the high dose groups (3.0×10¹³ GC, Groups 4 and 8).

With regard to the type of response observed, T cell responses to the transgene product were more prevalent than responses to the capsid. Of the 16/18 AAVhu68.CB7.CI.hARSAco.rBG (GTP-207)-treated animals exhibiting a T cell response, 15/16 animals had a response to the transgene product only, and 1/16 had a response to both the transgene product and the capsid. None of the AAVhu68.CB7.CI.hARSAco.rBG (GTP-207)-treated animals exhibited a T cell response to the capsid alone.

With regard to the magnitude of the response, T cell responses to the capsid were of a low magnitude (63-65 spot-forming units [SFU] per million cells), occurring in a single animal in the high dose group (3.0×10¹³ GC, Animal 18-225, Group 4). In contrast, T cell responses to the transgene product were of a higher magnitude (58-1703 SFU per million cells), with some cell populations exhibiting a greater response. Specifically, T cell response to the transgene product in liver lymphocytes were of a higher magnitude than that of PBMCs and other tissue-specific lymphocyte populations. T cell responses to the transgene product in liver lymphocytes were also of a generally higher magnitude in the mid-dose (1.5×10¹³ GC) and high dose (4.5×10¹³ GC) groups when compared to the responses in the low dose groups (4.5×10¹² GC).

With regards to prevalence, kinetics, and magnitude of the response, T cell responses to the transgene product appeared to have similar prevalence among all dose groups. T cell responses to the transgene product in PBMCs and tissue-specific lymphocytes were generally more prevalent at Day 90 than at Day 180 in the low dose (3.0×10¹² GC, Groups 2 and 6) and mid-dose groups (1.0×10¹³ GC, Groups 3 and 7). Of note, a higher prevalence of T cell responses in PBMCs at Day 28 (14/18 AAVhu68.CB7.CI.hARSAco.rBG (GTP-207)-treated animals) was observed than at necropsy on Day 90 or Day 180 in all dose groups.

Pre-existing NAbs against the capsid did not appear to impact T cell responses against the capsid, as none (0/3) of the GTP-207-treated animals with pre-existing NAbs against the capsid at baseline mounted a T cell response to the capsid during the study (3.0×10¹² GC, Animal 19-033 [Group 2] and Animal 18-222 [Group 6]; 1.0×10¹³ GC, Animal 19-024 [Group 3]).

T cell responses to the capsid and transgene product were not associated with abnormal clinical observations or changes in hematology, coagulation, and serum chemistry parameters, except a possible relationship between a transient ALT elevation and an anti-transgene product T cell response on Day 28.

### Gross Pathologic Findings

No test article-related gross findings were observed. All gross findings were considered incidental or procedurally related postmortem. Organ weights and organ to body weight ratios were within the expected values and inter-individual variability for the test species.

### Histopathologic Findings

Test article-related findings were observed primarily within sensory neurons of the peripheral nervous system in the DRG and in the trigeminal ganglia (TRG). The DRG/TRG findings consisted of neuronal degeneration/necrosis. Secondary axonal degeneration (i.e., axonopathy) was seen within the dorsal white matter tracts of the spinal cord and peripheral nerves, which contain central and peripheral axons, respectively, from DRG neurons.

Overall, findings of minimal to mild sensory neuron degeneration were observed across all AAVhu68.CB7.CI.hARSAco.rBG (GTP-207)-treated groups in the DRG. Less frequently, neuronal degeneration was observed in the TRG across GTP-207-treated groups with lower severity (minimal). For the DRG, three segments per animal (cervical, thoracic, lumbar) were analyzed, accounting for a total of 54 DRG segments in AAVhu68.CB7.CI.hARSAco.rBG (GTP-207)-treated animals. Minimal (Grade 1) and mild (Grade 2) neuronal degeneration was reported in 39% (21/54) and 7% (4/54) of the DRG segments evaluated, respectively, while 54% (29/54) of DRG did not display neuronal degeneration. For the TRG, one segment per animal was evaluated, accounting for a total of 18 TRG segments in AAVhu68.CB7.CI.hARSAco.rBG (GTP-207)-treated animals. Minimal (Grade 1) neuronal degeneration was reported in 44% (8/18) of the TRG segments evaluated, while 56% (10/18) of TRG did not display neuronal degeneration. To further evaluate the degeneration of DRG neurons, which project axons centrally and peripherally, three spinal cord segments were evaluated per animal (cervical, thoracic, lumbar) for a total of 54 spinal cord segments in AAVhu68.CB7.CJ.hARSAco.rBG (GTP-207)-treated animals, and 8 peripheral nerves were evaluated per animal (right and left proximal median, distal median, sciatic, peroneal, and tibial nerves) for a total of 144 peripheral nerves in AAVhu68.CB7.CJ.hARSAco.rBG (GTP-207)-treated animals.

A summary of the observed incidence and cumulative severity of DRG/TRG findings and associated secondary axonopathy in this study is discussed below.

DRG/TRG neuronal degeneration. Test article-related histopathologic findings consisted of neuronal cell body degeneration within the DRG (which project axons centrally into the dorsal white matter tracts of the spinal cord and peripherally to peripheral nerves) and/or TRG. The neuronal degeneration was characterized by central chromatolysis, cytoplasmic hypereosinophilia, and neuronophagia. Satellitosis and mononuclear cell infiltrates surrounding and infiltrating neuronal cell bodies was also observed. In more severe instances, ganglia contained nodules of compact, proliferating satellite cells lacking a central neuronal cell body were observed. The highest severity observed was Grade 2 (mild) in the DRG and Grade 1 (minimal) in the TRG.

At Day 90, a dose-dependent increase in the incidence and cumulative severity of neuronal degeneration within the DRG/TRG was observed across dose groups. The lowest incidence and cumulative severity (minimal, Grade 1) was observed in the low dose group (3.0×10¹² GC; 2/3 animals, 4/12 ganglia, Group 2). A higher incidence and cumulative severity (minimal to mild, Grade 1 to 2) was observed in the mid-dose group (1.0×10¹³ GC; 3/3 animals, 6/12 ganglia, Group 3), followed by the highest incidence and cumulative severity (minimal to mild, Grade 1 to 2) in the high dose group (3.0×10¹³ GC; 3/3 animals, 10/12 ganglia, Group 4). Mild (Grade 2) to marked (Grade 3) endoneurial fibrosis was also observed in the nerve root of the DRG of animals with mild (Grade 2) neuronal degeneration, including 2/3 animals in the mid-dose group (1.0×10¹³ GC, 2/3 animals; Animal 19-024, Animal 18-205; Group 3) and one animal in the high dose group (3.0×10¹³ GC, 1/3 animals Animal 18-225; Group 4). In some animals (Animal 18-205 and Animal 18-225), higher severity DRG degeneration with accompanying endoneurial fibrosis correlated with a reduction in SNAP amplitude on NCS assessment.

At Day 180, the incidence and severity of DRG/TRG neuronal degeneration did not appear dose-dependent, as a relatively similar incidence and severity (minimal, Grade 1) was observed at the low dose (3.0×10¹² GC, 3/3 animals, 4/12 ganglia, Group 6), mid-dose (1.0×10¹³ GC, 2/3 animals, 5/12 ganglia, Group 7), and high dose (3.0×10¹³ GC; 2/3 animals, 4/12 ganglia, Group 8).

Comparing across time points, the incidence and severity of DRG/TRG neuronal degeneration was similar at Day 180 compared to Day 90 at the low dose (3.0×10¹² GC) and reduced at Day 180 compared to Day 90 at the mid-dose (1.0×10¹³ GC) and high dose (3.0×10¹³ GC). This observation suggests the DRG/TRG findings did not progress or were potentially partially resolved from Day 90 to Day 180.

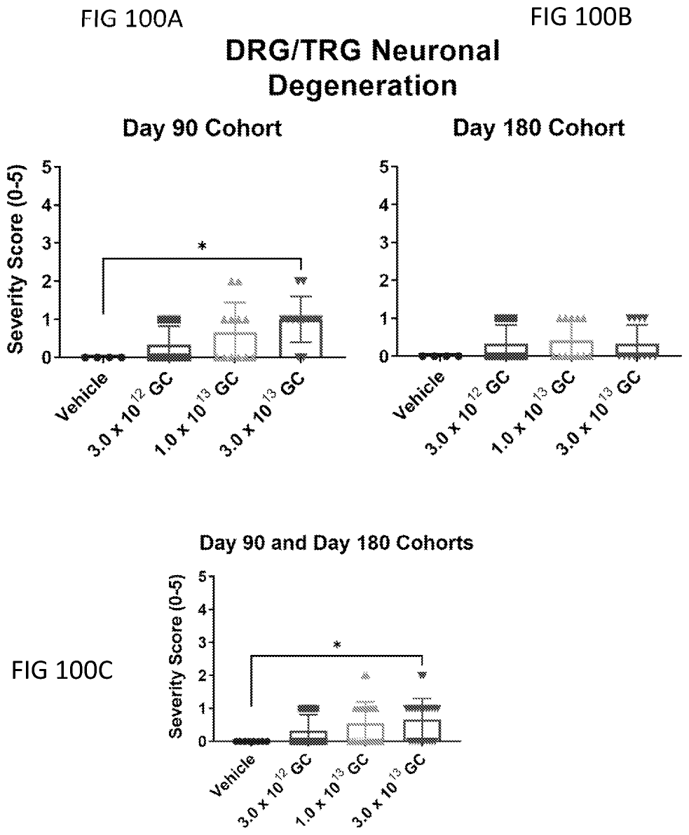

Individual DRG/TRG degeneration severity scores observed in the Day 90 and Day 180 necropsy cohorts are presented in FIG. 100.

Axonal degeneration in spinal cord. DRG degeneration resulted in secondary axonopathy of the dorsal white matter tracts of the cervical, thoracic, and lumbar spinal cord. The axonopathy was microscopically consistent with axonal degeneration. This test article-related axonal degeneration was characterized by dilated myelin sheaths with or without myelomacrophages and/or axonal debris. The severity of spinal cord axonopathy was generally low, with most findings ranging from minimal (Grade 1) to mild (Grade 2). At Day 90, moderate (Grade 3) axonopathy was observed in 2/3 animals in the mid-dose group (1.0×10¹³ GC; Animal 18-205, Animal 19-024; Group 3), and marked (Grade 4) axonopathy was observed in a single animal in the high dose group (3.0×10¹³ GC, 1/3 animals; Animal 18-225; Group 4).

At Day 90, the severity of spinal cord axonopathy was dose-dependent, increasing from minimal (Grade 1) to mild (Grade 2) at the low dose (3.0×10¹² GC), to minimal (Grade 1) to moderate (Grade 3) at the mid-dose (1.0×10¹³ GC), to minimal (Grade 1) to marked (Grade 4) at the high dose (3.0×10¹³ GC). However, the incidence of spinal cord axonopathy did not appear dose-dependent, as the incidence was similar among the low dose (3.0×10¹² GC; 8/9 segments, Group 2), mid-dose (1.0×10¹³ GC; 9/9 segments, Group 3), and high dose (3.0×10¹³ GC; 9/9 segments, Group 4) groups. The severity of the axonopathy at Day 90 was generally correlated with the abnormal decrease of median nerve SNAP amplitude in Animals 18-205 (mid-dose 1.0×10¹³ GC; Group 3) and 18-225 (high-dose 3.0×10¹³ GC; Group 4) that demonstrated the worse severity grade of 3 (moderate) and 4 (marked), respectively.

At Day 180, the incidence of axonopathy was similar between the low dose (3.0×10¹² GC; Group 6, 2/3 animals, 5/9 segments) and mid-dose (1.0×10¹³ GC; Group 7, 2/3 animals, 6/9 segments) groups and was highest in the high dose group (3.0×10¹³ GC; Group 8, 3/3 animals, 9/9 segments). The severity of axonal degeneration was similar between dose groups and was minimal (grade 1) to mild (grade 2).

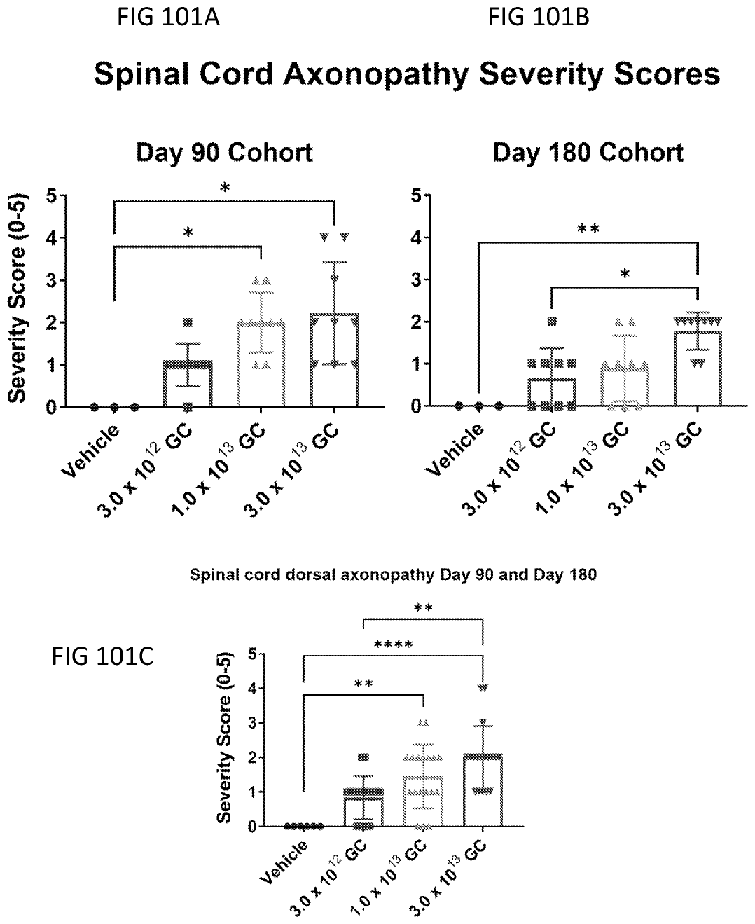

Comparing across time points, a time-dependent decrease in severity of dorsal white matter tract axonal degeneration was observed from Day 90 to Day 180 in the mid-dose groups (from minimal to moderate in Group 3 to minimal to mild in Group 7; 1.0×10¹³ GC) and in high dose groups (from minimal to marked in Group 4 to minimal to mild in Group 8; 3.0×10¹³ GC). The incidence of axonopathy was also decreased in low (3.0×10¹² GC; Groups 2 and 6) and mid-dose (1.0×10¹³ GC; Groups 3 and 7) groups across the Day 90 and Day 180 time points, indicative of time-dependent resolution. These findings, at minimum, indicate a lack of progression of the dorsal white matter tract axonal degeneration from Day 90 to 180. Severity scores for spinal cord axonopathy were observed in the Day 90 and Day 180 necropsy cohorts (FIG. 101)). Interestingly, the two animals with decreased median nerve SNAP amplitude from the Day 180 cohort did not show obvious correlation with histology findings in DRG and corresponding axons, unlike the Day 90 cohort animals. Given the fact that SNAP values were trending upwards after Day 90 and returned to within the normal range for this cohort of animals by Day 150 (Animal 19-015) or Day 180 (Animal 18-219), this supports the hypothesis of possible time-dependent resolution of the DRG-associated findings. As previously noted, Animal 18-219 suffered an injury to the second digit of its left hand on Day 28, which may have also contributed to the reduction in median nerve SNAP amplitudes on its left side while this injury was healing.

Individual spinal cord axonopathy severity scores observed in the Day 90 and Day 180 necropsy cohorts are presented in FIG. 101.

Axonopathy in peripheral nerves. DRG degeneration resulted in secondary axonopathy of the peripheral nerves (proximal and distal median, sciatic, peroneal and tibial), which was microscopically consistent with axonal degeneration. This axonal degeneration was similar in character to the axonal degeneration previously described with variably associated mononuclear cell infiltrates. Peripheral nerve axonal degeneration was generally bilateral, but variable within a given section.

At Day 90, no clear dose response of peripheral nerve axonal degeneration was observed. Severity across dose groups ranged from minimal to mild; however, the incidence was increased in the mid dose (1.0×10¹³ GC; Group 3, 3/3 animals, 21/30 segments) compared to both low (3.0×10¹² GC; Group 2, 3/3 animals, 16/30 segments) and high (3.0×10¹³ GC; Group 4, 3/3 animals, 17/30 segments) dose groups.

At Day 180, severity of peripheral nerve axonal degeneration was minimal across all dose groups. Incidence increased in a dose-dependent manner and was lowest in the low dose group (3.0×10¹² GC; Group 6, 2/3 animals, 2/30 segments), increased slightly in the mid-dose group (1.0×10¹³ GC; Group 7, 3/3 animals, 7/30 segments), and was highest in the high dose group (3.0×10¹³ GC; Group 8, 3/3 animals, 17/30 segments).

Comparing across time points, a time-dependent decrease in incidence of peripheral nerve axonal degeneration in the low and mid dose groups from Day 90 to 180 was observed. Incidences were similar between the Day 90 and Day 180 time points in the high dose groups. Additionally, there was a time-dependent decrease in severity at all doses from Day 90 (minimal [grade 1] to mild [grade 2]) to 180 (minimal [grade 1]). These findings at minimum suggest lack of progression of peripheral nerve axonal degeneration from Day 90 to 180 and possibly resolution.

Minimal (grade 1) to mild (grade 2) endoneurial fibrosis was observed sporadically in peripheral nerves from the mid dose (1.0×10¹³ GC; Group 3, 2/3 animals, 7/30 segments) and high dose (3.0×10¹³ GC; Group 4, 1/3 animals, 10/30 segments) groups at Day 90. It occurred with minimal severity (grade 1) in a single animal (Animal 19-015, 3.0×10¹³ GC, Group 8, 2/30 segments) from the high dose Group at Day 180, indicating a time-dependent decrease in the mid- and high-dose groups. Endoneurial fibrosis was considered secondary to axonal damage and most often associated with greater severity of axonal degeneration. It was generally correlated with an abnormal decrease in median SNAP amplitude as 3/3 animals with median nerve endoneurial fibrosis demonstrated decreased SNAP amplitude (Animals 18-205, 18-225, and 19-015).

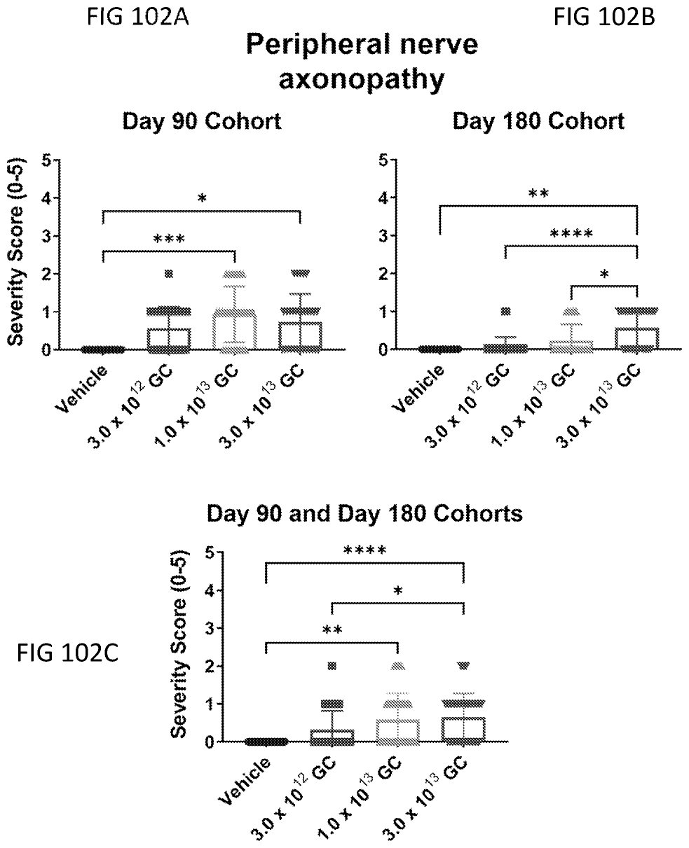

Individual peripheral nerves axonopathy severity scores observed in the Day 90 and Day 180 necropsy cohorts are presented in FIG. 102.

Mononuclear cell infiltrates, primarily composed of lymphocytes, plasma cells, and rarely macrophages, were observed within at least one peripheral nerve from all dose groups from Day 90 and Day 180 cohorts. While mononuclear cell infiltrates in peripheral nerves have been reported as background in nonhuman primates, the dramatically increased incidence and severity in the mid dose Group of the Day 90 cohort (1.0×10¹³ GC; Group 3, 3/3 animals, 15/30 segments, 16/150 cumulative severity score), was considered AAVhu68.CB7.CI.hARSAco.rBG (GTP-207)-related and coincided with the highest incidence/cumulative severity of axonal degeneration (1.0×10¹³ GC; Group 3, 3/3 animals, 21/30 segments, 28/150 cumulative severity score). The incidence and severity (mostly minimal, rarely mild) were considered relatively similar in the low and high dose groups from Day 90 (3.0×10¹² GC, Group 2, 2/3 animals, 6/30 segments, 6/150 cumulative severity score; 3.0×10¹³ GC; Group 4, 2/3 animals, 2/30 segments, 2/150 cumulative severity score) and Day 180 (3.0×10¹² GC, Group 6, 2/3 animals, 4/30 segments, 4/150 cumulative severity score; 3.0×10¹³ GC, Group 8, 2/3 animals, 4/30 segments, 4/150 cumulative severity score). A considerable decrease in peripheral nerve infiltrates was observed at the mid dose from Day 90 to Day 180, suggesting resolution of peripheral nerve infiltrates in the mid-dose Group from Day 90 (1.0×10¹³ GC; Group 3, 3/3 animals, 15/30 segments, 16/150 cumulative severity score) to Day 180 (1.0×10¹³ GC; Group 7, 1/3 animals, 1/30 segments, 1/150 cumulative severity score).

Injection site findings. At Day 90, GTP-207-related findings at the ICM injection/CSF collection site (with surrounding area) were similar across all doses in GTP-207-treated animals and included mild (grade 2) to moderate (grade 3) mononuclear cell infiltration of the skeletal muscle and/or adipose tissue with or without associated myofiber changes (e.g., degeneration and/or regeneration) and rarely minimal (grade 1) interstitial fibrosis. Minimal (grade 1) skeletal myofiber regeneration and moderate (grade 3) subacute hemorrhage was observed in the control animal and was likely procedural related to perimortem CSF collection given the lack of cellular response. Minimal superficial perivascular dermal infiltrate in a single Group 4 animal (3.0×10¹³ GC; Animal 18-225) was considered incidental.

At Day 180, similar, but less severe, GTP-207-related findings were observed at the ICM injection/CSF collection site across all doses, ranging from minimal (grade 1) to mild (grade 2) mononuclear cell infiltration of the skeletal muscle and/or adipose tissue without considerable myofiber changes or fibrosis. The reduction in severity of these findings from Day 90 to Day 180 was indicative of an ongoing resolution.

Brain findings. Minimal (grade 1) to mild (grade 2) mononuclear cell infiltrates were observed in the meninges in 17/18 test-article dosed animals and 1/2 control-article dosed animals. There was no impact of the dose on incidence or severity. This may represent background finding or procedural related finding. Sporadic minimal (grade 1) mononuclear infiltrates were seen in the brain parenchyma of 5/18 test-article dosed animals and 1/2 control-article dosed animal.

### Vector Pharmacokinetics and Excretion

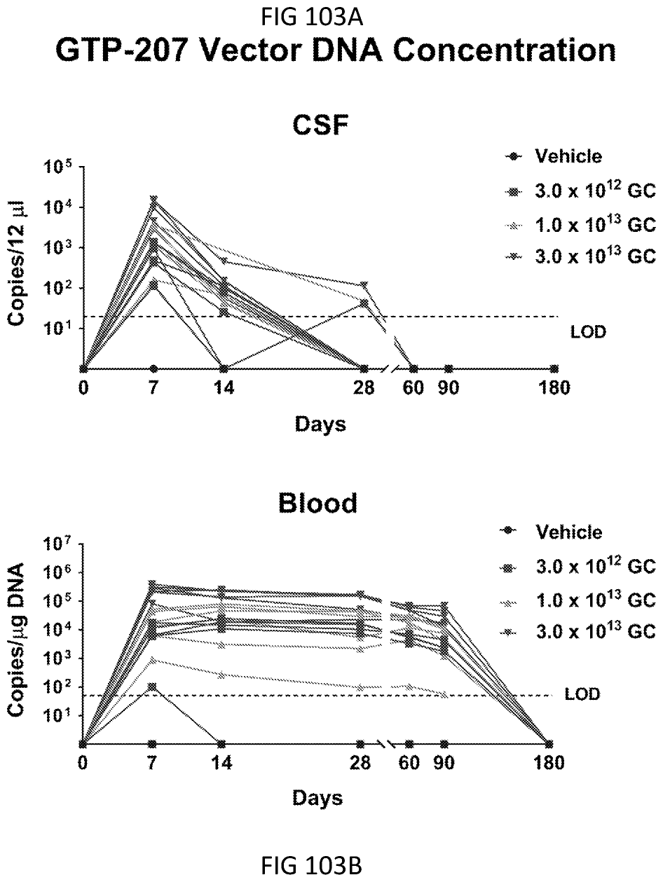

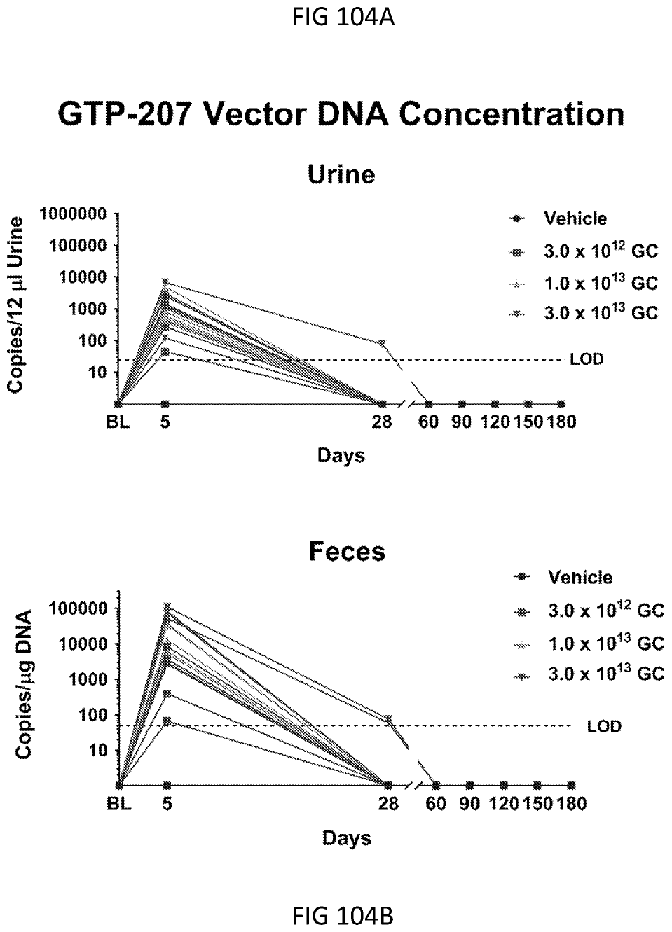

AAVhu68.CB7.CI.hARSAco.rBG (GTP-207) vector DNA was detectable in CSF and peripheral blood, with average peak concentrations in CSF and peripheral blood generally correlating with dose (FIG. 103). In CSF, the concentration of AAVhu68.CB7.CI.hARSAco.rBG (GTP-207) vector DNA rapidly declined following the first time point evaluated (Day 7) and was undetectable by Day 60 in all animals. In blood, AAVhu68.CB7.CI.hARSAco.rBG (GTP-207) vector DNA concentrations declined more slowly than in CSF, which may be attributed to transduction of peripheral blood cells. Detectable levels of AAVhu68.CB7.CI.hARSAco.rBG (GTP-207) vector DNA in the blood were observed in most animals up to Day 90, with no AAVhu68.CB7.CI.hARSAco.rBG (GTP-207) vector DNA detectable by Day 180.

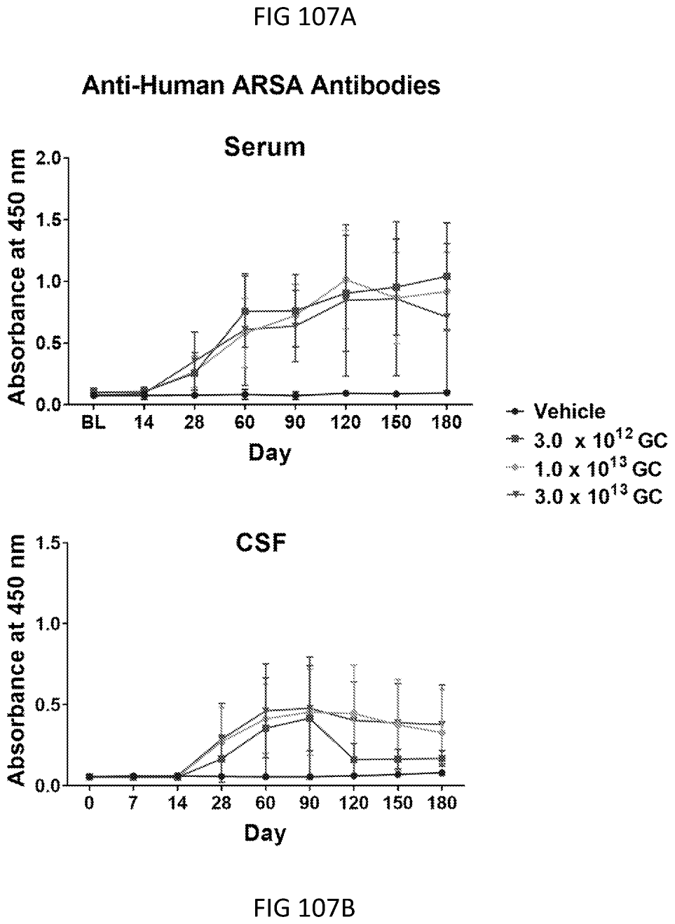

AAVhu68.CB7.CI.hARSAco.rBG (GTP-207) vector DNA was detectable in urine and feces on Day 5 after administration, with average peak concentrations correlating with dose (FIG. 107). In both feces and urine, AAVhu68.CB7.CI.hARSAco.rBG (GTP-207) vector DNA was undetectable in all animals by Day 60 after administration.

### Evaluation of Transgene Product Expression

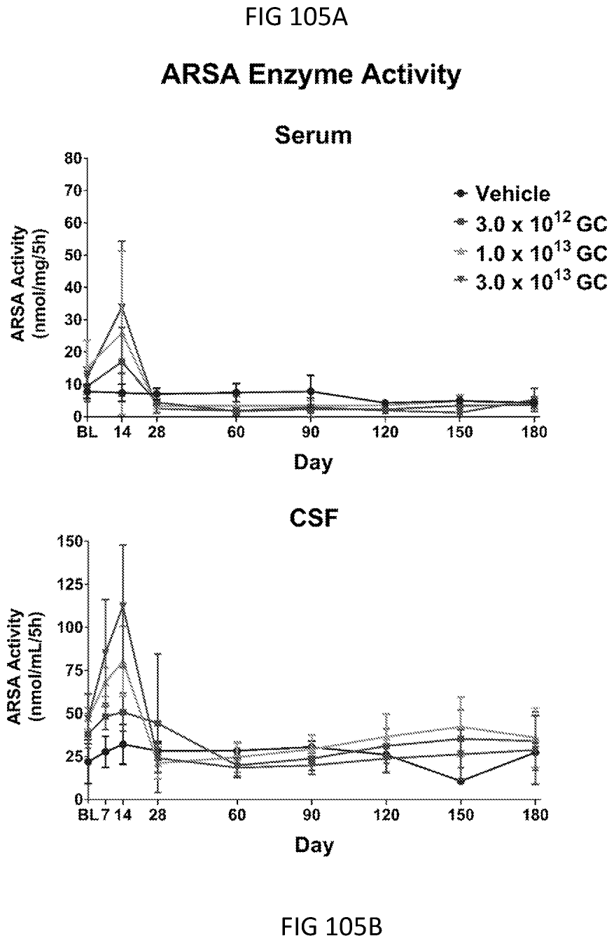

Transgene product expression (ARSA enzyme activity) was evaluated in serum and CSF (FIG. 105). It should be noted that this analysis was expected to be complicated by the rapid loss of measurable transgene product activity attributable to an NHP antibody response to the foreign human transgene product.

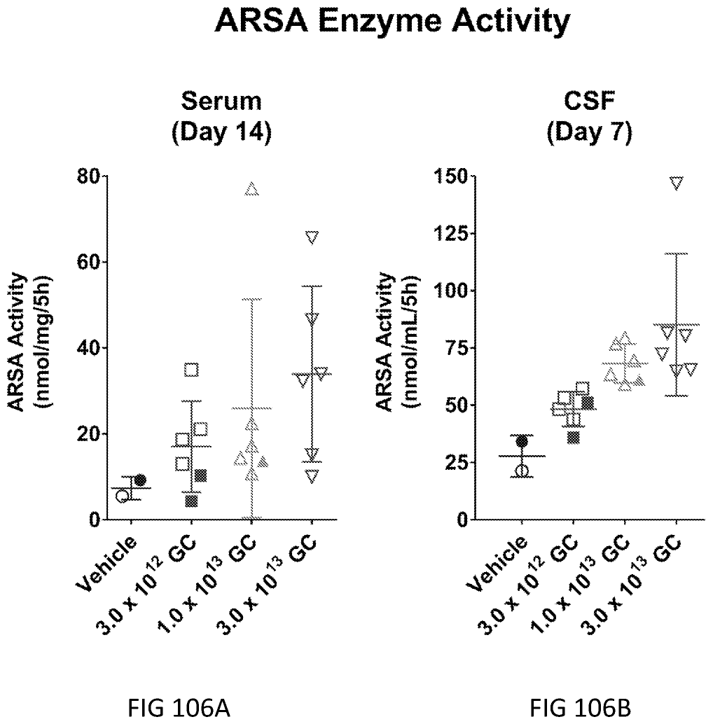

ARSA enzyme activity was detectable in serum and CSF of most animals from all dose groups by the first time point evaluated after AAVhu68.CB7.CI.hARSAco.rBG (GTP-207) administration (Day 14 for serum and Day 7 for CSF; FIG. 105). In serum, a dose-dependent increase in ARSA enzyme activity was observed at the first time point evaluated (Day 14), with an approximately 2.3-fold, 3.5-fold, and 4.6-fold increase over vehicle-treated control levels at the low dose (3.0×10¹² GC), mid-dose, (1.0×10¹³ GC), and high dose (3.0×10¹³ GC) of GTP-207, respectively (FIG. 106). As expected, a rapid decline in ARSA enzyme activity in serum after Day 14 was observed (FIG. 105), which correlated with the expression anti-human ARSA antibodies in serum by Day 28 (FIG. 107). In CSF, a dose-dependent increase in ARSA enzyme activity was also observed at the first time point evaluated (Day 7), with approximately 1.7-fold, 2.5-fold, and 3.1 fold increase over vehicle-treated control levels at the low dose (3.0×10¹² GC), mid-dose, (1.0×10¹³ GC), and high dose (3.0×10¹³ GC) of GTP-207, respectively (FIG. 106). As observed in the serum, ARSA enzyme activity in CSF rapidly declined after Days 14-28 (FIG. 105), which correlated with the expression of anti-human ARSA antibodies in CSF by Day 28 (FIG. 107).

The presence of pre-existing NAbs to the AAVhu68 capsid (denoted by the filled-in shapes in FIG. 106) did not appear to impact ARSA enzyme activity in CSF, supporting the potential to achieve therapeutic transgene expression in the target organ system (CNS and PNS) of MLD disease patients regardless of NAb status.

### Summary of Results

ICM administration of AAVhu68.CB7.CI.hARSAco.rBG (GTP-207) was well-tolerated at all doses evaluated. AAVhu68.CB7.CI.hARSAco.rBG (GTP-207) produced no adverse effects on clinical and behavioral signs, body weights, or neurologic and physical examinations. There were no abnormalities of blood and CSF clinical pathology related to GTP-207 administration except for an asymptomatic mild transient increase in CSF leukocytes, and a minimal self-limited increase in serum ALT in some animals on Day 28 that resolved without treatment.
AAVhu68.CB7.CI.hARSAco.rBG (GTP-207) administration resulted in minimal to mild degeneration of primarily dorsal root ganglia (DRG) and trigeminal ganglion (TRG) sensory neurons, which led to a secondary degeneration of the associated central and peripheral axons (axonopathy). The DRG lesions were present in a majority of animals and were minimal to mild in severity, while secondary spinal cord dorsal axonopathy was minimal to marked in severity. At Day 90, a dose-dependent increase in incidence and severity of DRG-associated findings was noted. At Day 180, the incidence and severity of DRG-associated findings was not dose-dependent and was relatively lower than at Day 90, suggesting the findings did not progress or that they partially resolved between the two necropsy time points.
In three animals (two from the Day 90 and one from the Day 180 cohort), the DRG-associated abnormalities noted in histopathology findings correlated with a reduction in median nerve SNAP amplitudes from baseline levels exceeding normal individual animal variability. Reduced amplitudes could be observed as early as Day 28, with a trend towards amelioration from Day 150 to Day 180 in the animal with longer follow-up, suggesting that the findings did not progress or partially resolved between the two necropsy time points, as suggested by histopathology.
Transgene expression (i.e., ARSA enzyme activity) in CSF and serum was detectable in animals from all dose groups by the first time point evaluated (Day 7 for CSF and Day 14 for serum). In serum, a dose-dependent increase in ARSA enzyme activity was observed at Day 14 with an approximately 2.3-fold, 3.5-fold, and 4.6-fold increase over vehicle-treated control levels at the low dose (3.0×10¹² GC), mid-dose, (1.0×10¹³ GC), and high dose (3.0×10¹³ GC), respectively. In CSF, Day 7 ARSA enzyme activity was approximately 1.7-fold, 2.5-fold, and 3.1-fold higher compared to vehicle-treated control levels at the low dose (3.0×10¹² GC), mid-dose, (1.0×10¹³ GC), and high dose (3.0×10¹³ GC), respectively. Transgene product expression in CSF was not affected by the presence of pre-existing NAbs to the vector capsid, supporting the potential to achieve therapeutic activity in the target organ systems (CNS and peripheral nervous system [PNS]) in MLD disease patients regardless of NAb status. However, rapid loss of measurable transgene product activity was observed, which was attributable to an NHP antibody response to the foreign human transgene product in CSF and serum. This humoral response to the foreign human transgene product was not associated with abnormal clinical or histopathological findings.
ICM administration of AAVhu68.CB7.CI.hARSAco.rBG (GTP-207) to juvenile NHPs resulted in vector distribution in the CSF and blood, with average peak concentrations in CSF and peripheral blood correlating with dose.
Evaluation of AAVhu68.CB7.CI.hARSAco.rBG (GTP-207) vector excretion demonstrated detectable vector deoxyribonucleic acid (DNA) in urine and feces 5 days after administration. In both feces and urine, AAVhu68.CB7.CI.hARSAco.rBG (GTP-207) vector DNA was undetectable in all animals by Day 60 after administration.
T cell responses to the human transgene product were detectable in the majority of AAVhu68.CB7.CI.hARSAco.rBG (GTP-207)-treated animals (16/18) in peripheral blood mononuclear cells (PBMCs) and/or tissue lymphocytes (liver, spleen, bone marrow, lymph nodes) at necropsy. T cell responses to the human transgene product were more frequent and of higher magnitude than T cell responses to the vector capsid that was only found in 1/18 animal. The T cell responses to the transgene in PBMCs were mostly transient, with a higher prevalence at Day 28 (14/18 animals) than at the terminal timepoint (5/18 animals). T cell responses to the vector capsid or human transgene product were generally not associated with any abnormal clinical or histopathological findings. There was a possible relation between transient anti-transgene T cell response at Day 28 and transient ALT elevation on Day 28 in 3 high dose and 1 mid-dose animals.
Pre-existing NAbs to the vector capsid were detectable in serum in 3/18 AAVhu68.CB7.CI.hARSAco.rBG (GTP-207)-treated animals at baseline and NAb responses to the AAVhu68 capsid were subsequently observed in 18/18 AAVhu68.CB7.CI.hARSAco.rBG (GTP-207)-treated animals by Day 28. Pre-existing NAbs to the vector capsid were not associated with T cell response to the capsid and were not associated with any abnormal clinical or histopathological findings.
In the absence of dose-limiting toxicity, the highest dose evaluated (3.0×10¹³ GC [3.3×10¹¹ GC/g brain]) was considered to be the maximum tolerated dose (MTD). Due to the presence of asymptomatic sensory neuron lesions in all dose groups, the NOAEL was not defined.

### Example 11—Sensory Neuron Toxicity in Non-Clinical Adeno-Associated Virus Studies

In order to reduce minimal to mild asymptomatic degeneration of DRG sensory neurons which appear in the AAV.CB7.CI.hARSAco.rBG toxicology study, vector genomes are constructed which include drg-detargetting miRNA sequences. A rAAV vector genome is constructed as, from 5′ to 3′, CB7 promoter, engineered hARSA coding sequence, four consecutive miRNA183 (with sequence of AGTGAATTCTACCAGTGCCATA, SEQ ID NO: 20) which are separated by a spacer and each spacer is independently selected from one or more of (A) GGAT; (B) CACGTG; or (C) GCATGC, and a rBG poly A. This vector genome is termed as AAV.CB7.CI.hARSAco.miRNA183.rBG while a rAAV comprising this vector genome and an AAVhu68 capsid is termed as AAVhu68.CB7.CI.hARSAco.miRNA183.rBG. Production of the rAAV vector comprising this vector genome is performed similar to the method described in Example 1. Efficacy and toxicity of the AAVhu68.CB7.CI.hARSAco.miRNA183.rBG are tested using methods and models described in Examples 2-10.

### Example 12—First-In-Human Trial

A Phase 1/2, multi-center, open-label, single-arm, dose escalation study of AAV.CB7.CI.hARSAco.rBG administered by a single ICM injection in pediatric patients (≥4 months of age) with early onset (late infantile or early juvenile) MLD caused by ARSA enzyme deficiency is performed. Safety and tolerability, pharmacodynamics, and clinical efficacy are assessed over 2 years, and all subjects are followed through 5 years post-administration of AAV.CB7.CI.hARSAco.rBG for the long-term evaluation of safety and tolerability, pharmacodynamics, disease progression, and clinical outcomes.

The study consists of a screening phase to determine eligibility of each potential subject from approximately Day −35 to Day −1. After confirmation of subject eligibility and parent's/guardian's willingness to have their child participate in the study, the subject undergoes baseline assessments, which include brain MRI, LP for CSF collection, blood draw, urine collection, vitals, ECG, a physical exam, a neurological exam, and clinical assessments. Baseline assessments occur on Days −1 and Day 0, and eligibility is reconfirmed at baseline prior to administration of AAV.CB7.CI.hARSAco.rBG.

During the treatment phase, subjects are admitted to the hospital on the morning of Day 0. Subjects receive a single ICM dose of AAV.CB7.CI.hARSAco.rBG on Day 0 and remain in the hospital for at least 24 hours after dosing for observation. Subsequent study visits occur at Day 7, Day 14, Day 30, 3 months, and 6 months after dosing, followed by every 6 months for the first 2 years after dosing. Long-term follow-up (LTFU) visits occur for an additional 3 years at a frequency of every 12 months through 5 years post-dosing.

A single dose of AAV.CB7.CI.hARSAco.rBG is administered at one of dose levels as indicated below.

Cohort 1 (Low Dose): Three eligible subjects (Subjects #1-3) are sequentially enrolled and administered the low dose of AAV.CB7.CI.hARSAco.rBG with a 4 week safety observation period between the first and second subject. If no SRTs are observed, all available safety data is evaluated by a safety board 4 weeks after the third subject in Cohort 1 is administered AAV.CB7.CI.hARSAco.rBG.

Cohort 2 (High Dose): Three eligible subjects (Subjects #4-6) are sequentially enrolled and administered the high dose of AAV.CB7.CI.hARSAco.rBG with a 4 week safety observation period between the fourth and fifth subject. If no SRTs are observed, the safety board evaluates all available safety data 4 weeks after Subject #6 is administered AAV.CB7.CI.hARSAco.rBG, including safety data from subjects in Cohort 1.

Cohort 3 (MTD, Maximum Tolerated Dose): With a positive recommendation by the safety board, 6 additional subjects (Subjects #7-12) are enrolled and administered a single ICM dose AAV.CB7.CI.hARSAco.rBG at the MTD. Dosing for subjects in this cohort are not staggered with a 4 week safety observation period between each subject.

A total enrollment of 9 subjects are enrolled in either the high dose or low dose cohort, and 12 subjects in total (across all doses). A safety margin is applied so that the high dose selected for human subjects is 30-50% of the equivalent MTD in NHPs. The low dose is typically 2-3-fold less than the selected high dose provided it is a dose that exceeds the equivalent scaled MED in the animal studies. With the understanding that if tolerated, the higher dose would be expected to be advantageous.

Since early onset MLD, is marked by a very rapid disease course once symptoms emerge, a study is performed to allow for concurrent enrollment of subjects 30 days after dosing of the first patient in Cohort 1 (low dose) and Cohort 2 (high dose) based on the Investigator's benefit-risk assessment for that subject. In this case, the dosing window between the second and third patient in the cohort would be at least 24 hours to observe the patient for acute toxicity, allergic reactions, and procedure-related events. Given the rarity of the disease, the probability that two subjects would present simultaneously for treatment is considered low. The rationale for the proposed approach is that the risk of missing the treatment window because the patient experienced rapid disease progression would outweigh the potential benefit of prolonged safety follow-up before dosing the next patient in the cohort. Such a scenario where patients experience substantial disease progression between enrollment and treatment was cited as a possible cause of the poor outcomes observed in some early onset MLD patients treated with HSC-GT (Sessa et al., 2016), highlighting the need for prompt identification and treatment of patients at risk of rapid disease progression.

The pediatric patients (≥4 months of age) with early onset (late infantile or early juvenile) MLD caused by ARSA enzyme deficiency represent the population with the highest unmet need. Those MLD patients enrolled in our proposed FIH trial may have a 0/0 (two null ARSA alleles with no detectable functional enzyme produced) or 0/R (Heterozygosity for one null ARSA allele and one “residual” ARSA allele (R) encoding enzyme with residual functional activity that can still degrade small amounts of sulfatide) genotype. They display a devastating disease course with rapid and predictable decline in both motor and cognitive impairment leading to death within a few years of disease onset. Disease-modifying treatments are unavailable for most early onset patients. Hematopoietic stem cell transplant (HSCT) does not provide benefit in this population, while hematopoietic stem cell with gene therapy (HSC-GT) is an investigational treatment that is only effective in pre-symptomatic patients, who constitute a small minority of the early onset population.

Primary endpoints assess the safety and tolerability of AAV.CB7.CI.hARSAco.rBG. Secondary or exploratory endpoints include pharmacokinetic and pharmacodynamic properties (transgene expression, biomarker activity, and imaging parameters) and clinical efficacy outcomes (gross and fine motor function, cognitive and language development, neurological exam findings, behavioral and milestone development, and parent-reported outcomes and quality of life assessments). Efficacy endpoints and timing of follow-up were selected to measure prevention or stabilization of disease progression.

Gallbladder pathologies are therefore monitored in our proposed FIH trial as both a safety signal and an exploratory endpoint.

To assess the safety and tolerability of AAV.CB7.CI.hARSAco.rBG through 24 months following administration of a single ICM dose through evaluation of: AEs and SAEs, Vital signs and physical examinations, Neurological examinations, Electrocardiograms (ECGs), Sensory nerve conduction studies (for evaluation of DRG toxicity), Laboratory assessments (serum chemistry, hematology, coagulation studies, liver function tests [LFTs], urinalysis, and CSF chemistry and cytology), and/or immunogenicity of the vector and transgene product.

To assess the effect of AAV.CB7.CI.hARSAco.rBG on gross motor function through 2 years post-treatment as measured by the Gross Motor Function Classification for Metachromatic Leukodystrophy (GMFC-MLD).

Additional measurements for efficacy of AAV.CB7.CI.hARSAco.rBG includes the following:

To assess the pharmacodynamics and biological activity of AAV.CB7.CI.hARSACO.RBG over 2 years following administration of a single ICM dose based on the following endpoints: Levels of ARSA in CSF and serum;

To assess the efficacy of AAV.CB7.CI.hARSAco.rBG through 2 years following administration of a single ICM dose as measured by: Gross Motor Function Measure (GMFM), Neuro-cognitive (Total Intelligence Quotient [IQ] and sub-domain IQ measured by the Bayley Scale of Infant Development [BSID-III], Wechsler Intelligence Scale for Children, Fifth Edition [WISC-V]), Survival, Neurological clinical exam (NCE), NCV of the ulnar, deep peroneal, median, sural nerves, Motor milestones achievement (as defined by World Health Organization [WHO] criteria) assessed by age at achievement, age at loss, and percentage of children maintaining or acquiring motor milestones;

To further assess the efficacy of AAV.CB7.CI.hARSAco.rBG through 2 years following administration of a single ICM dose as measured by: Age-at-onset and frequency of seizures captured by a seizure diary, Vineland Adaptive Behavior Scales, Third Edition (Vineland-III), Lansky Performance Index, Pediatric Quality of Life Inventory (PedsQL and PedsQL-IS), Caregiver/parent quality of life;

To further assess the pharmacodynamic effects of AAV.CB7.CI.hARSAco.rBG through 2 years following administration of a single ICM dose as measured by: CNS myelination (demyelination load and pattern) and white matter atrophy as measured by MRI, Neuronal metabolite N-acetylaspartate (NAA), myo-inositol (ml), choline (Cho) and lactate (Lac) levels as measured by proton magnetic resonance spectroscopy (MRS), CSF sulfatide and lyso-sulfatide levels, Visual evoked potentials (VEPs), Brainstem auditory evoked responses (BAERs), Ultrasound evaluation of gall-bladder wall thickening.

Inclusion Criteria comprises the following:
(i) Documented biochemical and molecular diagnosis of MLD based on ARSA activity below the normal range and identification of two disease-causing ARSA alleles, either known or novel mutations. In the case of a novel mutation(s), a 24 hour urine collection must show elevated sulfatide levels;
(ii) ≥4 months of age;
(iii) Pre-Symptomatic Subjects must have either
   An older sibling affected by MLD (index case) whose age of symptom onset was <7 years of age. Subjects are classified as late infantile, early juvenile, or intermediate late infantile/early juvenile based on age of symptom onset in the index case and their ARSA genotype as follows: Late infantile: symptom onset in index case ≤30 months of age and genotype typically 0/0; Early juvenile: symptom onset in index case >30 months and <7 years of age with genotype typically 0/R; Intermediate late infantile/early juvenile: symptom onset in index case <7 years of age, but unable to unambiguously characterize index case as late infantile or early juvenile
   or
   If MLD is diagnosed in a pre-symptomatic child without an older affected sibling (e.g., incidentally or via newborn screening when available), the totality of available data strongly suggest that the subject has an early onset variant of MLD likely to benefit from gene therapy, and the subject is <7 years of age, then the subject may be considered eligible after discussion and approval by the Sponsor Medical Monitor;
(iv) Symptomatic Late Infantile Subjects are eligible provided they have mild clinical or neurological manifestations of MLD manifested by:
   A delay in expected achievement of motor milestones such as a delay in achieving independent standing or walking (>95^{th} percentile on WHO milestone ranges)
   And/or
   Mild abnormalities on NCE including, but not limited to, increased tone, spasticity, or hyperreflexia. If the subject has achieved independent walking, then signs of mild ataxia are acceptable provided the subject can walk at least 10 steps independently;
(v) Symptomatic Early Juvenile Subjects are eligible if they have mild/moderate abnormalities on NCE including, but not limited to, increased tone, spasticity, hyperreflexia, or mild gait abnormalities not requiring aids for walking;
(vi) Parent/guardian signed and dated informed consent.
Exclusion Criteria comprises the following:
Evidence of regression of achieved motor milestones;
Ambulatory subjects requiring aids for walking;
EJ MLD subjects with cognitive deficit based on Total IQ <70;
Any clinically significant neurocognitive deficit not attributable to MLD that may, in the opinion of the Investigator, confound interpretation of study results;
Patients with a positive test result for human immunodeficiency virus (HIV) or Hepatitis C (HepC);
Any current or previous condition or physical exam or laboratory test finding that, in the opinion of the Investigator, would put the subject at undue risk or would interfere with evaluation and interpretation of the investigational product safety or efficacy results;
Any contraindication to the ICM administration procedure, including contraindications to fluoroscopic imaging;
Any contraindication to MRI or LP;
Enrollment in any other clinical study with an investigational product within 4 weeks prior to screening or within 5 half-lives of the investigational product used in that clinical study, whichever is longer;
Has previously undergone allogeneic HSCT and has evidence of residual cells of donor origin;
Previous gene therapy.

Route of Administration and Procedure are described in detail below.

AAV.CB7.CJ.hARSAco.rBG is administered as a single dose to hospitalized subjects on Day 0 via real-time CT-guided sub-occipital injection into the cisterna magna.

On Day 0, a syringe containing 5.6 mL of AAV.CB7.CI.hARSAco.rBG at the appropriate titer is prepared by the Investigational Pharmacy associated with the study and delivered to the procedure room.

Prior to study drug administration, the subject is anesthetized, intubated, and the injection site is prepped and draped using sterile technique. An LP is performed to remove a predetermined volume of CSF, after which iodinated contrast is IT injected to aid in visualization of relevant anatomy of the cisterna magna. IV contrast may be administered prior to or during needle insertion as an alternative to the IT contrast. The decision to use IV or IT contrast is at the discretion of the interventionalist performing the procedure. A spinal needle (22-25 G) is advanced into the cisterna magna under CT-fluoroscopic guidance. A larger introducer needle may be used to assist with needle placement. After confirmation of needle placement, the extension set is attached to the spinal needle and allowed to fill with CSF. At the discretion of the interventionalist, a syringe containing contrast material may be connected to the extension set and a small amount injected to confirm needle placement in the cisterna magna. After the needle placement is confirmed, the syringe containing AAV.CB7.CI.hARSAco.rBG is connected to the extension set. The syringe contents are slowly injected over 1-2 minutes, delivering a volume of 5.0 mL.

Safety assessments, including collection of Adverse Events (AEs) and Serious Adverse Events (SAEs), physical and neurologic examinations, vital signs, clinical laboratory tests (serum chemistry, hematology, coagulation, LFTs, urinalysis), ECGs, nerve conduction studies, and CSF cytology and chemistry (cell counts, protein, glucose) are performed. Safety evaluations after the first three subjects in Cohort 1 and after the first three subjects in Cohort 2 are conducted.

Statistical comparisons are performed for secondary and exploratory endpoints. Measurements at each time point are compared to baseline values for each subject, as well as data from age-matched healthy controls and natural history data from MLD patients with comparable cohort characteristics where available for each endpoint.

All data is presented in subject data listings. Categorical variables are summarized using frequencies and percentages, and continuous variables are summarized using descriptive statistics (number of non-missing observations, mean, standard deviation, median, minimum, and maximum). Graphical displays are presented as appropriate.

### Example 13—AAVhu68+Deamidation

AAVhu68 was analyzed for modifications. Briefly, AAVhu68 vectors were produced using vector genomes which are not relevant to this study, each produced using conventional triple transfection methods in 293 cells. For a general description of these techniques, see, e.g., Bell C L, et al., The AAV9 receptor and its modification to improve in vivo lung gene transfer in mice. J Clin Invest. 2011; 121:2427-2435. Briefly, a plasmid encoding the sequence to be packaged (a transgene expressed from a chicken β-actin promoter, an intron and a poly A derived from Simian Virus 40 (SV40) late gene) flanked by AAV2 inverted terminal repeats, was packaged by triple transfection of HEK293 cells with plasmids encoding the AAV2 rep gene and the AAVhu68 cap gene and an adenovirus helper plasmid (pAdΔF6). The resulting AAV viral particles can be purified using CsCl gradient centrifugation, concentrated, and frozen for later use.

Denaturation and alkylation: To 100 μg of the thawed viral preparation (protein solution), add 2 μl of 1M Dithiothreitol (DTT) and 21 of 8M guanidine hydrochloride (GndHCl) and incubate at 90° C. for 10 minutes. Allow the solution to cool to room temperature then add 5 μl of freshly prepared 1M iodoacetamide (IAM) and incubate for 30 minutes at room temperature in the dark. After 30 minutes, quench alkylation reaction by adding 1 μl of 1M DTT.

Digestion: To the denatured protein solution add 20 mM Ammonium Bicarbonate, pH 7.5-8 at a volume that dilutes the final GndHCl concentration to 800 mM. Add trypsin solution for a 1:20 trypsin to protein ratio and incubate at 37° C. overnight. After digestion, add TFA to a final of 0.5% to quench digestion reaction.

Mass Spectrometry: Approximately 1 microgram of the combined digestion mixture is analyzed by UHPLC-MS/MS. LC is performed on an UltiMate 3000 RSLCnano System (Thermo Scientific). Mobile phase A is MilliQ water with 0.1% formic acid. Mobile phase B is acetonitrile with 0.1% formic acid. The LC gradient is run from 4% B to 6% B over 15 min, then to 10% B for 25 min (40 minutes total), then to 30% B for 46 min (86 minutes total). Samples are loaded directly to the column. The column size is 75 cmx 15 um ID. and is packed with 2 micron C18 media (Acclaim PepMap). The LC is interfaced to a quadrupole-Orbitrap mass spectrometer (Q-Exactive HF, Thermo Scientific) via nanoflex electrospray ionization using a source. The column is heated to 35° C. and an electrospray voltage of 2.2 kV is applied. The mass spectrometer is programmed to acquire tandem mass spectra from top 20 ions. Full MS resolution to 120,000 and MS/MS resolution to 30,000. Normalized collision energy is set to 30, automatic gain control to 1e5, max fill MS to 100 ins, max fill MS/MS to 50 ins.

Data Processing: Mass spectrometer RAW data files were analyzed by BioPharma Finder 1.0 (Thermo Scientific). Briefly, all searches required 10 ppm precursor mass tolerance, 5 ppm fragment mass tolerance, tryptic cleavage, up to 1 missed cleavages, fixed modification of cysteine alkylation, variable modification of methionine/tryptophan oxidation, asparagine/glutamine deamidation, phosphorylation, methylation, and amidation.

In the following table, T refers to the trypsin and C refers to chymotrypsin.

Modification AAVhu68

| Enzyme | T | T | T | T | C | C | C | C | T | T | T | |
|---|---|---|---|---|---|---|---|---|---|---|---|---|
| % Coverage | 93.6 | 92 | 93.1 | 92.5 | 90.2 | 89.7 | 91.1 | 88.9 | 98.9 | 97 | 94.6 | 92.4 |

+Deamidation (Deamid)

| | | | | | | | | | | | | |
|---|---|---|---|---|---|---|---|---|---|---|---|---|
| ~N35 | | | | | | | | | | | | |
| N57 + Deamid | 87.6 | 95.5 | 893 | 88.2 | 90.5 | 96.3 | 86.4 | 84.8 | 100.0 | 100.0 | 99.0 | 92.7 |
| N66 + Deamid | 4.7 | | | | | | | | | | | |
| N94 + Deamid | 11.3 | 10.9 | 11.0 | 5.3 | 11.6 | 10.4 | 10.8 | 5.6 | 5.0 | 11.1 | 5.4 | 16.0 |
| N113 + Deamid | | | 1.8 | | | | | | | | | |
| ~N253 + Deamid | 17.7 | 22.0 | 21.1 | 15.0 | 17.0 | 22.6 | 20.5 | 15.6 | 4.2 | 5.5 | | |
| Q259 + Deamid | 35.2 | 25.6 | 21.0 | | 35.4 | 26.3 | 20.9 | 9.2 | | | | |
| ~N270 + Deamid | 16.4 | 25.1 | 23.2 | 16.6 | 15.9 | 24.9 | 235 | 16.1 | 0.2 | | | |
| % Coverage | 93.6 | 92 | 93.1 | 92.5 | 90.2 | 89.7 | 91.1 | 88.9 | 98.9 | 97 | 94.6 | 92.4 |
| ~N304 + Deamid | 2.6 | 2.9 | 2.8 | 1.3 | 2.5 | 2.8 | 2.9 | 1.3 | 16.6 | 10.3 | | |
| ~N314 + Deamid | | | | | | | | | 6.5 | | | |
| N319 + Deamid | 0.3 | 2.8 | 2.8 | 0.2 | | 2.9 | 2.8 | 0.2 | | | | |
| N329 + Deamid | 72.7 | 85.6 | 89.1 | 86.8 | 71.0 | 87.2 | 88.7 | 84.7 | 85.5 | 79.4 | 78.9 | 91.8 |
| N336 + Deamid | | 30.8 | 9.3 | 100.0 | | 31.0 | 9.2 | 95.7 | | | | |
| ~N409 + Deamid | 21.3 | 22.9 | 23.9 | 24.0 | 22.0 | 23.4 | 24.7 | 24.2 | | | | |
| N452 + Deamid | 98.8 | 99.7 | 99.2 | 100.0 | 98.9 | 97.3 | 98.1 | 95.2 | 98.2 | 68.7 | 67.4 | 49.4 |
| N477 + Deamid | 4.4 | 4.3 | 4.3 | 2.6 | 4.5 | 4.4 | 4.3 | 2.6 | | | 0.8 | |
| N512 + Deamid | 97.5 | 97.9 | 95.3 | 95.7 | 92.2 | 91.8 | 99.2 | 96.1 | 99.7 | 98.2 | 87.9 | 75.7 |
| ~N515 + Deamid | 8.2 | 21.0 | 16.0 | | 8.3 | 21.0 | 16.5 | 0.0 | 2.5 | 3.0 | | 15.1 |
| ~Q599 + Deamid | 4.0 | 15.4 | 10.1 | 13.6 | 4.0 | 15.5 | 10.0 | 13.8 | 15.8 | | | |
| N628 + Deamid | 5.3 | | 5.6 | | 5.4 | 0.0 | 5.4 | 0.0 | | | | |
| N651 + Deamid | 0.9 | 1.6 | 1.6 | | | | | | 0.5 | | | |
| N663 + Deamid | 3.4 | | 3.5 | 3.7 | 3.4 | 0.0 | 3.4 | 3.6 | | | | |
| N709 + Deamid | 0.6 | 0.8 | 20.2 | 0.6 | 0.6 | 0.8 | 19.8 | 0.6 | 0.3 | 1.3 | 0.1 | 0.2 |
| % Coverage | 93.6 | 92 | 93.1 | 92.5 | 90.2 | 89.7 | 91.1 | 88.9 | 98.9 | 97 | 94.6 | 92.4 |
| N735 | | | | | | | | | 25.0 | 42.7 | | 21.7 |

+ Acetylation (Ac):

| | | | | |
|---|---|---|---|---|
| K332 + Ac | | | | 100.0 |
| ~K693 + Ac | 13.0 | | 13.5 | |
| ~K666 + Ac | | | | 93.8 |
| ~K68 + Ac | | 59.2 | | |

+Isomerization (Iso):

| | | | | | | | | |
|---|---|---|---|---|---|---|---|---|
| D97 + Iso | 0.5 | 0.4 | 0.4 | 0.2 | 0.5 | | 0.4 | 0.2 |
| D107 + Iso | | 0.3 | | 0.3 | | 0.3 | | |
| D384 + Iso | 0.8 | | | | 0.9 | | | |

+Phosphorylation (Phos)

| | | | | | | | | |
|---|---|---|---|---|---|---|---|---|
| S149 + Phos | 5.8 | 5.7 | 5.2 | 9.8 | 5.7 | 5.9 | 5.2 | 9.9 |
| ~S499 + Phos | | | | 30.6 | | | | |
| ~T569 + Phos | 0.9 | | | | | | | |
| ~S586 + Phos | | 3.6 | | | | | | |

+Oxidation

| | | | | | | |
|---|---|---|---|---|---|---|
| ~W23 + Oxi | | 4.7 | 5.5 | | 4.8 | 5.5 |
| W247 + Oxi | 1.5 | 0.4 | 0.7 | 1.4 | | |

Modification AA Vhu68

| | | | | | | | | | | | | |
|---|---|---|---|---|---|---|---|---|---|---|---|---|
| % Coverage | 93.6 | 92 | 93.1 | 92.5 | 90.2 | 89.7 | 91.1 | 88.9 | 98.9 | 97 | 94.6 | 92.4 |
| W247 + Oxi to | | 0.1 | | | | 0.1 | | | | | | |
| kynurenine | | | | | | | | | | | | |
| W306 + Oxi | 0.7 | 0.9 | 1.6 | 1.8 | 0.7 | 1.0 | 1.6 | 1.8 | | | | |
| W306 + Oxidation to | | | 0.3 | | | | 0.3 | | | | | |
| kynurenine | | | | | | | | | | | | |
| M404 + Oxi | 0.1 | | 0.2 | | 0.1 | | 0.2 | | | | | |
| M436 + Oxi | 4.9 | | 10.2 | 23.0 | 4.8 | | 10.2 | 226 | | | | |
| ~M518 + Oxi | 299 | | 1.5 | 10.6 | 299 | | 1.5 | 10.5 | | | | |
| ~M524 + Ox | 18.8 | 31.6 | 52.7 | | 18.4 | 31.1 | 52.5 | 14.2 | | | | |
| M559 + Oxi | 19.0 | 21.6 | 19.6 | 20.9 | 19.6 | 21.3 | 20.1 | 20.9 | | | | |
| ~M605 + Oxi | 12.2 | 15.2 | | | 12.8 | 14.8 | | | | | | |
| W619 + Oxi | 1.0 | | 0.6 | 1.5 | 1.0 | | 0.6 | 1.5 | | | | |
| W619 + Oxidation | | | 20.3 | | | | | | | | | |
| ~M640 + Oxi | 23.5 | 64.2 | 24.6 | | 22.4 | 21.1 | 25.6 | | | | | |
| W695 + Oxi + Amidation | 0.3 | | 0.4 | 0.4 | 0.3 | | 0.4 | 0.4 | | | | |
| ~D297 + Amidation | | 72.9 | | 73.3 | | | | | | | | |

In the case of the AAVhu68 capsid protein, 4 residues (N57, N329, N452, N512) routinely display levels of deamidation >70% and it most cases >90% across various lots. Additional asparagine residues (N94, N253, N270, N304, N409, N477, and Q599) also display deamidation levels up to ˜20% across various lots. The deamidation levels were initially identified using a trypsin digest and verified with a chymotrypsin digestion.

Adult Rhesus macaques were ICM-administered AAVhu68.CB7.CI.eGFP.WPRE.rBG (3.00×10¹³ GC) and necropsied 28 days later to assess vector transduction. Transduction of AAVhu68 was observed in widespread areas of the brain. Thus, the AAVhu68 capsid provides the possibility of cross-correction in the CNS.

### REFERENCES

Abbott N. J. (2013). “Blood-brain barrier structure and function and the challenges for CNS drug delivery.” J Inherit Metab Dis. 36(3):437-49.
Absoud M., et al. (2011). “Paediatric UK demyelinating disease longitudinal study (PUDDLS).” BMC Pediatrics. 11(1):68.
Albers C. A. & Grieve A. J. (2007). “Test Review: Bayley, N. (2006). Bayley Scales of Infant and Toddler Development—Third Edition. San Antonio, TX: Harcourt Assessment.” J of Psychoed Assess. 25(2):180-190.
Alotaibi M., et al. (2014). “The efficacy of GMFM-88 and GMFM-66 to detect changes in gross motor function in children with cerebral palsy (CP): a literature review.” Disabil Rehabil. 36(8):617-27.
Arbour L. T., et al. (2000). “Variable onset of metachromatic leukodystrophy in a Vietnamese family.” Pediatr Neurol. 23(2):173-6.
Aubourg P. (2016). “Gene therapy for leukodystrophy: progress, challenges and opportunities.” Exp Opin Orph Drugs. 4(4):359-367.
Audentes (2018). Audentes announces continuing positive data from first dose cohort of ASPIRO, a Phase 1/2 clinical trial of AT 132 in patients with X-linked myotubular myopathy.
Bartus R. T., et al. (2014). “Parkinson's disease gene therapy: Success by design meets failure by efficacy.” Mol Ther. 22(3):487-497.
Batzios S. P. & Zafeiriou D. I. (2012). “Developing treatment options for metachromatic leukodystrophy.” Mol Genet Metab. 105(1):56-63.
Baumann N., et al. (2002). “Motor and psycho-cognitive clinical types in adult metachromatic leukodystrophy: genotype/phenotype relationships?” J Physiol Paris. 96(3-4):301-6.
Bell P., et al. (2015). “Motor neuron transduction after intracisternal delivery of AAV9 in a cynomolgus macaque.” Hum Gene Ther Methods. 26(2):43-4.
Bell P., et al. (2006). “Analysis of tumors arising in male B6C3F1 mice with and without AAV vector delivery to liver.” Mol Ther. 14(1):34-44.
Bell P., et al. (2005). “No evidence for tumorigenesis of AAV vectors in a large-scale study in mice.” Mol Ther. 12(2):299-306.
Bevan A. K., et al. (2011). “Systemic gene delivery in large species for targeting spinal cord, brain, and peripheral tissues for pediatric disorders.” Mol Ther. 19(11):1971-80.
Biffi A. (2017). “Hematopoietic stem cell gene therapy for storage disease: Current and new indications.” Mol Ther. 25(5):1155-1162.
Biffi A., et al. (2008). “Metachromatic leukodystrophy-mutation analysis provides further evidence of genotype-phenotype correlation.” Clin Genet. 74(4):349-57.
Biffi A., et al. (2004). “Correction of metachromatic leukodystrophy in the mouse model by transplantation of genetically modified hematopoietic stem cells.” J Clin Invest. 113(8):1118-29.
Biffi A., et al. (2013). “Lentiviral hematopoietic stem cell gene therapy benefits metachromatic leukodystrophy.” Science. 341(6148):1233158.
Bisgaard A. M., et al. (2009). “Chromosomal deletion unmasking a recessive disease: 22q13 deletion syndrome and metachromatic leukodystrophy.” Clin Genet. 75(2):175-9.
Boucher A. A., et al. (2015). “Long-term outcomes after allogeneic hematopoietic stem cell transplantation for metachromatic leukodystrophy: the largest single-institution cohort report.” Orphanet J Rare Dis. 10:94.
Bredius R. G., et al. (2007). “Early marrow transplantation in a pre-symptomatic neonate with late infantile metachromatic leukodystrophy does not halt disease progression.” Bone Marrow Transplant. 39(5):309-10.
Bryant L. M., et al. (2013). “Lessons learned from the clinical development and market authorization of Glybera.” Hum Gene Ther Clin Dev. 24(2):55-64.
Calcedo R., et al. (2009). “Worldwide epidemiology of neutralizing antibodies to adeno-associated viruses.” J Infect Dis. 199(3):381-90.
Cameron C. L., et al. (2004). “Multifocal slowing of nerve conduction in metachromatic leukodystrophy.” Muscle Nerve. 29(4):531-6.
Castle M. J., et al. (2014). “Long-distance axonal transport of AAV9 is driven by dynein and kinesin-2 and is trafficked in a highly motile Rab7-positive compartment.” Mol Ther. 22(3):554-566.
Cearley C. N., et al. (2008). “Expanded repertoire of AAV vector serotypes mediate unique patterns of transduction in mouse brain.” Mol Ther. 16(10):1710-8.
Cesani M., et al. (2009). “Characterization of new arylsulfatase A gene mutations reinforces genotype-phenotype correlation in metachromatic leukodystrophy.” Hum Mutat. 30(10):E936-45.
Cesani M., et al. (2016). “Mutation Update of ARSA and PSAP Genes Causing Metachromatic Leukodystrophy.” Hum Mutat. 37(1):16-27.
Chandler R. J., et al. (2015). “Vector design influences hepatic genotoxicity after adeno-associated virus gene therapy.” J Clin Invest. 125(2):870-80.
Chen X., et al. (2016). “Outcome of Early Juvenile Onset Metachromatic Leukodystrophy After Unrelated Cord Blood Transplantation: A Case Series and Review of the Literature.” J Child Neurol. 31(3):338-44.
Ciesielska A., et al. (2013). “Cerebral infusion of AAV9 vector-encoding non-self proteins can elicit cell-mediated immune responses.” Mol Ther. 21(1):158-66.
Clarke J. T., et al. (1989). “Marked clinical difference between two sibs affected with juvenile metachromatic leukodystrophy.” Am J Med Genet. 33(1):10-3.
Colle M. A., et al. (2010). “Efficient intracerebral delivery of AAV5 vector encoding human ARSA in non-human primate.” Hum Mol Genet. 19(1):147-58.
Consolaro A. & Ravelli A. (2016). “Chapter 5—Assessment Tools in Juvenile Idiopathic Arthritis.” Handbook of Systemic Autoimmune Diseases. R. Cimaz and T. Lehman: Elsevier. 11: 107-127.
Couto L., et al. (2004). “Direct exposure of mouse spermatozoa to very high concentrations of a serotype-2 adeno-associated virus gene therapy vector fails to lead to germ cell transduction.” Hum Gene Ther. 15(3):287-91.
Dali C. I., et al. (2015). “Sulfatide levels correlate with severity of neuropathy in metachromatic leukodystrophy.” Ann Clin Transl Neurol. 2(5):518-33.
Dali C. I., Hanson L. G., Barton N. W., Fogh J., Nair N., & Lund A. M. (2010). “Brain N-acetylaspartate levels correlate with motor function in metachromatic leukodystrophy.” Neurology. 75(21):1896-903.
de Hosson L. D., et al. (2011). “Adult metachromatic leukodystrophy treated by allo-SCT and a review of the literature.” Bone Marrow Transplant. 46(8):1071-6.
Dekaban A. S. (1978). “Changes in brain weights during the span of human life: relation of brain weights to body heights and body weights.” Ann Neurol. 4(4):345-56.
Deverman B. E., et al. (2016). “Cre-dependent selection yields AAV variants for widespread gene transfer to the adult brain.” Nat Biotechnol. 34(2):204-9.
Ding X. Q., et al. (2012). “Long-term neuroimaging follow-up on an asymptomatic juvenile metachromatic leukodystrophy patient after hematopoietic stem cell transplantation: evidence of myelin recovery and ongoing brain maturation.” Am J Med Genet A. 158a(1):257-60.
Donsante A., et al. (2001). “Observed incidence of tumorigenesis in long-term rodent studies of rAAV vectors.” Gene Ther. 8(17):1343-6.
Duyff R. F. & Weinstein H. C. (1996). “Late-presenting metachromatic leukodystrophy.” Lancet. 348(9038):1382-3.
Ellinwood N. M., et al. (2011). “Safe, efficient, and reproducible gene therapy of the brain in the dog models of Sanfilippo and Hurler syndromes.” Mol Ther. 19(2):251-9.
Ferla R., et al. (2017). “Non-clinical safety and efficacy of an AAV2/8 vector administered intravenously for treatment of Mucopolysaccharidosis Type VI.” Mol Ther Methods Clin Dev. 6:143-158.
Flanigan K. (2018). Voyager Therapeutics Corpoate Presentation. ASGCT Annual Meeting.
Foust K. D., et al. (2009). “Intravascular AAV9 preferentially targets neonatal neurons and adult astrocytes.” Nat Biotechnol. 27(1):59-65.
Frati G., et al. (2018). “Human iPSC-based models highlight defective glial and neuronal differentiation from neural progenitor cells in metachromatic leukodystrophy.” Cell Death Dis. 9(6):698.
Fumagalli F., et al. (2017). “Update on safety and efficacy of lentiviral hematopoietic stem cell gene therapy (HSC-GT) for metachromatic leukodystrophy (MLD).” Eur J of Paed Neurol. 21:e20.
Gallo S., et al. (2004). “Late onset MLD with normal nerve conduction associated with two novel missense mutations in the ASA gene.” J Neurol Neurosurg Psychiatry. 75(4):655-7.
Gao G., et al. (2004). “Clades of adeno-associated viruses are widely disseminated in human tissues.” J Virol. 78(12):6381-8.
Garavelli L., et al. (2009). “Massive hemobilia and papillomatosis of the gallbladder in metachromatic leukodystrophy: a life-threatening condition.” Neuropediatrics. 40(6):284-6.
Ghosh P., et al. (2003). “Mannose 6-phosphate receptors: new twists in the tale.” Nat Rev Mol Cell Biol. 4(3):202-12.
Gieselmann V. & Krageloh-Mann I. (2010). “Metachromatic leukodystrophy—an update.” Neuropediatrics. 41(1):1-6.
Gieselmann V., et al. (1998). “Metachromatic leukodystrophy: Molecular genetics and an animal model.” J Inherit Metab Dis. 21(5):564-74.
Gieselmann V., et al. (1989). “Arylsulfatase A pseudodeficiency: loss of a polyadenylylation signal and N-glycosylation site.” Proc Natl Acad Sci USA. 86(23):9436-40.
Gieselmann V. & von Figura K. (1990). “Advances in the molecular genetics of metachromatic leukodystrophy.” J Inherit Metab Dis. 13(4):560-71.
Gil-Farina I., et al. (2016). “Recombinant AAV integration is not associated with hepatic genotoxicity in nonhuman primates and patients.” Mol Ther. 24(6):1100-1105.
Giugliani R., et al. (2018). “Intrathecal delivery of recombinant human arylsulfatase A in children with late-infantile metachromatic leukodystrophy: A post hoc analysis of responders and non-responders.” Molec Genet Metab. 123(2):S54.
Godel T., et al. (2016). “Human dorsal-root-ganglion perfusion measured in-vivo by MRI.” Neuroimage. 141:81-87.
Gombash S. E., et al. (2017). “Systemic gene delivery transduces the enteric nervous system of guinea pigs and cynomolgus macaques.” Gene Ther. 24(10):640-648.
Gomez-Ospina N. (2017). “Arylsulfatase A Deficiency.” GeneReviews. M. P. Adam, H. H. Ardinger, R. A. Pagon et al. (eds). Seattle, WA: University of Washington, Seattle.
Graham F. L., et al. (1977). “Characteristics of a human cell line transformed by DNA from human adenovirus type 5.” J Gen Virol. 36(1):59-74.
Gray S. J., et al. (2011). “Preclinical differences of intravascular AAV9 delivery to neurons and glia: a comparative study of adult mice and nonhuman primates.” Mol Ther. 19(6):1058-69.
Gray S. J., et al. (2013). “Global CNS gene delivery and evasion of anti-AAV-neutralizing antibodies by intrathecal AAV administration in non-human primates.” Gene Ther. 20(4):450-9.
Greig J. A., et al. (2017). “Non-clinical study examining AAV8.TBG.hLDLR vector-associated toxicity in chow-fed wild-type and LDLR(+/−) Rhesus macaques.” Hum Gene Ther Clin Dev. 28(1):39-50.
Groeschel S., et al. (2011). “Metachromatic leukodystrophy: natural course of cerebral MRI changes in relation to clinical course.” J Inherit Metab Dis. 34(5):1095-102.
Gu Z., et al. (2012). “Absence of PTHrP nuclear localization and carboxyl terminus sequences leads to abnormal brain development and function.” PLoS One. 7(7):e41542.
Gurda B. L., et al. (2016). “Evaluation of AAV-mediated gene therapy for central nervous system disease in canine Mucopolysaccharidosis VII.” Mol Ther. 24(2):206-216.
Guyenet S. J., et al. (2010). “A simple composite phenotype scoring system for evaluating mouse models of cerebellar ataxia.” J Vis Exp(39).
Hageman A. T., et al. (1995). “Clinical symptoms of adult metachromatic leukodystrophy and arylsulfatase A pseudodeficiency.” Arch Neurol. 52(4):408-13.
Haurigot V., et al. (2013). “Whole body correction of mucopolysaccharidosis IIIA by intracerebrospinal fluid gene therapy.” J Clin Invest.
Heim P., et al. (1997). “Leukodystrophy incidence in Germany.” Am J Med Genet. 71(4):475-8.
Herndon J. G., et al. (1998). “Brain weight does not decrease with age in adult rhesus monkeys.” Neurobiol Aging. 19(3):267-72.
Hess B., et al. (1996). “Phenotype of arylsulfatase A-deficient mice: relationship to human metachromatic leukodystrophy.” Proc Natl Acad Sci USA. 93(25):14821-6.
Hinderer C., et al. (2018). “Evaluation of intrathecal routes of administration for adeno-associated viral vectors in large animals.” Hum Gene Ther. 29(1):15-24.
Hinderer C., et al. (2014). “Widespread gene transfer in the central nervous system of cynomolgus macaques following delivery of AAV9 into the cisterna magna.” Mol Ther Methods Clin Dev. 1:14051.
Hinderer C., et al. (2016). “Delivery of an adeno-associated virus vector into cerebrospinal fluid attenuates central nervous system disease in Mucopolysaccharidosis Type II mice.” Hum Gene Ther. 27(11):906-915.
Hordeaux J., et al. (2018). “Toxicology study of intra-cisterna magna adeno-associated virus 9 expressing human alpha-L-iduronidase in Rhesus macaques.” Mol Ther Methods Clin Dev. 10:79-88.
Hordeaux J., et al. (2019). “The GPI-Linked Protein LY6A Drives AAV-PHP.B Transport across the Blood-Brain Barrier.” Molec Ther.
Hyde T. M., et al. (1992). “Psychiatric disturbances in metachromatic leukodystrophy. Insights into the neurobiology of psychosis.” Arch Neurol. 49(4):401-6.
Iannaccone S. T., et al. (2009). “The PedsQL in pediatric patients with Spinal Muscular Atrophy: feasibility, reliability, and validity of the Pediatric Quality of Life Inventory Generic Core Scales and Neuromuscular Module.” Neuromuscular disorders: NMD. 19(12):805-812.
Jabbehdari S., et al. (2015). “The clinical features and diagnosis of Metachromatic leukodystrophy: A case series of Iranian Pediatric Patients.” Iran J Child Neurol. 9(3):57-61.
Jakob M., et al. (2005). “No evidence for germ-line transmission following prenatal and early postnatal AAV-mediated gene delivery.” J Gene Med. 7(5):630-7. Janson C., et al. (2002). “Clinical protocol. Gene therapy of Canavan disease: AAV-2 vector for neurosurgical delivery of aspartoacylase gene (ASPA) to the human brain.” Hum Gene Ther. 13(11):1391-412.
Kapaun P., et al. (1999). “Slow progression of juvenile metachromatic leukodystrophy 6 years after bone marrow transplantation.” J Child Neurol. 14(4):222-8.
Kaplitt M. G., et al. (2007). “Safety and tolerability of gene therapy with an adeno-associated virus (AAV) borne GAD gene for Parkinson's disease: an open label, phase I trial.” Lancet. 369(9579):2097-105.
Kay M. A., et al. (2000). “Evidence for gene transfer and expression of factor IX in haemophilia B patients treated with an AAV vector.” Nat Genet. 24(3):257-61.
Kehrer C., et al. (2011a). “The natural course of gross motor deterioration in metachromatic leukodystrophy.” Dev Med Child Neurol. 53(9):850-855.
Kehrer C., et al. (2011b). “Development and reliability of a classification system for gross motor function in children with metachromatic leucodystrophy.” Dev Med Child Neurol. 53(2):156-60.
Kehrer C., et al. (2014). “Language and cognition in children with metachromatic leukodystrophy: onset and natural course in a nationwide cohort.” Orphanet J Rare Dis. 9:18.
Kim J., et al. (2017). “Gallbladder abnormalities in children with metachromatic leukodystrophy.” J Surg Res. 208:187-191.
Krageloh-Mann I., et al. (2013). “Juvenile metachromatic leukodystrophy 10 years post transplant compared with a non-transplanted cohort.” Bone Marrow Transplant. 48(3):369-75.
Krivit W., et al. (1990). “Treatment of late infantile metachromatic leukodystrophy by bone marrow transplantation.” N Engl J Med. 322(1):28-32.
Kruse B., et al. (1993). “Alterations of brain metabolites in metachromatic leukodystrophy as detected by localized proton magnetic resonance spectroscopy in vivo.” J Neurol. 241(2):68-74.
Kumperscak H. G., et al. (2005). “Adult metachromatic leukodystrophy: disorganized schizophrenia-like symptoms and postpartum depression in 2 sisters.” J Psychiatry Neurosci. 30(1):33-6.
Largo R. H., et al. (1985). “Early development of locomotion: Significance of prematurity, cerebral palsy and sex.” Dev Med Child Neurol. 27(2):183-91.
Lawlor P. A., et al. (2009). “Efficient gene delivery and selective transduction of glial cells in the mammalian brain by AAV serotypes isolated from nonhuman primates.” Mol Ther. 17(10):1692-702.
Li C., et al. (2013). “Adeno-associated virus capsid antigen presentation is dependent on endosomal escape.” J Clin Invest. 123(3):1390-401.
Li H., et al. (2011). “Assessing the potential for AAV vector genotoxicity in a murine model.” Blood. 117(12):3311-9.
Lock M., et al. (2014). “Absolute determination of single-stranded and self-complementary adeno-associated viral vector genome titers by droplet digital PCR.” Hum Gene Ther Methods. 25(2):115-25.
Lugowska A., et al. (2011). “Population carrier rates of pathogenic ARSA gene mutations: is metachromatic leukodystrophy underdiagnosed?” PLoS One. 6(6):e20218.
Lundkvist Josenby A., et al. (2009). “Longitudinal construct validity of the GMFM-88 total score and goal total score and the GMFM-66 score in a 5-year follow-up study.” Phys Ther. 89(4):342-50.
Mahmood A., et al. (2010). “Metachromatic leukodystrophy: a case of triplets with the late infantile variant and a systematic review of the literature.” J Child Neurol. 25(5):572-80.
Mandel R. J. & Burger C. (2004). “Clinical trials in neurological disorders using AAV vectors: promises and challenges.” Curr Opin Mol Ther. 6(5):482-90.
Marcao A. M., et al. (2005). “Adult onset metachromatic leukodystrophy without electroclinical peripheral nervous system involvement: a new mutation in the ARSA gene.” Arch Neurol. 62(2):309-13.
Martin A., et al. (2012). “Toward a better understanding of brain lesions during metachromatic leukodystrophy evolution.” AJNR Am J Neuroradiol. 33(9):1731-9.
Martin H. R., et al. (2013). “Neurodevelopmental outcomes of umbilical cord blood transplantation in metachromatic leukodystrophy.” Biol Blood Marrow Transplant. 19(4):616-24.
Matthes F., et al. (2012). “Efficacy of enzyme replacement therapy in an aggravated mouse model of metachromatic leukodystrophy declines with age.” Hum Mol Genet. 21(11):2599-609.
Matzner U., et al. (2005). “Enzyme replacement improves nervous system pathology and function in a mouse model for metachromatic leukodystrophy.” Hum Mol Genet. 14(9):1139-52.
Matzner U., et al. (2009). “Enzyme replacement improves ataxic gait and central nervous system histopathology in a mouse model of metachromatic leukodystrophy.” Mol Ther. 17(4):600-6.
McCarty D. M., et al. (2004). “Integration of adeno-associated virus (AAV) and recombinant AAV vectors.” Annu Rev Genet. 38:819-45.
McFadden K. & Ranganathan S. (2015). “Pathology of the gallbladder in a child with metachromatic leukodystrophy.” Pediatr Dev Pathol. 18(3):228-30.
Melnick J. L., et al. (1965). “Association of 20 milli-micron particles with adenoviruses.” J Bacteriol. 90(1):271-4.
Mendell J. R., et al. (2017). “Single-dose gene-replacement therapy for spinal muscular atrophy.” N Engl J Med. 377(18):1713-1722.
Miller N. (2012). “Glybera and the future of gene therapy in the European Union.” Nat Rev Drug Discov. 11(5):419.
Mittermeyer G., et al. (2012). “Long-term evaluation of a Phase 1 study of AADC gene therapy for Parkinson's disease.” Hum Gene Ther. 23(4):377-81.
Nathwani A. C., et al. (2011). “Adenovirus-associated virus vector-mediated gene transfer in hemophilia B.” N Engl J Med. 365(25):2357-65.
Nault J. C., et al. (2016). “Wild-type AAV Insertions in Hepatocellular Carcinoma Do Not Inform Debate Over Genotoxicity Risk of Vectorized AAV.” Mol Ther. 24(4):660-1.
Navarro C., et al. (1996). “Late juvenile metachromatic leukodystrophy treated with bone marrow transplantation; a 4-year follow-up study.” Neurology. 46(1):254-6.
Palisano R. J., et al. (2006). “Stability of the gross motor function classification system.” Dev Med Child Neurol. 48(6):424-8.
Passini M. A., et al. (2006). “Intracranial delivery of CLN2 reduces brain pathology in a mouse model of classical late infantile neuronal ceroid lipofuscinosis.” J Neurosci. 26(5):1334-42.
Patil S. A. & Maegawa G. H. (2013). “Developing therapeutic approaches for metachromatic leukodystrophy.” Drug Des Devel Ther. 7:729-45.
Penaud-Budloo M., et al. (2008). “Adeno-associated virus vector genomes persist as episomal chromatin in primate muscle.” J Virol. 82(16):7875-85.
Peng L. & Suzuki K. (1987). “Ultrastructural study of neurons in metachromatic leukodystrophy.” Clin Neuropathol. 6(5):224-30.
Perusi C., et al. (1999). “Mutations associated with very late-onset metachromatic leukodystrophy.” Clin Genet. 55(2):130.
Piguet F., et al. (2012). “Correction of brain oligodendrocytes by AAVrh.10 intracerebral gene therapy in metachromatic leukodystrophy mice.” Hum Gene Ther. 23(8):903-14.
Pilz H., et al. (1977). “Adult metachromatic leukodystrophy. I. Clinical manifestation in a female aged 44 years, previously diagnosed in the preclinical state.” Eur Neurol. 15(6):301-7.
Pinto R., et al. (2004). “Prevalence of lysosomal storage diseases in Portugal.” Eur J Hum Genet. 12(2):87-92.
Polten A., et al. (1991). “Molecular basis of different forms of metachromatic leukodystrophy.” N Engl J Med. 324(1):18-22.
Pomerantz S. R., et al. (2005). Suboccipital puncture of the cisterna magna under CT-guidance with intravenous enhancement in order to circumvent anomalous course of posterior inferior cerebellar artery (PICA) American Society of Spine Radiology Symposium.
Poorthuis B. J., et al. (1999). “The frequency of lysosomal storage diseases in The Netherlands.” Hum Genet. 105(1-2):151-6.
Rafi M. A., et al. (2012). “Extended normal life after AAVrh10-mediated gene therapy in the mouse model of Krabbe disease.” Mol Ther. 20(11):2031-42.
Ramakrishnan H., et al. (2007). “Increasing sulfatide synthesis in myelin-forming cells of arylsulfatase A-deficient mice causes demyelination and neurological symptoms reminiscent of human metachromatic leukodystrophy.” J Neurosci. 27(35):9482-90.
Rangarajan S., et al. (2017). “AAV5-Factor VIII gene transfer in severe hemophilia A.” N Engl J Med. 377(26):2519-2530.
Rauschka H., et al. (2006). “Late-onset metachromatic leukodystrophy: genotype strongly influences phenotype.” Neurology. 67(5):859-63.
REGENXBIO (2019). REGENXBIO reports fourth quarter and full-year 2018 financial results and recent operational highlights.
Regis S., et al. (2002). “Contribution of arylsulfatase A mutations located on the same allele to enzyme activity reduction and metachromatic leukodystrophy severity.” Hum Genet. 110(4):351-5.
Rodriguez-Waitkus P. M., Byrd R., & Hicks J. (2011). “Metachromatic leukodystrophy and its effects on the gallbladder: a case report.” Ultrastruct Pathol. 35(6):271-6.
Rosas L. E., et al. (2012). “Patterns of scAAV vector insertion associated with oncogenic events in a mouse model for genotoxicity.” Mol Ther. 20(11):2098-2110.
Rosenberg J. B., et al. (2016). “Gene therapy for metachromatic leukodystrophy.” J Neurosci Res. 94(11):1169-79.
Russell D. J., et al. (1989). “The gross motor function measure: a means to evaluate the effects of physical therapy.” Dev Med Child Neurol. 31(3):341-52.
Samaranch L., et al. (2013). “Strong cortical and spinal cord transduction after AAV7 and AAV9 delivery into the cerebrospinal fluid of nonhuman primates.” Hum Gene Ther. 24(5):526-32.
Sands M. S. (2014). “A Hitchhiker's guide to the blood-brain barrier: in trans delivery of a therapeutic enzyme.” Mol Ther. 22(3):483-484.
Saunders H. C. & Riordan T. J. (1929). “Cisternal or suboccipital puncture: A report of 2019 punctures.” New England Journal of Medicine. 201:166-168.
Schuster D. J., et al. (2014). “Biodistribution of adeno-associated virus serotype 9 (AAV9) vector after intrathecal and intravenous delivery in mouse.” Front Neuroanat. 8:42.
Sessa M., et al. (2016). “Lentiviral haemopoietic stem-cell gene therapy in early-onset metachromatic leukodystrophy: an ad-hoc analysis of a non-randomised, open-label, phase 1/2 trial.” Lancet. 388(10043):476-87.
Sevin C., et al. (2007). “Enzyme, cell and gene-based therapies for metachromatic leukodystrophy.” J Inherit Metab Dis. 30(2):175-83.
Sevin C., et al. (2018). “Intracerebral gene therapy in children with metachromatic leukodystrophy: Results of a phase I/II trial.” Molec Genet Metab. 123(2): S129.
Skorupa A. F., et al. (1999). “Sustained production of beta-glucuronidase from localized sites after AAV vector gene transfer results in widespread distribution of enzyme and reversal of lysosomal storage lesions in a large volume of brain in mucopolysaccharidosis VII mice.” Exp Neurol. 160(1):17-27.
Smith N. J., et al. (2010). “Haematopoietic stem cell transplantation does not retard disease progression in the psycho-cognitive variant of late-onset metachromatic leukodystrophy.” J Inherit Metab Dis. 33 Suppl 3:S471-5.
Solders M., et al. (2014). “Hematopoietic SCT: a useful treatment for late metachromatic leukodystrophy.” Bone Marrow Transplant. 49(8):1046-51.
Spark (2018). European Commission approves Spark Therapeutics' LUXTURNA® (voretigene neparvovec), a one-time gene therapy for inherited retinal disease caused by confirmed biallelic RPE65 mutations.
Strolin M., et al. (2017). “Demyelination load as predictor for disease progression in juvenile metachromatic leukodystrophy.” Ann Clin Transl Neurol. 4(6):403-410.
Stroobants S., et al. (2011). “Intracerebroventricular enzyme infusion corrects central nervous system pathology and dysfunction in a mouse model of metachromatic leukodystrophy.” Hum Mol Genet. 20(14):2760-9.
Taber C. & Thomas C. (1997). Taber's Cyclopedic Medical Dictionary. Philadelphia, F. A. Davis.
Tardieu M., et al. (2014). “Intracerebral administration of adeno-associated viral vector serotype rh.10 carrying human SGSH and SUMF1 cDNAs in children with mucopolysaccharidosis type IIIA disease: results of a phase I/II trial.” Hum Gene Ther. 25(6):506-16.
Toda K., et al. (1990). “Lysosulfatide (sulfogalactosylsphingosine) accumulation in tissues from patients with metachromatic leukodystrophy.” J Neurochem. 55(5):1585-91.
Tylki-Szymanska A., et al. (1996). “Late juvenile metachromatic leukodystrophy (MLD) in three patients with a similar clinical course and identical mutation on one allele.” Clin Genet. 50(5):287-92.
van Egmond M. E., et al. (2013). “Improvement of white matter changes on neuroimaging modalities after stem cell transplant in metachromatic leukodystrophy.” JAMA Neurol. 70(6):779-82.
van Rappard D. F., et al. (2015). “Metachromatic leukodystrophy: Disease spectrum and approaches for treatment.” Best Pract Res Clin Endocrinol Metab. 29(2):261-73.
van Rappard D. F., et al. (2016). “Gallbladder and the risk of polyps and carcinoma in metachromatic leukodystrophy.” Neurology. 87(1):103-11.
van Rappard D. F., et al. (2018). “Slowly Progressive Psychiatric Symptoms: Think Metachromatic Leukodystrophy.” J Am Acad Child Adolesc Psychiatry. 57(2):74-76.
Vari J. W., et al. (2011). “The PedsQL Infant Scales: feasibility, internal consistency reliability, and validity in healthy and ill infants.” Qual Life Res. 20(1):45-55.
Virgens M. Y., et al. (2015). “Genotypic characterization of Brazilian patients with infantile and juvenile forms of metachromatic leukodystrophy.” Gene. 568(1):69-75.
Vite C. H., et al. (2005). “Effective gene therapy for an inherited CNS disease in a large animal model.” Ann Neurol. 57(3):355-64.
Von Figura K., et al. (2001). “Metachromatic leukodystrophy.” The Metabolic and Molecular Bases of Inherited Disease. S. C. R., B. A. L., S. W. S. and V. D. (eds). New York, NY: McGraw-Hill.
Wang R. Y., et al. (2011). “Lysosomal storage diseases: diagnostic confirmation and management of presymptomatic individuals.” Genet Med. 13(5):457-84.
WHO (2006). Reliability of motor development data in the WHO Multicentre Growth Reference Study. Acta Paediatr Suppl. 450: 47-55.
Wijnhoven T. M., et al. (2004). “Assessment of gross motor development in the WHO Multicentre Growth Reference Study.” Food Nutr Bull. 25 (1 Suppl): 537-45.
Worgall S., et al. (2008). “Treatment of late infantile neuronal ceroid lipofuscinosis by CNS administration of a serotype 2 adeno-associated virus expressing CLN2 cDNA.” Hum Gene Ther. 19(5):463-74.
Zafeiriou D. I., et al. (1999). “Neurophysiology and MRI in late-infantile metachromatic leukodystrophy.” Pediatr Neurol. 21(5):843-6.
Zerah M., et al. (2015). “Intracerebral Gene Therapy Using AAVrh.10-hARSA Recombinant Vector to Treat Patients with Early-Onset Forms of Metachromatic Leukodystrophy: Preclinical Feasibility and Safety Assessments in Nonhuman Primates.” Hum Gene Ther Clin Dev. 26(2):113-24.

All documents cited in this specification are incorporated herein by reference, as are priority documents, U.S. Provisional Patent Application No. 63/341,636, filed May 13, 2022; U.S. Provisional Patent Application No. 63/331,367, filed Apr. 15, 2022, and U.S. Provisional Patent Application No. 63/297,958, filed Jan. 10, 2022. While the invention has been described with reference to particular embodiments, it will be appreciated that modifications can be made without departing from the spirit of the invention. Such modifications are intended to fall within the scope of the appended claims.

1. A pharmaceutical composition for use in treating metachromatic leukodystrophy or a disease associated with an arylsulfatase A (ARSA) gene mutation, said pharmaceutical composition comprising
recombinant adeno-associated virus (rAAV) comprising an AAVhu68 capsid; and a vector genome), comprising: a 5′ AAV inverted terminal repeats (ITR), a CB7 promoter comprising a CMV IE enhancer and a CB promoter, and a nucleic acid sequence encoding a functional human Arylsulfatase A (hARSA) operably linked to regulatory sequences comprising the CB7 promoter which direct the hARSA expression, a polyA signal, and a 3′ AAV ITR wherein the hARSA coding sequence comprises a sequence of nucleotide (nt) 1 to nt 1521 of SEQ ID NO: 1, or a sequence at least 95% identical thereto which encodes a functional hARSA; and
at least one aqueous buffer, at least one carrier, at least one excipient and/or a least one preservative,
said pharmaceutical composition being deliverable in a single therapeutic dose via intrathecal administration.

2. The pharmaceutical composition of claim 1, wherein the regulatory elements further comprise one or more of a Kozak sequence, an intron, a further enhancer, and/or a TATA signal.

3. The pharmaceutical composition of claim 1, wherein the hARSA coding sequence is SEQ ID NO: 1 or SEQ ID NO: 3.

4. The pharmaceutical composition of claim 1, wherein the vector genome comprises a 5′ AAV ITR, an expression cassette having the sequence of SEQ ID NO: 28, and a 3′ AAV ITR.

5. The pharmaceutical composition of claim 1, wherein the AAV 5′ ITR has the sequence of SEQ ID NO: 25 and/or the AAV 3′ ITR has the sequence of SEQ ID NO: 26.

6. The pharmaceutical composition of claim 1, wherein the vector genome comprises nt 1 to nt 3883 of SEQ ID NO: 5 (SEQ ID NO: 27).

7. The pharmaceutical composition of claim 1, wherein the AAVhu68 capsid is produced from a sequence encoding the amino acid sequence of SEQ ID NO: 7.

8. The pharmaceutical composition of claim 1, wherein the composition comprises an artificial cerebrospinal fluid comprising buffered saline and one or more of sodium, calcium, magnesium, potassium, or mixtures thereof, and a surfactant, wherein the surfactant is optionally present at 0.0005% to about 0.001% of the pharmaceutical composition, and/or wherein the composition is at a pH in the range of 6.5 to 8.5.

9. The pharmaceutical composition of claim 1, wherein the composition is suitable for an intra-cisterna magna injection (ICM) or intracerebroventricular administration.

10. The pharmaceutical composition of claim 1, wherein the single dose comprises 3×10¹⁰ genome copies (GC)/gram of brain mass to 3.5×10¹¹ GC/gram of brain mass.

11. The pharmaceutical composition of claim 10, wherein the dose is:
(a) about 3.3×10¹⁰ genome copies (GC)/gram of brain mass;
(b) about 1.1×10¹¹ genome copies (GC)/gram of brain mass; or
(c) about 3.3×10¹¹ genome copies (GC)/gram of brain mass.

12-14. (canceled)

15. A method of treating Metachromatic Leukodystrophy or a disease associated with a Arylsulfatase A (ARSA) gene mutation in a subject in need thereof, the method comprising administering a single dose of a recombinant AAV to the subject by ICM injection, wherein the recombinant AAV comprises an AAVhu68 capsid and a vector genome packaged therein, said vector genome comprising AAV ITRs, an hARSA coding sequence comprising SEQ ID NO: 1, or a sequence at least 95% identical thereto that encodes a functional hARSA, and regulatory sequences which direct expression of the functional hARSA in a target cell, wherein the single dose is
(i) about 3.3×10¹⁰ genome copies (GC)/gram of brain mass;
(ii) about 1.1×10¹¹ GC/gram of brain mass; or
(iii) about 3.3×10¹¹ GC/gram of brain mass.

16. The method of claim 15, wherein the rAAV comprises an AAVhu68 capsid; and a vector genome comprising: a 5′ AAV inverted terminal repeats (ITR), a CB7 promoter comprising a CMV IE enhancer and a CB promoter, and a nucleic acid sequence encoding a functional human Arylsulfatase A (hARSA) operably linked to regulatory sequences comprising the CB7 promoter which direct the hARSA expression, a polyA signal, and a 3′ AAV ITR wherein the hARSA coding sequence comprises a sequence of nucleotide (nt) 1 to nt 1521 of SEQ ID NO: 1, or a sequence at least 95% to identical thereto which encodes a functional hARSA.
